SEQUENCE LISTING (83 sequences)

SEQ ID NO: 1 - DNA, 474 bp, Oryza sativa
  misc_feature (394)...(474): segment of the T-DNA insert in line 1B-115-22
aagcttaatatattaggagtagtaagctagcgtgtgaggaagattcataaccatatatcatcttaattagcctttgtgatttagcttaatcacatggctaaggcacaaccacccatccacttaactcttcattactacgctagctacacgaggagagtagctagctagtacaggccccggcaagcataaatagcagcgtcccctgcttcctttcttcatcgtcctcagctcatcatctgcatgcagttcactgcacaccacacagcttagcttgctcagcttcactgatcttcttagctgcagctacttcactttgcatagtttgatcgaactaaataactcaccaagttagctgtaatggccaagctgatcctcgccaccttcgccgtcgtgttaccaggtaccaggtgagttccattcttactaccacggtgctattttttttgctatgtggctaattacatgactaacttg

SEQ ID NO: 2 - DNA, 194 bp, Oryza sativa
  misc_feature (104)...(194): segment of the T-DNA insert in line 1B-164-43
atcgatgtatccatactccgagatagtaggctcggtccttaacttgtcgcgccgccgtgtgtaattttggtttgattttctccgcagccatgggagacctacacggatccgaggtaccaggtaccaggtgagttccattcttactaccacggtgctattttttttgctatgtggctaattacatgactaacttg

SEQ ID NO: 3 - DNA, 325 bp, Oryza sativa
  misc_feature (255)...(325): segment of the T-DNA insert in line 1B-192-40
ctccataccactagaacttggtagcttgataaatcttgcttctttgaacatttcccacaacaaactcacaggcaggatcccatctacacttggttcttgcgtccggttggaatcgcttcgtgtaggaggcaacttcctagagggaagtattccacaatcattagcaaatctcaggggcaccaaagtgctggatttctcccagaacaatttatctggtgcaataccggatttctttgggactttcacctcattacacaggtgagttccattcttactaccacggtgctattttttttgctatgtggctaattacatgactaacttg

SEQ ID NO: 4 - DNA, 650 bp, Oryza sativa
  misc_feature (560)...(650): segment of the T-DNA insert in line 1B-207-27
ccattccttctctccattccctatatcttcagcataataacctatccgggatcataccgacttcccttacttccactttaacatttctagatctgtcatacaacacttttgatggagaaatcccattgagagtgcaaaatctcactcaacttactgcattgcttctccagaataactctctttctggacccatccctgacctccaactcccaaaattgaggcatttgaatttgagcaacaataacctcagtgggccaataccaccttccttgcaaagattcccagccaattccttcttggggaatgcttttctatgtgggtttcctttacaaccttgccctgggactgcaccttcaccttctccttctccaacatcaccatcacccggcaaggccaagaagggcttctggaaaaggatcagaactggtgttataattgcacttgctgccgcaggaggggtattgttgctaatcttgattgttttactcttgatatgtattttcaagagaaagaaaagcacagaacctactacagcttcttcgtcaaaaggaaaaactgttggatccgaggtaccaggtaccaggtgagttccattcttactaccacggtgctattttttttgctatgtggctaattacatgactaacttg

SEQ ID NO: 5 - DNA, 6721 bp, Oryza sativa
  misc_feature (1311)...(1400): segment of the T-DNA insert in line 1B-138-07
cgatgctccgcgccgccctcctccgctcaggtaggtcgccgccgcctcctcctcctcctcctcccttctccgcggcgactcctctgacccttctcggccccgcctccccacgcaggctccggcctccgccggcctcccatggccgcgccgctctccaccgccgccgccgcctcgtggctctccgacagcgcctcgtctccggtaagcacccgcgcgcggctcatccattcccaatttcccatccgtgtgaagtgtgattgacctctctctctctctcactctcccggcgcagcctagggtgcgcctcctcatcggcggggagttcgtcgagtcgcgggccgacgagcacgtcgacgtcaccaatccggtaaagcccgccgtgagcctccccacgatcgatcgatttgggaggtgtggtgtgggggcatgtgacagtgattctctgggtgtgcgcaggcgacgcaggaggtggtgtcgcggatcccgctcaccaccgccgacgagttcagggccgccgtggacgccgccaggaccgccttccccgggtggcggaacacgccggtgaccacgcggcagcgcatcatgctcaagtaccaggagctcatccgggccaacatggtaattagttgatagatggattgaccttccttccatcgttgggggattggagagctagaagcttcactcgatgatgtggttggatcatgttatagtgattctgttctttttttgtgatgtcaggataaactggcagagaacattacaaccgaacaggggaagacactgaaggatgcttggggcgatgtattccgtgggctaggttggttttcagacccttatagtacctggtttcactgcttcattagatagtgataatctcagcatttcaaatctatgagtagtcgttatgttctttcaatctactaatcttaatctatggcttgattcaaatgagatgatatttgtttgcaaggaatatttcttatactggatgtttgacacgctggttgcaagatctgattaactcctggttttgtagaggtggtggaacatgcttgtggaatggggacactgcagatgggtgaatatgtatcaaatgtttctaacgggattgacacctttagcattagggagccacttggtgtatgtgctggaatatgtccattcaattttccagctatgatccccctatgggtacgcacaacttcacttcttaatatcaaaatttgttattattacgactcttagcccaattatcaatataccatgcaatacatcttactttagtcaacacgccatgatttgatcgtggatccgaggtaccaggtaccaggtgagttccattcttactaccacggtgctattttttttgctatgtggctaattacatgactaacttgttcatttgtttcttctttccgtttggctgcagatgttcccaatagcggtcacttgtggcaatacttttgttctaaagccatcagaaaaagatccaggtgctttgctttatcatcatttttttacccagtttattcactacacccgctgttgttgcattgacgacaatttaaagccagataaaaatatatggttaaatgcttatctgatattacagagcgggttaaccaagctgtacttttctccacaacatataagatcattgattctatgtattcactatatacatatccatttggtactataactttgggggtgtgtttttactataacatatggtgaagttaaacatttggtgcacagttatctacccaaattggacatttccactacctactgctataaaaaaaccagcagtaagttttaactctttggaggtagtagcaagacaaccttttacaattccattgagatattaataaaaacagattacatgttgctttacaatctttgtgtacatggcaaggacttaagatggagtgaaaatatgacatgtttttgtgaactggcactagtagtatttctaaggcatgaaaaagatatattataaagtagcaattgttgttcatgtccttgaagaggctgtaatcttagtctagtagaacaaaagaaggcacatgacatgttcctgttagtaatgtgatggcatactttgtaattattggttactacttcaggggctgctatgatgcttgcggagctagcaatggaggctggtttaccaaagggtgtgttgaacattgttcatggtacccatgtacggtaaccttggttactcttcgtccttcctttttttttttccattttttgagctcgatactagattaaataatttatttgttgtgcctgttggtgccaagtgttgctacagcacaaatagatattcgatgattgatattttctggctaacatgacaataattttactctttcttggaacactggcaaaaaaaaaaagttgccttgaaacttcaccttaccatgcttagctctgtagggattgtttggtcttcatggttatatgatgctatttttactgctaaacattcatgaatctgttcatctttagccgccacacttgctaagctgctttctgattctaccttactatttcaggatgttgtgaacaacatttgtgatgacgaggacatcaaggcagtttcctttgttggttccaatatagtaagcggatccaaatgatctgtctatttctcgatttgtggtaccatttttgcatcaatcatctgtctattttgctctgcataatactttgcaaatctggtattcatcctattctgtttcagtctttaggtcattaacttggcatctaggccattgcattatcacacacttctacatgtttgagatcttttcaatcttgtattcttatgttgtcataatggttcaaattctgattatgatctaacttataaaaattataaatttaccagttaccatgatggtgataaactgataataaacatgtatacacagcatgaggatgttttcatgatgttaggacattcggttgtaaaatttacttgtaaacaattatgttagatccgtatagcacatgtcacattacactcatgatacctgaatcattctttattttatttttgcccaaagctagtcatttgtttcagcagatgatataattatgatctctctctcactctcaggctggtatgcatatatattctagagcatcggcaaaggggaagcgtgttcaggttagtttgcacctgttcctgacatgcagaacctttccttcgaaaacttctgcctggtgtgctgtaagcagcatgttacagaatgcctttttgtttattttcagtctaatatgggtgcaaagaatcatgcaattatccttcctgatgctgaccgagatgccacactgaatgcccttattgctgctggttttggtgctgctgggcaaaggtgtatggcattgagcactgctgtttttgttggaggttcagagccatggtatatacttctgtcctgtttgtttcttcaggataagacctggcattaggtaatgaagtataaaaataaccatttgtcttgtagggaggatgagctagtcaaacgtgcaagcagccttgtggttaattcaggaatggctagtgatgcagaccttggtccagtgatcagcaaacaggtaaaccatctgcttttttaccaacgaaaaggttgttgacctgattttaagtaacctaaatatgaagtggaatcctacttgtacttccctgttacttggaacactgaactggatttgtgctcccccccccccccccaaaaaaaaaaaaacttgaatggtcctctaatcgtgtatacacattgaaatcatctagttcagatattattttggtacctaaacttatcctaatgtggacttctcttggtacttatgtaggataggttatgaacatttatccttttatgccatgatttctatgttgactcaacacgtgaacttaacatccccagatttcaagagtgttaacattacatagccaagtaatgacttgtaaagtttctgtattttttgatctcgtctgttcatgaatgaacctgttgtgttgagattgattacagtttagcgatagtgatagtgcatgaaattgcatttgaaattcaaaaaaggctacttctgtttttttttggttctgatgagactgttggtatgtggtatatattttctggaaatatatattaccttttggggcttggctgcgagtattattagcttcagattcataatgattgtggctactacattggttacgctcacagtttttatgctattttcttgtaaatataaatttataacattttctaccttgtcatttctgttttcctttttctcaatatatcctgtcttaacagtttactatcaacaggctaaggaacgtatctgcaaattaatacaaagtggtgctgataatggtgctcgtgtgctgcttgatggaagagatattgtggtaattttagttcagttgtttccttgcctgcagttctcccctggttcttatgcagcaatgcataatagtgctaaactgtgctaaattttgcaggttcctaacttcgagaatggtaattttgttggtccaacactccttgctgatgttaaaagtgaaatggaatgttacaaggtattattctgaaatttgtcattttaaactctgcattaggactgatgtttcttgtttgaagagccatgaaaggaaggaaggttgcattattagcctcctgcaaagctatttgttggattagtgctgtctgtgttctaaaaattaaaacatggaatgcatttaaggatatattattttctaattgaagtgctaactgtggacctatttgatactccctccgtcccaaaatataagcatttttagactctgatacgatctttgatatgctactttgaccaacaatatctgtgaaagtaagatgttttaaataaaaggagttacatattatgatagattgtttaatgataaatctagtaatatcaattttacatgattgattgttttttttttgctattaatagtcaaagttaaaaatgtttgacttgttactatgctaaaaatgcttatattttgggacagagagagtattaatttatttgggactatgatgtatctttcttttcaggagtgtatttgacctatgcttttttaaaagaattatatgattttgtctggagaccaatgtaattaacaatccaaaacaaggaaatggcaaagcagataactcgcctattgcatggtatcaaatctcagtattgctcactttcccatttataacttgcaagacactgttctcctcaagtagacatccttgttgcagtggccgacttttatttgttgtcctattgttgtttcttcctcctaatgccccctatgatcacgagctgagacaattgtcacagatacctagtatagctgtaccagtttatgattcatgaacattgtcatatgttgcagtgacagccgttgcatacttagaagccgcatatgcattatattttaactgaactttgctaaattgaaacaggaggagatttttggtccagttcttctcttgatgaaggtatgtgaaaattgtttcccttgctatgcacaataaaagatctgttacactgctgaccattttctctgttacaggctgagagcctagatgatgctatccaaattgtgaacagaaacaagtaatgctttattgatttattccttccatgtactagatagtcctgattgagcacatgttgaaattttgaaaatcttcctacatttctttgtgtgtcagatatggcaatggagcatccatatttacaacatctggtgtgtctgcaaggaaatttcaaacagacattgaagctggccaggtattttgtagtctctttcttacaatacactacatctctcccacgttcgagaaataaaaacgcgcggacatttcatgcatgctaactaggcctagctagacagggcctgaaaaacaggtttggagaaaatatgataactaaagtaactgaaataaacccagatttttgccatttgctaaatcaaattcaaacacatatataaagtgagaagggccccaagcccagctataacttggagcctgaactttgaatttgcatgaattctaagttagcagaaagcaaatctccactgggcactgatgcaattgataaatttgtcgatatgaattaaccttcacaagaacttggattgttcacagtttgtggcttctcccctttgcacttcgcaggtgggcatcaacgtgccgattccagtacccctgccgttcttctccttcaccggcagcaaagcctcctttgcaggagacttgaatttctacggtaagagatcaacactctcgatctaatctcactttgatcctttcccaacaattgcaatgggctcaaagctatatccaactacggccgtatcgatcatctgaaaagaaactcgctcaatgaaacattgccgcgtggcgtgcaggcaaggcgggcgtgcagttcttcacccagatcaagacggtcacgcagcagtggaaggagtcgccggctcagcgcgtctccctctccatgcccacctcgcagaagtgaggtgaaaaaaaagaaaggcatcttgtacgttcttcatttgcccatccatggaaatagacacatgcaccaatgcgaaatgaacagaagaataatcgataggcgcgggatgccatgccatgccaataaaggtagctggtct

SEQ ID NO: 6 - DNA, 232 bp, Oryza sativa
  misc_feature (1)...(101): segment of the T-DNA insert in line 1D-059-12
cgtaccgttaacgagctctagaagcttcagtactacgcgtctcgagaattcagtacattaaaaacgtccgcaatgtgttattaagttgtctaagcgtcaaggagagcgaggatggccgtaaggattcgtctcctctcctttttgttcatgatcctgtctgtgtgtgattctggacttctggtgctaagtctggtgttgtttatgggtgtgttgggattgggcagtggtgagc

SEQ ID NO: 7 - DNA, 246 bp, Oryza sativa
  misc_feature (148)...(246): segment of the T-DNA insert in line 1C-087-40
aggtgcatgtctttttaggcttatttaataatcctgtcaacaatgacatttctttagagaagaacttttttattcttatgaatgtcatttttgtttcatgtaagttctattaaaattactaccaattaatgtttgatgttcatttcattcaaacacggatccgaggtaccaggtaccaggtgagttccattcttactaccacggtgctattttttttgctatgtggctaattacatgactaacttg

SEQ ID NO: 8 - DNA, 370 bp, Oryza sativa
  misc_feature (1)...(218): segment of the T-DNA insert in line 1C-017-14
cgtaccgttaacgagctctagaagcttcagtactacgcgtctcgagaattcagtacattaaaaacgtccgcaatgtgttattaagttgtctaagcgtcaagttgtctcgagaattcagcgggcgaccccggctagaacaggatgtaggggaggagacggggtggagggaagagatggccgggagaaaataaattgatcaatatattaatataggaagggtgtagctatatttatggcgccatatttttcacaaaaaatagtcggtatgtatttacattgtaactccttaaattacatatgtaattttaatgtatttataatgtaagtctcaaaattacatatgtaagtcctaaaattacatatgtaaatt

SEQ ID NO: 9 - DNA, 214 bp, Oryza sativa
  misc_feature (126)...(214): segment of the T-DNA insert in line 1C-038-56
taaatatatgctaatttaagtaggtgtggtatggggggcttgatttggtgcaaatctagagttctgaagatggacctgacctgttggttggtctcttcagagtttttaggagttgagacaaagcctatccgaggtaccaggtaccaggtgagttccattcttactaccacggtgctattttttttgctatgtggctaattacatgactaacttg

SEQ ID NO: 10 - DNA, 514 bp, Oryza sativa
  misc_feature (1)...(113): segment of the T-DNA insert in line 1C-041-47
gtgaagaaaattacttccttcatttcacaatgtaagttattttagcattttccacattcatattgatatcaataaatttagatagatatatatgtctgaatttttgcttttcctcttctcatgagtagtattcctataaattcttgcttgcttgaatttctgtccatgcaaaccttttttcctaaccaactatgtgtgcttgtccgctaggtattggaggtcaaacagctctgaaacttattcgtcaacatgggtccatagaaagcattctggaaaacataaataaagacaggttaatcttttgaactatttgaatatttcccacttgactgagaactcaactgagacatgtatatcagctgtgtggcaaaggcagcccaaaattttctatatgtgctactttatttcatttcttggtcgcattatcttacttagatgtaatgatgtattgtaatgtgatgaagaccatttcctatgctaatttgtgctaaattttgatcatatctatcgccta

SEQ ID NO: 11 - DNA, 603 bp, Oryza sativa
  misc_feature (512)...(603): segment of the T-DNA insert in line 1C-064-20
ccgatcccgatttgaggagctgaacaaggatctcttcagcaagtgcatggaggctatggagaattgcctccgcgacgccaaggtggacaagtggagtgttgacgatgtcgtcctcgtgggtggctccacccgcatacccaaggtgcagaagatgctgagtgagttcttcgacgggaaggagctctgccgcagcatcaaccccgatgaagccgtcgcgtatggcgccgccatccaggcctccattttatgtggtggaaccgatgataagaggttggttgatatgcttctccgcgaagtcacgccgctctcgctaggtgttgagactgaagataattgtacaatgagcgtggtgatcccaaggaacactgcaatcccgaccaagaaggtgaaaaacttcactactctctacgacaaccagatcaacgtgagctttccggtgtacgagggtgagagcgcaaacaccaaggacaacaacctgctcggcgagttcacgctatatggcatccccccggcggatccgaggtaccaggtaccaggtgagttccattcttactaccacggtgctattttttttgctatgtggctaattacatgactaacttg

SEQ ID NO: 12 - DNA, 401 bp, Oryza sativa
  misc_feature (1)...(96): segment of the T-DNA insert in line 1C-109-35
cgtaccgttaacgagctctagaagcttcagtactacgcgtctcgagaattcagtacattaaaaacgtccgcaatgtgttattaagttgtctaagcagccaagaaaagcacactgaaaatgaaagtagaatattttacagcctcccattccgtcggtgacttcgttcaaagtctatttacgcggcgaggatggactacagtactccatagcaattgtcgatgacatcaggcactgtcactgctatcgcaaaggatccgtgtccttatttatgacacacatgacacacctacaccccttatcattatatttgtttttagatttgtgcgtataatacagatttcaatagagcataacattatgatttttcttttccgtacaacacacgaatctcaataaaatat

SEQ ID NO: 13 - DNA, 628 bp, Oryza sativa
  misc_feature (528)...(628): segment of the T-DNA insert in line 1C-109-51
gtggcggaacacctttccttcttggttcagaagatgagatggatgctttcactggtggtatcagtgatgaaacacttaaatatactctgaaatgtggtgtaggctacttgcatgaaggtttaagtgatctggaacaggaagttgtaactcagctgtttctcagtgggaggatccaagtgtgtgttgcgagtagcactgtgtgctggggaagatcattgcctgcccatctggtggttgtgatgggaacccagtattatgatggccgggagaatgctcatactgattatccaatcactgatctactccaaatgatgggtcatgctagcaggcctcttcaagataactcagggaaatgtgttatattgtgtcatgcgcctcgcaaggaatactacaagaagttcctttttgaggccttccctgttgagagccatcttcaccacttcttgcatgatcatatgaacgctgaggtggtggttggtgtcatagaaaacaagcaagatgctgtggattaccttacttggaccttcatgtcaaacacggatccgaggtaccaggtaccaggtgagttccattcttactaccacggtgctattttttttgctatgtggctaattacatgactaacttg

SEQ ID NO: 14 - DNA, 377 bp, Oryza sativa
  misc_feature (1)...(61): segment of the T-DNA insert in line 1C-056-07
acaagccgtaagtgcaagtgccaaaatttgtgaagaaaattacttccttcatttcacaattttcttcagcattaacacagtgtactacatggcaccctttgccaccatgatactggctctaccagcagtgttacttgaaggaggcggtgtggttacctggttctacacacatgactccattgcttctgcactagttatcatcataggctctggagtgcttgcattttgcctaaacttctccattttctacgtgatccattcaaccactgcagtgaccttcaatgttgctggcaacctgaaagtagcaaccatttggcctatgctttgttcttttcatatattctgctcgatcatttcttagaactgatcattgcgct

SEQ ID NO: 15 - DNA, 422 bp, Oryza sativa
  misc_feature (351)...(422): segment of the T-DNA insert in line 1C-100-32
ctaaactgaccactcttataagttttattactttatactgtagatgtcttgctgtcctgtaactcaatatgctacccactaatatctaggagttaccattgtactaaaaatgtataaaacatggttgataggagttcagaaagtgcatttatctgggtacatccgatcactggtagaatatgttaccacttgaaccaaactttatgtaatttataatggtatatttgcaactacggaatcttttactgctcatgcaggatcacaaaggaatttcaaatgctgcaccagctgattgtccattgatcccactattggtgccaaaagtagatcaatcagaaggtacagtggataccaggtgagttccattcttactaccacggtgctattttttttgctatgtggctaattacatgactaacttg

SEQ ID NO: 16 - DNA, 335 bp, Oryza sativa
  misc_feature (259)...(335): segment of the T-DNA insert in line 1C-142-27
taagtgagtccatctgctatctagctctagtttccttttgttaaacactttgaaaggactttggttcgatagcaaccgctgcagttgcaaatttgcaatattgtttagcgagtagcagcctgcactgtcgttgattgaggattcgttgcttgttttgcctaccgtcatgggcctttttcaggtcaccgaccgcatcgaggataagagccaagattctgtacgcgacgtcgaagcaagggctgaggcgggtgcttgacggaggtaccaggtgagttccattcttactaccacggtgctattttttttgctatgtggctaattacatgactaacttg

SEQ ID NO: 17 - DNA, 425 bp, Oryza sativa
  misc_feature (332)...(425): segment of the T-DNA insert in line 1C-140-04
gctttccaggtatatatacataaaatccatcgatcatgcatgtcagaagctcatcatgctcagatgctcatcctgctcacgctcaatgctcatgctctgctgaaatttggcggaatttgttgcatcggatttgtgtttccgtattgcatcatgtttgtatatgccaagatatgtgcttactaccgtgtgagcaatgctgttcaagaactgaatttgtttttgcaaatgtgtcacacctggagtaggtggaaggggcagctgcagaggatgggaggaagcccagcatttgggacaccttcatccatcaaggtccaaaaaaatggagtagatgacacggatccgaggtaccaggtaccaggtgagttccattcttactaccacggtgctattttttttgctatgtggctaattacatgactaacttg

SEQ ID NO: 18 - DNA, 1149 bp, Oryza sativa
  misc_feature (0)...(0): line 1B-115-22 - genomic DNA without T-DNA insert
aagcttaatatattaggagtagtaagctagcgtgtgaggaagattcataaccatatatcatcttaattagcctttgtgatttagcttaatcacatggctaaggcacaaccacccatccacttaactcttcattactacgctagctacacgaggagagtagctagctagtacaggccccggcaagcataaatagcagcgtcccctgcttcctttcttcatcgtcctcagctcatcatctgcatgcagttcactgcacaccacacagcttagcttgctcagcttcactgatcttcttagctgcagctacttcactttgcatagtttgatcgaactaaataactcaccaagttagctgtaatggccaagctgatcctcgccaccttcgccgtcgtgttcatggcgctcgccgccacctccctcgccggcgacccggacatgctccaggacgtctgcgtcgccgactacaagtccctcaaaggccgtaagtgttggaccattattgttcatcgattcataaaatatgaattgacctatttttttttaacgacccgtacaagacagtgcgaagattcatattaacgtatatatgtatgtcgtgcagcgctgcggctgaacgggttcccgtgcaagaggatagagaacgtgacggcgaacgacttcttcttcgatgggctgatgaaggccgggaacacggggaacgcggtggggtcggtggtgacggcggcgagcgtggagagcctgccggggctgaacacgatgggggtgtccatggcgcgtatcgactacgcgccatgggggctgaacccaccgcacacgcatccccgtgccaccgagatcatcttcgtcgtcgagggctccctcgacgtcggcttcgtcaccaccgccaacaagctcttcacccgcaccgtctgcaagggggaggtgttcgtcttcccgcgggggctcgtccacttccagaagaacaacggcaacacgccggcgttcgccatcgccgccttgaacagccagctccccgggacgcagtccatcgccgccgcgctcttcggcgccgcgccgccgctgccgtcggacacgctggccagggcgttccaggtcgacggcggcatggtcgagttcatcaagtccaagttcgtcccgcccaaatactag

SEQ ID NO: 19 - DNA, 2971 bp, Oryza sativa
  misc_feature (0)...(0): line 1B-164-43 - genomic DNA without T-DNA insert
caaaaaggcgcctctaagaattcctcccaaactttttggtgttgtcaagccgtgcgtttcggggtacatcaacggcggcgatgagctcccggatggccggatcggcgatcctccgtcacgtcggcggcgtccgcctcttcaccgcgtcggcgacctctccggcggcggcggcggcggcggcggcgaggccgttccttgcaggcggagaagccgtccccggggtgtgggggttgcggctgatgtccacgtcgtccgtggcgtcgacggaggcggcggccaaggcggaggccaagaaggcggacgcggagaaggaggtggtggtcaacagctactggggcatcgagcagtcgaagaagctggtgcgggaggacggcacggagtggaagtggtcttgctttagggtacttgcgcttcgctttgctcctgatcggtttcctgtttcgattctgtttgtcccgtagattgatcgccagatcgatgtatccatactccgagatagtaggctcggtccttaacttgtcgcgccgccgtgtgtaattttggtttgattttctccgcagccatgggagacctacaccgcggacacttcgatcgatctgacgaagcaccacgtgcccaagacgctgctcgacaagatcgcctactggaccgtcaagtcgctgcgcttccccactgatatcttcttccaggtataattgtgcttactcgatcaccatgcaccgcccgacggccgtttgattctccgggtgattagcgctgtgtgtttagttcaacgtttttttcgaagggaaaaactgagaggccgcgtgactcttggcgtgtcataagaggcaaagtagcattgcaaccagggcacttgagaattttggtttccttgcttccgggaagatgccatttgtatttaggacgcctgggttctacctgatccacctgtgtgattgattttattttcggaaagtattttctacacctaggtaacatggcacctatgtgtatgcatgcaatttaaaccgttataaaaaaatttacaaacattatacaaacatgtaagtctaaattcgatcaacatatgaagaaacaaaaataataaattttatatgcgctcttcgggtactattcactgtctgattttattatttttgtttctctagttatagatcaaatttagtcatgcatgtttgtgtatagacttatatcatcacaatcgatgttactaatttttttttcaattttttctataactatttgagtcgcatgcaaatgatgggtgtcatagcacttaggtgtagaaaatccctgtccttttattttccgctatactatctgataacaacggctaagaaaaggacacgcattgtgagaaggactcaactttgatttgttaatctcttgcccattaaatcaatcataggcttattactgtttgcactacaatgactggtgatccaattgtttcctagtgcaaattaatctatatatgattctaatgtcttatgactaatttggaagaacaattatattacatgatatccatttctagtagtatgtggaaatgatctctagtcggtcgctaattgctgtaactgtcaatggttaacactgcaaattgggttgggtgcagaggaggtatggctgccgcgcgatgatgctggagacggtggcggcggtgccggggatggtgggcggcatgctgctccacctccggtccctccggcgcttcgagcagagcggcggctggatccgcacgctgctggaagaggccgagaacgagcgcatgcacctgatgaccttcatggaggtggcgaacccaaagtggtacgagcgcgccctcgtcatcaccgtccagggcgtcttcttcaacgcctacttcctgggttacctcctctcccccaagttcgcgcaccgcgtcgtcggctacctcgaggaggaggccatccactcgtacaccgagttcctcaaggacctcgaggccggcaagatcgacaacgtccctgccccggccatcgccatcgactactggcgcctccccgccaacgccacgctcaaggacgtcgtcaccgtcgtgcgcgccgacgaggctcaccaccgcgacgtcaatcacttcgcatcggtacgcatgcatgatcagcgatcttgctctttgatctcgccttttggggatgtatgttgaattggttctgacgtgtgatctggtctggcttgtgatgtgcaggacatccattaccagggcatggagctgaagcagacccctgcgccgatcggatatcactgaggatgtttgtctactgccgaggatttgcatcactaataatcaaatcaaatgagtatacttcgtttttcagggaaagcaaatgagagctctttagctgtatcggatttggtaattgctactagtaactactaagtactgaaaaataaggagtctccttgtttgaagtacaaatctaggagattttttgaaccaactctatctgaattgggcctgtttttcgtacgccgcactgcttcatgtacatatattccatcccctatcttatgtgtggcaagtgtctttctaaattcttggatgtaatgacacctgaatttctttcgttgaactattggcagctgagttatatatactacataccattcttttgttaaagcaggtcagagcatagttttgtccatttgatgtctgtcacaattctggtggtaatgttccaacttcgagaaaattgcatatctaccatcccataagaatggcttcgctagaataccattatgaaaacgggcttcgctggaataccttcctcaaccagagtgtcttcgccaaaatgccatcccggacagaaatgcccctggctcttcttcttcctccatctcgcgcgtgaagagctcgccggcggccatggctagctccctccctgccatggctagctcgccacgggctccggcggagggctgcgcggtggcggcaggcgcggtcgac

SEQ ID NO: 20 - DNA, 4566 bp, Oryza sativa
  misc_feature (0)...(0): line 1B-192-40 - genomic DNA without T-DNA insert
tgtcggtataactaataaggtagtattacttattttgctgaaaattaaggtctgaaagtttgcatagtcaacctcttccatcttaattcaacaagatctccgtaactaagtccgtacacacaaactagtcatgttcaacaggaatgcattgactattcgaccaagaatatggcaccatccaacaatcaacatgggtaaaagatgtataaacgcctattaatataaatgtcctagtccactataagaatcaaaagcatctaaaaactgaaaccatattgtccaaaatacacaccagcaaagatggtatgtgcagctatacacattgcagtcgtagcaatgctcgtctcccttacagcattagcaatagctgacgaatccgacaataaccaacgggaagctctgctctgcatcaaatcacacctctcaagcccagagggaggcgccctcaccacatggaacaatacctcgctcgacatgtgcacctggcgcggcgtgacatgctccagcgagctcccaaagcctcgtttggtcgtggccttggacatggaggcacagggcctcagcggagaaatcccaccctgcatctccaacctctcgtccctcacgagaatccacctccccaacaatggcctctccggtggcctcgcatccgcagccgatgttgccgggctccgatacctcaacctcagcttcaacgcgatcggcggtgcgatcccaaaacgccttggtacgcttcgcaacctttcgtccctggacttaacaaacaacaacattcatggcgagatcccgccgttgcttgggagctcatctgccttggaatccgtcggtctcgccgacaactacctgaccggaggaatcccattgttcttggctaatgcgtcctcgctccgctatctttccttgaagaacaatagcctctatgggagcatccctgcagcacttttcaacagctcaaccatcagggaaatataccttggagaaaacaatctttctggtgcaattccacccgtaacaatattcccttcccagatcaccaaccttgatcttacaacgaatagcctgacaggaggcataccaccatctctagggaatctctcatcgcttacagcactcttagctgcagaaaaccagttgcagggaagtataccggattttagtaaactatcggcgctacggtatcttgacctctcttacaacaatctttctggaactgtaaatccctccgtttacaacatgtcgtcaatcacatttctcggtttggccaacaacaatcttgaggggataatgcctcctggtataggaaacactcttcctaacatccaagttctgattatgtctgacaatcattttcatggagaaatccctaaatctctagcaaatgcttccaacatgcagttcctgtatctggccaacaactctttgagaggggtgattccttctttcggcttgatgacagatttgcgggtcgtcatgctgtactcaaaccagttagaagctggagactgggcattcctgtcatccttgaagaattgctccaatttgcagaaactgcactttggcgaaaacaacctacgcggggacatgcccagctctgtagccgaattgccgaaaacattaacttcactagctcttccgtcaaactacatatctggcaccattcctttggagattggaaacctatctagcatatccctactttatcttggtaacaatctattgacagggagcatacctcatactcttggccaactaaacaatcttgttgtccttagcctttcacaaaacatattctctggagaaataccacaatccattggtaacttgaatcgattgactgaactctatttagcagaaaatcaactgactggaagaatacctgcaactttatcgaggtgccaacaattgttggcattgaacctttcatgtaatgcccttactggaagcataagcggagacatgtttatcaagctaaatcaattgagctggctactcgatttatcacacaaccaattcataaactccataccactagaacttggtagcttgataaatcttgcttctttgaacatttcccacaacaaactcacaggcaggatcccatctacacttggttcttgcgtccggttggaatcgcttcgtgtaggaggcaacttcctagagggaagtattccacaatcattagcaaatctcaggggcaccaaagtgctggatttctcccagaacaatttatctggtgcaataccggatttctttgggactttcacctcattacaatatctaaatatgtcatacaacaactttgaggggccaattccagtagatggaatctttgcggacagaaataaggtgtttgtccaaggaaatccacacctttgcaccaatgttccaatggatgagttgactgtctgctctgcttcagcatccaaaagaaagaataagctcattattccaatgttggcagctttttcatctataatcttactttcttcaatcctcggattatatttcttgatcgtcaatgttttcctgaaaaggaagtggaaatccaatgagcacatggatcacacctacatggaactgaaaacgttaacatatagtgatgtaagcaaagcaacaaataatttttcagcagccaacatagttggctctgggcattttgggacagtctatagaggtatactgcatacagaagacactatggtcgctgttaaggtgttcaagctcgatcaatgtggtgcattggacagctttatggcagagtgcaaagcactgaagaacatccgtcaccgaaatcttgtcaaggttataactgcatgctcaacttatgatccgatgggcagtgagttcaaggctctagtatttgaatatatggctaatggtagccttgagagccggcttcacacaaagtttgatcgatgtggtgatttgagtttgggagaaaggattagcatagcatttgatattgcttctgctctagagtaccttcacaaccaatgcattccacctgttgttcactgcgatttgaagccaagcaatgtacttttcaacaatgatgatgttgcatgcgtctgcgactttggtctggcaaggtcaattcgtgtttattcatctggaactcaaagcatatcaacaagtatggctgggccaagggggtctattgggtacattgctcctggtgagtaggttttctgagcatactgcagagtatcatacaatataaagaaattactattatatctcttatgattataatttggtactcctatgttttgctggttatctgaacctttttgttttcgaacaaagcagagtattatttaatagaaggaataagtattttatgattaacactatggttcattttactggtgatctgaactttttggtttcaaaacattgcagagtatggcatgggcagtcaaatctcaactgagggtgatgtctatagttatggcattattcttctggaaatgctaacaggaagacaccctaccaatgaaatatttactgatggcttgacactccgcatgtatgtcaatgcatcactttcacaaatcaaagacatacttgaccccaggcttattccagaaatgacagagcaaccttctaatcataccctgcaattgcatgaacacaagaagacaggtataatggacatctgtgctctacaactccttaaactcggtctgcaatgctctgaagagtcgccaaaagatcggccatttatacatgatatatatagtgaagtaacgtcaataaaagaagcattttttgcaacgagcatttgacacaataaaagaggaacaccaaagaatgaagcactgccaaatttatcttccaaagtttctttactactgcagtaagctacatatgtttttaccgctacaacaggatacatttgcacactgttatttaccgatgcatttcaagcagcaatttctatcatgtaataaaaattcttgtacagttcaatgatttgttgccgagatgcagcttgatctgtttttgaagtactgttactagtaaagccttcacgccgcgggaataaagaaaaatatataaatagtatagcatattctatcttaatttcccatgattttggggtaaaattcgggcataaactgaaggctgagcatgtatatgaaatcggatttgagaggaaatcatgtttactactcaaacgtacacctgaatttgttttatacattgattttcagttccatctcgatgcaaactgggaggggtggagggtattctcacatgtacgataagagaaattgcatcaaaattaggagggcttagattatctatgctaacgtgatggataaaacaaatttctttaaaatagcattgtaattacagagatatatcgactaaaacagaagatcggatctagctaagacagtcccaactaaaccaatgcttccctgaacacgatgaagttaaagatagaattaaaatatccagtaagcaaatataccaatctaactagagcaatccaagagatccaaacgatgcggtcttgaagaatgccaataaagccgatacaattgccaaggcctacgg

SEQ ID NO: 21 - DNA, 1914 bp, Oryza sativa
  misc_feature (0)...(0): line 1B-207-27 - genomic DNA without T-DNA insert
atgcaagatcatatacttacagcttttctggttgtttctctcctctttgcatgcattcctcctgccaagagtgctgatctgaactccgacaagcaggctcttcttgcatttgctgcctcactgccacatggcaggaagctcaactggagctctgcagccccagtctgcacttcatgggttggggtgacttgcacaccagacaatagccgcgtgcaaacactacgcctaccagcagtagggctctttggcccgctaccctcagacacgcttggcaagcttgatgccctggaggtattgagccttaggtccaatcgcatcactgttgatctccctcctgaagtaggatccattccttctctccattccctatatcttcagcataataacctatccgggatcataccgacttcccttacttccactttaacatttctagatctgtcatacaacacttttgatggagaaatcccattgagagtgcaaaatctcactcaacttactgcattgcttctccagaataactctctttctggacccatccctgacctccaactcccaaaattgaggcatttgaatttgagcaacaataacctcagtgggccaataccaccttccttgcaaagattcccagccaattccttcttggggaatgcttttctatgtgggtttcctttacaaccttgccctgggactgcaccttcaccttctccttctccaacatcaccatcacccggcaaggccaagaagggcttctggaaaaggatcagaactggtgttataattgcacttgctgccgcaggaggggtattgttgctaatcttgattgttttactcttgatatgtattttcaagagaaagaaaagcacagaacctactacagcttcttcgtcaaaaggaaaaactgttgctggtggaaggggagaaaatcctaaggaggagtacagcagtggtgttcaggaagctgagaggaataaattggttttctttgagggctgttcatataattttgacctagaggatttgctgagagcttcagctgaagtccttggaaaagggagttacggaactacttataaagctgttcttgaggatggcaccacagttgtggtcaagagattgaaggaggtggtcgtggggaagaaggattttgaacagcagatggagatagttggcagggttggccagcaccagaatgttgtcccattgcgtgcctactattactccaaggatgagaagctactggtgtatgactatatcccatctggtagccttgctgttgttttgcatgggaataaagctactggaaaagctccattggattgggagacgagggtaaagatatctcttggtgtggctcgtggaattgctcatcttcatgctgagggaggcgggaagttcatccatggcaacctcaagtcatcaaacatccttctgtcacagaacctcgacggctgtgtctctgagtttgggctggcacagcttatgaccattccaccagctccagcacgccttgtcggatatcgtgcaccagaagtcctcgagactaaaaagccaacccagaagtctgatgtctacagcttcggtgtgttggtcctcgaaatgctgacaggaaaagcgcctctgagatctcctggacgagaggactccattgagcacttgccaaggtgggtgcagtctgtggtccgggaagaatggaccgcggaggtttttgacgtcgacttgttaaggcatcccaacattgaggatgagatggttcagatgctccaggttgcaatggcatgtgttgccgccccccctgatcaacggccaaaaatggacgaggtgatcaggaggatcgttgagatccggaattcctactccgggtcaagaacaccaccggaggagaagcagaaggatgaatctgcagcgccatga

SEQ ID NO: 22 - DNA, 6631 bp, Oryza sativa
  misc_feature (0)...(0): line 1B-138-07  - genomic DNA without T-DNA insert
cgatgctccgcgccgccctcctccgctcaggtaggtcgccgccgcctcctcctcctcctcctcccttctccgcggcgactcctctgacccttctcggccccgcctccccacgcaggctccggcctccgccggcctcccatggccgcgccgctctccaccgccgccgccgcctcgtggctctccgacagcgcctcgtctccggtaagcacccgcgcgcggctcatccattcccaatttcccatccgtgtgaagtgtgattgacctctctctctctctcactctcccggcgcagcctagggtgcgcctcctcatcggcggggagttcgtcgagtcgcgggccgacgagcacgtcgacgtcaccaatccggtaaagcccgccgtgagcctccccacgatcgatcgatttgggaggtgtggtgtgggggcatgtgacagtgattctctgggtgtgcgcaggcgacgcaggaggtggtgtcgcggatcccgctcaccaccgccgacgagttcagggccgccgtggacgccgccaggaccgccttccccgggtggcggaacacgccggtgaccacgcggcagcgcatcatgctcaagtaccaggagctcatccgggccaacatggtaattagttgatagatggattgaccttccttccatcgttgggggattggagagctagaagcttcactcgatgatgtggttggatcatgttatagtgattctgttctttttttgtgatgtcaggataaactggcagagaacattacaaccgaacaggggaagacactgaaggatgcttggggcgatgtattccgtgggctaggttggttttcagacccttatagtacctggtttcactgcttcattagatagtgataatctcagcatttcaaatctatgagtagtcgttatgttctttcaatctactaatcttaatctatggcttgattcaaatgagatgatatttgtttgcaaggaatatttcttatactggatgtttgacacgctggttgcaagatctgattaactcctggttttgtagaggtggtggaacatgcttgtggaatggggacactgcagatgggtgaatatgtatcaaatgtttctaacgggattgacacctttagcattagggagccacttggtgtatgtgctggaatatgtccattcaattttccagctatgatccccctatgggtacgcacaacttcacttcttaatatcaaaatttgttattattacgactcttagcccaattatcaatataccatgcaatacatcttactttagtcaacacgccatgatttgatcgtttcatttgtttcttctttccgtttggctgcagatgttcccaatagcggtcacttgtggcaatacttttgttctaaagccatcagaaaaagatccaggtgctttgctttatcatcatttttttacccagtttattcactacacccgctgttgttgcattgacgacaatttaaagccagataaaaatatatggttaaatgcttatctgatattacagagcgggttaaccaagctgtacttttctccacaacatataagatcattgattctatgtattcactatatacatatccatttggtactataactttgggggtgtgtttttactataacatatggtgaagttaaacatttggtgcacagttatctacccaaattggacatttccactacctactgctataaaaaaaccagcagtaagttttaactctttggaggtagtagcaagacaaccttttacaattccattgagatattaataaaaacagattacatgttgctttacaatctttgtgtacatggcaaggacttaagatggagtgaaaatatgacatgtttttgtgaactggcactagtagtatttctaaggcatgaaaaagatatattataaagtagcaattgttgttcatgtccttgaagaggctgtaatcttagtctagtagaacaaaagaaggcacatgacatgttcctgttagtaatgtgatggcatactttgtaattattggttactacttcaggggctgctatgatgcttgcggagctagcaatggaggctggtttaccaaagggtgtgttgaacattgttcatggtacccatgtacggtaaccttggttactcttcgtccttcctttttttttttccattttttgagctcgatactagattaaataatttatttgttgtgcctgttggtgccaagtgttgctacagcacaaatagatattcgatgattgatattttctggctaacatgacaataattttactctttcttggaacactggcaaaaaaaaaaagttgccttgaaacttcaccttaccatgcttagctctgtagggattgtttggtcttcatggttatatgatgctatttttactgctaaacattcatgaatctgttcatctttagccgccacacttgctaagctgctttctgattctaccttactatttcaggatgttgtgaacaacatttgtgatgacgaggacatcaaggcagtttcctttgttggttccaatatagtaagcggatccaaatgatctgtctatttctcgatttgtggtaccatttttgcatcaatcatctgtctattttgctctgcataatactttgcaaatctggtattcatcctattctgtttcagtctttaggtcattaacttggcatctaggccattgcattatcacacacttctacatgtttgagatcttttcaatcttgtattcttatgttgtcataatggttcaaattctgattatgatctaacttataaaaattataaatttaccagttaccatgatggtgataaactgataataaacatgtatacacagcatgaggatgttttcatgatgttaggacattcggttgtaaaatttacttgtaaacaattatgttagatccgtatagcacatgtcacattacactcatgatacctgaatcattctttattttatttttgcccaaagctagtcatttgtttcagcagatgatataattatgatctctctctcactctcaggctggtatgcatatatattctagagcatcggcaaaggggaagcgtgttcaggttagtttgcacctgttcctgacatgcagaacctttccttcgaaaacttctgcctggtgtgctgtaagcagcatgttacagaatgcctttttgtttattttcagtctaatatgggtgcaaagaatcatgcaattatccttcctgatgctgaccgagatgccacactgaatgcccttattgctgctggttttggtgctgctgggcaaaggtgtatggcattgagcactgctgtttttgttggaggttcagagccatggtatatacttctgtcctgtttgtttcttcaggataagacctggcattaggtaatgaagtataaaaataaccatttgtcttgtagggaggatgagctagtcaaacgtgcaagcagccttgtggttaattcaggaatggctagtgatgcagaccttggtccagtgatcagcaaacaggtaaaccatctgcttttttaccaacgaaaaggttgttgacctgattttaagtaacctaaatatgaagtggaatcctacttgtacttccctgttacttggaacactgaactggatttgtgctcccccccccccccccaaaaaaaaaaaaacttgaatggtcctctaatcgtgtatacacattgaaatcatctagttcagatattattttggtacctaaacttatcctaatgtggacttctcttggtacttatgtaggataggttatgaacatttatccttttatgccatgatttctatgttgactcaacacgtgaacttaacatccccagatttcaagagtgttaacattacatagccaagtaatgacttgtaaagtttctgtattttttgatctcgtctgttcatgaatgaacctgttgtgttgagattgattacagtttagcgatagtgatagtgcatgaaattgcatttgaaattcaaaaaaggctacttctgtttttttttggttctgatgagactgttggtatgtggtatatattttctggaaatatatattaccttttggggcttggctgcgagtattattagcttcagattcataatgattgtggctactacattggttacgctcacagtttttatgctattttcttgtaaatataaatttataacattttctaccttgtcatttctgttttcctttttctcaatatatcctgtcttaacagtttactatcaacaggctaaggaacgtatctgcaaattaatacaaagtggtgctgataatggtgctcgtgtgctgcttgatggaagagatattgtggtaattttagttcagttgtttccttgcctgcagttctcccctggttcttatgcagcaatgcataatagtgctaaactgtgctaaattttgcaggttcctaacttcgagaatggtaattttgttggtccaacactccttgctgatgttaaaagtgaaatggaatgttacaaggtattattctgaaatttgtcattttaaactctgcattaggactgatgtttcttgtttgaagagccatgaaaggaaggaaggttgcattattagcctcctgcaaagctatttgttggattagtgctgtctgtgttctaaaaattaaaacatggaatgcatttaaggatatattattttctaattgaagtgctaactgtggacctatttgatactccctccgtcccaaaatataagcatttttagactctgatacgatctttgatatgctactttgaccaacaatatctgtgaaagtaagatgttttaaataaaaggagttacatattatgatagattgtttaatgataaatctagtaatatcaattttacatgattgattgttttttttttgctattaatagtcaaagttaaaaatgtttgacttgttactatgctaaaaatgcttatattttgggacagagagagtattaatttatttgggactatgatgtatctttcttttcaggagtgtatttgacctatgcttttttaaaagaattatatgattttgtctggagaccaatgtaattaacaatccaaaacaaggaaatggcaaagcagataactcgcctattgcatggtatcaaatctcagtattgctcactttcccatttataacttgcaagacactgttctcctcaagtagacatccttgttgcagtggccgacttttatttgttgtcctattgttgtttcttcctcctaatgccccctatgatcacgagctgagacaattgtcacagatacctagtatagctgtaccagtttatgattcatgaacattgtcatatgttgcagtgacagccgttgcatacttagaagccgcatatgcattatattttaactgaactttgctaaattgaaacaggaggagatttttggtccagttcttctcttgatgaaggtatgtgaaaattgtttcccttgctatgcacaataaaagatctgttacactgctgaccattttctctgttacaggctgagagcctagatgatgctatccaaattgtgaacagaaacaagtaatgctttattgatttattccttccatgtactagatagtcctgattgagcacatgttgaaattttgaaaatcttcctacatttctttgtgtgtcagatatggcaatggagcatccatatttacaacatctggtgtgtctgcaaggaaatttcaaacagacattgaagctggccaggtattttgtagtctctttcttacaatacactacatctctcccacgttcgagaaataaaaacgcgcggacatttcatgcatgctaactaggcctagctagacagggcctgaaaaacaggtttggagaaaatatgataactaaagtaactgaaataaacccagatttttgccatttgctaaatcaaattcaaacacatatataaagtgagaagggccccaagcccagctataacttggagcctgaactttgaatttgcatgaattctaagttagcagaaagcaaatctccactgggcactgatgcaattgataaatttgtcgatatgaattaaccttcacaagaacttggattgttcacagtttgtggcttctcccctttgcacttcgcaggtgggcatcaacgtgccgattccagtacccctgccgttcttctccttcaccggcagcaaagcctcctttgcaggagacttgaatttctacggtaagagatcaacactctcgatctaatctcactttgatcctttcccaacaattgcaatgggctcaaagctatatccaactacggccgtatcgatcatctgaaaagaaactcgctcaatgaaacattgccgcgtggcgtgcaggcaaggcgggcgtgcagttcttcacccagatcaagacggtcacgcagcagtggaaggagtcgccggctcagcgcgtctccctctccatgcccacctcgcagaagtgaggtgaaaaaaaagaaaggcatcttgtacgttcttcatttgcccatccatggaaatagacacatgcaccaatgcgaaatgaacagaagaataatcgataggcgcgggatgccatgccatgccaataaaggtagctggtct

SEQ ID NO: 23 - DNA, 3937 bp, Oryza sativa
  misc_feature (0)...(0): line 1D-059-12 - genomic DNA without T-DNA insert
aaaaagatatgaaggttgttgtttatacataagtggtgtgacttttaaaaatgataaaaataaaaaccctatatttgatttattttaatatgctcagacaacgaaacaaccgattttacattttttgaggatttgcttaatataataaagttcaggcccacatgacagcgggagattagtggcatatcctcccctcgcctcacgaggggcatatttcgtaattacgaaattccccttaagaaaacccccgagctgtcctcgtaaaaccctaacaccttctcccccaaattcgctagagccatggccgccgccgccaccgccgccgtccccctcgacgaggccaaagccaaggaggttctccgccaggttcgcgattcctaccccgttcccggatcctgcccgttctggcccctcaaattatgggggaatttttcagatcgaagtcgcttagcgttcttttgttctgcaccgcaggtggagttctacttcagcgacagcaacctcccccgcgacaacttcctgcggaagacagtcgaggagagcgaggatggccgtaaggattcgtctcctctcctttttgttcatgatcctgtctgtgtgtgattctggacttctggtgctaagtctggtgttgtttatgggtgtgttgggattgggcagtggtgagcttggcactcatctgctccttctcgcggatgaagaagcacctaggcctggacgcggacgtgaagcaggagaccatgccggaggagacggtgctcgccgttgctgaggtgctgcggcgttcctcggccctccgtgtcaccgaggatggtaaggaagttattttgtcacatacgtgcaccattacacattcgaagttcatgtgttgacagcaaaagaggcttaacttcattctgtttagattttttagagtgaaatttgttgctcatattgacaatgtcagcttaaattccaataccatgttttttttttcgccccctttaactgtttttttgacagggaagaaagttggtagatcaattgagttgtcgaaactggatgagatcatggagcaagtggactctaggacaattgctgcatcaccatttccttacaatgtaaagctggaagatgttcagtctttctttgctcagtatggcaaggtatacatgactagcatttagtagcaaagcaaatatcacatatggtctgcaaaactgttaagattgatgcatagggtagtcaagaattcagcaacccccaacaacatgggacatttatgagtcctggaatttaaatgatgatgccatgatgatgatgataatgatgctatgctgcatctgaactgtttgcatcacaggtgaacagtgtgaggctacctcgacatattgccgacaaacgacacttctgtggcactgctttagtcgaattttcagaagaagaggaagcaaatgctgtattaaagaatactcttgtttttgcaggagcagatctggaaataaaaccaaagtaagccaactactgctcaagcttctttcctcgtactatactgattagctagagatttatgtctccatgctttgggcaaattcttcatttcttttttgaaacggcaatttttcatttcttatttagttattttttatgttatgcttctatagtaattgcagttgttgattgcaacttgcttagctgcaatatgtcctgttgtttcttcatcatatttgataaatgttaactgaaccagttcctcatttcacatagttgaatctatttattaactgaatatcattctgataaccgtcatgcaatcctgaaaaaaatgaaggaaagaattcgatactgaaagagaggctaagaaagaagcttatgagaagtcacaacctactaagaatggtcatgatgaggggtaaacatttctttactaacaactttacaaatggttttttcaatttccttgagttgaagataaccgagtcataaaaattggttatagatatccaaaaggtctaattgtggccttcaagctgaagataattcaaattgatggtggcatggcagaaaatggtggggacaaagagggtgaaactgatgatgccaataaatcaagaacagggcatgacgagaaaatccctgagaacagtgatatcaaggaagacttgtcagatgatgttgagaagtcaaaagaggcagctgctcaatctgttaagaaaggagaaagtccttcagaaaatgcggatgatccaatttcaagggaagattttaaagaagaatttggcaaattcggcacagtgcgggtaacttttccaataattgatgatgtttgtttttcatattgattttgtttcaccaggagtaacctgaggttgtaaatctggtgtgcattgcataaaatatccagaatagataggttgcatatttcatttggccgactattgtttgctgtagattgtgagactgttatgatgcatacaactgagcttagcttgttagtttttttttaatgagaagtgcttgttggcttcaatgacggagtcatacagttatgtgatcacaatagcatgctgttatgttatatttattcatgctatttttgtaggatttggtaaatgatagcaacacttagttcaatgtcgatcatattacctttgtgctaattggacaaacctaatctagtcaaacgtgtagagctacagtgagatgatccaatagttcgtgttcttactcttactgccttttgtaagcacacatttgtctggagagaaagcaagagaaataatgcttcatcttgtaaagacatctacaccatgcttttatgcaattctgtatcttgctttctaatttctacaagtaaacataatctcttttatcagacaacttatgctgattcaaaactctttcttttttattgttccattttccccccttgaagtatgtggacttcagcataggggaggattcaggatacattcggttcgaggattctaaggcagctgaaaaggcccgtgcacttgcggctatttcagatgaaggtggtttgattatgaagggccatcttgttactttggaacctgtgtctggtaagaactgcactttctttcagttaattattattttttagcttttgttgaactgagtaactaaatcgaccacaaaattgatgccttattgatatgtcatatggttggcattcctgctttaggtgtatttaggtgtactgtttaaccatgagttttaagcagaagctgtcttgatgtattgatgcttgacttcaaaaaatcgctaatgatccatcaaattggtatagaaaaagcataacacttcagcggatttgaatgctatgttttatcactattttgatgatttactgttcctttcattcaggtcaagctgagaaggattattggagtgcgataaagggcggtcaaggaaaatatagagacaatagaagtaacaggggaaggtatttcattttacctcagaatcattcttcctgtctgtttgctctgttagagccgatcctcttaaatatcataatgcacctagaatgaatcttaggagtgggctgaaacttaaaatagaagttgaagccttatggcacacaagaatgccaacacatatatttggagttatattatcatggaactggcagtaaccagtttatctgtcaataggattgccatcccctatttaatcactcactagctacaaacctatggtcctttttcaaatatgtactacaaaaaaagcaacaggcatgtcatgaaatgcaatctcatgtagtatatgtacagtatacactttgataagctgttggcagaagataatgctggttgctttgcatgaacttttctagggactggaagaataacaggggcgggaggcacttcggtgggaagcgtggtcgccactcggacggccatgaaagggcaaataaagctcgaaaagtcgatgc

SEQ ID NO: 24 - DNA, 6809 bp, Oryza sativa
  misc_feature (0)...(0): line 1C-087-40 - genomic DNA without T-DNA insert
cagattagctaggagttgtagtaatatagtagtaagtctaaatcaacatttttttttgaaaataaataaaagggaaaccagctactccagtttgtttgacatcagattcatagaaaaggtgggcttactgggttcggcccattaaccaacccacggacgcttccacctaggagtagtagtttgggtttgggttcctcactcgtctcgtctgttttgttttcccctccgactccgactccgatcgatctcaaccccgaatccctcctcctcaccgccggcgagcttccggctgcggcggagatggccacacgttattggatcgtgtctcttcccgtgcagactcctggctccaccgccaattctctctgggcgcgcctccaggactccatctcgcgccactccttcgacacgccgctctaccgggtaagcatcctccccgccttccttccttccttccttccttccgccaccaccatcctcaccctcctcctccgcctcccgcccgcagttcaacgtccccgatctccgcgtcggcacgctcgactccctcctcgccctcagcgacgatctcgtcaaggtaacccatctcttctcttctccccgatctcgatctcccttgcccccccgctctcacgcgattcctattattcctgccgctgctgcagtccaacgtcttcatcgagggggtctcgcacaagatccggaggcagatcgaggagctagagcgcgccgggggtgtcgagagtggggctctcaccgttgacggcgtccccgtcgacacctacctcaccaggtcgctcgctcgctcctgcccctgctgcctctcagcttagcgattccaactcttttgctaaagctctctacctcttccttcctttgcttcttcttcttttctaggtttgtgtgggatgagggcaaatacccaacgatgtcaccgctcaaggagattgtcggcagcatccaatcacaggtctccaagattgaagatgacatgaaggtgatgacctttttcccttgcgaccacataatcactagtcaagacccaacctacatatagtgctgttaatatgtttcgaccctaatgctgtctctagtcattaattaacgacacttggtgtattcttattcttcttgtacataagacataaagttttgccgttcagcgggaattgtttgcttgctttgcaacaagtaggcaagttttgacattatctccataacgtgcaaagttcaagctagtgagctttgttctcctgccaagtgtgtttttacagggtctagcttggtttctgttttggtatgttctgttgttctagccagctatcaacacaaccatattcttgtgattaggcatccacgtctccttttttagcattgagttatcgcgcacaacactacagcgtgacctgttgaatttacaacagctgagctgaatgtgttttttaccttcaggttcgaggagcggaatacaataatgtaaggagccagcttagtgcgatcaacagaaagcaaactggaaggtagattgcatgccactgagtttttggctttaagccaattctttcttgttttatttcaaaggactcagaaatgcgagggtatggtttcatcttttgttagtcaactctaattagtttgttttccttctgtatagcttagcagttcgtgatctttccaatctggtaaaaccagaggatatggtcacatcagaacatctagtgacactccttgcagttgttcctaagtactctcaaaaggattggttgtcaagctatgagtcccttgacacatttgtggtaagtatatgctcttttatagtgacatggttagtaatagactgtcatttcaactatttatatgattttcgtcctttcagcaagttgcttattttacttttgtagtttttgttcaatccaacaatgtttagtttgttaccacttgtggcgttaatagttctactagcaggagatgttacccctgtagaaaccacttaaagtaaagatctgtttgacctgtaaattctttactcagattgttttatttagcccttataaggaccttgtttttttttccagcactacatgttgatgtgattagtattcgtaattggttgttatatttgagtgcttttttccttgaactaattattgatttataggtaccgagatcgtctaaaaaactttatgaggacaatgagtatgctctctacacggtaacattgtttgctaaggttgttgacaactttaaggtccgtgcacgtgaaaaaggtttccaggtaaatttgccgtcctaactttttcatcaaaaacctttgtttacacttcacatttagcttttatccctagagccttaggagttccaaataacttgggacagaaattcagtcttgtttttctgtccccatcaatctggcaattctctgtcacccattaacttgtattacgttgaaatacaggttcgcgattttgagtatagttctgaagcacaggaaagtaggaaggaagagctggaaaagctaatgcaagaccaggaagcaatgagggcatcacttctgcaatggtgctatgccagctacagtgaggtgcatgtctttttaggcttatttaataatcctgtcaacaatgacatttctttagagaagaacttttttattcttatgaatgtcatttttgtttcatgtaagttctattaaaattactaccaattaatgtttgatgttcatttcatcattttgcaccagaatagagataaatatgttttcatctcctttagtagcctgacggtcttaattgtccttaaaagcatccaattatgcggaatctgaaccagtaagaagatattgtctggtctcagttgccacttgccagcaatagatagatgccacgctgtgttttatgtttaaattttacataaggattttagtaagttataacttcatggcatactgcaagttcttcctactgctgttattatctcactagaaccttattattcagggataaatccttgtttctgggatgtattaataagcttgtgtttattatccataattgtctattttaatttgtgcagaataccatttatgtacgtcatgctaaagtgcaaaactttgtgcttctctatgtgcgtaaaagaaaataattcgtgctgttgtttatctctgcttatgcagtgaattgctgtcatgaaggatttgtttactgaactgagttagttagagcctagctctataggctatagacatacttattacatattcactatgtcccaaaaaaaaaaccaacctagtcaaggatgggatacaacctagtcatgtccagattcgttgtactaggttgtctcatcatgtcctagtcattgacggagggagtagcacatatgtttcttgattgttgggttttgtggtttgctttttcttagattcattcctcgtgaacttgtgttttcatggtgctcagttgtaattgcttacataaaactattctgcaggtattcagttcctggatgcacttctgtgctgtgcgtgtctttgtagagagcattcttagatatggtcttcccccatcattcctggtaatatattctactgttacgtagtatatgacagtcaaatttgtactttgatgcctgatctaatatcatatgctgtttatgttttcatcagtctgctgttctagcaccttctcaaaagggtgaaaagaaagtaaggagcatcctggaggaactctgtggcaatgtccataggtaattcagcgtttgagtttgtgttcattgcattgttagtcacaacatgcacctgcttttgtcattaattgagcctctgaattgcagtatttactggaaatctgaagacgatgttggtgtagctggtctgggaggtgaaaccgaggctcacccctatgtttccttcacaataaattttatctgagcacagtatgttccttgctacctgtggccaggcaaggcagtgttttggagtaattactactttggcaaacaaaaaaccagcgaatggttcggtgtatattataattgtgatacgtcaattctttcttgatttatttacccagcttatatgatcctgtagtttgaaccgatcaataatgtagctttttgggggaacctggactattttcctggcacaatagggtatctgtttggactattttatccccggcaactacaatattccaactgtttcctttagtatatctattgtgctttgagagcaccgtgtaagatgttgtggatattatgtttttgtaactttgagcagccaggttcgtaataaaagatcaatataaataattcaggtcgccgagcctgattgtaaacagtttgttgttgaattcgagctgtttccttttttctttgacgattacgactgttgcactcatatataaatagaaagatggttgtgttttcacattcagtagggtggtgcaccgtaccccgtacatgataatactgatggtgttgtgacagaaggacaagttgggggttatgtatagaacgtcaatccttggtgacccagttcccactgatgatagtagagtagacaagacacaagaggagatgatggtcaaggtgagaggtgagggtgggtgaaattggatcagcatcaaagtctcatgatgttatcattaacatatgtgagtctgatgaattattgattcactgaagaggatcccaatggaggtgatcgcctcattttgatttgagtaggaccgcgttatgaggtcggcaaggagagtgatggatcagaatagtcatagatggaaaaaaaaatgcaaggagtaattaagattaataggagtaatgtggatgaagaagaaacagctaggctaggcacataagaagatgattaatgggcaaaatcaatccaatccccctcatcgcgtcgcgtgagaagagaggaggaaatggcatcctcgagattgcattttgtcgtcgatgatccggtccattccggcgccggcgttcacattacacacccaaccaatatcctcaccagtactactattccattctgctactagtatactccttcagtagtgcagtatttttcttgtataaataatactcctagagtagtcttgtttcacctacccccgttccgttccttgtatgaatccattgccttgcttctgcttgcccatttgatgatttgatttgatttgattcaatctccaaatcgatcgatttctcctcctccataggagaaggagatacacgtgatgtccaaccggcgtcgcaagcgcggcgaatccaagccccaagcccacgacgccggcgacaccacgcccatcgacaacatactcacctccctcgacgacgccccgccaccgccacctccaccgcgccgcgagctccccctcctccagctcaacgaccaccaagaggcagatgacgctgacgcctcgtcgtcgtcgtcgccgcaccagcagcggcggctgtgggtgaaggaccggtcgcgcgcgtggtgggagctgtgcagcagcgccgactacccggaggccgacttccgccgcgccttccgcatgtcccgccccaccttccacttcctctgcgacgcgctcgccgccgccgtcgccaaggaggacaccgccctccgcgccgccatccccgtccgccagcgcgtcgccgtctgcgtctggcgcctcgccacgggggagccactccgcgtcgtctccaagcgcttcggcctcggcatctccacctgccacaagctcatcctcgaggtctgcgccgccatccgcaacctcctcatgccgcgcttcctccactggcccgaccaccccacctccaccgcctacaagacgcgcttcgaggccacctcgggtgtctcgggggtggtgggcgccatgtacaccacgcacatccccatcatcgcccccaaggtctccgtcgccgcctacttgaaccgccgccacacggagcgcaaccacaagacctcctactccatcaccctccagggcgtcgtcggccccgacggcaccttcaccgacgtctgcatcgggtggccggggtccatgagcgacgagcaggtgctccgcaagtcggcgctgcaccagcgggcgtcggcggcggctgggtcgatgtcgtgggtggtgggcggcgcgagctacccgctgacggagtggatgctggtcccctacgcgcagcgcaacctgacgtggacgcagcacgccttcaacgagaaggtgggcgaggtccggcgcgtggccacggaggcgttcgtccgcctcaaggggcggtgggcgtgcctgcagaagcgcacggaggtgaagctgcaggacctccccgcggtgctcgccgcctgctgcgtcctccacaacatctgcgagaccaggggggaggacatggaccccgacctccgctgcgacctcccccccgacgaggaggaggacgacaccgtgcttgtgcagtcggagtccgccaacaaggtgagggacgacatcgcgcacaacctcctccaccgcggattggccggcaccgccttcttctgatcatttcgatttccgtcccaatctgcactacaaacactgtattgaggtttccctcctcccctcctgtttgttcttgttattgctatagattagtttaggtgctccactcaacaacaacaataacacaagattgaaatgtgatgctttgccctcctcgttaataaatcactggaatacatcacttcgtttcaaaatttgctttgctttttggactattctttatcctggtactggtaaattacacatacaagctgaatatttactcctataagtaagcacgacctacagtatttgggtagg

SEQ ID NO: 25 - DNA, 4941 bp, Oryza sativa
  misc_feature (0)...(0): line 1C-017-14 - genomic DNA without T-DNA insert
tgtacctgctctcttagcaccttctttccctattggcaaatggcaatgagtgcaaaaatgcttggaaaaataaccccacccccacatgattatttccagtagggccaaaatccgggcctacgtccgcaacccatgtgggccccacatcccccacaccaaccctctgcacccaaaatccccatccccccactatatataatccccgccgttggatcatcgccctcagcagagcagcgcatctgcatccaaaaccaaacccaaactcgtcttctccaccggagcagagcagcggcggcggcaatggacgccctcctcgtggagaaggtcctcctgggcctgttcgtggcggcggtgctggccctagtggtggccaagctcaccgggaagaggctccgcctcccgcccggccccgccggcgcgcccatcgtcggcaactggctccaggtcggcgacgacctcaaccaccgcaacctgatggcgctggcgcggcggttcggcgacatcctcctcctccgcatgggcgtccgcaacctggtggtggtgtccagcccggacctcgccaaggaggtgctccacacccagggcgtcgagttcggctcccgcacccgcaacgtggtgttcgacatcttcaccgggaaggggcaggacatggtgttcaccgtgtacggcgaccactggcgcaagatgcggcggatcatgacggtgcccttcttcaccaacaaggtggtggcccagaaccgcgcgggttgggaggaggaggcgaggctggtggtggaggacgtccgccgcgaccccaccgcggcgacctccggcgtggtgatccggcgaaggttgcagctgatgatgtacaacgacatgttccgcatcatgttcgaccgccgtttcgacagcgtggacgacccgctcttcaacaagctcaaggccttcaacgcggagcgcagccgcctctcgcagagcttcgagtacaactacggtgacttcatccccgtcctccgccccttcctccgccgctacctcgcacgctgccaccagctcaagtcccagcgcatgaagctcttcgaggaccacttcgtccaggaacgcaagtaagttactatctcaattaattaccttacctgcactgcatatccccaagaatccgcaagaacaaatcctacctaattagtcccagattattatactaccccatttgatctctccttcaatttttttaaaaattttattcgtcatcttgcgtatccaaattgtgtgtggtggtaggtgaggtgaaggtgactagtttggttgcatcttgcatgcatgatgcatttgcatcggtgacatgaactggatgagcgaaaagtttggtagtaaaagcaatattctacactagtagtagggatgtgcacatgccacgcgaggaagaggattaaggccccctttgattcaaaggaaattcataggaattttaaaggatttaatttctatatgaatttttcctatatagccctttgaatcaaaggaatgaatcctatggaatcctataaaattcctatggaatgcctcttcccatgcaaattttgaaggaaatttaacatgaggttctacctcatgttaagaatcctttgagtctttctctctcctcaaattcctgtgtttttcctgcggctcaattaaacggtcattccaatgtttttcctgtgttttgtaatcatctgttttacacttgtattcctattagaatcctatgtttttcttattcctccgtttttttattcctgtgattcaaagggggcctaatttggcagcagcctcagatctgtttgggtaggaggaggaatcaaatcactttgctgttgctgcttgctgtgctgagcggtgacagcacgaggtggtagctgcgagcctgcgaggtcaaatttctggttgtgcaacggcttgactcgggctcgacctgtgccgcggagcccagctaccaccccgtgccaaagccaatgtccacgtcacggttgtttaaactctggagcctaattaaagcggttagtttttggcatgttgggatacatccaaccaaggttgagaattagcggttaaaattcctgtgtcacaaatcttatagattaggtaagcttcaaaacttttaaggcttagttcggcaccccaggttcccaatccaaacatccagtatgcaaaacggaacaatccattagcacgcgattaattaagtgttagctatttttttaaaaaaatggattaattttatttttttaagcaactttcgcatagaaacttttttaaaaaaacacatcatttaacagtttgaaaagcgtacgcgtggaaaacgagagaaaggggttaggaacatgggttgcccaacacaccctaagtattcatatctttccaatatttacatgggagcaatatgtatagatttatctcgaaacttaattactttcatagtattagtactaacaaaaatcattagtttaagtaacactagggtaccagtaccatgtcgatgtccaaaatgacttggtttagtagtgacaacaggagattaatctgtaagaactggacttgcctaatgttcaaagcttccttttcctaaacatgcaaataagtctgacgagtaggtgagccgcacatcatgggttgcttttcgtgccagccagctgaaagcttacgatttgggcaaagtcctgcctcatcacggaagcaccgacggcgacagaaactctctaccagtaacctaaccgatcggaaattcatttcgttctcagcgaattgagcagatctactagctcccccatgtgctcaatgactgaaaaggtcgcaagaacttcagacgcttcccctgttttcttctcgatcagtacactgcataactgagtaatataatcccgtttgattggatcgatcaatggtgccaaacaacaacatgcagctatccctttgattagaagatatacaatttgcagcttgcatttttttttccggtttctggatcgatttgcgattgagaaactgctggcctttttggtagtagctgtgttgggaagcttcggatgaactgttcgactgacaaatcggcgtggacgtgtttgtgtcttttttgcaggagagtgatggagcagactggtgagatccggtgcgccatggaccacatcctcgaggccgagaggaagggcgagatcaaccacgacaacgtcctctacatcgtcgagaacatcaacgttgctggtacgtttttgtttattccgcgcaggatttgacttttagtttatcctcagcttaattttctttggatcaggcctaaagtatatatcgtagtacatcatacgtatccatgtcatagttttgacaaagctggagttttactagtacttgaaaggagctgagtttgtggagaatagaaggttcagaaccaggccaaattgcaatgctagcatttttctaccaacacatcacatcgttagtgtggctaaaaagaaccttaattaagtatgcatcatatataatattgtcatgataggctatggttaaagtatggcttcattaaatacgagcttttatatgatcaggttgccatgttcttggtcttgaggaccagttctttgggtaagaagtagtagccacgtcttaatcagagtaaccattacctaacatacaattgtattttttttattatccggcacatgcccaatgcacaaagtcgtgtgggtacatatggtaggcggctatctcataccttatgattcaaaccatatgaactaccgtagatacgaaacatttgagtcaaaaccagtagtaaaaaaaaaaaggtgtagctatatttatggcgccatatttttcacaaaaaatagtcggtatgtatttacattgtaactccttaaattacatatgtaattttaatgtatttataatgtaagtctcaaaattacatatgtaagtcctaaaattacatatgtaaattcaaaaattacatactaactacatatgtaagtaggatgtaagtgtggtataagtgaggtgtaatttctgtgtaagtggcttaaaaaagtcgattatagcatgtgtcgacataattttagctaacccgatccggtgtcgtgcttgattcatatcatgtaaattatcacggcacgaatctttgtggggatcgagtgatccagaaatcgactgtaacgaagcaaaaaatgtggcgacagtagtttagcaaaacccaataaaaacgccaattttagttggattattattttttagaaaagagtacaaaatgttaattaacaagaagcgtacttttttttgtgcgaaatgtgcagctatcgagacgacgctgtggtcgatcgaatggggaatcgcggagctggtgaaccacccgagcatccagtcgaaggtgcgggaggagatggcgtcggtgctgggcggcgcggcggtgacggagccggacctggagcggctgccgtacctgcaggcggtggtgaaggagacgctgcggttgcgcatggcgatcccgctgctggtgccgcacatgaacctcgccgacggcaagctcgccggctacgacatccccgccgagtccaagatcctggtgaacgcgtggttcctcgccaacgaccccaagcggtgggtgcgccccgacgagtttaggccggagaggttcctggaggaggagaaggccgtggaggcgcacggcaacgacttccgcttcgtgcccttcggcgtcggccgccgcagctgccccgggatcatcctcgcgctgcccatcatcgggatcacgctcggccgcctcgtccagagcttcgacctgctgccgccgcccgggatggacaaggtggacaccactcgagaagcccggccagttcagcacccagatcctcaagcacgccaccgtcgtctgtcaagcccatcgacgcctaggtcgatcgatcgacctactcaaatcaatcatgtgctgcgtta

SEQ ID NO: 26 - DNA, 2034 bp, Oryza sativa
  misc_feature (0)...(0): line 1C-038-56 - genomic DNA without T-DNA insert
taaatatatgctaatttaagtaggtgtggtatggggggcttgatttggtgcaaatctagagttctgaagatggacctgacctgttggttggtctcttcagagtttttaggagttgagacaaagccttgtcttttagcctcctaggatccatctatgttggtccaaatttcctttcttataagctgtgaaagaaaaaactagtaattttccaaccttcagatagagttcagaaacaggaaccaattcccaatacagcaaaagtacctagtactgtaaccctgaaacaacaaaatggaaataaatatttttttctctctctaaatttgcactcatggatagtatcagataattacaaatatgtggtgatgttaaccatagcctgtacagttgtatcctcctgatttatacccaaaaaaaggctaaaaaatttacagtgtttatcaagaactatagtaattatcacttcttcagaactgatcaactaatttgactgttgctaatttgcaggatgttcagcagcatagtggcagtagcagcagctcaaccgaatccgacgtccaagaaaccgccgctgtcgccgtcgccgacccctccccgcggtcggaggtcgtcgacggcgagagcccgccgcagccgggcggcgaggcggcgagccatcagcagcagcagaaggagatgaagctgaagaagccggacaagatcctgccatgcccgcggtgcagcagcatggacaccaagttctgctacttcaacaactacaacgtcaaccagcctcgccacttctgcaagcactgccagcgctactggaccgccggcggcgccatgcgcaacgtccccgtcggcgccggccgccgcaagaacaagaacgccaccgccgccgcccacttcctccaccgcgtccgcgcctgcgccgccgccgccgccatgcccgcggcgccccacgacgccaccaacgccaccgtgctcagcttcggcggcggcggaggcggacacgacgcgctgccggtcaccctggacctcgccgacaagatgacgcgcctcggcaaggaggggctcgtcgcccatgcccggaacgccgacgccgccgccgcgtgcagcgaggtgtcgagcaacagggacgacgagcagatcggcaacactgtagcaaaacctgcaaacgggttgcagcagcatcctcctcctcctcatcatcatcatcattcagccatgaacggtggcggcatctggccctactacacctcggggatcgcgatcccgatatacccggcggcgccggcgtactggggctgcatgattccacctcctggagcttggagcctcccatggccggccacagtccagtctcaggccatctcatcatcatcaccacctacaagtgctacaccttcagtctcctccttcacactaggcaagcatcctagagagggtggtgatcatgaggcaagagatcaccatggcaatggtaaagtgtgggtgccgaagacgatccggatcgacaacgccgacgaggttgcccggagctcaatccggtcactcttcgccttcagaggcggcgacaaggtggacgataacaacgacgacgatggcaccagcgtgcacaagctcgccaccacggtgttcgagccaaagagggacggcaagacggcgaaacatccggcgatcacgagcttgccgctcttgcacaccaaccccgtcgcgcttacccgatccgcgaccttccaggagggatcttgaaggccatcttgcaagatttttttttttttgccttcatcctctctgctgcttctgaactgaacacgaagttagtagcctgacagtgctgatttttttgtgaatcagacagagtatttgtatactaggggtatttgggggaagtgtatgacatagctacttgtggagatatagttgtaaccataggtagcatgtaaatactatttagaaaatagctgtttaaatggatatagctagtatttgggtttggagccaggagataagtaaggcccctggttttgtagataaataagaatcaattag

SEQ ID NO: 27 - DNA, 4284 bp, Oryza sativa
  misc_feature (0)...(0): line 1C-041-47 - genomic DNA without T-DNA insert
agaacagcgccaccgctcagcacgtcacctgattaggctttcaattgggccgaaatgagactgcagatcacgtgggccgcacgttgcgttgcagtccaatttcagcccgacccggcccgtactcgaggcccaactagaccggttctacccagcccagccttagcccagaccggcccgagaacgggaagtgaaaacgcaaaggcggggggccgagcacgaaaactcgaaatagcccgcgacctctctctctgtctccgccgccgccgcctccgccgctcgccgcatccgtcggaggcgagatgggcatcaaggtgagcgcgggaggggggggggtgccgcgccgtcctcgttcggatttgtgaccggtggtgtgggttgtagctgtttttgcggccgattcgtgatgggttttcgtctcgattttgtgcgggtttttttttgttagggtttgacgaagcttttggcggacaatgcgcccaaggcgatgaaggagcagaagttcgagagctacttcggccggcgcattgccgtcgacgccagcatgagcatctaccagttcctcgtatgcccccatgtctcctctcacattctgcgagttggctttgaaatgtgctcacttacttggggtttagggtttcttaagcctttgattgcgcgagagagattgtatttttttctatttaactgcccagtgttgtaaggattcctcatttcaaattgctctcttagaccaaatcgggttgggtgaaattgggatacctccattgtgcctcatattttcttatttggtcggctatatttattaacattaaactaaacacctctagcattggatgttttgctttaaaaatgggagtagagcatagcaattccatattacattcactatatgttctgtgtgcaaatgcttgacaagttttggtgaggtattaggaaatccttacccactgctttataagttgggacagctttttatgttttacttgtatagattgtagttggaaggacggggatggaaaccctcacgaacgaggccggtgaagtcaccaggttctaaatattaatcttacaccgtttctagtgctaggattctgttaattcttttataaattagaactttttgctgtgcagtcatttgcaaggtatgttcaaccggacaataaggttgctggaggcaggaatcaagccagtgtaaacccaaattcctcatgtgttgttattgaaattatccatataaatacacttaaaattgtgacactcatgcagatatgtttttgacggcaagcctcctgatctgaagaaacaagagctcgcaaaaaggcatgatatgtttctttcatccttagattgtttttcctgctgctacttataacttgtcctatatcccattttgggctatgatctgtttctaacatggcatattctgcaggtactcgaagagggaggatgccaccaaagaactaacagaggcagtagaggtgagctaacagtttgttgtgctatttacaatgttctattatgttctgtcctgtgcttgttctccttccttccttgatccccagagaaatgacttttaatgtgcaggaaggagataaagacgcaattgaaaaattcagcaagagaactgtgaaggtgagaaggggggttgatcttacacactaaaagaaatgtaaaaaaaaaaaaaatctatgatgtttgagggacaacagattcattcgctgagaaaaatatcaatgaaaacagttggtttgaataacatgatatggaagatgcaaaaaaggtaagtatgagagtaaccttatctttgcatcttctttttccttgagcaggtcacgaagcagcacaatgaagaatgtaaacgactattaagacttatgggggttcctgttgtagaggtaaatccaattttctttggtgccacatgctttttcagggaatgagtcaatatataatcgttggatttgtttattttactatgctagcataaaatctatgatttatcatgtatttgccatgctctcaatctcatccaaagacatcttctttccggtaccaataggcgccatgtgaagcagaagcagaatgtgctgcactttgcataaatgatatggtaatgttaacatagtggacttctgccttcttgtttccatctcttgcttccatttttattaattaatgttttttttataggtttatgcagttgcatcagaagatatggactctcttacttttggagctccacggtttcttcgtcatttgatggatccaagttccaagaaaattccagttatggaatttgaagtcgcgaaagtatgcagcactgtgatttgcttggtacagacactgatgttgtacacacttgaccaattttcatattgcaggttttggaggagcttgaactcactatggaccaattcattgacttgtgcatcctatctggatgtgactattgtgatagcattaaaggtatttttgcttttcctcttctcatgagtagtattcctataaattcttgcttgcttgaatttctgtccatgcaaaccttttttcctaaccaactatgtgtgcttgtccgctaggtattggaggtcaaacagctctgaaacttattcgtcaacatgggtccatagaaagcattctggaaaacataaataaagacaggttaatcttttgaactatttgaatatttcccacttgactgagaactcaactgagacatgtatatcagctgtgtggcaaaggcagcccaaaattttctatatgtgctactttatttcatttcttggtcgcattatcttacttagatgtaatgatgtattgtaatgtgatgaagaccatttcctatgctaatttgtgctaaattttgatcatatctatcgcctactgcagatatcagattcctgaggactggccttatcaagaagctcgacgcttgttcaaggaacccaatgttacattggatattcctgagctgaaatggaatgctcccgatgaggaagtatgcctgtcacctttttttccttgccttcaaattttacaagtgatgtcgtcccaatactttaattctttctccagggtcttgtacagtttctggtgaaagaaaatggtttcaaccaagatagagtgacaaaggcatgcacttttcttctgacttactatgttatattcttgaaatatttttatcagcaatttgtttctccggtttatgtaggccatagagaagattaaatttgccaagaacaaatcttcccaaggaaggtgagacgtgagcattgtggtacatttttatactttggttctaagagtgttgcatgcctatatcatttgttaaaccataccaactcttgaattaatgtgaccaggctcgaatccttcttcaagccagttgttagcacatcagtgcctctgaaaagaaaggtaccttcacctgaatgccatcaatcttgtcgtgcttaaatttagaacttttagccttctccaggttcatccattgtagcatgatatacaaccaattcatgtcattgaattgcgtactttatctgtccattcaactaacaatgttgaatcacgtggtttgatgttttgattgtccaatgtacctgccaaataacagatcacagatgaatgataaaaatatgtccattagaccagtactcaacaatatatttgcaagcagccagaagtaggcagatacaatagccaaagctgaatcttgtaagccatgctaaaatccttctactatgagaaggttatagccttgtttggcatgattttctcgcagatcttgtactgttccatgtaaaactgcaaccatagtgctatactatatgtaggcttgcaaatgttgtggccataattatatttttgtagcaatattatgtctcatgatttgtttctatgatataatggcaggacacttcagaaaaaccaaccaaagcagtcgctaacaagaaaacaaagggagccggcggaaagaagaaataagttcgattgtgccattcatcaacaagacctcctgcacgtgtcatttcaattttaactcccatgtttgtaactcatcctccatctgatgttttctatgttcctagtaaaccagaagtccagaacttcgtctaatactagttcatctatgtagtacgtctctcttctctactagggatttgtagtgttggtttggttgctgcgtagtgtatgaagaaccaacaaaattggctttgccgcgcaaaattaactggttccgattcggaatcccggacagtttccgtaacacggggctctatttaa

SEQ ID NO: 28 - DNA, 3234 bp, Oryza sativa
  misc_feature (0)...(0): line  1C-064-20 - genomic DNA without T-DNA insert
atatatatatatatatatatatatcgttaaattattaatagaaattaaataatatatatgagaaaatatgaatgataaattcatgaacaagaatcccaaatatataaatgacaaattacataaaagatttctcatttctcaattccgttgggccatgacctcgaggctcgttcaatattccttggtccatttcagagcgtcagcccaaacgctctcgttggttgccttatataaggacgacgtgcaagctagcgttcgttctcccgagaggccgatatactcgcctcagattgtggagagatggcgtcggcccccggcgacggcaagcagggcggcggcggcggcgggccggcggtgggcatcgacctcggaacgacctactcgtgcgtggcggtgtggcggcacgaccgcggcgaggtcatcgccaacgaccagcgcaaccgcctcacgccctcctgcgtcgccttcaccgccgacgacgacgacagcttcgtcggcgacgccgccttcaaccagtccgcactcaacccaaccaataccatctttggtgagaaatagtagctgttttctactcaatcagtaaaaaaaaacaacaaatctaaaatatgatgtgatacattataacacggtactacacatctaatactggattgactttttattcaacggttggagcagcttgtttccttcctcactcttggtgtcttctgctttcgtcacaaggagtgtcaatacaattcaagtaacaatcaatagtctcaatataatcaaagtggcaacgaatagtatgtgtgtcagtcaaatcaaaatttggtcattaatttatcctgattttctgaggtattcatgtagacattttttttctctcttatgagtgtatagatatattcttagactagattattagatgtgaagtggatttgtctactacaccacataaaaaaaataccgatcataccattgaaaccattccaagaactagcaggaacgagtatagcaagcaagtataatatattatatttgtacggttgcagggtatacaagcctcttaaaatttctctttctgccttttcacatttctgggggttttcttttttacctttgtttttactcgtctataggtctaccttttcattatacgagactctttagttcccaatacgactatttagtttccctatgcacttctagtaattaaacacttctgccatataaacctgtcgttcttcaaagcatgtttcgcaagtcattgaattgttgcaagcaccaaagcaaaacttgacaattaattagagcaaattaagataatgtagccttcaaatttcagggaaaagaaaagaatctggtggattagattcgtagctgcaagatcaatatttttacagaaatggtataggagtctcataaaaaaattatcgcaactagctaggctatttatatttcacccgattgcttgttaccgtatgattcaaagttaattattactactgcagaagtgaagcgactgattggccgccgattcagcgatgattctgtacaaaaagatatcaagctttggcctttcaaagtcgtggcaggtcaagaggacaggccgatgatcgtggtgaggcatgaaggcgaggaaaggcagttcatgcccgaggagatctcctccatggtgctcgccaagatgagggagacggccgaggtgtacctcggcaagacggtcacgaaggccgtcatcactgtcccggtctacttcaacaacgcgcagcggcaggccaccatggacgccggcgccatcgccggcctcaacgtgatgcgcatcatcaacgagcccaccgccgccgccctcgcctactgtctcgagaagatgcccgtcagcaacaaggggaggatggtgctcgtgttcgatctcggtggcggcacattcgacatctcccttctcaacatcgatcccggtgtcaacatcgatatgggcctcttcgaggtgaaggcaccgccggtgacactcaccttggcggagcggatttcgacaacgagttggtgaagcactccttgcgagagttcaatcggaaacatgggtcgatggacattgaaagcaatcagaaggcattaaggagattgaggaccgcctgcgagagagcgaagaggatgctgtcatccacgatgcagaccaccattgaggtagactcgctccatcaaggcatcgacttccgcgtcaccctcacccgatcccgatttgaggagctgaacaaggatctcttcagcaagtgcatggaggctatggagaattgcctccgcgacgccaaggtggacaagtggagtgttgacgatgtcgtcctcgtgggtggctccacccgcatacccaaggtgcagaagatgctgagtgagttcttcgacgggaaggagctctgccgcagcatcaaccccgatgaagccgtcgcgtatggcgccgccatccaggcctccattttatgtggtggaaccgatgataagaggttggttgatatgcttctccgcgaagtcacgccgctctcgctaggtgttgagactgaagataattgtacaatgagcgtggtgatcccaaggaacactgcaatcccgaccaagaaggtgaaaaacttcactactctctacgacaaccagatcaacgtgagctttccggtgtacgagggtgagagcgcaaacaccaaggacaacaacctgctcggcgagttcacgctatatggcatccccccggcacccaagagagtgccatctatcgatgtcactttcgacattgatgccaacggggtcttgaacgtttctgccgagcacaaggtcaccggacagaagaacagcatcaccatcactaaccgcagcggacggctgaaccaggaggaaatcgatcgcatggctctggagcccgagaggcacaagatgaagcggatcaagcttcacgaagtagtctaaatgaactgaacattgaaagtgattctgcgacctaccatgatcaaagaagtatcatcggcatcctaatgcaatgggctagctgctacaggggtttgattattgggtgcttaacgctagtattgtcatgcaacttctggcttggcacatgtatggccaaatcctagcttaagcttttattaattgtttctgggacaatgttttaattgctgatattggagtaatcttctgtatctgtttagtgtcttgcatttgctgatctttggcagtagtagtttcagttttccttcgatacacccagtt

SEQ ID NO: 29 - DNA, 3677 bp, Oryza sativa
  misc_feature (0)...(0): line 1C-109-35 - genomic DNA without T-DNA insert
aaaattaaaaacataagtcacgcataaaatacattcatgttttatcatattataacaataaaatactaattgtaaaaaagttttaaataaaacggacaatcaaaattagacatggaaacttatggttgaaatgaggcagagggagtacagtaccaggctactagccaagcgagtcaaaatggttaatcgatcgccacctggaccatgcatcctctctatataagctgccctctaactagctagctaggataccaaaagaaaggcataccaagcttagtgtgtgttgaatcagcgacgaagatgtcgtcgtcggcgacggtggtgccgctggcgtaccaggggaacacgtcggcgtcggtggcggactggctgaacaagggggacaacgcgtggcagctggtggcggcgacgctggtggggctgcagagcgtgccgggcttggtggtgctgtacggcggcgtggtgaagaagaagtgggcggtgaactcggcgttcatggcgctctacgccttcgccgccgtgtggatctgctgggtcacctgggcgtacaacatgtcgttcggggagaagctcctcccgatctgggggaaggcgcggccggcgctggaccagggcctcctcgtcggccgcgccgcgctgccggcgacggtccactaccgcgccgacggcagcgtggagacggcggcggtggagccgctgtacccgatggcgacggtggtgtacttccagtgcgtgttcgccgccatcaccctcatcctcgtcgccggctccctcctcggccgcatgagcttcctcgcctggatgatcttcgtcccgctctggctcaccttctcctacaccgtcggcgccttctccctctggggcggcggcttcctcttccactggggcgtcatcgactactgcggcggctacgtcatccacgtctccgccggcatcgccggcttcaccgccgcttactgggtccgtactaacagcctcatctttttgcttatacttatactcatcagccagtatttaaattttcaatcataaatttaaagctaattttagagtttttttcatcgaagtttatttttcatcatttgtttttaggtcgctaagaacacgtatataaaagttttattcacaaattacttatcgttcacgactaagctaaacaatagtactccacttttgccgccgccgccgacgtcgacgtagctctgacaagtgtacatatcaatgtgtggctggcaggtggggccaagggcgcagaaggacagggagaggttcccgccgaacaatatactgttcacgctgacgggggcagggctgctgtggatggggtgggcagggttcaacggcggcggtccgtacgccgccaactccgtcgcctctatggccgtcctcaacaccaacatctgcaccgccatgagcctcatcgtctggacatgcctcgacgtcatcttcttcaagaagccctccgtcgtcggcgccgtccagggcatgatcaccggcctcgtttgcatcacccccgctgcaggtacgtaaatcatcacactacacggaaaacgaagccttattaattaagtattagctaaaataacttaaaagtaaattaatgtgatattttttcttagaaaaattctgcgaaaatataccatttagtaattttgcaatagtgtgtaagaaaaacgagagttgaagttgaaaaaactgagtaccgaacacagccttatattaatcaaattgttgtgtagtactctaattaactttgggatcgatgaactataacgtgatggcgcgcgtgacgagcgcggatcgattcttaattagattagattatcccggtttgcagattaatgatccttcccgcccaggttgttttgactttgaattctaaactgtctgcgttgccaacaatacaggaggttggttaccttgcctgccaaataatctgcattttccccgtagattacgccgtttaccattttgtggcaactaattaagcacgcacacgcaactgattcatcatatctctctctcactctcttgtagtggaagagtcgtaagtttttcatctctttgtcaagttgttgattgatgtagtggagtagtggtttaatttccggtcactagtcactacagtacctaatgtgaacaaatacaaacgttcatcggaatctaggttggttgatgcatacatatttcgtccgaaaccaggaatcttatttgtcgttcgtgcttatgtcaatagacaatacctatcactagctcgtagtactactaaagtcaaagtcgtctgaaactgccaatttgtttttttctgagtttttcttgtacgcaagcagccaagaaaagcacactgaaaatgaaagtagaatattttacagcctcccattccgtcggtgacttcgttcaaagtctatttacgcggcgaggatggactacagtactccatagcaattgtcgatgacatcaggcactgtcactgctatcgcaaaggatccgtgtccttatttatgacacacatgacacacctacaccccttatcattatatttgtttttagatttgtgcgtataatacagatttcaatagagcataacattatgatttttcttttccgtacaacacacgaatctcaataaaatatatactcttttcgtatcaaaacatagtaactttgtactgaattactatctctgtcctaaaataaattcatttttaaatttctatgtccaacatttgatcatccgttttatttaaaaaaattataatttttttataaaatagtcatacataaagtattatttatattttattatctaataacaataagaatattaactattaaaaaataaataagacgaagaatcaaacggtgtatggattggtattgcaggggtggtgcaggggtgggcggcgctggtgatgggggtgctcgccggcagcatcccgtggtacaccatgatgatcctccacaagcgctccaagatcctgcagcgcgtcgacgacaccctcggcgtcttccacacccacggcgtcgccggcctcctcggcggcctcctcaccggcctcttcgccgagcccaccctctgcaacctcttcctccccgtcgccgactcccggggcgccttctacggcggcgccggcggcgcccagttcggcaagcagatcgccggcggcctcttcgtcgtcgcctggaacgtcgtcgtcacctccctcatctgcctcgccatcaacctcctcgtcccgctccgcatgcccgacgacaagctcgaggtcggcgacgacgccgtccacggcgaggaggcctacgcgctctggggcgacggcgagatgtacgacgtcaccaagcacggctccgacgccgccgttgcccccgtcgtcgtatgatcgatcacattaattgcatgtgctgtcacctgatgaaaacctcaagaaattcatacacacagaaattaatactacgtgtttgcttctactcgttaattatgaaaatctatgttcaagttttacgtgtatttaacagcgcgcgtgtggagttaggcattgcactttgcagattcaggggagtccggttctgtatttttgttagtttgtaaaaatgtatacgtagttttgtatccttactggatctgaatgaacacaagtgtaatattatattttgttaattaagctccatattttccaaaa

SEQ ID NO: 30 - DNA, 8093 bp, Oryza sativa
  misc_feature (0)...(0): line 1C-109-51 - genomic DNA without T-DNA insert
tctccctaacgaaccgacctgagttagcttatgtcgtcatcttttagggctcgcctcgattcgttccgcgagaaatccactccctcccatcccatcgccgccttttccttttcctccaaatccaaccccctcttctcccccttctccggcgcggcggcggcggcggcatcaccggaggcgccgaccctccctcggctcgccaaaaggtaaacaccagctatccctcttctcctcctctcctgcttctccttccgcttccgcacgaaaaaaccaactgaaaaaccctatcgatctgcagccatggcgaacctgggcggcggcgccgaggcgcacgcgcgcttcaagcagtacgagtaccgcgccaactccagcctcgtcctcaccaccgactcgcgcccccgcgacacccacgagcccaccggcgagcccgagacgctctggggcaggatcgaccccaggagcttcggcgaccgcgccgtccaggccaagccccccgagctcgaggagaagctcaccaagtcccgcaagaagaaggccgccgcctctgaccccgacgacctccaccgccgcgacgccaagcgcaggcgccgcgccgcagccgcccagagtgaggtcagcgtcctctcgctcaccgacgacgtcgtctacaagccccagaccaaggagacgcgcgccgcctacgaggccctgctcagcgtcatccagcaacagttcggcggacagccgctcgacgtcctcggcggcgctgccgacgaggtgctcgccgtcctcaagaatgacaagatcaagagccctgacaagaagaaggagatcgagaagctcctcaaccctatctccaaccagatgttcgaccagatcgtctccatagggaagctcatcacggatttccatgatgcctcggctggtgattcagctgctgcgccatctggtgatggcatggacacagcgctggatgatgacatcggcgttgctgttgaatttgaagagaacgaagatgacgaggagagcgatttcgatcaggtacatttggtgtttctctttacaattcttttcctgcatatggctaaagcatgttatgctagtgtttacttacaaggccttcattatttctcctcgtcaggtgcaagatgatttggatgaagatgaagatgatgacttgcctgagtcgaatgcccctggtgctatgcaaatgggtggcgagttagatgatgatgatatgcagaactccaatgaggggctgaccataaatgtacaagacattgatgcttactggcttcagaggaaggtctcgcaagcatacgaagatattgatccgcagcacagtcagaagcttgctgaggagatcttgaagataattgctgagggtgatgacagagatgtcgagaatcggcttgtcatgctgttggactatgaaaagtttgatctcattaaactgctactacgcaaccgactcaagattgtttggtgtacccgtttagcgagggctgaagatcaggaacagcggaagaagattgaggaagatatgatgggtaacccaactttaactccgatattggagcagctacatgcaacgagggcatctgcaaaggagaggcagaagaatctagagaaaagtatcagggatgaggccaagaggcttactaaaagcgaaaacactggcattgatggtgcgagggatcgtagggcagttgaccgggatatggagagtggatggttgaaaggccagaggcagctgcttgatcttgacagcctgtccttccaccaaggtggtctcttgatggccaacaagaaatgtgagcttcctccaggatcattcagaacccctcataaggggtatgaggaagtccatgtgccggcgctaaaagctaagccatatgaaactggagagaagattgtcaagatatctgatatgccagagtgggctcaaccagcttttgctaagatgacacagctgaacagggttcagagcaaggtttatgagactgcccttttcaaaccagataatattctcctctgtgctccaacaggtgctgggaaaaccaatgtggctgtgctcacaatccttcagcagattgggttgcatatgaaggatggagtgtttgacaataccaagtacaaaattgtctatgtggccccaatgaaagctttggttgccgaagttgttggaaatttgtcggctcggctgagtgcgtatggtattactgtaagagagctcagtggagaccagaacctgaccaaacagcagattgatgaaacacagataattgtcaccacacctgagaaatgggacattgtcacaagaaaatcaggtgacagaacctatactcaaatggtaaagcttctaatcattgatgagatccatctacttcatgacaacagagggcctgttctggagagcattgtttctagaactgtccggcagattgagacgaccaaagaacatatccgtctagttggtctctcagcaactcttccgaactatgaagatgtcgcggttttcctgcgtgttcgctctgatggcctcttccatttcgataacagctacagaccttgcccccttgctcagcaatacattgggatcactgtgaggaagccactacagaggtttcagctgatgaatgagatttgctacgagaaggttatggcatctgctggaaagcatcaagtgcttatattcgtgcactcgaggaaggagacagcgaaaactgcccgtgccatccgagatactgcattggctaatgacacgttaaaccgcttcttgaaggatgatagtgcaagccaagagattcttgggagtcaggcagaactagtaaaaagcagtgaccttaaagaccttttgccttatgggtttgctattcatcatgctgggttggcaagggtagaccgtgagcttgtagaggaactttttgccgataagcatatacaggtccttgtctcaacagccacccttgcatggggtgtcaatttgcctgcacacactgttataataaagggtacccagatttacaatcctgaaaagggtgcgtggacagagctgagtcctctagatgtcatgcagatgcttggccgtgctggcaggccacagtatgatacacatggagagggaataatcctaactggccacagtgaattgcaatattacctgtctctaatgaatcaacaactgcctattgagagtcagttcatatccagattggctgatcaattaaatgcagagattgttcttgggactattcagaatgctcgggaggcatgctcatggcttggctacacctacctctacatccggatgctccggaatccaacattgtatggtctaccggcagatatcatggagactgataaaacactagatgaaaggagagctgacttggtaagaggataccatacttatgttctatcttctatagagttgttatacttatcccttcatccttagcgtttgttcacatattatccttgtacatattcactaattgccacaatgttctctaattcgttgtccaagttttctttaactcttatctgaatattggtgttcaagagttacatgaatctaatatagcatttactgaggtactcaattctcagtattggaatgctgtttacagaaagttaattccctcacactggaatagggtatttttaatcatcactagcatcatagactcattagcatgtactagtgtataatagaaataggataatggccttggattttatttgtccgaatgctctttgcagtgtagctttatattttgttgctggacacttaatagttgtatatttgctctctttgaccaataacaacatgatacaagccagtctcttctattgaaatgtatgcgcatctttgactactgatgtagcattgtacatcaatttagcactcgaactacagggtaaggtagcatgtaaaaaatgcgtaaaaagtgcgcaacttcaactcattatttttgttggctgaattccatagctgaagcatctagttgacaattgattcatgcttgtgttgttcatattgtatggtttctctgtatgctctcttgatgtttaactgttttgttaccccttctagtccatgctcaatcaattgttgattcttgtacttctgtgcttctgcaggtgcactctgctgcaaatctactcgataggaacaatttgataaagtatgacaggaaaacaggatacttccaggttactgacctaggaaggattgctagttattactatattagccacgggactatttcaacttacaatgagtacctgaagcctacaatgggtgatattgagctatgtcggctcttttcactcagtgaagaattcaagtatgtcagtgttagacaagatgagaaaatggagctggctaagcttttggatcgtgtgcctattcctgtgaaagaaagcttggaggagcccagtgcaaagatcaatgttctgctgcaagcatatatttctaggctgaaattggagggtctttcgcttagctccgatatggtctacatcagacaggtaacatgttctccctgctgaataagcttcctgaatttgaattgattgcttattattatctgttttatgacaattgattgcttattattatctgttttatgacacagagtgctggccgtctcttacgagcgttgtttgagatagttctgaagagaggatgggcccaactagcagagaaggctttgaatctttgtaagatgatagataagcagatgtggaatgtccaaactcccctgcgtcaattccctggtattccgaaggaaatcctaatgaaactggagaaaaaagaattggcttgggagaggtactacgacctatcatctcaggaaattggtgaactaattcggttcccgaagatgggcaggcaactgcacaagtgcatccaccagttaccaaagttgaatctttcagcccatgttcagccaattactcgtacggttttgggttttgagttgactataactcctgatttccaatgggatgataaggtacatggatatgtggagcctttctgggttattgttgaggacaacgatggcgagaacattcttcaccatgagtacttcatggttaagaaacaatacgtagatgaagatcatacactcaacttcacagtgccaatatacgagccactgccccctcaatactttattcgtgttgtatctgataagtggcttggttctcagacaattcttcctgtctgttttaggcacttaattcttccagaaaaatatgctccgccaactgaattgcttgatctgcagccactgcctgttactgcattgagaaatgcacgatacgaaggcctttatagtgccttcaaacatttcaacccaatccagactcaagtgttcactgttctgtacaacactgatgacagtgttttggttgctgcgccaacaggcagtggaaagaccatctgtgcagagtttgctatactaagaaaccatcagaaggctgtatctggtgagagcaacatgcgagttgtgtatattgctcctattgaagcccttgcaaaagaaaggtacagggattgggagcagaaatttggagaatttgctcgggtagttgagctaacaggtgaaactgcagctgacttgaagcttctggataaaggggagattataattagtactcctgagaagtgggatgcactctctcgccggtggaaacagcggaaacaagtccaacaggtcagcctattcattgttgatgagcttcatctgattggatctgaaaagggacatgttctggaagtcatcgtctctaggatgaggcgtattgcaagtcatattggcagtaacatccggattgtggctctttcagcgtcacttgcaaatgctaaagatctcggagaatggattggtgctacctctcatggccttttcaactttcctccagctgtccggccggtgccattagaaatacacattcagggtgtggatatagcaaattttgaggcaaggatgcaggcaatgacgaagccgacatacactgctattacccaacatgcaaagaatggtaaacctgccctggtgtttgtgcctacacggaagcatgcaaggttgactgcattggacttgtgtgcgtactcgagtgctgagggtggcggaacacctttccttcttggttcagaagatgagatggatgctttcactggtggtatcagtgatgaaacacttaaatatactctgaaatgtggtgtaggctacttgcatgaaggtttaagtgatctggaacaggaagttgtaactcagctgtttctcagtgggaggatccaagtgtgtgttgcgagtagcactgtgtgctggggaagatcattgcctgcccatctggtggttgtgatgggaacccagtattatgatggccgggagaatgctcatactgattatccaatcactgatctactccaaatgatgggtcatgctagcaggcctcttcaagataactcagggaaatgtgttatattgtgtcatgcgcctcgcaaggaatactacaagaagttcctttttgaggccttccctgttgagagccatcttcaccacttcttgcatgatcatatgaacgctgaggtggtggttggtgtcatagaaaacaagcaagatgctgtggattaccttacttggaccttcatgtacaggcggctgaccaagaaccctaactactacaatctgcagggtgtgagccacaggcatttgtcggaccatttatctgagttggttgagactgtattgaatgacctagaatcaagcaagtgtgtggctatagaggaggatatgtacctgaagcccctcaaccttggtctgattgcttcgtactactacattagctacacaactattgagaggttcagttcaatgctaactcaaaagactaagatgaaagggctcctggagattctagcctctgcttcagaatatgctgagcttcccagtcgccctggtgaggaggatttcattgagaagcttgttcgccaccagagattttccatcgagaagcccaggtatggtgatccacatgtcaaggctaacgcactgctgcaagctcatttttcaaggcacactatcttagggaacctggcagccgaccagcgggagatactcctttctgctcatagattgctccaggcaatggttgatgttatctccagcaatggatggcttacgcttgctcttaatgcaatggagctgagtcaaatggtgacacaaggcatgtgggatcgtgattctgttcttttacaacttccacatttcaccaaggagttggcacggagatgtcaggagaatgaagggaggcccattgagagcatctttgatctggctgagatgagcatcgatgagatgcgggatctgttgcagcaatcaaaccctcagctgcaggatatcattgaattcttcaagcgtttccccaatgtcgatatggcttacgaagttcgcgagggtgatgatatcagggctggtgacaatgtaactgtacaggtgaccctggagcgtgacatgacgaacttgccttctgaggtggggccagttcatgcccccaggtacccaaagcccaaggaagaaggctggtggctggtaattggtgatagctctaccaatcagttgcttgcaatcaagagggttgcacttcagaagagggctcgagtgaagcttgagttcaccgctgcttcagaagctgggagaaaggagtacatgatctacctgatgtccgattcttatctgggctgcgatcaggagtacgagttcaccgtcgatgtcatggatgctggaggggattgatcccaaggccagaaagaggatgaaagctttgcccagcccccaggtgtgtatggagctgggtggttgcttgtacgctcgtagcaattcctaatgtcctatccacacttgtatgtgtttgtactcgtttttatctttagccattttcttagtgtaactgatgctcacctctgctgctacctgtggtcatctttggggatctttattagctagcctatggctgtgcttcttgtgatcatccttcttattgtgtttttgtcttcctgtttcatcaaagcaagtcactaacgttttgattcttat

SEQ ID NO: 31 - DNA, 1383 bp, Oryza sativa
  misc_feature (0)...(0): line 1C-056-07 - genomic DNA without T-DNA insert
ttatgcagactatcaaatctttcacccctgcaaccacgggtagagtcattttgcacctccttacatcatcactattccatattcattttcgttttcttagcattatcatattgctacagcatgtaacaccctccctgatggtctgcttccaatccatgtcatgcagttattctgcagtggttagtttggagcaagcattttgagtggcgcatatgggcttcgctggtcccaatagttgggggaatactcctaacttcaataacagagcttagtttcaacatgtttggtttctgtgctgccatggttggctgccttgctacatctaccaagaccattttggcagagtctctactccacggatacaaatttgacaggtaggtgctcatcttatggcgggtgtaatgactggtgctttacttctgctgtgatcttacagttataatactgtatggtaaactggattttttttgtcgcagtgttaccagcaaagtaaatcatgttatttatgacgtttccttgtgttttcttcagcattaacacagtgtactacatggcaccctttgccaccatgatactggctctaccagcagtgttacttgaaggaggcggtgtggttacctggttctacacacatgactccattgcttctgcactagttatcatcataggctctggagtgcttgcattttgcctaaacttctccattttctacgtgatccattcaaccactgcagtgaccttcaatgttgctggcaacctgaaagtagcaaccatttggcctatgctttgttcttttcatatattctgctcgatcatttcttagaactgatcattgcgctgcaggttgctgttgctgtattggtgtcatggttgatcttccggaaccctatctctcctatgaatgcaatcggatgcgcgatcacgctcgttggctgtactttttatgggtatgtgaggcatttgatctctcaacagcaggctgtagctccaggaacaggaagcccaacaacatcgcaaacaaattcgccgagaagtcggatggagatgctcccccttgtaggcgacaagcaagaaaaggtttaacaaaagaatacactttgtactatgcaatagtcattatggtctggatacccttgtagttcgaactattgtgttccttcaagttactgttttcaaattggccagaaactgccgcacgctaaacttcctttctgtgtagtgcatacggaattttttctactactaggatgtcacgcaagtccagcgtcattttctgttgctagacgtgtagcgcactgaatgaacagttggatatctcatctaaccaatttgtcttaattgtgttgggaatgtgactaagctaaccaattcgtagtaattgtg

SEQ ID NO: 32 - DNA, 7726 bp, Oryza sativa
  misc_feature (0)...(0): line 1C-100-32  - genomic DNA without T-DNA insert
gaaaaattgtttgaataacactgttacatagtgtatgtactgtttcaatagtacttcacttgtactcgcttagtctcgaaatatatttatgtaaccagctctacaaatatttgaatattggatgaagaagaaagaggctggcaaaaaaaaggccgaaagtaaagtaaggtgaagaccgataaataaatgggccgaaagcccatttagttagttttgctccgctgaatgccctgaaaccacacgaacacgcggctttgcttcggtttcgttgcccctccgccgccgccgccgccgccacccatggagatggagatggaggacaacaaggcgccctcgccgccatcacctatggagtccgacgtcgctgaggagaagcgcaagagggaggacgatgcatcctcctccgctgtacttgccgccgccaacaacaccggcggtgcccagcaccccatgtggaagaccagcctctgctccttcttccgccgccgcgccgcctcctccgccgacggctgcagccacggcgactcctgccgctacgcgcactccgaggaggagctccgcccgcgccccgacggcacctgggaccccacctccgaccgcgccaagaagctccgcaaggtcgccgccgacgaggtggaggaagaggtggtcaccattgacgacaaggccctggacaagtgcctcgtcggcctcccgaggggatgggccaacgacaggctcaagactttcctccaagacaaggcaagaactaactactcctccattcttcctcctgctctactactagttttttcatcgttctttcttgttttaccactgtctactccaactagtacaaaattctcacactctttcgattatatcttcctgttttgcaccaacagatattggttcagaatgagtttagttgataagctctagaatcgaaactattgatcaacacatgattcaagtccttacagctgatatcacttgagttagcttggtccaaaagtcctttgacaagcaagaaagtaacttttcatcaccttagcttagtctggtatttttgtataggttgaaatagattactatacgtcgcaccattatattattttaagataagagcatctcaaatacattgaacaagtaacttttcaaattggtgagattataataagtttatctgttttgcaacatgtggatgtggctatgatgacattaaaatgaaggtatgccatggtatattggtcatctcgtaatgttgtgtcattttgattcatagaaacttgaatggatgaattgcattttttaaaaaaaaagtcaaaccttgatgtttgttaatagattatgacatgaacacatgacacatggaagtacactgtttgagattttgagtaatactggaatcattgatccagtcataaataccttaatacaagtcatcccaataattcctgtataccagtgattctacctaaaattcgaatccgagtagctggttagatgatagcatgaaccaaattattccattcagcatactccaattaatgcatgcttgtacttgtagtaagtatacatgaacgatggaattcttttgcattacgtccctcaactcttgttgatattgtggacaaatagaagcatggtcaaatattggaagcttgtctcaaagcttcatcttgctctcccagatataaattcaacttttcttgcactaatgctgaaatactactgcttggttcaaccttgagttattgaaaaatgtgcgatgttcgaaaataattcagaaactaggactataatttttagggttatgataattaattttgagaaatctttatcaaggtctgacatggtcaatactaaaagagaatcggtttagtttacagatatttcacagtggaaaggagtttttttcgtaattttaagttgtgtggtatgcatatttatttagaggcttctgaaatctgtacttgtttcatttcttttgtacaaatacacttgattagtttggctagtcttttataaatatataacagtacttacagtatccctagtgctaggaaagaaatcatcttcagtacctgtatgaagcacatttagaagtaatggattaagggacaaagaggtgatgataataaccatgcagatatcatgtttatctggtttgtaaaaatattgaaaaagtaaactcccaattttggctttttaaaaaaacttagaattgcatgcttatcttgtgtaatactccttccattttactattttttgtccaaggctttcatgcccattatgcacatagaccaagcagtggtagtctactagatttaaaataaagtgggattgtataaaccaatcaaatgacatatggaatctgctcaatcgtttggctctttttcagcactaaagtagtagagagtgcaaaaactataaatatttggatagcttcattttttattttctcagtagacgaatgtgaaatgacactttccaaggaaatagtcacttgcaagacagagggaacatattttcattcctagaaagaagcatgcagtttgcagctttaaatcatgttagattatgccaacctaaatgctgtaacttcattgatggtgatgcagggaatctcgtatgcaacagcgaagaagaagaagggaatgactgttggttttgtaacttttgaaaatattgaacagctgaagaatgctatcgaggtaattattggtatgttcatattggaacgtgtttgagcaactgggaagtgacaatgactcttgcatattaacttgagtaggtacttacagagaaccaatctggtggaaaggaaataaagatagcagatgccaatcgtagatctcatcaaaagctgcacacagaaaagcctgtatctgacaatggagtgacaacagaaaatggtactagtgttgatgttcctcctggggagacatctgcacctgaagcagcaatatcaaataaaaaaagtgtccgcgatgcagttactccccttgcccacatgtcttataatgatcagctagagcacaaaaataattctgttgcgcagatactgaagaggcttgtaagatcaaaaccataatcttcttctacctttttgtttgcttgaatcattagtaacttttcttgttatctacttccgcagactcgcaatgctaggaaagcttgccctactggcattccccttccagattgggtttttaaatccaaagaaattggtagcctcctactctcattatgttgtgtttgatataactaagatatgcttagtaattgtggctctaaacatatgttctgtgataagattgtttgggtagaacacgataattttatgtactatttattcttctggtggctaataggaaaagtgccaaaattgtagtggggatttggatgatccaaatgcataaattttcttataatttccaacctaataacagttaaaactagtattatatatggcaggtttttagttaaagacttaaagttagtaaaatttcataatgcaagttctagttaaatgaaagtgctatgctcctcacttataggggaatgagtatatgtatttgattaggagtttaataccttctgtccaagcatttgtacatttcatttattccatttttcctctttagtttgtcatgattatattttaaatcatgcaggtggtcttccttgcaagcttgaaggcattctggagtccccagtgattaatggataccgtaacaagtgtgaattctctgtgggattttctttggagggaaaaaagacggttggatttatgcttggaaattttaggttggtttccctatttgttttctgtttgttgttcgtggtcagcactgatatgattgacaaaacaaaatccagggaaggtgtgactgctgttgaggaacctgtggactgcccaaatgtctcagaaatttcctgcaaatatgctctgatgttccaagactttctgcagtcatcaagcttgcctgtgtggaacagagttaataattgtggattttggcgtcaattcacagtatgatctctcaaagcttttcaactctttcagtgggtgcttatatgttcatttcctgttggtatattatacactttatttgaactattttagttctgggataataatttctcagactatggctgctgtataggttcgggagggaagatgtcgagctcaagctgttgcacagaatgcagaaacccaaatatcagaagtcatgcttattgttcaggtagtacctcattctcaaatagatggtgctttagaatgtgctctatcaaacctcataaacttttggtggagaatataaaaaattaatacattaagacttctcatgtcaaaatgacattagtagatctgtgacatctgtcatgaaaaatatatttttagtagttttgaaaaacataatactataataagtactggttcaaaggtgcgtgacaagggaacagaaggattataacatcaagaatcaaaatgcattgtatactacgagtcctgtagatcacatcagtgcaatacatgttcctactggcagtattgtctgacttcctatgttcaggtttgctccacaggtgttgatgatgcagtaatgaaagatgaatttgacaagttgaccgttgccctgcaacaaggagcagcaacatgctcacctccattacctctaacaactatagtagtgcaagtaagtttgagttctttgtctggaagtaagacttctatttatagtttcctccattgcctcaacatttcagcttgtctaaactgaccactcttataagttttattactttatactgtagatgtcttgctgtcctgtaactcaatatgctacccactaatatctaggagttaccattgtactaaaaatgtataaaacatggttgataggagttcagaaagtgcatttatctgggtacatccgatcactggtagaatatgttaccacttgaaccaaactttatgtaatttataatggtatatttgcaactacggaatcttttactgctcatgcaggatcacaaaggaatttcaaatgctgcaccagctgattgtccattgatcccactattggtgccaaaagtagatcaatcagaaggtacagtggataaaacaagaatccatgaccacatcggtaatctgtggttctccatatcaccaacagcattttttcaggttagctaaaactgcagacagctttgtccattggctatcacggttcaatttggtaatgactcactattacacaggtcaatactctcgctgcagaaagattgtatacccttgctggtgattgggccaacctcaattcgggcacattactttttgacgtatgttgtggaacaggaacaattggactgaccttggcacaccgtgttggaatggtgatttcctccattcatttcatgtgattgtgcatgatacttcttattacccgtataatgctcagccacagctgggctctttattaacttatgtagaattttggaaatgtcatggtagagaagtcaatgaatgatttagcgaattccttgtcaaatgtgcaggttgttggaatcgaaatgaatgaatcagcagtttctgatgctgagagaaatgcgctcattaacggtgtatcaaattgtcgctttgtctgtgggaaggtgaggcttatgcccttgtattcctagaagtttctatcattctattgtcactgactcactgtagccaccatggtgtcaatcctattcagtttttcttatgtcctactttatatactcaccacatcaggctcatctgaattttacatcttgggattaaacaggccgaagatgtgatggggtctcttctcactgaatatcttggttcaccgcagcaggacattcctgtttctgaaggtgcagtaagtggtactgtgaaagatgaggaagttattgacagttctaagaatagtggcgaaaatttggacagctcaatgcagaaaaatgacaatggcaaaagccagcagctcggggatgcaccagctgattcttcctcttctgccatagatgagataaaggggaattccaatgatagggttggcaatgggttggaaggcagccatgatgaatacaatgaggttgctggagaagatattcatggggaagcatcattgatcaatgagtctgttgacctgaaagtatcggactgtttggaggacagaaagacatctgatgatggttcttccatttctaacaatgatgtgactgcagctactgcatgtcagttcgaagacattgttgctattgtggatcctcctcgtgttgggcttcaccctactgtgagcaaatttcaatatcttagtccattcttgttacaattgtttgcccctttatattccaaatgcttccatgcaaacttcaaaacatgcccacaaaattgatatcctttatttttattgtccattattctaatctacatatcctaataaatttaaccaaaactatggattttatgtatatgttctttaattatgcaataaaattagtggttttgtgttttttgtgtgagatttatacctaacttttactgattcctactggttaaagttgagcatatatgcctgagtaggtataacatggttcaaagcaggcaatggaagaaccatgacctaaactggcctagatccttttctggtccatttttttactagttaaacagccaatccattcccatttcgcatgattttgatcttcaatagagatatcactaaagtgaagaaatgaagaatctagagaaaatcaagacaacatgcaactaacagataatgtaaatataatgagcttcctgtgacttttcactgtctgcatactgtgaagcattaaaccacattttttgtcacttacaatgaaactagatttgtatcccatatgcatatgaacctatccatacacatacatgtgcccaataaatgcatttctgaatgcgattgagctgtgtaccccatcatctgagaaacaggaaaagaacaaaggaaaccggggatggcgaaccatgagcagcgctggcctcgcgaggcagcgtacaaagtcgatgcccaactctgagccatttgttcccaagagagccatggctgttgatttgttcccccacacctcgcattgtgagatggttatgcttttcgagaggtgaacaagtagccggctcatcgaattctttctagagatttttgatctttgttgaaattttcatcatgttgtatgatgcattacaagacccagaagccatgacaattgattgttgctgctggcctagaacatcaatttttcatgttaaatgaattgcactcaaaaaggaccacaaccacaaagtaggcacactggaagctgaaacttaacatgtgacatgcatctgtcttatttttaggtaaaccccaaaagaggacagggagttcctgttttcatttaagaagaagagagtgacccgcatctgtcataactagagctttgatgtattggcaatcttgtcagggaaaaaccgtactttagtacaggatccagtgtttgccccatccatggcaaacaacagtgctggtagtactaatatttgtgctcagattgtaatattgtcaatagggtgataaaaaagaggttcagttccttgctactcattggctgcatctggtaaaggatggcctaacatagactaataaatatctaaatgcatggagatggagtcaatttcttgtggtgcccactagacaagaaa

SEQ ID NO: 33 - DNA, 2417 bp, Oryza sativa
  misc_feature (0)...(0): line 1C-142-27 - genomic DNA without T-DNA insert
cgggggaaaaaatgcgttgtacccaatgcattatctcctttttctttcatttagaaaaggcgggtgagggaattttatgctggttggcaccctttttccctcttttataaagtccacccgacctcacctaatgtcaaaagaaaccaacggcaggaggagggagacacctcgaggaaaccacagcacccaaagaacaagaaaggcgataccccttctcccaccaccaccgtttccgtccatctccatcctcaccgccggcgacgctcacctccgacgaccgatcggcgacctgcttcagccatggcaatggcttacaagatggtaatccagtctgtcacacaccttggttattgagtccatgggtcgtcgtcgttgcggcgttctgatggagtatgtgggttgggtgcgtctgtgcaggcgacggaggggatgaacgtgaaggaggagtgccagaggtggttcatggagatgaagtggaagaaggtgcaccggttcgtggtgtacaagatcgacgagcggtcgcgcgccgtgctggtggacaaggtgggcggccccggcgaagggtacgaggagctcgtcgccgcgctgcccaccgacgactgccgctacgccgtcttcgacttcgacttcgtcaccgtcgacaactgccagaagagcaagatcttcttcatcgcctggtaattaattaacccaacccagctcctccctctaattaatctcttatcaagtgattcgattccatcttattccatgtctgatctcgattaactagcggtaattcaggatcttgttaggaaaggtaaacacggtgagctgtatccatcgttagatgccatatctcgagcgttcttatataaggtgcatattaaatcgtctcttctgctgtccaaaccgcaacggcacaagttgttctctgaaagaactcgtgctttggaagatacagtttggaagatatgcaagcgattgataaactgcgggctgcggcaactagctaaagagaacaaatcccttttcgccttttgcactaatgaagaactctaagttcatgcaattacacaatgtacgcgcaacttacttaatacctaaacctcgattgatcatagtcgtttcgtaccataagaaaagatgtcacatatcacaatcactcatcgcgtcatgattgagaaatcaaattaattctggcaccttacatactattgtacactggcagacaagtcctcctgagccttattaggtgctgacgagcatcataaaaacgactaaaaatctgcagggcgaatgactgttccaatcaatgccgcatccttatcacaaagaaggatggataggtcaccaagctttctctattttccacaaaaagtttgcttgcctctcagacgcatccccgttgccttgtcaaacgaagctttagtcgaaatcatgacggggacccatcatctttgttaatcactgttgtctttagtacacttcatgcataattttgcttaattaacctccactgccaaggccggttcttttatcagatctgatctggcctgaatccacctgttgtcgatctgtctgtccttgtatattcatagatccatcatcaaaagcaaggttcgtgcgcacccacgccagtagtagccacagctgcagtattattgtccattcaacatttcactgtctttagtgcagttcaattacttctttcctggcatgtccttttgtcaagaaacaaagattatcagataagatcgagaggtatactttaacacccaacaaacaacacaattagagtactgttaagtgagtccatctgctatctagctctagtttccttttgttaaacactttgaaaggactttggttcgatagcaaccgctgcagttgcaaatttgcaatattgtttagcgagtagcagcctgcactgtcgttgattgaggattcgttgcttgttttgcctaccgtcatgggcctttttcaggtcaccgaccgcatcgaggataagagccaagattctgtacgcgacgtcgaagcaagggctgaggcgggtgcttgacggggtccactacgaggtgcaagccacggactcctccgagatgggctacgacgtcatccgaggccgcgctcagtgagctgagctcctccggcgccggcgcgccgccgcgacgcgatgagtcgcgggcggggttgcggcgctgtacttagcacgtactcacccccagtgtgtgtggtataatcggacggatgctaattggctctctcgttctcgtagtagggcggctgatgatgcgatctgacgagggtttgtgaatttggtttagagtttggcgtggcgtggcgtgctgtttcgtttggagagttggaggggtgtgtgtagcgtggcgtgttgctgagggagtggcagcggattagtgaaacgtcccatgtcggtg

SEQ ID NO: 34 - DNA, 4941 bp, Oryza sativa
  misc_feature (0)...(0): line 1C-140-04 - genomic DNA without T-DNA insert
ataatccaaaatttaatccacacttttaaatggattcgaacggggccatagtactgtcacttttcttatttaagatatagaatttacatcggttttcattaacaagctttaatttcacctctgaatccaaaattctccaaattatctccactctccatcctctaatacaataagaacaagcagcccttctaaaaaaaaataaaaagaaagaaaaagaaagaaaaaaaaaagaaagaagaaggagcaagcctcgcctacttaagcacaacagcagccttgctccagctgcgagctagagccatggcgaccggagagctcgctctcgtgtcctcgctgttcatcgtcgtcgtgttcctcttgcttggtgctgtggcgagagaggcttctgcgctcacccggcatgacttccccgagggcttcgtcttcggcgcaggctcctccgctttccaggtatatatacataaaatccatcgatcatgcatgtcagaagctcatcatgctcagatgctcatcctgctcacgctcaatgctcatgctctgctgaaatttggcggaatttgttgcatcggatttgtgtttccgtattgcatcatgtttgtatatgccaagatatgtgcttactaccgtgtgagcaatgctgttcaagaactgaatttgtttttgcaaatgtgtcacacctggagtaggtggaaggggcagctgcagaggatgggaggaagcccagcatttgggacaccttcatccatcaaggtccaaaaaaatggagtagatgataatcttctcctcatgcttttattctagaacattttttttcttgtatgttctttatttacgcatgcatatagttgcattgttctagaatgaatatgtccatgattgatgcttacagggtacatgcctgacggctccaatgcagatgtctcagcagatcagtatcaccattacaaggtcagtgataagttcagatatgacacaaaatctataaaaaagatcagtttaatctacctttttttattttcaatgatttcaggaggatgtaaagcttatgtatgacatggggctggatgcgtacagattttccattgcttggcctcgtcttattccgggtagacataatcacataaataaggtagtactactgaatatcttctgaacacttatgacagtgagtttcaaattgtttttagatggaagaggagagatcaaccccaagggcttggagtactacaacaatctgatagacgaattgataatgcatggtacacattacttgctgcaccttggatttattcctagctgaatcttgatcggtagactcatcttctgtcatgtattggcaggaatacaacctcatgtcaccatctaccattttgatctccctcaggcccttcaggatgagtatggtggaatactcagccccagattcatgtaggcaccctgagaaactccacaaaatcctatgtaatctaaatgttttcatgacacaagctgatccaaatggtgatgaattcaggcaattccctaaaattttttttatatttttttagagatgggtattttttacccggtctctacatccaaccggatatatgcaacctttttaaattaggaacttagcctatcaagcagggaacttagccctcaaataacccaatctgaaattcgctcctttgaagatttgaactcaggaccatagggtgctactcaggtccaattccctgacatgttgccacatattatatttcagagaagattactcggcttatgccgaggtgtgcttcaagaacttcggtgacagggtgaagcactgggccacctttaaccagccgaacatcgagcccatcggcggcttcgacgccggcgaccggccgccgcggcggtgctcctaccccttcggcaccaactgcaccggcggcgactcctcgacggagccgtacatcgtggctcaccacctgctgctcgctcatgcctcggcagtgtccatctacagacagaaataccaggtcacaatactgaattcatactgtgttcattagtatgtctttggtgtgtctttcacataccaagaaatggtccctgaacattgaattggacatgacagcaggcaattcaaggaggccagatagggatcactctcatggttcggtggcatgagccatacaccgacaaaacagccgatgcagctgctgccattaggatgaatgaattccatattggatggttagtactgctacttaaattgcagtaccaactagccatgtcataactaactactccactaactactactccctccgtttcaaaatgtttgacaccgtcgactttttaagtacgtgtttgatcattcgtcttattcaaaaaatttaaataattatttattcttttcatatcatttgattcattgttaaatatactttaatgtacatatagttttacatatttcacaaaattttttgaataagacgaacggttaaatacgtactgtgtcaaacattttgaaacggagggagtactccgtagtacttaaattctcggcattgctatctcaaatcccaagtacactcatgacaaacttaaaacaatgtttatacccggacttttcaagcataaaacaacaagtgtttgtgatatgaaactttattcggttcagcaatttgttagtcaagtactccctcattcccaaattgatcatcatatatgtttatgtaccaagaccaaagataatttaaatcacatcaatcaagatatcatgcacatattctcccacaatgcatgcatcgattaaaataccataccaattacaccttaatatgcagacattcttccatgttgtattaattatgttatgatgaaatctatgtccatgtggttatatgatattcaatttaagaaattttgaattccattatatgatgatcaatttggaaaggagggagtatttctgtatcttctgaccgtgtatatccacttgtgcgagcctgtgaggtcagatttctatcaattatcttagaaaacaaattgtcggtctcctgttttttcaatgtactgatctccgtctgaaaatgcaggtttttgcatcctttggtgcacggagactaccctccggtgatgaggagtcgcgtgggcggtcggttgccatccataacggcgtcagattcagagaaaatacgcggatcgttcgacttcatcggcatcaaccattactacgttatctttgtgcaatccatcgacgcgaatgagcagaaactacgggactactacatcgacgcaggtgttcaaggtaagaatctctgaacaccagatgttcccaagctaagtaatcagtcaaactttgcatgcattacccatggctttctacaatcaactctcaactgcattagttttttgagttccataaaccgatcagctgatcagttgctgctcatctggtgcaggagaagacgacaaggtaatatatagatagtcgagctgcagtggagcagagttgtttttgaaaatgtttcaaggcattagaaattcagtcttgatagtcctttcgagatggtgctctctactgacactattttactatcctcgctttggaagtgatctcaaccattgatttataagttttactattagtagttaattaatgtggtaacataaatagatatgagtaaaacacctcttctttcgatgctccctttgcaggagaacattcagtgtcattcttggtctcttggcaaggtgctgaatcacctgaaactcgagtatggcaaccccccggtcatgatccacgaaatggtaggtccgaattctgcgtgggaaatgtttttaatccctgaggggatattccttcgtttcttatatgtcacctaaaaagttataaactttttttaaaaaatgataagatagattaatatgaaatataccatccacatacgtgtaagttaaaaatttaacttctataagttggtaacagaaataatgaatataactgtgaatatatgttaactatttttagtttaatttatttttttttcctaacttgttgaaatcaaatttgatcttgcatgtttgtaaatgatatatattaatatattttgttaatttttttaaaaaaagcaaattgtaactatttaggtgacattcaagaaacgaacagatctcatctagagggatgaaaatccatcttccatagttcttggtagctttaaaatggcatttgtcactggcttagtacgatgtaaattatgttttttcctcgatgcctcaggttattcagattcaccggatatcttcggaaagatcaactacaacgatgacttcagatcggagttcttgcagggctacctggaagctctgtatttgtccgtacggtgcgctcactctccttgctacttacttcctttgtgctggtaaaggaagttgacggagcgtggggctcctcaccgggagaccgtgcaggcccccctttgccggttcggccggggaccctgggtgaggttctaagctcttggtatgtggaaggttcgcgagagtggaaacacacaagacacgggcgaagtatacaggttcgggccgctgagaagcataataccctactcctgtgttttggtggatctgtgtatgaagaagctacaaagtgcgagccagcccccgatcgttctgggatcgttctttttttttccccccaagtgggcaaggtcctccttttatatctcaaggggataccacatgcaccatctctctcttttgtgtggggacttatcctctccctttccaaactggacttgccttctcttcataaatggacggagatttgtatggttgccgtctgaatgaccttctgatgggacggcccatacctacctccacttccgccggaggcaggcgcgacgtgggaacgtggctgtctgctgacgacatgaccagtgtcagaccggttacaaatcggtcatt

SEQ ID NO: 35 - DNA, 672 bp, Oryza sativa
  misc_feature (0)...(0): line 1B-115-22 coding sequence
atggccaagctgatcctcgccaccttcgccgtcgtgttcatggcgctcgccgccacctccctcgccggcgacccggacatgctccaggacgtctgcgtcgccgactacaagtccctcaaaggcccgctgcggctgaacgggttcccgtgcaagaggatagagaacgtgacggcgaacgacttcttcttcgatgggctgatgaaggccgggaacacggggaacgcggtggggtcggtggtgacggcggcgagcgtggagagcctgccggggctgaacacgatgggggtgtccatggcgcgtatcgactacgcgccatgggggctgaacccaccgcacacgcatccccgtgccaccgagatcatcttcgtcgtcgagggctccctcgacgtcggcttcgtcaccaccgccaacaagctcttcacccgcaccgtctgcaagggggaggtgttcgtcttcccgcgggggctcgtccacttccagaagaacaacggcaacacgccggcgttcgccatcgccgccttgaacagccagctccccgggacgcagtccatcgccgccgcgctcttcggcgccgcgccgccgctgccgtcggacacgctggccagggcgttccaggtcgacggcggcatggtcgagttcatcaagtccaagttcgtcccgcccaaatactag

SEQ ID NO: 36 - DNA, 999 bp, Oryza sativa
  misc_feature (0)...(0): line 1B-164-43 coding sequence
atgagctcccggatggccggatcggcgatcctccgtcacgtcggcggcgtccgcctcttcaccgcgtcggcgacctctccggcggcggcggcggcggcggcggcgaggccgttccttgcaggcggagaagccgtccccggggtgtgggggttgcggctgatgtccacgtcgtccgtggcgtcgacggaggcggcggccaaggcggaggccaagaaggcggacgcggagaaggaggtggtggtcaacagctactggggcatcgagcagtcgaagaagctggtgcgggaggacggcacggagtggaagtggtcttgctttaggccatgggagacctacaccgcggacacttcgatcgatctgacgaagcaccacgtgcccaagacgctgctcgacaagatcgcctactggaccgtcaagtcgctgcgcttccccactgatatcttcttccagaggaggtatggctgccgcgcgatgatgctggagacggtggcggcggtgccggggatggtgggcggcatgctgctccacctccggtccctccggcgcttcgagcagagcggcggctggatccgcacgctgctggaagaggccgagaacgagcgcatgcacctgatgaccttcatggaggtggcgaacccaaagtggtacgagcgcgccctcgtcatcaccgtccagggcgtcttcttcaacgcctacttcctgggttacctcctctcccccaagttcgcgcaccgcgtcgtcggctacctcgaggaggaggccatccactcgtacaccgagttcctcaaggacctcgaggccggcaagatcgacaacgtccctgccccggccatcgccatcgactactggcgcctccccgccaacgccacgctcaaggacgtcgtcaccgtcgtgcgcgccgacgaggctcaccaccgcgacgtcaatcacttcgcatcggacatccattaccagggcatggagctgaagcagacccctgcgccgatcggatatcactga

SEQ ID NO: 37 - DNA, 3216 bp, Oryza sativa
  misc_feature (0)...(0): line 1B-192-40 coding sequence
atggtatgtgcagctatacacattgcagtcgtagcaatgctcgtctcccttacagcattagcaatagctgacgaatccgacaataaccaacgggaagctctgctctgcatcaaatcacacctctcaagcccagagggaggcgccctcaccacatggaacaatacctcgctcgacatgtgcacctggcgcggcgtgacatgctccagcgagctcccaaagcctcgtttggtcgtggccttggacatggaggcacagggcctcagcggagaaatcccaccctgcatctccaacctctcgtccctcacgagaatccacctccccaacaatggcctctccggtggcctcgcatccgcagccgatgttgccgggctccgatacctcaacctcagcttcaacgcgatcggcggtgcgatcccaaaacgccttggtacgcttcgcaacctttcgtccctggacttaacaaacaacaacattcatggcgagatcccgccgttgcttgggagctcatctgccttggaatccgtcggtctcgccgacaactacctgaccggaggaatcccattgttcttggctaatgcgtcctcgctccgctatctttccttgaagaacaatagcctctatgggagcatccctgcagcacttttcaacagctcaaccatcagggaaatataccttggagaaaacaatctttctggtgcaattccacccgtaacaatattcccttcccagatcaccaaccttgatcttacaacgaatagcctgacaggaggcataccaccatctctagggaatctctcatcgcttacagcactcttagctgcagaaaaccagttgcagggaagtataccggattttagtaaactatcggcgctacggtatcttgacctctcttacaacaatctttctggaactgtaaatccctccgtttacaacatgtcgtcaatcacatttctcggtttggccaacaacaatcttgaggggataatgcctcctggtataggaaacactcttcctaacatccaagttctgattatgtctgacaatcattttcatggagaaatccctaaatctctagcaaatgcttccaacatgcagttcctgtatctggccaacaactctttgagaggggtgattccttctttcggcttgatgacagatttgcgggtcgtcatgctgtactcaaaccagttagaagctggagactgggcattcctgtcatccttgaagaattgctccaatttgcagaaactgcactttggcgaaaacaacctacgcggggacatgcccagctctgtagccgaattgccgaaaacattaacttcactagctcttccgtcaaactacatatctggcaccattcctttggagattggaaacctatctagcatatccctactttatcttggtaacaatctattgacagggagcatacctcatactcttggccaactaaacaatcttgttgtccttagcctttcacaaaacatattctctggagaaataccacaatccattggtaacttgaatcgattgactgaactctatttagcagaaaatcaactgactggaagaatacctgcaactttatcgaggtgccaacaattgttggcattgaacctttcatgtaatgcccttactggaagcataagcggagacatgtttatcaagctaaatcaattgagctggctactcgatttatcacacaaccaattcataaactccataccactagaacttggtagcttgataaatcttgcttctttgaacatttcccacaacaaactcacaggcaggatcccatctacacttggttcttgcgtccggttggaatcgcttcgtgtaggaggcaacttcctagagggaagtattccacaatcattagcaaatctcaggggcaccaaagtgctggatttctcccagaacaatttatctggtgcaataccggatttctttgggactttcacctcattacaatatctaaatatgtcatacaacaactttgaggggccaattccagtagatggaatctttgcggacagaaataaggtgtttgtccaaggaaatccacacctttgcaccaatgttccaatggatgagttgactgtctgctctgcttcagcatccaaaagaaagaataagctcattattccaatgttggcagctttttcatctataatcttactttcttcaatcctcggattatatttcttgatcgtcaatgttttcctgaaaaggaagtggaaatccaatgagcacatggatcacacctacatggaactgaaaacgttaacatatagtgatgtaagcaaagcaacaaataatttttcagcagccaacatagttggctctgggcattttgggacagtctatagaggtatactgcatacagaagacactatggtcgctgttaaggtgttcaagctcgatcaatgtggtgcattggacagctttatggcagagtgcaaagcactgaagaacatccgtcaccgaaatcttgtcaaggttataactgcatgctcaacttatgatccgatgggcagtgagttcaaggctctagtatttgaatatatggctaatggtagccttgagagccggcttcacacaaagtttgatcgatgtggtgatttgagtttgggagaaaggattagcatagcatttgatattgcttctgctctagagtaccttcacaaccaatgcattccacctgttgttcactgcgatttgaagccaagcaatgtacttttcaacaatgatgatgttgcatgcgtctgcgactttggtctggcaaggtcaattcgtgtttattcatctggaactcaaagcatatcaacaagtatggctgggccaagggggtctattgggtacattgctccttatggcatgggcagtcaaatctcaactgagggtgatgtctatagttatggcattattcttctggaaatgctaacaggaagacaccctaccaatgaaatatttactgatggcttgacactccgcatgtatgtcaatgcatcactttcacaaatcaaagacatacttgaccccaggcttattccagaaatgacagagcaaccttctaatcataccctgcaattgcatgaacacaagaagacaccatctcgatgcaaactgggaggggtggagggtattctcacatgtacgataagagaaattgcatcaaaattaggagggcttagattatctatgctaacgtga

SEQ ID NO: 38 - DNA, 1914 bp, Oryza sativa; identical to SEQ ID NO: 21
  misc_feature (0)...(0): line 1B-207-27 coding sequence

SEQ ID NO: 39 - DNA, 1571 bp, Oryza sativa
  misc_feature (0)...(0): line 1B-138-07 coding sequence
atggccgcgccgctctccaccgccgccgccgcctcgtggctctccgacagcgcctcgtctccgcctagggtgcgcctcctcatcggcggggagttcgtcgagtcgcgggccgacgagcacgtcgacgtcaccaatccggcgacgcaggaggtggtgtcgcggatcccgctcaccaccgccgacgagttcagggccgccgtggacgccgccaggaccgccttccccgggtggcggaacacgccggtgaccacgcggcagcgcatcatgctcaagtaccaggagctcatccgggccaacatggataaactggcagagaacattacaaccgaacaggggaagacactgaaggatgcttggggcgatgtattccgtgggctaggtggtggaacatgcttgtggaatggggacactgcagatgggtgaatatgtatcaaatgtttctaacgggattgacacctttagcattagggagccacttggtgtatgtgctggaatatgtccattcaattttccagctatgatccccctatggatgttcccaatagcggtcacttgtggcaatacttttgttctaaagccatcagaaaaagatccaggggctgctatgatgcttgcggagctagcaatggaggctggtttaccaaagggtgtgttgaacattgttcatggtacccatgatgttgtgaacaacatttgtgatgacgaggacatcaaggcagtttcctttgttggttccaatatagctggtatgcatatatattctagagcatcggcaaaggggaagcgtgttcagtctaatatgggtgcaaagaatcatgcaattatccttcctgatgctgaccgagatgccacactgaatgcccttattgctgctggttttggtgctgctgggcaaaggtgtatggcattgagcactgctgtttttgttggaggttcagagccatggataagacctggcattagggaggatgagctagtcaaacgtgcaagcagccttgtggttaattcaggaatggctagtgatgcagaccttggtccagtgatcagcaaacagggctaaggaacgtatctgcaaattaatacaaagtggtgctgataatggtgctcgtgtgctgcttgatggaagagatattgtggttcctaacttcgagaatggtaattttgttggtccaacactccttgctgatgttaaaagtgaaatggaatgttacaaggaggagatttttggtccagttcttctcttgatgaaggctgagagcctagatgatgctatccaaattgtgaacagaaacaaatatggcaatggagcatccatatttacaacatctggtgtgtctgcaaggaaatttcaaacagacattgaagctggccaggtgggcatcaacgtgccgattccagtacccctgccgttcttctccttcaccggcagcaaagcctcctttgcaggagacttgaatttctacggcaaggcgggcgtgcagttcttcacccagatcaagacggtcacgcagcagtggaaggagtcgccggctcagcgcgtctccctctccatgcccacctcgcagaagtga

SEQ ID NO: 40 - DNA, 1323 bp, Oryza sativa
  misc_feature (0)...(0): line 1D-059-12 coding sequence
atggccgccgccgccaccgccgccgtccccctcgacgaggccaaagccaaggaggttctccgccaggtggagttctacttcagcgacagcaacctcccccgcgacaacttcctgcggaagacagtcgaggagagcgaggatggcgtgagcttggcactcatctgctccttctcgcggatgaagaagcacctaggcctggacgcggacgtgaagcaggagaccatgccggaggagacggtgctcgccgttgctgaggtgctgcggcgttcctcggccctccgtgtcaccgaggataagaaagttggtagatcaattgagttgtcgaaactggatgagatcatggagcaagtggactctaggacaattgctgcatcaccatttccttacaatgtaaagctggaagatgttcagtctttctttgctcagtatggcaaggtgaacagtgtgaggctacctcgacatattgccgacaaacgacacttctgtggcactgctttagtcgaattttcagaagaagaggaagcaaatgctgtattaaagaatactcttgtttttgcaggagcagatctggaaataaaaccaaaagaattcgatactgaaagagaggctaagaaagaagcttatgagaagtcacaacctactaagaatggtcatgatgagtatccaaaaggtctaattgtggccttcaagctgaagataattcaaattgatggtggcatggcagaaaatggtggggacaaagagggtgaaactgatgatgccaataaatcaagaacagggcatgacgagaaaatccctgagaacagtgatatcaaggaagacttgtcagatgatgttgagaagtcaaaagaggcagctgctcaatctgttaagaaaggagaaagtccttcagaaaatgcggatgatccaatttcaagggaagattttaaagaagaatttggcaaattcggcacagtgcggtatgtggacttcagcataggggaggattcaggatacattcggttcgaggattctaaggcagctgaaaaggcccgtgcacttgcggctatttcagatgaaggtggtttgattatgaagggccatcttgttactttggaacctgtgtctcaagctgagaaggattattggagtgcgataaagggcggtcaaggaaaatatagagacaatagaagtaacaggggagccgatcctcttaaatatcataatgcacctagaatgaatcttaggagtgggctgaaacttaaaatagaagttgaagccttatggcacacaagaatgccaacacatatatttggagttatattatcatggaactggcagtaa

SEQ ID NO: 41 - DNA, 2568 bp, Oryza sativa
  misc_feature (0)...(0): line 1C-087-40 coding sequence
atggccacacgttattggatcgtgtctcttcccgtgcagactcctggctccaccgccaattctctctgggcgcgcctccaggactccatctcgcgccactccttcgacacgccgctctaccggttcaacgtccccgatctccgcgtcggcacgctcgactccctcctcgccctcagcgacgatctcgtcaagtccaacgtcttcatcgagggggtctcgcacaagatccggaggcagatcgaggagctagagcgcgccgggggtgtcgagagtggggctctcaccgttgacggcgtccccgtcgacacctacctcacctttgtgtgggatgagggcaaatacccaacgatgtcaccgctcaaggagattgtcggcagcatccaatcacaggtctccaagattgaagatgacatgaaggttcgaggagcggaatacaataatgtaaggagccagcttagtgcgatcaacagaaagcaaactggattagcagttcgtgatctttccaatctggtaaaaccagaggatatggtcacatcagaacatctagtgacactccttgcagttgttcctaagtactctcaaaaggattggttgtcaagctatgagtcccttgacacatttgtggtaccgagatcgtctaaaaaactttatgaggacaatgagtatgctctctacacggtaacattgtttgctaaggttgttgacaactttaaggtccgtgcacgtgaaaaaggtttccaggttcgcgattttgagtatagttctgaagcacaggaaagtaggaaggaagagctggaaaagctaatgcaagaccaggaagcaatgagggcatcacttctgcaatggtgctatgccagctacagtgagaataccatttatgtacgtcatgctaaagtgcaaaactttgtgcttctctatgtattcagttcctggatgcacttctgtgctgtgcgtgtctttgtagagagcattcttagatatggtcttcccccatcattcctgtctgctgttctagcaccttctcaaaagggtgaaaagaaagtaaggagcatcctggaggaactctgtggcaatgtccatatttactggaaatctgaagacgatgttggtgtagctggtctgggacaccgtgtaagatgttgtggatattatgtttttgtaactttgagcagccagaaggacaagttgggggttatgtatagaacgtcaatccttggtgacccagttcccactgatgatagtagagtagacaagacacaagaggagatgatggtcaaggagaaggagatacacgtgatgtccaaccggcgtcgcaagcgcggcgaatccaagccccaagcccacgacgccggcgacaccacgcccatcgacaacatactcacctccctcgacgacgccccgccaccgccacctccaccgcgccgcgagctccccctcctccagctcaacgaccaccaagaggcagatgacgctgacgcctcgtcgtcgtcgtcgccgcaccagcagcggcggctgtgggtgaaggaccggtcgcgcgcgtggtgggagctgtgcagcagcgccgactacccggaggccgacttccgccgcgccttccgcatgtcccgccccaccttccacttcctctgcgacgcgctcgccgccgccgtcgccaaggaggacaccgccctccgcgccgccatccccgtccgccagcgcgtcgccgtctgcgtctggcgcctcgccacgggggagccactccgcgtcgtctccaagcgcttcggcctcggcatctccacctgccacaagctcatcctcgaggtctgcgccgccatccgcaacctcctcatgccgcgcttcctccactggcccgaccaccccacctccaccgcctacaagacgcgcttcgaggccacctcgggtgtctcgggggtggtgggcgccatgtacaccacgcacatccccatcatcgcccccaaggtctccgtcgccgcctacttgaaccgccgccacacggagcgcaaccacaagacctcctactccatcaccctccagggcgtcgtcggccccgacggcaccttcaccgacgtctgcatcgggtggccggggtccatgagcgacgagcaggtgctccgcaagtcggcgctgcaccagcgggcgtcggcggcggctgggtcgatgtcgtgggtggtgggcggcgcgagctacccgctgacggagtggatgctggtcccctacgcgcagcgcaacctgacgtggacgcagcacgccttcaacgagaaggtgggcgaggtccggcgcgtggccacggaggcgttcgtccgcctcaaggggcggtgggcgtgcctgcagaagcgcacggaggtgaagctgcaggacctccccgcggtgctcgccgcctgctgcgtcctccacaacatctgcgagaccaggggggaggacatggaccccgacctccgctgcgacctcccccccgacgaggaggaggacgacaccgtgcttgtgcagtcggagtccgccaacaaggtgagggacgacatcgcgcacaacctcctccaccgcggattggccggcaccgccttcttctga

SEQ ID NO: 42 - DNA, 1413 bp, Oryza sativa
  misc_feature (0)...(0): line 1C-017-14 coding sequence
atggacgccctcctcgtggagaaggtcctcctgggcctgttcgtggcggcggtgctggccctagtggtggccaagctcaccgggaagaggctccgcctcccgcccggccccgccggcgcgcccatcgtcggcaactggctccaggtcggcgacgacctcaaccaccgcaacctgatggcgctggcgcggcggttcggcgacatcctcctcctccgcatgggcgtccgcaacctggtggtggtgtccagcccggacctcgccaaggaggtgctccacacccagggcgtcgagttcggctcccgcacccgcaacgtggtgttcgacatcttcaccgggaaggggcaggacatggtgttcaccgtgtacggcgaccactggcgcaagatgcggcggatcatgacggtgcccttcttcaccaacaaggtggtggcccagaaccgcgcgggttgggaggaggaggcgaggctggtggtggaggacgtccgccgcgaccccaccgcggcgacctccggcgtggtgatccggcgaaggttgcagctgatgatgtacaacgacatgttccgcatcatgttcgaccgccgtttcgacagcgtggacgacccgctcttcaacaagctcaaggccttcaacgcggagcgcagccgcctctcgcagagcttcgagtacaactacggtgacttcatccccgtcctccgccccttcctccgccgctacctcgcacgctgccaccagctcaagtcccagcgcatgaagctcttcgaggaccacttcgtccaggaacgcagagtgatggagcagactggtgagatccggtgcgccatggaccacatcctcgaggccgagaggaagggcgagatcaaccacgacaacgtcctctacatcgtcgagaacatcaacgttgctatcgagacgacgctgtggtcgatcgaatggggaatcgcggagctggtgaaccacccgagcatccagtcgaaggtgcgggaggagatggcgtcggtgctgggcggcgcggcggtgacggagccggacctggagcggctgccgtacctgcaggcggtggtgaaggagacgctgcggttgcgcatggcgatcccgctgctggtgccgcacatgaacctcgccgacggcaagctcgccggctacgacatccccgccgagtccaagatcctggtgaacgcgtggttcctcgccaacgaccccaagcggtgggtgcgccccgacgagtttaggccggagaggttcctggaggaggagaaggccgtggaggcgcacggcaacgacttccgcttcgtgcccttcggcgtcggccgccgcagctgccccgggatcatcctcgcgctgcccatcatcgggatcacgctcggccgcctcgtccagagcttcgacctgctgccgccgcccgggatggacaag

SEQ ID NO: 43 - DNA, 1227 bp, Oryza sativa
  misc_feature (0)...(0): line 1C-038-56 coding sequence
gatgttcagcagcatagtggcagtagcagcagctcaaccgaatccgacgtccaagaaaccgccgctgtcgccgtcgccgacccctccccgcggtcggaggtcgtcgacggcgagagcccgccgcagccgggcggcgaggcggcgagccatcagcagcagcagaaggagatgaagctgaagaagccggacaagatcctgccatgcccgcggtgcagcagcatggacaccaagttctgctacttcaacaactacaacgtcaaccagcctcgccacttctgcaagcactgccagcgctactggaccgccggcggcgccatgcgcaacgtccccgtcggcgccggccgccgcaagaacaagaacgccaccgccgccgcccacttcctccaccgcgtccgcgcctgcgccgccgccgccgccatgcccgcggcgccccacgacgccaccaacgccaccgtgctcagcttcggcggcggcggaggcggacacgacgcgctgccggtcaccctggacctcgccgacaagatgacgcgcctcggcaaggaggggctcgtcgcccatgcccggaacgccgacgccgccgccgcgtgcagcgaggtgtcgagcaacagggacgacgagcagatcggcaacactgtagcaaaacctgcaaacgggttgcagcagcatcctcctcctcctcatcatcatcatcattcagccatgaacggtggcggcatctggccctactacacctcggggatcgcgatcccgatatacccggcggcgccggcgtactggggctgcatgattccacctcctggagcttggagcctcccatggccggccacagtccagtctcaggccatctcatcatcatcaccacctacaagtgctacaccttcagtctcctccttcacactaggcaagcatcctagagagggtggtgatcatgaggcaagagatcaccatggcaatggtaaagtgtgggtgccgaagacgatccggatcgacaacgccgacgaggttgcccggagctcaatccggtcactcttcgccttcagaggcggcgacaaggtggacgataacaacgacgacgatggcaccagcgtgcacaagctcgccaccacggtgttcgagccaaagagggacggcaagacggcgaaacatccggcgatcacgagcttgccgctcttgcacaccaaccccgtcgcgcttacccgatccgcgaccttccaggagggatcttga

SEQ ID NO: 44 - DNA, 861 bp, Oryza sativa
  misc_feature (0)...(0): line 1C-041-47 coding sequence
atgggcatcaagggtttgacgaagcttttggcggacaatgcgcccaaggcgatgaaggagcagaagttcgagagctacttcggccggcgcattgccgtcgacgccagcatgagcatctaccagttcctctcatttgcatactcgaagagggaggatgccaccaaagaactaacagaggcagtagaggaaggagataaagacgcaattgaaaaattcagcaagagaactgtgaaggtcacgaagcagcacaatgaagaatgtaaacgactattaagacttatgggggttcctgttgtagaggtttatgcagttgcatcagaagatatggactctcttacttttggagctccacggtttcttcgtcatttgatggatccaagttccaagaaaattccagttatggaatttgaagtcgcgaaagttttggaggagcttgaactcactatggaccaattcattgacttgtgcatcctatctggatgtgactattgtgatagcattaaaattggaggtcaaacagctctgaaacttattcgtcaacatgggtccatagaaagcattctggaaaacataaataaagactatcagattcctgaggactggccttatcaagaagctcgacgcttgttcaaggaacccaatgttacattggatattcctgagctgaaatggaatgctcccgatgaggaagccatagagaagattaaatttgccaagaacaaatcttcccaaggactcgaatccttcttcaagccagttgttagcacatcagtgcctctgaaaagaaaggacacttcagaaaaaccaaccaaagcagtcgctaacaagaaaacaaagggagccggcggaaagaagaaataa

SEQ ID NO: 45 - DNA, 1653 bp, Oryza sativa
  misc_feature (0)...(0): line 1C-064-20 coding sequence
atggcgtcggcccccggcgacggcaagcagggcggcggcggcggcgggccggcggtgggcatcgacctcggaacgacctactcgtgcgtggcggtgtggcggcacgaccgcggcgaggtcatcgccaacgaccagcgcaaccgcctcacgccctcctgcgtcgccttcaccgccgacgacgacgacagcttcgtcggcgacgccgccttcaaccagtccgcactcaacccaaccaataccatctttgtgaagcgactgattggccgccgattcagcgatgattctgtacaaaaagatatcaagctttggcctttcaaagtcgtggcaggtcaagaggacaggccgatgatcgtggtgaggcatgaaggcgaggaaaggcagttcatgcccgaggagatctcctccatggtgctcgccaagatgagggagacggccgaggtgtacctcggcaagacggtcacgaaggccgtcatcactgtcccggtctacttcaacaacgcgcagcggcaggccaccatggacgccggcgccatcgccggcctcaacgtgatgcgcatcatcaacgagcccaccgccgccgccctcgcctactgtctcgagaagatgcccgtcagcaacaaggggaggatggtgctcgtgttcgatctcggtggcggcacattcgacatctcccttctcaacatcgatcccgaaggcaccgccggtgacactcaccttggcggagcggatttcgacaacgagttggtgaagcactccttgcgagagttcaatcggaaacatgggtcgatggacattgaaagcaatcagaaggcattaaggagattgaggaccgcctgcgagagagcgaagaggatgctgtcatccacgatgcagaccaccattgaggtagactcgctccatcaaggcatcgacttccgcgtcaccctcacccgatcccgatttgaggagctgaacaaggatctcttcagcaagtgcatggaggctatggagaattgcctccgcgacgccaaggtggacaagtggagtgttgacgatgtcgtcctcgtgggtggctccacccgcatacccaaggtgcagaagatgctgagtgagttcttcgacgggaaggagctctgccgcagcatcaaccccgatgaagccgtcgcgtatggcgccgccatccaggcctccattttatgtggtggaaccgatgataagaggttggttgatatgcttctccgcgaagtcacgccgctctcgctaggtgttgagactgaagataattgtacaatgagcgtggtgatcccaaggaacactgcaatcccgaccaagaaggtgaaaaacttcactactctctacgacaaccagatcaacgtgagctttccggtgtacgagggtgagagcgcaaacaccaaggacaacaacctgctcggcgagttcacgctatatggcatccccccggcacccaagagagtgccatctatcgatgtcactttcgacattgatgccaacggggtcttgaacgtttctgccgagcacaaggtcaccggacagaagaacagcatcaccatcactaaccgcagcggacggctgaaccaggaggaaatcgatcgcatggctctggagcccgagaggcacaagatgaagcggatcaagcttcacgaagtagtctaa

SEQ ID NO: 46 - DNA, 1437 bp, Oryza sativa
  misc_feature (0)...(0): line 1C-109-35 coding sequence
atgtcgtcgtcggcgacggtggtgccgctggcgtaccaggggaacacgtcggcgtcggtggcggactggctgaacaagggggacaacgcgtggcagctggtggcggcgacgctggtggggctgcagagcgtgccgggcttggtggtgctgtacggcggcgtggtgaagaagaagtgggcggtgaactcggcgttcatggcgctctacgccttcgccgccgtgtggatctgctgggtcacctgggcgtacaacatgtcgttcggggagaagctcctcccgatctgggggaaggcgcggccggcgctggaccagggcctcctcgtcggccgcgccgcgctgccggcgacggtccactaccgcgccgacggcagcgtggagacggcggcggtggagccgctgtacccgatggcgacggtggtgtacttccagtgcgtgttcgccgccatcaccctcatcctcgtcgccggctccctcctcggccgcatgagcttcctcgcctggatgatcttcgtcccgctctggctcaccttctcctacaccgtcggcgccttctccctctggggcggcggcttcctcttccactggggcgtcatcgactactgcggcggctacgtcatccacgtctccgccggcatcgccggcttcaccgccgcttactgggtggggccaagggcgcagaaggacagggagaggttcccgccgaacaatatactgttcacgctgacgggggcagggctgctgtggatggggtgggcagggttcaacggcggcggtccgtacgccgccaactccgtcgcctctatggccgtcctcaacaccaacatctgcaccgccatgagcctcatcgtctggacatgcctcgacgtcatcttcttcaagaagccctccgtcgtcggcgccgtccagggcatgatcaccggcctcgtttgcatcacccccgctgcagtggtgcaggggtgggcggcgctggtgatgggggtgctcgccggcagcatcccgtggtacaccatgatgatcctccacaagcgctccaagatcctgcagcgcgtcgacgacaccctcggcgtcttccacacccacggcgtcgccggcctcctcggcggcctcctcaccggcctcttcgccgagcccaccctctgcaacctcttcctccccgtcgccgactcccggggcgccttctacggcggcgccggcggcgcccagttcggcaagcagatcgccggcggcctcttcgtcgtcgcctggaacgtcgtcgtcacctccctcatctgcctcgccatcaacctcctcgtcccgctccgcatgcccgacgacaagctcgaggtcggcgacgacgccgtccacggcgaggaggcctacgcgctctggggcgacggcgagatgtacgacgtcaccaagcacggctccgacgccgccgttgcccccgtcgtcgtatga

SEQ ID NO: 47 - DNA, 6534 bp, Oryza sativa
  misc_feature (0)...(0): line 1C-109-51 coding sequence
atggcgaacctgggcggcggcgccgaggcgcacgcgcgcttcaagcagtacgagtaccgcgccaactccagcctcgtcctcaccaccgactcgcgcccccgcgacacccacgagcccaccggcgagcccgagacgctctggggcaggatcgaccccaggagcttcggcgaccgcgccgtccaggccaagccccccgagctcgaggagaagctcaccaagtcccgcaagaagaaggccgccgcctctgaccccgacgacctccaccgccgcgacgccaagcgcaggcgccgcgccgcagccgcccagagtgaggtcagcgtcctctcgctcaccgacgacgtcgtctacaagccccagaccaaggagacgcgcgccgcctacgaggccctgctcagcgtcatccagcaacagttcggcggacagccgctcgacgtcctcggcggcgctgccgacgaggtgctcgccgtcctcaagaatgacaagatcaagagccctgacaagaagaaggagatcgagaagctcctcaaccctatctccaaccagatgttcgaccagatcgtctccatagggaagctcatcacggatttccatgatgcctcggctggtgattcagctgctgcgccatctggtgatggcatggacacagcgctggatgatgacatcggcgttgctgttgaatttgaagagaacgaagatgacgaggagagcgatttcgatcaggtgcaagatgatttggatgaagatgaagatgatgacttgcctgagtcgaatgcccctggtgctatgcaaatgggtggcgagttagatgatgatgatatgcagaactccaatgaggggctgaccataaatgtacaagacattgatgcttactggcttcagaggaaggtctcgcaagcatacgaagatattgatccgcagcacagtcagaagcttgctgaggagatcttgaagataattgctgagggtgatgacagagatgtcgagaatcggcttgtcatgctgttggactatgaaaagtttgatctcattaaactgctactacgcaaccgactcaagattgtttggtgtacccgtttagcgagggctgaagatcaggaacagcggaagaagattgaggaagatatgatgggtaacccaactttaactccgatattggagcagctacatgcaacgagggcatctgcaaaggagaggcagaagaatctagagaaaagtatcagggatgaggccaagaggcttactaaaagcgaaaacactggcattgatggtgcgagggatcgtagggcagttgaccgggatatggagagtggatggttgaaaggccagaggcagctgcttgatcttgacagcctgtccttccaccaaggtggtctcttgatggccaacaagaaatgtgagcttcctccaggatcattcagaacccctcataaggggtatgaggaagtccatgtgccggcgctaaaagctaagccatatgaaactggagagaagattgtcaagatatctgatatgccagagtgggctcaaccagcttttgctaagatgacacagctgaacagggttcagagcaaggtttatgagactgcccttttcaaaccagataatattctcctctgtgctccaacaggtgctgggaaaaccaatgtggctgtgctcacaatccttcagcagattgggttgcatatgaaggatggagtgtttgacaataccaagtacaaaattgtctatgtggccccaatgaaagctttggttgccgaagttgttggaaatttgtcggctcggctgagtgcgtatggtattactgtaagagagctcagtggagaccagaacctgaccaaacagcagattgatgaaacacagataattgtcaccacacctgagaaatgggacattgtcacaagaaaatcaggtgacagaacctatactcaaatggtaaagcttctaatcattgatgagatccatctacttcatgacaacagagggcctgttctggagagcattgtttctagaactgtccggcagattgagacgaccaaagaacatatccgtctagttggtctctcagcaactcttccgaactatgaagatgtcgcggttttcctgcgtgttcgctctgatggcctcttccatttcgataacagctacagaccttgcccccttgctcagcaatacattgggatcactgtgaggaagccactacagaggtttcagctgatgaatgagatttgctacgagaaggttatggcatctgctggaaagcatcaagtgcttatattcgtgcactcgaggaaggagacagcgaaaactgcccgtgccatccgagatactgcattggctaatgacacgttaaaccgcttcttgaaggatgatagtgcaagccaagagattcttgggagtcaggcagaactagtaaaaagcagtgaccttaaagaccttttgccttatgggtttgctattcatcatgctgggttggcaagggtagaccgtgagcttgtagaggaactttttgccgataagcatatacaggtccttgtctcaacagccacccttgcatggggtgtcaatttgcctgcacacactgttataataaagggtacccagatttacaatcctgaaaagggtgcgtggacagagctgagtcctctagatgtcatgcagatgcttggccgtgctggcaggccacagtatgatacacatggagagggaataatcctaactggccacagtgaattgcaatattacctgtctctaatgaatcaacaactgcctattgagagtcagttcatatccagattggctgatcaattaaatgcagagattgttcttgggactattcagaatgctcgggaggcatgctcatggcttggctacacctacctctacatccggatgctccggaatccaacattgtatggtctaccggcagatatcatggagactgataaaacactagatgaaaggagagctgacttggtgcactctgctgcaaatctactcgataggaacaatttgataaagtatgacaggaaaacaggatacttccaggttactgacctaggaaggattgctagttattactatattagccacgggactatttcaacttacaatgagtacctgaagcctacaatgggtgatattgagctatgtcggctcttttcactcagtgaagaattcaagtatgtcagtgttagacaagatgagaaaatggagctggctaagcttttggatcgtgtgcctattcctgtgaaagaaagcttggaggagcccagtgcaaagatcaatgttctgctgcaagcatatatttctaggctgaaattggagggtctttcgcttagctccgatatggtctacatcagacagagtgctggccgtctcttacgagcgttgtttgagatagttctgaagagaggatgggcccaactagcagagaaggctttgaatctttgtaagatgatagataagcagatgtggaatgtccaaactcccctgcgtcaattccctggtattccgaaggaaatcctaatgaaactggagaaaaaagaattggcttgggagaggtactacgacctatcatctcaggaaattggtgaactaattcggttcccgaagatgggcaggcaactgcacaagtgcatccaccagttaccaaagttgaatctttcagcccatgttcagccaattactcgtacggttttgggttttgagttgactataactcctgatttccaatgggatgataaggtacatggatatgtggagcctttctgggttattgttgaggacaacgatggcgagaacattcttcaccatgagtacttcatggttaagaaacaatacgtagatgaagatcatacactcaacttcacagtgccaatatacgagccactgccccctcaatactttattcgtgttgtatctgataagtggcttggttctcagacaattcttcctgtctgttttaggcacttaattcttccagaaaaatatgctccgccaactgaattgcttgatctgcagccactgcctgttactgcattgagaaatgcacgatacgaaggcctttatagtgccttcaaacatttcaacccaatccagactcaagtgttcactgttctgtacaacactgatgacagtgttttggttgctgcgccaacaggcagtggaaagaccatctgtgcagagtttgctatactaagaaaccatcagaaggctgtatctggtgagagcaacatgcgagttgtgtatattgctcctattgaagcccttgcaaaagaaaggtacagggattgggagcagaaatttggagaatttgctcgggtagttgagctaacaggtgaaactgcagctgacttgaagcttctggataaaggggagattataattagtactcctgagaagtgggatgcactctctcgccggtggaaacagcggaaacaagtccaacaggtcagcctattcattgttgatgagcttcatctgattggatctgaaaagggacatgttctggaagtcatcgtctctaggatgaggcgtattgcaagtcatattggcagtaacatccggattgtggctctttcagcgtcacttgcaaatgctaaagatctcggagaatggattggtgctacctctcatggccttttcaactttcctccagctgtccggccggtgccattagaaatacacattcagggtgtggatatagcaaattttgaggcaaggatgcaggcaatgacgaagccgacatacactgctattacccaacatgcaaagaatggtaaacctgccctggtgtttgtgcctacacggaagcatgcaaggttgactgcattggacttgtgtgcgtactcgagtgctgagggtggcggaacacctttccttcttggttcagaagatgagatggatgctttcactggtggtatcagtgatgaaacacttaaatatactctgaaatgtggtgtaggctacttgcatgaaggtttaagtgatctggaacaggaagttgtaactcagctgtttctcagtgggaggatccaagtgtgtgttgcgagtagcactgtgtgctggggaagatcattgcctgcccatctggtggttgtgatgggaacccagtattatgatggccgggagaatgctcatactgattatccaatcactgatctactccaaatgatgggtcatgctagcaggcctcttcaagataactcagggaaatgtgttatattgtgtcatgcgcctcgcaaggaatactacaagaagttcctttttgaggccttccctgttgagagccatcttcaccacttcttgcatgatcatatgaacgctgaggtggtggttggtgtcatagaaaacaagcaagatgctgtggattaccttacttggaccttcatgtacaggcggctgaccaagaaccctaactactacaatctgcagggtgtgagccacaggcatttgtcggaccatttatctgagttggttgagactgtattgaatgacctagaatcaagcaagtgtgtggctatagaggaggatatgtacctgaagcccctcaaccttggtctgattgcttcgtactactacattagctacacaactattgagaggttcagttcaatgctaactcaaaagactaagatgaaagggctcctggagattctagcctctgcttcagaatatgctgagcttcccagtcgccctggtgaggaggatttcattgagaagcttgttcgccaccagagattttccatcgagaagcccaggtatggtgatccacatgtcaaggctaacgcactgctgcaagctcatttttcaaggcacactatcttagggaacctggcagccgaccagcgggagatactcctttctgctcatagattgctccaggcaatggttgatgttatctccagcaatggatggcttacgcttgctcttaatgcaatggagctgagtcaaatggtgacacaaggcatgtgggatcgtgattctgttcttttacaacttccacatttcaccaaggagttggcacggagatgtcaggagaatgaagggaggcccattgagagcatctttgatctggctgagatgagcatcgatgagatgcgggatctgttgcagcaatcaaaccctcagctgcaggatatcattgaattcttcaagcgtttccccaatgtcgatatggcttacgaagttcgcgagggtgatgatatcagggctggtgacaatgtaactgtacaggtgaccctggagcgtgacatgacgaacttgccttctgaggtggggccagttcatgcccccaggtacccaaagcccaaggaagaaggctggtggctggtaattggtgatagctctaccaatcagttgcttgcaatcaagagggttgcacttcagaagagggctcgagtgaagcttgagttcaccgctgcttcagaagctgggagaaaggagtacatgatctacctgatgtccgattcttatctgggctgcgatcaggagtacgagttcaccgtcgatgtcatggatgctggaggggattga

SEQ ID NO: 48 - DNA, 543 bp, Oryza sativa
  misc_feature (0)...(0): line 1C-056-07 coding sequence
atggttggctgccttgctacatctaccaagaccattttggcagagtctctactccacggatacaaatttgacattaacacagtgtactacatggcaccctttgccaccatgatactggctctaccagcagtgttacttgaaggaggcggtgtggttacctggttctacacacatgactccattgcttctgcactagttatcatcataggctctggagtgcttgcattttgcctaaacttctccattttctacgtgatccattcaaccactgcagtgaccttcaatgttgctggcaacctgaaagttgctgttgctgtattggtgtcatggttgatcttccggaaccctatctctcctatgaatgcaatcggatgcgcgatcacgctcgttggctgtactttttatgggtatgtgaggcatttgatctctcaacagcaggctgtagctccaggaacaggaagcccaacaacatcgcaaacaaattcgccgagaagtcggatggagatgctcccccttgtaggcgacaagcaagaaaaggtttaa

SEQ ID NO: 49 - DNA, 2436 bp, Oryza sativa
  misc_feature (0)...(0): line 1C-100-32 coding sequence
atggagatggagatggaggacaacaaggcgccctcgccgccatcacctatggagtccgacgtcgctgaggagaagcgcaagagggaggacgatgcatcctcctccgctgtacttgccgccgccaacaacaccggcggtgcccagcaccccatgtggaagaccagcctctgctccttcttccgccgccgcgccgcctcctccgccgacggctgcagccacggcgactcctgccgctacgcgcactccgaggaggagctccgcccgcgccccgacggcacctgggaccccacctccgaccgcgccaagaagctccgcaaggtcgccgccgacgaggtggaggaagaggtggtcaccattgacgacaaggccctggacaagtgcctcgtcggcctcccgaggggatgggccaacgacaggctcaagactttcctccaagacaaggcaagaactaactactcctccattcttcctcctgctctactactaggaatctcgtatgcaacagcgaagaagaagaagggaatgactgttggttttgtaacttttgaaaatattgaacagctgaagaatgctatcgaggtacttacagagaaccaatctggtggaaaggaaataaagatagcagatgccaatcgtagatctcatcaaaagctgcacacagaaaagcctgtatctgacaatggagtgacaacagaaaatggtactagtgttgatgttcctcctggggagacatctgcacctgaagcagcaatatcaaataaaaaaagtgtccgcgatgcagttactccccttgcccacatgtcttataatgatcagctagagcacaaaaataattctgttgcgcagatactgaagaggcttactcgcaatgctaggaaagcttgccctactggcattccccttccagattgggtttttaaatccaaagaaatttttcatttattccatttttcctctttagtttgtcatgattatattttaaatcatgcaggtggtcttccttgcaagcttgaaggcattctggagtccccagtgattaatggataccgtaacaagtgtgaattctctgtgggattttctttggagggaaaaaagacggttggatttatgcttggaaattttactgatatgattgacaaaacaaaatccagggaaggtgtgactgctgttgaggaacctgtggactgcccaaatgtctcagaaatttcctgcaaatatgctctgatgttccaagactttctgcagtcatcaagcttgcctgtgtggaacagagttaataattgtggattttggcgtcaattcacagttcgggagggaagatgtcgagctcaagctgttgcacagaatgcagaaacccaaatatcagaagtcatgcttattgttcaggtttgctccacaggtgttgatgatgcagtaatgaaagatgaatttgacaagttgaccgttgccctgcaacaaggagcagcaacatgctcacctccattacctctaacaactatagtagtgcaagatcacaaaggaatttcaaatgctgcaccagctgattgtccattgatcccactattggtgccaaaagtagatcaatcagaaggtacagtggataaaacaagaatccatgaccacatcggtaatctgtggttctccatatcaccaacagcattttttcaggtcaatactctcgctgcagaaagattgtatacccttgctggtgattgggccaacctcaattcgggcacattactttttgacgtatgttgtggaacaggaacaattggactgaccttggcacaccgtgttggaatggttgttggaatcgaaatgaatgaatcagcagtttctgatgctgagagaaatgcgctcattaacggtgtatcaaattgtcgctttgtctgtgggaaggccgaagatgtgatggggtctcttctcactgaatatcttggttcaccgcagcaggacattcctgtttctgaaggtgcagtaagtggtactgtgaaagatgaggaagttattgacagttctaagaatagtggcgaaaatttggacagctcaatgcagaaaaatgacaatggcaaaagccagcagctcggggatgcaccagctgattcttcctcttctgccatagatgagataaaggggaattccaatgatagggttggcaatgggttggaaggcagccatgatgaatacaatgaggttgctggagaagatattcatggggaagcatcattgatcaatgagtctgttgacctgaaagtatcggactgtttggaggacagaaagacatctgatgatggttcttccatttctaacaatgatgtgactgcagctactgcatgtcagttcgaagacattgttgctattgtggatcctcctcgtgttgggcttcaccctactgtaaaccccaaaagaggacagggagttcctgttttcatttaa

SEQ ID NO: 50 - DNA, 429 bp, Oryza sativa
  misc_feature (0)...(0): line 1C-142-27  coding sequence
atggcaatggcttacaagatggcgacggaggggatgaacgtgaaggaggagtgccagaggtggttcatggagatgaagtggaagaaggtgcaccggttcgtggtgtacaagatcgacgagcggtcgcgcgccgtgctggtggacaaggtgggcggccccggcgaagggtacgaggagctcgtcgccgcgctgcccaccgacgactgccgctacgccgtcttcgacttcgacttcgtcaccgtcgacaactgccagaagagcaagatcttcttcatcgcctcaccgaccgcatcgaggataagagccaagattctgtacgcgacgtcgaagcaagggctgaggcgggtgcttgacggggtccactacgaggtgcaagccacggactcctccgagatgggctacgacgtcatccgaggccgcgctcagtga

SEQ ID NO: 51 - DNA, 1344 bp, Oryza sativa
  misc_feature (0)...(0): line 1C-140-04 coding sequence
atggcgaccggagagctcgctctcgtgtcctcgctgttcatcgtcgtcgtgttcctcttgcttggtgctgtggcgagagaggcttctgcgctcacccggcatgacttccccgagggcttcgtcttcggcgcaggctcctccgctttccaggtggaaggggcagctgcagaggatgggaggaagcccagcatttgggacaccttcatccatcaatacatgcctgacggctccaatgcagatgtctcagcagatcagtatcaccattacaaggaggatgtaaagcttatgtatgacatggggctggatgcgtacagattttccattgcttggcctcgtcttattccgggaagaggagagatcaaccccaagggcttggagtactacaacaatctgatagacgaattgataatgcatatacaacctcatgtcaccatctaccattttgatctccctcaggcccttcaggatgagtatggtggaatactcagccccagattcgaagattactcggcttatgccgaggtgtgcttcaagaacttcggtgacagggtgaagcactgggccacctttaaccagccgaacatcgagcccatcggcggcttcgacgccggcgaccggccgccgcggcggtgctcctaccccttcggcaccaactgcaccggcggcgactcctcgacggagccgtacatcgtggctcaccacctgctgctcgctcatgcctcggcagtgtccatctacagacagaaataccagcaggcaattcaaggaggccagatagggatcactctcatggttcggtggcatgagccatacaccgacaaaacagccgatgcagctgctgccattaggatgaatgaattccatattggatttttgcatcctttggtgcacggagactaccctccggtgatgaggagtcgcgtgggcggtcggttgccatccataacggcgtcagattcagagaaaatacgcggatcgttcgacttcatcggcatcaaccattactacgttatctttgtgcaatccatcgacgcgaatgagcagaaactacgggactactacatcgacgcaggtgttcaagaagacgacaaggagaacattcagtgtcattcttggtctcttggcaaggtgctgaatcacctgaaactcgagtatggcaaccccccggtcatgatccacgaaatggtagttattcagattcaccggatatcttcggaaagatcaactacaacgatgacttcagatcggagttcttgcagggctacctggaagctctgtatttgtccgtacggtgcgctcactctccttgctacttacttcctttgtgctggtaaaggaagttga

SEQ ID NO: 52 - protein, 223 aa, Oryza sativa
  PEPTIDE (0)...(0): line 1B-115-22 polypeptide sequence
MAKLILATFAVVFMALAATSLAGDPDMLQDVCVADYKSLKGPLRLNGFPCKRIENVTANDFFFDGLMKAGNTGNAVGSVVTAASVESLPGLNTMGVSMARIDYAPWGLNPPHTHPRATEIIFVVEGSLDVGFVTTANKLFTRTVCKGEVFVFPRGLVHFQKNNGNTPAFAIAALNSQLPGTQSIAAALFGAAPPLPSDTLARAFQVDGGMVEFIKSKFVPPKY

SEQ ID NO: 53 - protein, 332 aa, Oryza sativa
  PEPTIDE (0)...(0): line 1B-164-43 polypeptide sequence
MSSRMAGSAILRHVGGVRLFTASATSPAAAAAAAARPFLAGGEAVPGVWGLRLMSTSSVASTEAAAKAEAKKADAEKEVVVNSYWGIEQSKKLVREDGTEWKWSCFRPWETYTADTSIDLTKHHVPKTLLDKIAYWTVKSLRFPTDIFFQRRYGCRAMMLETVAAVPGMVGGMLLHLRSLRRFEQSGGWIRTLLEEAENERMHLMTFMEVANPKWYERALVITVQGVFFNAYFLGYLLSPKFAHRVVGYLEEEAIHSYTEFLKDLEAGKIDNVPAPAIAIDYWRLPANATLKDVVTVVRADEAHHRDVNHFASDIHYQGMELKQTPAPIGYH

SEQ ID NO: 54 - protein, 1073 aa, Oryza sativa
  PEPTIDE (0)...(0): line 1B-192-40 polypeptide sequence
MVCAAIHIAVVAMLVSLTALAIADESDNNQREALLCIKSHLSSPEGGALTTWNNTSLDMCTWRGVTCSSELPKPRLVVALDMEAQGLSGEIPPCISNLSSLTRIHLPNNGLSGGLASAADVAGLRYLNLSFNAIGGAIPKRLGTLRNLSSLDLTNNNIHGEIPPLLGSSSALESVGLADNYLTGGIPLFLANASSLRYLSLKNNSLYGSIPAALFNSSTIREIYLGENNLSGAIPPVTIFPSQITNLDLTTNSLTGGIPPSLGNLSSLTALLAAENQLQGSIPDFSKLSALRYLDLSYNNLSGTVNPSVYNMSSITFLGLANNNLEGIMPPGIGNTLPNIQVLIMSDNHFHGEIPKSLANASNMQFLYLANNSLRGVIPSFGLMTDLRVVMLYSNQLEAGDWAFLSSLKNCSNLQKLHFGENNLRGDMPSSVAELPKTLTSLALPSNYISGTIPLEIGNLSSISLLYLGNNLLTGSIPHTLGQLNNLVVLSLSQNIFSGEIPQSIGNLNRLTELYLAENQLTGRIPATLSRCQQLLALNLSCNALTGSISGDMFIKLNQLSWLLDLSHNQFINSIPLELGSLINLASLNISHNKLTGRIPSTLGSCVRLESLRVGGNFLEGSIPQSLANLRGTKVLDFSQNNLSGAIPDFFGTFTSLQYLNMSYNNFEGPIPVDGIFADRNKVFVQGNPHLCTNVPMDELTVCSASASKRKNKLIIPMLAAFSSIILLSSILGLYFLIVNVFLKRKWKSNEHMDHTYMELKTLTYSDVSKATNNFSAANIVGSGHFGTVYRGILHTEDTMVAVKVFKLDQCGALDSFMAECKALKNIRHRNLVKVITACSTYDPMGSEFKALVFEYMANGSLESRLHTKFDRCGDLSLGERISIAFDIASALEYLHNQCIPPVVHCDLKPSNVLFNNDDVACVCDFGLARSIRVYSSGTQSISTSMAGPRGSIGYIAPEYGMGSQISTEGDVYSYGIILLEMLTGRHPTNEIFTDGLTLRMYVNASLSQIKDILDPRLIPEMTEQPSNHTLQLHEHKKTVPSRCKLGGVEGILTCTIREIASKLGGLRLSMLT

SEQ ID NO: 55 - protein, 637 aa, Oryza sativa
  PEPTIDE (0)...(0): line 1B-207-27 polypeptide sequence
MQDHILTAFLVVSLLFACIPPAKSADLNSDKQALLAFAASLPHGRKLNWSSAAPVCTSWVGVTCTPDNSRVQTLRLPAVGLFGPLPSDTLGKLDALEVLSLRSNRITVDLPPEVGSIPSLHSLYLQHNNLSGIIPTSLTSTLTFLDLSYNTFDGEIPLRVQNLTQLTALLLQNNSLSGPIPDLQLPKLRHLNLSNNNLSGPIPPSLQRFPANSFLGNAFLCGFPLQPCPGTAPSPSPSPTSPSPGKAKKGFWKRIRTGVIIALAAAGGVLLLILIVLLLICIFKRKKSTEPTTASSSKGKTVAGGRGENPKEEYSSGVQEAERNKLVFFEGCSYNFDLEDLLRASAEVLGKGSYGTTYKAVLEDGTTVVVKRLKEVVVGKKDFEQQMEIVGRVGQHQNVVPLRAYYYSKDEKLLVYDYIPSGSLAVVLHGNKATGKAPLDWETRVKISLGVARGIAHLHAEGGGKFIHGNLKSSNILLSQNLDGCVSEFGLAQLMTIPPAPARLVGYRAPEVLETKKPTQKSDVYSFGVLVLEMLTGKAPLRSPGREDSIEHLPRWVQSVVREEWTAEVFDVDLLRHPNIEDEMVQMLQVAMACVAAPPDQRPKMDEVIRRIVEIRNSYSGSRTPPEEKQKDESAAP

SEQ ID NO: 56 - protein, 523 aa, Oryza sativa
  PEPTIDE (0)...(0): line 1B-138-07 polypeptide sequence
MAAPLSTAAAASWLSDSASSPPRVRLLIGGEFVESRADEHVDVTNPATQEVVSRIPLTTADEFRAAVDAARTAFPGWRNTPVTTRQRIMLKYQELIRANMDKLAENITTEQGKTLKDAWGDVFRGLEVVEHACGMGTLQMGEYVSNVSNGIDTFSIREPLGVCAGICPFNFPAMIPLWMFPIAVTCGNTFVLKPSEKDPGAAMMLAELAMEAGLPKGVLNIVHGTHDVVNNICDDEDIKAVSFVGSNIAGMHIYSRASAKGKRVQSNMGAKNHAIILPDADRDATLNALIAAGFGAAGQRCMALSTAVFVGGSEPWIRPGIREDELVKRASSLVVNSGMASDADLGPVISKQAKERICKLIQSGADNGARVLLDGRDIVVPNFENGNFVGPTLLADVKSEMECYKEEIFGPVLLLMKAESLDDAIQIVNRNKYGNGASIFTTSGVSARKFQTDIEAGQVGINVPIPVPLPFFSFTGSKASFAGDLNFYGKAGVQFFTQIKTVTQQWKESPAQRVSLSMPTSQK

SEQ ID NO: 57 - protein, 446 aa, Oryza sativa
  PEPTIDE (0)...(0): line 1D-059-12 polypeptide sequence
MAAAATAAVPLDEAKAKEVLRQVEFYFSDSNLPRDNFLRKTVEESEDGLVSLALICSFSRMKKHLGLDADVKQETMPEETVLAVAEVLRRSSALRVTEDGKKVGRSIELSKLDEIMEQVDSRTIAASPFPYNVKLEDVQSFFAQYGKVNSVRLPRHIADKRHFCGTALVEFSEEEEANAVLKNTLVFAGADLEIKPKKEFDTEREAKKEAYEKSQPTKNGHDEGYPKGLIVAFKLKIIQIDGGMAENGGDKEGETDDANKSRTGHDEKIPENSDIKEDLSDDVEKSKEAAAQSVKKGESPSENADDPISREDFKEEFGKFGTVRYVDFSIGEDSGYIRFEDSKAAEKARALAAISDEGGLIMKGHLVTLEPVSGQAEKDYWSAIKGGQGKYRDNRSNRGRADPLKYHNAPRMNLRSGLKLKIEVEALWHTRMPTHIFGVILSWNWQ

SEQ ID NO: 58 - protein, 859 aa, Oryza sativa
  PEPTIDE (0)...(0): line 1C-087-40 polypeptide sequence
MATRYWIVSLPVQTPGSTANSLWARLQDSISRHSFDTPLYRFNVPDLRVGTLDSLLALSDDLVKSNVFIEGVSHKIRRQIEELERAGGVESGALTVDGVPVDTYLTRFVWDEGKYPTMSPLKEIVGSIQSQVSKIEDDMKVRGAEYNNVRSQLSAINRKQTGSLAVRDLSNLVKPEDMVTSEHLVTLLAVVPKYSQKDWLSSYESLDTFVVPRSSKKLYEDNEYALYTVTLFAKVVDNFKVRAREKGFQVRDFEYSSEAQESRKEELEKLMQDQEAMRASLLQWCYASYSENTIYVRHAKVQNFVLLYVFSSWMHFCAVRVFVESILRYGLPPSFLSAVLAPSQKGEKKVRSILEELCGNVHSIYWKSEDDVGVAGLGEHRVRCCGYYVFVTLSSQKDKLGVMYRTSILGDPVPTDDSRVDKTQEEMMVKEKEIHVMSNRRRKRGESKPQAHDAGDTTPIDNILTSLDDAPPPPPPPRRELPLLQLNDHQEADDADASSSSSPHQQRRLWVKDRSRAWWELCSSADYPEADFRRAFRMSRPTFHFLCDALAAAVAKEDTALRAAIPVRQRVAVCVWRLATGEPLRVVSKRFGLGISTCHKLILEVCAAIRNLLMPRFLHWPDHPTSTAYKTRFEATSGVSGVVGAMYTTHIPIIAPKVSVAAYLNRRHTERNHKTSYSITLQGVVGPDGTFTDVCIGWPGSMSDEQVLRKSALHQRASAAAGSMSWVVGGASYPLTEWMLVPYAQRNLTWTQHAFNEKVGEVRRVATEAFVRLKGRWACLQKRTEVKLQDLPAVLAACCVLHNICETRGEDMDPDLRCDLPPDEEEDDTVLVQSESANKVRDDIAHNLLHRGLAGTAFF

SEQ ID NO: 59 - protein, 473 aa, Oryza sativa
  PEPTIDE (0)...(0): line 1C-017-14 polypeptide sequence
MDALLVEKVLLGLFVAAVLALVVAKLTGKRLRLPPGPAGAPIVGNWLQVGDDLNHRNLMALARRFGDILLLRMGVRNLVVVSSPDLAKEVLHTQGVEFGSRTRNVVFDIFTGKGQDMVFTVYGDHWRKMRRIMTVPFFTNKVVAQNRAGWEEEARLVVEDVRRDPTAATSGVVIRRRLQLMMYNDMFRIMFDRRFDSVDDPLFNKLKAFNAERSRLSQSFEYNYGDFIPVLRPFLRRYLARCHQLKSQRMKLFEDHFVQERKRVMEQTGEIRCAMDHILEAERKGEINHDNVLYIVENINVAAIETTLWSIEWGIAELVNHPSIQSKVREEMASVLGGAAVTEPDLERLPYLQAVVKETLRLRMAIPLLVPHMNLADGKLAGYDIPAESKILVNAWFLANDPKRWVRPDEFRPERFLEEEKAVEAHGNDFRFVPFGVGRRSCPGIILALPIIGITLGRLVQSFDLLPPPGMDK

SEQ ID NO: 60 - protein, 408 aa, Oryza sativa
  PEPTIDE (0)...(0): line 1C-038-56 polypeptide sequence
DVQQHSGSSSSSTESDVQETAAVAVADPSPRSEVVDGESPPQPGGEAASHQQQQKEMKLKKPDKILPCPRCSSMDTKFCYFNNYNVNQPRHFCKHCQRYWTAGGAMRNVPVGAGRRKNKNATAAAHFLHRVRACAAAAAMPAAPHDATNATVLSFGGGGGGHDALPVTLDLADKMTRLGKEGLVAHARNADAAAACSEVSSNRDDEQIGNTVAKPANGLQQHPPPPHHHHHSAMNGGGIWPYYTSGIAIPIYPAAPAYWGCMIPPPGAWSLPWPATVQSQAISSSSPPTSATPSVSSFTLGKHPREGGDHEARDHHGNGKVWVPKTIRIDNADEVARSSIRSLFAFRGGDKVDDNNDDDGTSVHKLATTVFEPKRDGKTAKHPAITSLPLLHTNPVALTRSATFQEGS

SEQ ID NO: 61 - protein, 290 aa, Oryza sativa
  PEPTIDE (0)...(0): line 1C-041-47 polypeptide sequence
MGIKGLTKLLADNAPKAMKEQKFESYFGRRIAVDASMSIYQFLSFARYSKREDATKELTEAVEEGDKDAIEKFSKRTVKVTKQHNEECKRLLRLMGVPVVEVYAVASEDMDSLTFGAPRFLRHLMDPSSKKIPVMEFEVAKVLEELELTMDQFIDLCILSGCDYCDSIKGIGGQTALKLIRQHGSIESILENINKDRYQIPEDWPYQEARRLFKEPNVTLDIPELKWNAPDEEAIEKIKFAKNKSSQGRLESFFKPVVSTSVPLKRKDTSEKPTKAVANKKTKGAGGKKK

SEQ ID NO: 62 - protein, 552 aa, Oryza sativa
  PEPTIDE (0)...(0): line 1C-064-20 polypeptide sequence
MASAPGDGKQGGGGGGPAVGIDLGTTYSCVAVWRHDRGEVIANDQRNRLTPSCVAFTADDDDSFVGDAAFNQSALNPTNTIFEVKRLIGRRFSDDSVQKDIKLWPFKVVAGQEDRPMIVVRHEGEERQFMPEEISSMVLAKMRETAEVYLGKTVTKAVITVPVYFNNAQRQATMDAGAIAGLNVMRIINEPTAAALAYCLEKMPVSNKGRMVLVFDLGGGTFDISLLNIDPGEGTAGDTHLGGADFDNELVKHSLREFNRKHGSMDIESNQKALRRLRTACERAKRMLSSTMQTTIEVDSLHQGIDFRVTLTRSRFEELNKDLFSKCMEAMENCLRDAKVDKWSVDDVVLVGGSTRIPKVQKMLSEFFDGKELCRSINPDEAVAYGAAIQASILCGGTDDKRLVDMLLREVTPLSLGVETEDNCTMSVVIPRNTAIPTKKVKNFTTLYDNQINVSFPVYEGESANTKDNNLLGEFTLYGIPPAPKRVPSIDVTFDIDANGVLNVSAEHKVTGQKNSITITNRSGRLNQEEIDRMALEPERHKMKRIKLHEVV

SEQ ID NO: 63 - protein, 479 aa, Oryza sativa
  PEPTIDE (0)...(0): line 1C-109-35 polypeptide sequence
MSSSATVVPLAYQGNTSASVADWLNKGDNAWQLVAATLVGLQSVPGLVVLYGGVVKKKWAVNSAFMALYAFAAVWICWVTWAYNMSFGEKLLPIWGKARPALDQGLLVGRAALPATVHYRADGSVETAAVEPLYPMATVVYFQCVFAAITLILVAGSLLGRMSFLAWMIFVPLWLTFSYTVGAFSLWGGGFLFHWGVIDYCGGYVIHVSAGIAGFTAAYWVGPRAQKDRERFPPNNILFTLTGAGLLWMGWAGFNGGGPYAANSVASMAVLNTNICTAMSLIVWTCLDVIFFKKPSVVGAVQGMITGLVCITPAAGVVQGWAALVMGVLAGSIPWYTMMILHKRSKILQRVDDTLGVFHTHGVAGLLGGLLTGLFAEPTLCNLFLPVADSRGAFYGGAGGAQFGKQIAGGLFVVAWNVVVTSLICLAINLLVPLRMPDDKLEVGDDAVHGEEAYALWGDGEMYDVTKHGSDAAVAPVVV

SEQ ID NO: 64 - protein, 2177 aa, Oryza sativa
  PEPTIDE (0)...(0): line 1C-109-51 polypeptide sequence
MANLGGGAEAHARFKQYEYRANSSLVLTTDSRPRDTHEPTGEPETLWGRIDPRSFGDRAVQAKPPELEEKLTKSRKKKAAASDPDDLHRRDAKRRRRAAAAQSEVSVLSLTDDVVYKPQTKETRAAYEALLSVIQQQFGGQPLDVLGGAADEVLAVLKNDKIKSPDKKKEIEKLLNPISNQMFDQIVSIGKLITDFHDASAGDSAAAPSGDGMDTALDDDIGVAVEFEENEDDEESDFDQVQDDLDEDEDDDLPESNAPGAMQMGGELDDDDMQNSNEGLTINVQDIDAYWLQRKVSQAYEDIDPQHSQKLAEEILKIIAEGDDRDVENRLVMLLDYEKFDLIKLLLRNRLKIVWCTRLARAEDQEQRKKIEEDMMGNPTLTPILEQLHATRASAKERQKNLEKSIRDEAKRLTKSENTGIDGARDRRAVDRDMESGWLKGQRQLLDLDSLSFHQGGLLMANKKCELPPGSFRTPHKGYEEVHVPALKAKPYETGEKIVKISDMPEWAQPAFAKMTQLNRVQSKVYETALFKPDNILLCAPTGAGKTNVAVLTILQQIGLHMKDGVFDNTKYKIVYVAPMKALVAEVVGNLSARLSAYGITVRELSGDQNLTKQQIDETQIIVTTPEKWDIVTRKSGDRTYTQMVKLLIIDEIHLLHDNRGPVLESIVSRTVRQIETTKEHIRLVGLSATLPNYEDVAVFLRVRSDGLFHFDNSYRPCPLAQQYIGITVRKPLQRFQLMNEICYEKVMASAGKHQVLIFVHSRKETAKTARAIRDTALANDTLNRFLKDDSASQEILGSQAELVKSSDLKDLLPYGFAIHHAGLARVDRELVEELFADKHIQVLVSTATLAWGVNLPAHTVIIKGTQIYNPEKGAWTELSPLDVMQMLGRAGRPQYDTHGEGIILTGHSELQYYLSLMNQQLPIESQFISRLADQLNAEIVLGTIQNAREACSWLGYTYLYIRMLRNPTLYGLPADIMETDKTLDERRADLVHSAANLLDRNNLIKYDRKTGYFQVTDLGRIASYYYISHGTISTYNEYLKPTMGDIELCRLFSLSEEFKYVSVRQDEKMELAKLLDRVPIPVKESLEEPSAKINVLLQAYISRLKLEGLSLSSDMVYIRQSAGRLLRALFEIVLKRGWAQLAEKALNLCKMIDKQMWNVQTPLRQFPGIPKEILMKLEKKELAWERYYDLSSQEIGELIRFPKMGRQLHKCIHQLPKLNLSAHVQPITRTVLGFELTITPDFQWDDKVHGYVEPFWVIVEDNDGENILHHEYFMVKKQYVDEDHTLNFTVPIYEPLPPQYFIRVVSDKWLGSQTILPVCFRHLILPEKYAPPTELLDLQPLPVTALRNARYEGLYSAFKHFNPIQTQVFTVLYNTDDSVLVAAPTGSGKTICAEFAILRNHQKAVSGESNMRVVYIAPIEALAKERYRDWEQKFGEFARVVELTGETAADLKLLDKGEIIISTPEKWDALSRRWKQRKQVQQVSLFIVDELHLIGSEKGHVLEVIVSRMRRIASHIGSNIRIVALSASLANAKDLGEWIGATSHGLFNFPPAVRPVPLEIHIQGVDIANFEARMQAMTKPTYTAITQHAKNGKPALVFVPTRKHARLTALDLCAYSSAEGGGTPFLLGSEDEMDAFTGGISDETLKYTLKCGVGYLHEGLSDLEQEVVTQLFLSGRIQVCVASSTVCWGRSLPAHLVVVMGTQYYDGRENAHTDYPITDLLQMMGHASRPLQDNSGKCVILCHAPRKEYYKKFLFEAFPVESHLHHFLHDHMNAEVVVGVIENKQDAVDYLTWTFMYRRLTKNPNYYNLQGVSHRHLSDHLSELVETVLNDLESSKCVAIEEDMYLKPLNLGLIASYYYISYTTIERFSSMLTQKTKMKGLLEILASASEYAELPSRPGEEDFIEKLVRHQRFSIEKPRYGDPHVKANALLQAHFSRHTILGNLAADQREILLSAHRLLQAMVDVISSNGWLTLALNAMELSQMVTQGMWDRDSVLLQLPHFTKELARRCQENEGRPIESIFDLAEMSIDEMRDLLQQSNPQLQDIIEFFKRFPNVDMAYEVREGDDIRAGDNVTVQVTLERDMTNLPSEVGPVHAPRYPKPKEEGWWLVIGDSSTNQLLAIKRVALQKRARVKLEFTAASEAGRKEYMIYLMSDSYLGCDQEYEFTVDVMDAGGD

SEQ ID NO: 65 - protein, 181 aa, Oryza sativa
  PEPTIDE (0)...(0): line 1C-056-07 polypeptide sequence
MVGCLATSTKTILAESLLHGYKFDSINTVYYMAPFATMILALPAVLLEGGGVVTWFYTHDSIASALVIIIGSGVLAFCLNFSIFYVIHSTTAVTFNVAGNLKVAVAVLVSWLIFRNPISPMNAIGCAITLVGCTFYGYVRHLISQQQAVAPGTGSPTTSQTNSPRSRMEMLPLVGDKQEKV

SEQ ID NO: 66 - protein, 793 aa, Oryza sativa
  PEPTIDE (0)...(0): line 1C-100-32 polypeptide sequence
MEMEMEDNKAPSPPSPMESDVAEEKRKREDDASSSAVLAAANNTGGAQHPMWKTSLCSFFRRRAASSADGCSHGDSCRYAHSEEELRPRPDGTWDPTSDRAKKLRKVAADEVEEEVVTIDDKALDKCLVGLPRGWANDRLKTFLQDKARTNYSSILPPALLLGISYATAKKKKGMTVGFVTFENIEQLKNAIEVLTENQSGGKEIKIADANRRSHQKLHTEKPVSDNGVTTENGTSVDVPPGETSAPEAAISNKKSVRDAVTPLAHMSYNDQLEHKNNSVAQILKRLTRNARKACPTGIPLPDWVFKSKEIGGLPCKLEGILESPVINGYRNKCEFSVGFSLEGKKTVGFMLGNFSTDMIDKTKSREGVTAVEEPVDCPNVSEISCKYALMFQDFLQSSSLPVWNRVNNCGFWRQFTVREGRCRAQAVAQNAETQISEVMLIVQVCSTGVDDAVMKDEFDKLTVALQQGAATCSPPLPLTTIVVQDHKGISNAAPADCPLIPLLVPKVDQSEGTVDKTRIHDHIGNLWFSISPTAFFQVNTLAAERLYTLAGDWANLNSGTLLFDVCCGTGTIGLTLAHRVGMVVGIEMNESAVSDAERNALINGVSNCRFVCGKAEDVMGSLLTEYLGSPQQDIPVSEGAVSGTVKDEEVIDSSKNSGENLDSSMQKNDNGKSQQLGDAPADSSSSAIDEIKGNSNDRVGNGLEGSHDEYNEVAGEDIHGEASLINESVDLKVSDCLEDRKTSDDGSSISNNDVTAATACQFEDIVAIVDPPRVGLHPTVNPKRGQGVPVFI

SEQ ID NO: 67 - protein, 143 aa, Oryza sativa
  PEPTIDE (0)...(0): line 1C-142-27 polypeptide sequence
MAMAYKMATEGMNVKEECQRWFMEMKWKKVHRFVVYKIDERSRAVLVDKVGGPGEGYEELVAALPTDDCRYAVFDFDFVTVDNCQKSKIFFIAWSPTASRIRAKILYATSKQGLRRVLDGVHYEVQATDSSEMGYDVIRGRAQ

SEQ ID NO: 68 - protein, 452 aa, Oryza sativa
  PEPTIDE (0)...(0): line 1C-140-04 polypeptide sequence
MATGELALVSSLFIVVVFLLLGAVAREASALTRHDFPEGFVFGAGSSAFQVEGAAAEDGRKPSIWDTFIHQGYMPDGSNADVSADQYHHYKEDVKLMYDMGLDAYRFSIAWPRLIPDGRGEINPKGLEYYNNLIDELIMHGIQPHVTIYHFDLPQALQDEYGGILSPRFIEDYSAYAEVCFKNFGDRVKHWATFNQPNIEPIGGFDAGDRPPRRCSYPFGTNCTGGDSSTEPYIVAHHLLLAHASAVSIYRQKYQQAIQGGQIGITLMVRWHEPYTDKTADAAAAIRMNEFHIGWFLHPLVHGDYPPVMRSRVGGRLPSITASDSEKIRGSFDFIGINHYYVIFVQSIDANEQKLRDYYIDAGVQGEDDKENIQCHSWSLGKVLNHLKLEYGNPPVMIHEMVIQIHRISSERSTTTMTSDRSSCRATWKLCICPYGALTLLATYFLCAGKGS

SEQ ID NO: 69 - DNA, 6282 bp, Artificial Sequence
  misc_feature (0)...(0): T-DNA sequence artificial sequence synthetic nucleic acid
gtttacccgccaatatatcctgtcaaacacggatccgaggtaccaggtaccaggtgagttccattcttactaccacggtgctattttttttgctatgtggctaattacatgactaacttggggtgctaaatcttacaggttatatgcaggttatatgcaggtcccgggtaggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaattcatcgataggctagtcatggtgactgtacgttgtaagtgcagcaaactgccgacgcgatgcaaactgtacacgttaacatgccactcacctggaacgcacaatggccactaggtgcggccgtagtgtggatttcaaagagagagagagagagagagagctaatcacgtaaacgtaaacacagcagatagcagagatgttgattaggcaaaacagtataaaagccaatccaataaactacatttagcgaagtgctatactaatgcactaataacgaactgttcttttcttaagatcggagccagtaatgggttgtcagcaggagaagcacgtaaaccttgaaacatactaagttccacagtcgagagtaaaccgtaatcaacacaagaaacaaacataaaattgaacaaacgcgcatattataagtgacgaagcggtctcacataaaacagggcacacaggttacaacaacgagggttgtaagcccattaagccccaaacatcagatcaccacaagcaaatgtctcgaagacacacgcacacggcaacaggataactccacactggcagatcatgggatagcagcagttatcaatcaggccttgacacacagaacatcaagcccccagacgacgacgactcctctagatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatccgtcgagtcagcctgaaaggacaaaatacatgttagcgccttagtggtacattattatttcagtacagcaagataacacaattcaaaagactgacccataataaataactagtcctcaatttaaaatttgagttcctaaatagacatctatgaatatgctgtacatcggcactacagaaaatacgattcccaataattgaacaattgtactttatttagttgttactacaacaatggaagatacaagatcgtttcaaaactaccatacatgcatggagtatttgttccacagatctggaaaaaacagatctgacgggcagtgtcaccaaacactagacatatgtatttgtattaggtggatgacgtgtacaaacatgactaccagatctagaattagaacgcggcgtgctttggaaatgttaagtagatccaaatacatcggtaacaaatgaacatattcatatgacatagctgtaaaacatcatgatctatcatatcaactagaggggatctcggcatgtatcattctattgcatctagaaccatagcatttccatgtgacaaacaattttgaaacatacattgctcagatctaccataagaacaccatgttcatgacagcatcgaccatgatttccacatttaacagcatcccgaggtcagatccagtgtttagaactatcatttcagcaaaattcacaaaataaatcctccaattcgcctacatatatccattccacgcatcctaggaccagatccaccataccagtatacacgaacactaaacatttcatcagatccgctaactacgacagagaaaacgcagatccagacaaccagatccaccgacaaataaacacagccccccacatccaacaaccgcgaatccacccagatctgacccagaagcaggcacgaagaacacggggggtgagggagatgggacgcggatccaagcttggcggcggattgggttgatgctgcgacggcggcggagaagggagagaggggagaggaggaaaagcgcggaggcgcggagagaggcgagtacgaagacgcctttctctgcgttgtctcggcttagggtttgcgatccccgcactccgcccgttttatagggccagacagcctggacctctcaggagcggaagcgaagggatgggggagtttttcgttttaccccctttgacgtcttcaaaattcactcgcatctcacatcgtgcctgcaaaggtcgggtggttggatcatatcgagaatatttaataagttagggagctttctgataatatccaccgcaaggaggccatttcgtttgcactcggttgaagtccatacccgcctctcgttagtttcactgacaggtgggtccagaggcttccttcgtgtgcggttagcgagtgcacgcgtcgctccaagaaaaagcagtaggtgacctggccacctcgttgtaggtgcgaccgatgcagacgtcccgcttgccggtgggcccacgttacagctgggtcccacatatcggtgggtgcgatgagttgttcggtgcgtaggagtagattggatcccaacgggtggttgcgtcgtcggcggccctgccgattttattttcgatttttgaaatgcgagagcgggagaacggcaccgttggcttggctgtggatgccgttagccgcaccggactattcggcccagttccattttggcccaacttgaaacagcgcggagcacaagacgggggagcccatttaggcccgtaatctcagccctgtagaaagtccgtgtcgttgcgatggaccagaaagcccataaaccttggaggcgttctctgctgggaataaaataagttcacactctgtccctcaactttacgtcgagtttgtttgacatcgctaatgcccagtaccagaaatcttggatacaaataaggtttgatataaagtggcacgtggtgaatggcatcgtttgaaaaatcttcagatcgagcgggcttcacaagccgtaagtgcaagtgccaaaatttgtgaagaaaattacttccttcatttcacaatgtaagttattttagcattttccacattcatattgatatcaataaatttagatagatatatatgtctgaattcgtaccgttaacgagctctagaagcttcagtactacgcgtctcgagaattcagtacattaaaaacgtccgcaatgtgttattaagttgtctaagcgtcaatttgtttacaccacaatatatcctgcca

SEQ ID NO: 70 - DNA, 43 bp, Artificial Sequence
  synthetic oligonucleotide
gggtcgacgaggtacaaggtacaaggtacagacttgtatcctt

SEQ ID NO: 71 - DNA, 50 bp, Artificial Sequence
  synthetic oligonucleotide
cgggtaccacctgcatataacctgcatataacctgcacattagcaataaa

SEQ ID NO: 72 - DNA, 40 bp, Artificial Sequence
  synthetic oligonucleotide
ggatccgaggtaccaggtaccaggtgagttccattcttac

SEQ ID NO: 73 - DNA, 47 bp, Artificial Sequence
  synthetic oligonucleotide
cccgggacctgcatataacctgcatataacctgtaaagatttagcac

SEQ ID NO: 74 - DNA, 27 bp, Artificial Sequence
  synthetic oligonucleotide
gaagatctagaggagtcgtcgtcgtct

SEQ ID NO: 75 - DNA, 24 bp, Artificial Sequence
  synthetic oligonucleotide
ccatcgataggctagtcatggtga

SEQ ID NO: 76 - DNA, 22 bp, Artificial Sequence
  synthetic oligonucleotide
gccgtaatgagtgaccgcatcg

SEQ ID NO: 77 - DNA, 22 bp, Artificial Sequence
  synthetic oligonucleotide
atctgcatcggcgaactgatcg

SEQ ID NO: 78 - DNA, 22 bp, artificial sequence
  misc_feature (0)...(0): synthetic oligonucleotide
cacgggttggggtttctacagg

SEQ ID NO: 79 - DNA, 21 bp, Artificial Sequence
  synthetic oligonucleotide
gcttggactataatacctgac

SEQ ID NO: 80 - DNA, 23 bp, Artificial Sequence
  synthetic oligonucleotide
ttggggtttctacaggacgtaac

SEQ ID NO: 81 - DNA, 24 bp, Artificial Sequence
  synthetic oligonucleotide
caagttagtcatgtaattagccac

SEQ ID NO: 82 - DNA, 23 bp, Artificial Sequence
  synthetic oligonucleotide
ccatgtagtgtattgaccgattc

SEQ ID NO: 83 - DNA, 23 bp, Artificial Sequence
  synthetic oligonucleotide
tcgtctggctaagatcggccgca

(19) United States
(12) Patent Application Publication
(10) Pub. No.: US 2006/0107344 A1
(43) Pub. Date: May 18, 2006

(54) Organ preferential genes identified by T-DNA insertional mutagenesis of rice

(76) Inventors: Gynheung An, Pohang (KR); Choong-Hwan Ryu, Pohang (KR); Jong-Jin Han, Pohang (KR); Hong-Gyu Kang, Pohang (KR); Kyungsook An, Pohang (KR)

Correspondence Address: JHK Law, P.O. Box 1078, La Canada, CA, 91012-1078, US

(21) Appl. No.: 10/713,648

(22) Filed: Nov. 14, 2003

Related U.S. Application Data
(60) Provisional application No. 60/427,166, filed on Nov. 15, 2002.

(51) Int. Cl. A01H 5/00; C12Q 1/68; C07H 21/04; C12N 15/82; C12N 5/04; A01H 1/00

(52) U.S. Cl. 800/278; 800/320.2; 435/6; 435/69.1; 435/468; 435/419; 536/23.2

(57) ABSTRACT

The present invention provides a method of producing rice lines that carry genes that have been modified by T-DNA/GUS based insertional mutagenesis. The GUS portion of the insert is promoterless, so that the GUS gene is expressed only when it is inserted into an active gene. In this way, organ preferential expression of various rice genes can be determined. The invention is also directed to the organ-preferential genes found by the T-DNA/GUS insertional mutagenesis method, as well as the proteins encoded by them. The invention also involves a database having information about the rice lines, such as the genes having the insert, the encoded proteins, the phenotypic characteristics of the mutant lines, and promoter activity of the tagged genes.

### BACKGROUND OF THE INVENTION

1. Field of the Invention

The present invention relates to the field of plant biology and specifically rice-lines having heterologous marker genes inserted downstream of organ preferentially-expressed promoters. The invention also relates to the native genes found by the insertional mutagenesis procedure, as well as the polypeptides encoded by them.

2. Description of the Related Art

There has been much progress in the development of strategies to discover the function of plant genes. Development of the strategies has been largely based on genetic approaches such as mutant identification and map-based gene isolation (reviewed in Martin, 1998). Gene inactivation by insertion of a transposon has been employed for functional studies in several plant species. The use of transfer DNA (T-DNA) as a mutagen has also been developed for tagging genes in Arabidopsis (Babiychuk et al., 1997, Proc. Natl. Acad. Sci. USA 94: 12722-12727; Feldmann, 1991, Plant Jour. 1:71-82; and Krysan et al., 1999, Plant Cell 11:2283-2290; the disclosures of which are hereby incorporated by reference in their entireties). It is believed that T-DNA insertion is a random event, and that the inserted genes are stable through multiple generations (reviewed in Azpiroz-Leehan and Feldmann, 1997, Trends Genet. 13:152-156 the disclosure of which is hereby incorporated by reference in its entirety).

Insertional mutagenesis is a useful method for functional analysis due to the development of several strategies for screening T-DNA or transposon insertions in a known gene and recovering sequences flanking the insertions (Cooley et al., 1996, Mol. Gen. Genet. 152:184-194; Couteau et al., 1999, Plant Cell 11:1623-1634; Frey et al., 1998, Plant Jour. 13:717-721; Koes et al., 1995, Proc. Natl. Acad. Sci. USA, 92:8149-8153; Krysan et al., 1999, supra; Liu and Whittier, 1995, Genomics, 25, 674-681, the disclosures of which are hereby incorporated by reference in their entireties). Through sequencing PCR-amplified fragments adjacent to the inserted element, a flanking sequence database has been constructed in Arabidopsis (Parinov et al., 1999, Plant Cell 11:2263-2270; Tissier et al., 1999, Plant Cell 11:1841-1852, the disclosures of which are hereby incorporated by reference in their entireties).

Reporter genes as insertional elements have been utilized to aid in the identification of insertions within functional genes (Campisi et al., 1999, Plant Jour. 17:699-707; Kertbundit et al., 1991, Proc. Natl. Acad. Sci. USA, 88:5212-5216; Kertbundit et al., 1998, Plant Mol. Biol. 36:205-217; Sundaresan et al., 1995, Genes Dev. 9:1797-1810; Topping et al., 1991, Development 112:1009-1019, the disclosures of which are hereby incorporated by reference in their entireties). An enhancer trap contains a weak minimal promoter fused to a reporter gene, and a gene trap contains multiple splicing sites fused to a reporter gene. The GUS gene is the most frequently used as a reporter gene because of the accurate detection of its gene products and the tolerance of N-terminal translational fusions in its enzyme activity (Jefferson et al., 1987, EMBO J. 6:3901-3907, the disclosure of which is hereby incorporated by reference in its entirety).

Rice is a model plant of cereal species because of its relatively small genome size, efficient tools for plant transformation, construction of physical maps, large-scale analysis of expressed sequence tags (ESTs) and international genome sequencing projects, as well as economic importance. Therefore, development of insertional mutant lines will be extremely valuable for the functional genomics of rice.

Methods for transforming rice are described, for example, in European Patent Specification EP0539563 to Christou, and U.S. Pat. No. 6,215,051 to Yu, both of which are herein incorporated by reference in their entireties. Other general methods for transformation of monocotyledonous plants are described, for example, in U.S. Pat. No. 6,037,522 to Dong, and U.S. Pat. No. 5,591,616 to Hiei, both of which are herein incorporated by reference in their entireties.

### SUMMARY OF THE INVENTION

Aspects of the invention include an isolated or purified nucleic acid having a nucleotide sequence selected from the group consisting of SEQ ID NOS:18-34 and the nucleotide sequences complementary to SEQ ID NOS:18-34, or fragments comprising at least 10, 15, 20, 25, 30, 35, 40, 50, 75, 100, 150, 200, 300, 400, 500, 750, 1000, 1250, or 1500 consecutive nucleotides. Additional aspects of the invention include an isolated or purified nucleic acid comprising a nucleotide sequence having at least 70%, or 80%, or 85%, or 90%, or 95%, or 97% homology selected from the group consisting of SEQ ID NOS:18-34 and the nucleotide sequences complementary to SEQ ID NOS:18-34 or fragments comprising at least 10, 15, 20, 25, 30, 35, 40, 50, 75, 100, 150, 200, 300, 400, 500, 750, 1000, 1250, or 1500 consecutive nucleotides.

Another aspect of the invention includes an isolated or purified nucleic acid comprising a nucleotide sequence selected from the group consisting of SEQ ID NOS:35-51 and the nucleotide sequences complementary to SEQ ID NOS:35-51, or fragments comprising at least 10, 15, 20, 25, 30, 35, 40, 50, 75, 100, 150, 200, 300, 400, 500, 750, 1000, 1250, or 1500 consecutive nucleotides. Further aspects of the invention include an isolated or purified nucleic acid comprising a nucleotide sequence having at least 70%, or 80%, or 85% or 90%, or 95%, or 97% homology with a nucleotide sequence selected from the group consisting of SEQ ID NOS:35-51 and the nucleotide sequences complementary to SEQ ID NOS:35-51, or fragments comprising at least 10, 15, 20, 25, 30, 35, 40, 50, 75, 100, 150, 200, 300, 400, 500, 750, 1000, 1250, or 1500 consecutive nucleotides.

A further aspect of the invention includes an isolated or purified nucleic acid encoding a polypeptide comprising an amino acid sequence selected from the group consisting of SEQ ID NOS:52-68, or fragments comprising at least 5, 10, 15, 20, 25, 30, 35, 40, 50, 75, 100, 200, 400, 600, 800, or 1000 consecutive amino acids thereof. Additional aspects of the invention include an isolated or purified nucleic acid encoding a polypeptide having at least 25%, or 40%, or 50%, or 60%, or 70%, or 80%, or 85%, or 90%, or 95%, or 99% amino acid identity with an amino acid sequence selected from the group consisting of SEQ ID NOS:52-68, or fragments comprising at least 5, 10, 15, 20, 25, 30, 35, 40, 50, 75, 100, 200, 400, 600, 800, or 1000 consecutive amino acids.

Other aspects of the invention include an isolated or purified polypeptide comprising an amino acid sequence selected from the group consisting of SEQ ID NOS:52-68, or fragments comprising at least 5, 10, 15, 20, 25, 30, 35, 40, 50, 75, 100, 200, 400, 600, 800, or 1000 consecutive amino acids thereof. Further aspects of the invention include an isolated or purified polypeptide having at least 25%, or 40%, or 50%, or 60%, or 70%, or 80%, or 85%, or 90%, or 95%, or 99% amino acid identity (as measured by BLASTP, BLASTX, or TBLASTN set at default parameters) with an amino acid sequence selected from the group consisting of SEQ ID NOS:52-68, or fragments comprising at least 5, 10, 15, 20, 25, 30, 35, 40, 50, 75, 100, 200, 400, 600, 800, or 1000 consecutive amino acids.

An additional aspect of the invention includes a recombinant nucleic acid having a nucleotide sequence which encodes a polypeptide selected from SEQ ID NOS:52-68, operably linked to a promoter.

An additional aspect of the invention includes a genetically modified rice plant having a gene selected from germin-like protein, alternative oxidase (AOX1a) protein, XA21-like protein kinase gene, receptor-like protein kinase, methylmalonate semi-aldehyde dehydrogenase (MMSDH1), homolog of the RNA-binding protein LAH1, vacuolar ATP synthase subunit C, cinnamic acid 4-hydroxylase, H-protein promoter binding factor-2a, flap endonuclease (FEN-1), heat shock protein Hsp70, ammonium transporter, ATP-dependent RNA helicase, glucose-6-phosphate/phosphate transporter, RNA methyltransferase, actin depolymerizing factor 5, and beta-glucosidase, that has been disrupted.

An additional aspect of the invention includes a genetically modified rice plant having a gene which has a nucleotide sequence selected from SEQ ID NOS:18-34 which has been disrupted.

An additional aspect of the invention includes a genetically modified rice plant wherein the gene encoding a polypeptide which is selected from the group consisting of SEQ ID NOS:52-68: has been disrupted. Embodiments of the invention also include a genetically modified rice plant selected from line designations b-115-22, 1b-164-43, 1b-192-40, 1b-207-27, 1b-138-07, 1d-059-12, 1c-087-40, 1c-017-14, 1c-038-56, 1c-041-47, 1c-064-20, 1c-109-35, 1c-109-51, 1c-056-07, 1c-100-32, 1c-142-27, and 1c-140-04. Further embodiments include a genetically modified rice plant which overexpresses or underexpresses a polypeptide having an amino acid sequence selected from SEQ ID NOS:52-68.

Aspects of the invention include a method of screening a rice plant for a desirable characteristic by first obtaining a rice plant having a gene selected from SEQ ID NOS:18-34 which has been disrupted; and then exposing the plant to conditions which permit the characteristic to be identified. The desirable characteristic may be selected from: altered photosynthetic capacity, altered response to biotic stress, allelopathy, altered response to abiotic stress, altered morphology, altered grain yield, altered nutritional content of grain, altered growth rates, altered secondary product pathways, altered pesticide resistance, altered grain characteristics such as grain shape or taste, cooking quality, altered harvesting qualities, altered optimal growth temperatures, altered resistance to herbicides, altered flowering time, altered seed fill characteristics, altered hormone biosynthetic/degradation pathways, or altered responses to hormones.

Further aspects of the invention include a method of producing a genetically modified plant having an altered phenotype as compared to a wild-type plant, by first contacting a plant cell with a nucleic acid sequence which increases or decreases the expression or activity of a protein selected from SEQ ID NOS:52-68 relative to a wild type plant to obtain a transformed plant cell, then producing a plant from the transformed plant cell, then selecting a plant which expresses the protein. The contacting step may be performed by physical or chemical means. In some embodiments, the plant cell may be from protoplasts, gamete producing cells, or cells which regenerate into whole plants. In some embodiments, the nucleic acid sequence may be linked to a constitutive promoter, a tissue specific promoter, an organ specific promoter, a developmentally specific promoter, an inducible promoter, and the promoter may also be endogenous or heterologous. In further embodiments, the amino acid sequence may have at least 90%, and more preferably at least 95% amino acid identity to a polypeptide selected from SEQ ID NOS:52-68. In some embodiments, the nucleic acid sequence encoding the protein is selected from SEQ ID NOS:18-34 and SEQ ID NOS:35-51.

Aspects of the invention include a genetically modified seed, into which a nucleic acid sequence encoding a polypeptide having at least 80%, or at least 85%, or at least 90%, or at least 95% amino acid identity as measured by BLASTP, BLASTX, or TBLASTN set at default parameters to an amino acid sequence selected from the group consisting of SEQ ID NOS:52-68 has been introduced.

Additional aspects of the invention include an antibody to an amino acid sequence selected from SEQ ID NOS:52-68.

Further aspects of the invention include a method of expressing a gene in a desired tissue or organ of a rice plant, by first obtaining the promoter which directs the transcription of a sequence selected from SEQ ID NOS:18-34; then linking the promoter to the gene to be expressed; and then introducing the promoter operably linked to the gene into a rice plant.

Other aspects of the invention include a computer readable medium having a nucleotide sequence selected from SEQ ID NOS:18-34, the nucleotide sequences complementary to SEQ ID NOS:18-34, or fragments having at least 10, 15, 20, 25, 30, 35, 40, 50, 75, 100, 150, 200, 300, 400, 500, 750, 1000, 1250, or 1500 consecutive nucleotides stored on it. The computer readable medium may also have data indicating the tissue or organ in which the nucleic acid sequences are transcribed.

Additional aspects of the invention include a computer readable medium having stored on it a nucleotide sequence selected from SEQ ID NOS:35-51, the nucleotide sequences complementary to SEQ ID NOS:35-51, or fragments having at least 10, 15, 20, 25, 30, 35, 40, 50, 75, 100, 150, 200, 300, 400, 500, 750, 1000, 1250, or 1500 consecutive nucleotides. The computer readable medium may further have data indicating the tissue or organ in which mRNA having the coding sequence is expressed.

Additional aspects of the invention include a computer readable medium having stored on it an amino acid sequence selected from SEQ ID NOS:52-68, the nucleotide sequences complementary to SEQ ID NOS:52-68, or fragments having at least 5, 10, 15, 20, 25, 30, 35, 40, 50, 75, 100, 200, 400, 600, 800, or 1000 consecutive amino acids. The computer readable medium may further have data indicating the tissue or organ in which the amino acid sequence is present.

### BRIEF DESCRIPTION OF THE DRAWINGS

FIG. 1 is a diagrammatic view of the T-DNA inserts used for gene trap vectors. All three inserts have a promoterless GUS reporter gene that encodes the enzyme β-glucuronidase (GUS). GUS is expressed when inserted downstream of an endogenous active promoter region. The T-DNA also carries a gene encoding the selectable marker hygromycin phosphotransferase (HPH), which confers resistance to the antibiotic hygromycin. pGA1633 and pGA2707 also have an intron carrying three putative splicing acceptor and donor sites adjacent to the 5′ end of the GUS gene. These altered splice sites allow for the GUS gene to be translated in the correct reading frame independently of its site of gene insertion. The DNA sequence of the T-DNA from pGA2707 is shown in SEQ ID NO:69.

FIG. 2 is a graphical presentation of the frequency of expression of the GUS gene using the method of the present invention. The percentage of GUS expression in leaves, roots, flowers, and seeds (determined as a percentage of total plants, flowers, or seeds subjected to the transformation procedure) ranged from about 1.6% to 4.0%. In 5,353 seedlings, 106 leaves and 113 roots showed GUS⁺. In 20,000 flowers, 800 lines were GUS⁺. In 5,400 developing seeds, 86 were positive.

FIGS. 3A-3E display the expression characteristics (A-D) and T-DNA insertion site (E) of tagging line 1b-115-22. Germin (oxalate oxidase)-like protein carries out important functions for development, stress response and defense against pathogens.

FIGS. 4A-4B display the expression characteristics (A) and T-DNA insertion site (B) of tagging line 1b-164-43. The alternative oxidase is used as a second terminal oxidase in the mitochondria as electrons are transferred directly from reduced ubiquinol to oxygen forming water. This is not coupled to ATP synthesis and is not inhibited by cyanide. This pathway is a single step process. In rice, the transcript levels of the alternative oxidase are increased by low temperature.

FIGS. 5A-5B display the expression characteristics (A) and T-DNA insertion site (B) of tagging line 1b-192-40. Xa21-like protein kinase gene is important for disease resistance.

FIGS. 6A-6C display the expression characteristics (A-B) and T-DNA insertion site (C) of tagging line 1b-207-27. This protein encoded by receptor-like protein kinase gene may function as a receptor of various environmental and developmental stimuli.

FIGS. 7A-7B display the expression characteristics (A) and T-DNA insertion site (B) of tagging line 1b-138-07. Methylmalonate semi-aldehyde dehydrogenase (MMSDH1) catalyzes the irreversible oxidative decarboxylation of malonate and methyl-malonate semialdehydes to acetyl- and propionyl-CoA, respectively. MMSDH is the only aldehyde dehydrogenase known to require CoA. In wheat, this gene is cold-inducible.

FIGS. 8A-8B display the expression characteristics (A) and T-DNA insertion site (B) of tagging line 1d-059-12. Inserted sequence is second exon of RNA-binding protein, which is involved in RNA-binding.

FIGS. 9A-9B display the expression characteristics (A) and T-DNA insertion site (B) of tagging line 1c-087-40. Inserted sequence is eighth intron of vacuolar H+-ATPase subunit C, which is involved in ovulation and embryogenesis.

FIGS. 10A-10B display the expression characteristics (A) and T-DNA insertion site (B) of tagging line 1c-017-14. Inserted sequence is second intron of cinnamic acid 4-hydroxylase, which plays an essential role in the regulation of the phenylpropanoid pathway controlling the synthesis of lignin, flower pigments, signaling molecules, and a large spectrum of compounds involved in plant defense against pathogens and UV light.

FIGS. 11A-11B display the expression characteristics (A) and T-DNA insertion site (B) of tagging line 1c-038-56. Inserted sequence is the last intron of H-protein promoter binding factor-2a, which is involved in transcription, affecting the photorespiration of mitochondria.

FIGS. 12A-12C display the expression characteristics (A-B) and T-DNA insertion site (C) of tagging line 1c-041-47. Inserted sequence is sixth intron of flap endonuclease, which is involved in DNA repair system in response to external damage.

FIGS. 13A-13B display the expression characteristics (A) and T-DNA insertion site (B) of tagging line 1c-064-20. Inserted sequence is third exon of heat shock protein 70, which is molecular chaperone that is expressed under conditions of high temperature and many other stresses.

FIGS. 14A-14B display the expression characteristics (A) and T-DNA insertion site (B) of tagging line 1c-109-35. Inserted sequence is second intron of ammonium transporter, which is involved in nutrition transport.

FIGS. 15A-B display (A) the expression characteristics and (B) T-DNA insertion site of tagging line 1c-109-51. Inserted sequence is fourth exon of ATP-dependent RNA helicase, which is involved in many RNA metabolic pathways and ribosome biosynthesis, and essential for cell viability and is important for early assembly steps leading to 60S ribosomal subunits.

FIGS. 16A-16B display the expression characteristics (A) and T-DNA insertion site (B) of tagging line 1c-056-07. Inserted sequence is first intron of glucose 6-phosphate/phosphate translocator, which is involved in carbohydrate metabolism.

FIGS. 17A-17C display the expression characteristics (A-B) and T-DNA insertion site (C) of tagging line 1c-100-32. Inserted sequence is ninth exon of RNA methyltransferase, which is involved in aminophosphonate metabolism.

FIGS. 18A-18B display the expression characteristics (A) and T-DNA insertion site (B) of tagging line 1c-142-27. Inserted sequence is third exon of actin depolymerizing factor 5, which is essential for rapid F-actin turnover, stabilizing a preexisting F-actin angular conformation.

FIGS. 19A-19B display the expression characteristics (A) and T-DNA insertion site (B) of tagging line 1c-140-04. Inserted sequence is second intron of beta-glucosidase, which is involved in defense mechanisms against pests based on storing and releasing toxic chemicals, secondary plant biochemical pathways, and lignin biosynthesis.

### BRIEF DESCRIPTION OF THE SEQUENCE LISTING

SEQ ID NOS:1-17: For each indicated rice line, the junction region which links the rice gene sequence with the inserted T-DNA sequence is shown. A segment of the rice gene is present, along with a segment of the T-DNA. The positions of the nucleotides comprising the T-DNA segment are indicated in the “miscellaneous features” section of the sequence listing.

SEQ ID NOS:18-34: For each indicated rice line, the genomic DNA sequence of the gene in which the T-DNA was inserted is shown.

SEQ ID NOS:35-51: For each indicated rice line, the nucleic acid coding sequence encoding the protein whose expression was altered by insertion of the T-DNA is shown.

SEQ ID NOS:52-68: For each indicated rice line, the amino acid sequence of the protein whose expression was altered by the T-DNA insertion is shown.

SEQ ID NO:69. The DNA sequence of the T-DNA insert derived from the binary vector pGA2707 is shown.

SEQ ID NOS:70-83: These sequences are synthetic oligonucleotides for use as PCR primers as described in examples 1 through 14.

### DETAILED DESCRIPTION OF THE PREFERRED EMBODIMENT

The present invention provides rice genes that are expressed in an organ preferential manner, rice lines containing T-DNA insertions in these genes, as well as a database containing the information about these lines and genes. The genes were found by screening a large population of rice lines that were tagged with T-DNA based gene trap system.

Genomic DNA gel-blot and PCR analyses have shown that approximately 65% of the population contains more than one copy of the inserted T-DNA. Hygromycin resistance tests revealed that transgenic plants contain an average of 1.4 loci of T-DNA inserts. Therefore, it can be estimated that approximately 25,700 taggings have been generated. The binary vector used in the insertion contained the promoterless β-glucuronidase (GUS) reporter gene with an intron and multiple splicing donors and acceptors immediately next to the right border. Therefore, this gene trap vector is able to detect a gene fusion between GUS and an endogenous gene, which is tagged by T-DNA. Histochemical GUS assays were carried out in the leaves and roots from 5353 lines, mature flowers from 7026 lines, and developing seeds from 1948 lines. The data revealed that 1.6-2.1% of tested organs were GUS-positive in the tested organs, and that their GUS expression patterns were organ- or tissue-specific or ubiquitous in all parts of the plant. The large population of T-DNA-tagged lines will be useful for identifying insertional mutants in various genes and for discovering new genes in rice.

The number of T-DNA-tagged lines that would be required for saturating the rice genome can be estimated using the formula suggested in Krysan et al., 1999, supra). The following three facts determine the number. First the mean size of rice genes can be deduced from the 1766754 bp of genomic sequence that has been published in the DDBJ/EMBL/GenBank databases (AB023482, AB026295, AP000391, AP000399, AP000492, AP000559, AP000616, AP157903, AP000815, AP000816, AP000836, AP000837). Within these reported sequences, there are 331 putative genes that have been identified functionally or by exon prediction algorithms. The mean size of the rice genomic DNAs between the start and stop codons including introns is 2.6 kb. Because the upstream and downstream sequences flanked by the start and stop codons were not included, an average length of rice genes should be at least 3.0 kb. Second, the mean number of T-DNA loci distributed among the transgenic rice population was 1.4. Third, the haploid genome size of rice is 4.3×108 bp (Arumuganathan and Earle, 1991, Plant Mol. Biol. Rep. 9:208-218). If we consider a 99% probability that a T-DNA is located within a given gene, we would require approximately 660000 insertions or 471 000 tagging lines. Therefore, it would be difficult to generate a transgenic rice population in which every gene has been mutated. As the probability is lowered, the number of transgenic plants required becomes exponentially lower (Krysan et al., 1999, Plant Cell 11:2283-2290, the disclosure of which is hereby incorporated by reference in its entirety). It can be estimated that the tagged lines described herein provide a 20% probability of finding a T-DNA insertion within a given gene of size 3 kb.

The GUS activation frequency ranged between 1.6 and 2.1% in various organs. Since GUS activity was observed from more than one organ in a number of lines, the GUS activation frequency of the T-DNA-tagged lines is smaller than the sum of the values obtained from each organ. About 7% of transgenic calli showed GUS staining. Because GUS activity was not examined after induction by certain environmental conditions or chemicals such as growth substances, the total GUS tagging efficiency is actually much higher. Analysis of the reported 1766754 bp genomic sequence indicated that up to 50% of the genomic DNA is intragenic. Considering that insertion could occur in both orientations, the maximum GUS tagging efficiency would be 25% of the total population.

Insertional lines that exhibit a particular GUS staining pattern should facilitate identification of genes that are regulated spatially and temporally for plant development. For example, the Arabidopsis LRP1 (lateral root primordium 1) gene, which may play a role in lateral root development, was identified by expression of promoterless GUS expression in tagging plants (Smith and Fedoroff, 1995, Plant Cell, 7:735-745). The Arabidopsis PROLIFERA gene, which is related to the MCM2-3-5 family of yeast genes, was also cloned by gene trap transposon mutagenesis (Springer et al., 1995, Science, 268:877-880).

An important aspect of the invention is the generation of a collection (i.e., a library) of mutant seeds transformed with the T-DNA/GUS insertional mutagen that may be stored and repeatedly accessed for different purposes, particularly for directed screens. In this aspect, the T2 seed is collected from TI plants and is stored in indexed (e.g., bar coded) storage containers that identify the seed by plant identification number recorded in the electronic database. The seed library is stored under conditions that allow the long-term recovery of the seeds and generation of T2 plants therefrom. As used herein, “long-term” refers to a period of at least one year, preferably at least two years, more preferably at least five years, and more preferably at least ten years. Typical conditions for the long-term storage of seeds are a temperature of approximately 4° C. and low humidity. Each time seeds from the library are analyzed, e.g., in a screen, data regarding novel mutant traits observed in the transformed plant are recorded in the database and linked to the plant identification number.

In a preferred embodiment, production of T2 seed is repeated to the point where the seeds in the indexed library collectively represent a mutation in essentially every gene in the plant genome (i.e., “saturation of the genome”), preferably a mutation in at least 90% of genes in the genome, more preferably at least 95%, more preferably at least 99%. Using a collection of seeds which collectively represent saturation of the genome in a directed screen to allow the evaluation of the contribution of every gene in the genome to the particular mutant trait.

It is expected that the genome sequence of rice will be completed in the near future. This will produce a large number of genes whose function is unknown. One of the most efficient ways to obtain information on the function of a gene is to create a loss-of-function mutation and to study the phenotype of the resulting mutant. If a large population of mutagenized plants is available, it is possible to detect an insertion within the gene of interest by PCR using oligonucleotide primers from the insertional element and the gene of interest (Couteau et al., 1999, supra; Krysan et al., 1999, supra; Sato et al., 1999, EMBO J. 18:992-1002; the disclosures of which are hereby incorporated by reference in their entireties). Identification of the desired mutant could be accomplished efficiently using a super-pooling strategy as suggested by Krysan et al., 1999, supra. They estimated that the maximum useful pool size is 2350 lines in Arabidopsis based upon the sensitivity for detecting a specific T-DNA insert and the total amount of template DNA. We are performing experiments to determine the upper size limit on DNA pools of the rice tagged lines.

Trait Analysis of Rice Lines

The transformed rice lines are typically analyzed for altered traits over several generations. As used herein, the term “TO” refers to the generation of plant tissue that is subjected to transformation. The term “T1” refers to the generation of plants that are derived from the seed of TO plants and in which transformed plants can first be selected by application of a selection agent (e.g., an antibiotic or herbicide) for which the transgenic plant contains the corresponding resistance gene. The term “T2” refers to the generation of plants by self-fertilization of the flowers of T1 plants previously selected as being transgenic. In practicing the method, a large number of TO plants or plant cells are transformed by generating random genomic insertions of the T-DNA/GUS insertional mutagen such that the marker gene encoded by the insertion fragment is expressed. Plant cells are generally selected by their ability to grow in the presence of an amount of selective agent that is toxic to non-transformed plant cells, then regenerated to yield mature plants.

In one exemplary approach, T1 plants are observed closely on a regular basis, e.g., twice monthly, with observations entered into a notebook and/or observations and/or measurements recorded, preferably into a computer database. Bulk or individual leaf tissue may be collected from T1 plants. Observations may also be documented by photography of pools and interesting individual plants using a digital camera. Identification of mutant traits may also take place in the T2 generation and is further described below. A fraction of the plants in which the expression of native genes is modified will exhibit a visually detectable mutant trait. In practicing the invention, T2 seed is collected from T1 plants, which have survived selection, and sown to yield T2 plants. Bulk or individual leaf tissue may be collected from T2 plants, and further analysis may be done on whole plants or plant tissues. In general, T2 plants that display mutant traits are also grown until they produce seed; T3 seed is collected and sown to yield T3 plants. Similar to the treatment of T2 plants, the invention is directed to a method of producing rice lines that carry genes that have been modified by T-DNA/GUS based insertional mutagenesis. The GUS portion of the insert is promoterless, so that the GUS gene is expressed only when it is inserted into an active gene. In this way, organ preferential expression of various rice genes can be determined. The invention is also directed to the organ-preferential genes found by the T-DNA/GUS insertional mutagenesis method, as well as the proteins encoded by them. T3 plants are observed, observations recorded, and tissue collected. This cycle may be repeated multiple times. The invention also involves a database having information about the rice lines, such as the genes having the insert, the encoded proteins, the phenotypic characteristics of the mutant lines, and promoter activity of the tagged genes.

One embodiment of the present invention is a rice line in which one of the genomic sequences of SEQ ID NOS:18-34 or one of the coding sequences of SEQ ID NOS:35-51 has been disrupted. The genomic sequences of SEQ ID NOS:18-34 or the coding sequences of SEQ ID NOS:35-51 may be disrupted by insertion of T-DNA or by any other desired method.

Another embodiment of the present invention is a rice line in which the expression of one of the polypetides of SEQ ID NOS:52-68 has been disrupted. Expression of the polypeptides of SEQ ID NOS:52-68 may be disrupted by insertion of T-DNA or by any other desired method.

A further embodiment of the present invention is a rice line selected from the group consisting of 1b-115-22, 1b-164-43, 1b-192-40, 1b-207-27, 1b-138-07, 1d-059-12, 1c-087-40, 1c-017-14, 1c-038-56, 1c-041-47, 1c-064-20, 1c-109-35, 1c-109-51, 1c-056-07, 1c-100-32, 1c-142-27, and 1c-140-04.

The invention provides methods for the evaluation and characterization of mutant traits of the transformed rice lines. Exemplary phenotypic evaluations include, but are not limited to morphology, biochemical analysis, herbicide tolerance testing, herbicide target identification, fungal resistance testing, bacterial resistance testing, insect resistance testing, and screening for increased drought, salt, temperature, or other environmental stress tolerance. As set forth above, plants are observed closely by eye on a regular basis, e.g., twice monthly, for morphological traits, with observations entered into a notebook and/or recorded using a hand-held electronic data entry device. Whole plants or plants tissues may also be analyzed for altered biochemical composition and pathogen, stress, and herbicide resistance. The invention provides methods for the tracking and managing data from analysis of mutant traits. Data from analyses of mutant traits are entered into an electronic database and linked to the specific identification number for the plant or group of plants tested.

The rice lines of the present invention are useful, for example, for elucidating the biochemical pathways in which the proteins encoded by the sequences of SEQ ID NOS:18-34 and SEQ ID NOS:35-51 are involved, for obtaining promoters which direct transcription in a desired tissue or organ, for identifying promoters having a desired level of activity (by quantitating GUS expression) in a desired tissue or organ, for identifying plants having a desired characteristic and for determining the effect of a loss of function mutation in a particular gene. For example, the rice lines of the present invention may be screened to identify a line exhibiting pesticide resistance or resistance screening.

The rice lines of the present invention may be used as a basis for a screening process to select for desirable characteristics such as altered photosynthetic capacity, altered response to biotic stress, allelopathy, altered response to abiotic stress, altered morphology, altered grain yield, altered nutritional content of grain, altered growth rates, altered secondary product pathways, altered pesticide resistance, altered grain characteristics such as grain shape or taste, cooking quality, altered harvesting qualities, altered optimal growth temperatures, altered resistance to herbicides, altered flowering time, altered seed fill characteristics, altered hormone biosynthetic/degradation pathways, or altered responses to hormones.

The lines may also be used as a starting point for a secondary round of mutations (for example, for gain of function mutations), to find genes that may be of interest for overexpression, underexpression, or modification of expression in rice or other plant species (e.g., crop plants, plants of pharmaceutical interest, etc.), or to find genes that may control several aspects of plant growth (i.e., transcription factors, signaling molecules, and further to determine the localization of their action).

The mutant lines containing the T-DNA/GUS inserts may be screened to identify lines having desirable characteristics. Examples of desirable characteristics that may be found include but are not limited to, altered photosynthetic capacity, an altered responses to biotic stress (e.g., insects, nematodes, fungi, bacteria, viruses), protection from weedy species (i.e. allelopathy), altered responses to abiotic stress (cold, heat, salt, or low oxygen), altered morphology, altered grain yield, altered nutritional content of grain, altered growth rates, altered secondary product pathways, altered pesticide resistance, altered grain characteristics such as grain shape or taste, cooking quality, altered harvesting qualities (i.e., easier harvesting, or better storage qualities), altered optimal growth temperatures, altered resistance to herbicides (a high percentage of rice crop loss is evidently due to contamination of the crop with weeds), altered flowering time, altered seed fill characteristics, altered levels of hormone biosynthetic or degradation pathways or responses (i.e., ABA, SA, etc.). Many other possible screens can be performed, based on any desirable characteristic that can be observed in some fashion.

Types of Screening Methods

Screens for Morphological Traits

The transformed rice lines may be screened for altered morphological traits. Morphological traits are those traits that are observed by eye, with or without aid of a magnification device, under normal growth conditions. Exemplary morphological traits include plant size, organ size, leaf number, leaf pigmentation, leaf shape, seed size, seed shape, pattern or distribution of leaves or flowers, flower number or arrangement, time of flowering (early or late), dwarf or giant stature, stem length between nodes, root mass and root development characteristics.

Directed Screens

In other aspects of the invention a directed screen is used to analyze mutant traits of the transformed rice lines. By “directed screen” is meant the employment of particular equipment, analytical techniques, and/or conditions to identify a single type of mutant trait or class of mutant traits. Exemplary directed screens analyze changes in the biochemical composition of plant tissues, and in resistance to pathogens, herbicides, and stress.

Biochemical Analyses

The transformed rice lines of the invention may be screened for altered biochemical or metabolic characteristics. Exemplary metabolic characteristics of interest include altered biochemical composition of leaves, seeds, fruits and roots and flowers and seedlings which result in a change in the level of vitamins, minerals, oils, elements, amino acids, carbohydrates, lipids, nitrogenous bases, isoprenoids, phenylpropanoids or alkaloids.

Metabolic characteristics of interest include but are not limited to altered biochemical composition of vegetative (e.g. leaves, stems, roots) and reproductive tissues (e.g. seeds, fruits, and flowers) which result in a change in the level of vitamins, minerals, oils, elements, amino acids, carbohydrates, polymers, lipids, waxes, nitrogenous bases, isoprenoids, phenylpropanoids or alkaloids. Metabolic characteristics of interest may also include the relative abundance of various metabolite classes (e.g. high protein, low carbohydrate), and quantitative physiological descriptors such as Harvest Index, Fresh Weight, Dry Weight Ratio, seed mass, and seed density.

A variety of techniques may be used for analyzing these metabolites (see, for example, International Patent Application Number PCT/US01/13886, which is herein incorporated by reference in its entirety). Appropriate general techniques may include but are not limited to, enzymatic methods, chromatography (high-performance liquid chromatography HPLC, gas-chromatography GC, thin layer chromatography) electrophoresis (e.g. capillary, PAGE, activity gels), spectroscopy (e.g. UV-Visible, Mass-spectroscopy MS, Infrared and Near-Infrared IR/NIR, Atomic Absorption AA, Nuclear Magnetic Resonance NMR), and hybrid methodologies (e.g. HPLC-MS, GC-MS, CE-MS).

Commercially available chemical analysis software can be used for the accumulation and interpretation of chemical data and the derived results can be exported to a database where correlations may be examined between metabolic changes and other observed phenotypes. One example of such a chemical analysis software package is Waters Millennium Software (Waters Corp., Millford, Mass.). An example of a method for the analysis of lipid components is that of Browse et al. (Biochem. J. 235:25-31, 1986, the disclosure of which is hereby incorporated by reference in its entirety). Taungbodhitham and colleagues (Food Chemistry 63, 4:577-584, 1998, the disclosure of which is hereby incorporated by reference in its entirety) optimized a method for the extraction and analysis of carotenoids from fruits and vegetables. Other investigators have reported analysis conditions for the simultaneous analysis of a variety of pigment components from plant tissues (Barua and Olsen, Journal of Chromatography 707:69-79, 1998; Siefermann-Hanns, J. of Chromatography 448:411-416.1988, the disclosures of which are hereby incorporated by reference in their entireties). General seed compositional analyses are described in a number of references (e.g. Approved Methods of the American Association of Cereal Chemists 10^{th} Edition, 2000, ISBN 1-891127-12-8. American Assoc. of Cereal Chem., the disclosure of which is hereby incorporated by reference in its entirety).

Herbicide Tolerance Targets

The control of weeds is of economic importance to optimal crop production. A directed screen to identify altered resistance to an herbicide can identify both gene targets for herbicides (which are useful for the development of novel herbicidal compounds) and plant genes that can be altered to yield plants with increased resistance (tolerance) to herbicides. Assays for herbicide activity/resistance include petri-dish assays, soil assays and whole-plant assays. Exemplary endpoints indicative of herbicidal activity include inhibition of seed germination; stunting of shoots; development of abnormal seedlings that do not emerge from soil; inhibition of main and lateral roots; late emergence; newer leaf tissue that is yellow (chlorotic) or brown (necrotic); leaf tissue that lacks proper pigmentation; malformation or necrosis of terminal meristematic areas; stem twisting and epinasty; early petioles that turn down; abnormal growth responses, e.g. abnormal leaf, flower or seed formation; and rough or crumbly leaves.

Weed targets of interest include, but are not limited to, Wild Oat, Green Foxtail, Chickweed, Cleavers, Kochia, Lamb's Quarters, Canola, Leafy Spurge, Canada Thistle, Field Bindweed And Russian Knapweed, Crabgrass, Goosegrass, Annual Bluegrass, Common Chickweed, Smartweed, Wild Buckwheat, Henbit, Lawn Burweed, Com Speedwell, Alfalfa, Clover, Dandelion, Dock, Dollarweed, Woodsorrel, Betony, Daisy, Shepherd's-Purse, Thistles, Knapweeds, Vetch, Violets, Yarrow and Wild Mustard.

Plant Pathogen Resistance Testing

The control of infection by plant pathogens is of significant economic importance, given that pathogenic infection of plants can inhibit production of seeds, foliage and flowers, in addition to causing a reduction in the quality and quantity of the harvested crop. In general, most crops are treated with agricultural anti-fungal, anti-bacterial agents and/or pesticidal agents. However, damage due to infection by pathogens still results in revenue losses to the agricultural industry on a regular basis. Furthermore, many of the agents used to control such infection or infestation cause adverse side effects to the plant and/or to the environment. Plants with enhanced resistance to infection by pathogens would decrease or eliminate the need for application of chemical anti-fungal, anti-bacterial and/or pesticidal agents. For a discussion of the value of identifying insect resistance loci in plants, see Yencho G C et al., Annu Rev Entomol., 45:393-422, 2000, the disclosure of which is hereby incorporated by reference in its entirety.

Fungal Resistance

The transformed plant lines may be screened for increased fungal resistance. An exemplary screen for fungal resistance includes testing for resistance to infection by the following fungal pathogens: Albugo candida (white blister), Alternaria brassicicola (leafspot), Botrytis cinerea (gray mold), Erysiphe cichoracearum (powdery mildew), Peronospora parasitica (downy mildew), Fusarium oxysporum (vascular wilt), Plasmodiophora brassicae (clubroot), Rhizoctonia solani (root rot), Pythium spp. (damping off), Colletotrichum coccode (anthracnose), and Phytopthora infestans (late blight). Plants are susceptible to attack by a variety of additional fungi, including, but not limited to species of Sclerotinia, Aspergillus, Penicillium, Ustilago, and Tilletia.

Bacterial Resistance

The transformed rice lines of the invention may be screened for increased bacterial resistance. Exemplary screens for bacterial resistance include testing for resistance to infection by the following bacterial pathogens: Agrobacterium tumefaciens (crown gall); Erwinia tracheiphila (cucumber wilt); Erwinia stewartii (corn wilt); Xanthomonas phaseoli (common blight of beans); Erwinia amylovora (ftreblight); Erwinia carotovora (soft rot of vegetables); Pseudomonas syringae (bacterial canker); Pelargonium spp., Pseudomonas cichorii (black leaf spot); Xanthomonas fragariae (angular leaf spot of strawberry); Pseudomonas syringae (angular leaf spot of cucumber, gherkin, muskmelon, pumpkin, squash, vegetable marrow, and watermelon); and Pseudomonas morsprunorum (bacterial canker of stone fruit); Xanthomonas campestris (bacterial spot, bacteriosis, shot hole, or black spot of peach, nectarine, prune, plum, apricot. cherry or almond). The plants are evaluated in a manner that allows for easy scoring of symptoms (resistant vs. susceptible phenotype) and recording of results, e.g., digital imaging of each individual plant.

Viral Resistance

The transformed rice lines of the invention may be screened for increased viral resistance. Viral pathogens continue to be a significant problem in agriculture. Approaches to viral resistance include targeting, establishment of infection, virus multiplication, and/or viral movement. An exemplary screening assay for virus resistance involves testing for susceptibility to rice viral attack.

Insect/Nematode Resistance

In general, most crops are treated with chemical pesticides and insecticides have been effective in controlling many harmful insects. However, damage due to insect infestation remains a problem and results in revenue losses to the agricultural industry on a regular basis. In addition, many insecticides are expensive; they require repeated applications for effective control and cause adverse side effects to the plant and/or the environment. Further, there are concerns that insects have or will become resistant to many of the chemicals used in controlling them. Plants with enhanced insect resistance would decrease or eliminate the need for application of such chemical pesticides.

Exemplary screens for plant resistance to insects include assays that target insect species of the orders Lepidoptera, Hemiptera, Orthoptera, Coleoptera, Psocoptera, Isoptera, Thysanoptera and Homoptera. In general such assays are used to detect the actual killing of insects, the interruption of insect growth and development so that maturation is slowed or prevented (e.g., anti-feedant activity), and/or the prevention of ovaposition or hatching of insect eggs.

An exemplary screening assay for insect resistance involves testing for susceptibility to attack by a variety of insect species that attack different parts of the plant. For example, the stem, the leaves and the roots. Since it expected that many resistance mutations will be loss-of function (recessive) it is important that enough transformed plants (which have survived application of the selective agent) are evaluated to insure that a homozygous mutant is tested. Each individual surviving plant is tested separately and if insect/nematode resistance is detected, the individual plant is retained for seed collection. For each test, the interaction of the insects or nematodes with a mutant plant is compared to the interaction of the same species of insect or nematode with wild type plants.

Stress Resistance

The transformed rice lines may be screened for increased stress resistance. Directed screens to identify altered stress resistance (e.g., to drought, salt, cold, toxins, metal, heat, or other environmental and biological stresses) may identify rice genes that can be altered to yield plants with increased stress resistance (tolerance). Such discoveries may ultimately result in an ability to cultivate rice crops in a broader areas, such as arid and/or saline land. Directed screens performed to identify genes involved in stress response use laboratory conditions that simulate the particular stress, such as water deprivation or high salt concentration. The invention also involves a database having information about the rice lines, such as the genes having the insert, the encoded proteins, the phenotypic characteristics of the mutant lines, and promoter activity of the tagged genes.

Database for Storage and Manipulation of Information Relating to the Rice Lines, the Genes and Polypeptides, Phenotype and Other Characteristics

The nucleic acid sequences, amino acid sequences, expression pattern, protein function, chromosomal location, and other relevant information can be entered into a database for storage and manipulation. It will be appreciated by those skilled in the art that the data could be stored and manipulated on any medium which can be read and accessed by a computer. Computer readable media include magnetically readable media, optically readable media, or electronically readable media. For example, the computer readable media may be a hard disc, a floppy disc, a magnetic tape, CD-ROM, RAM, or ROM as well as other types of other media known to those skilled in the art.

In addition, the data may be stored and manipulated in a variety of data processor programs in a variety of formats. For example, the sequence data may be stored as text in a word processing file, such as MICROSOFT WORD or WORDPERFECT or as an ASCII file in a variety of database programs familiar to those of skill in the art, such as DB2, SYBASE, or ORACLE.

The computer readable media on which the sequence information and other information is stored may be in a personal computer, a network, a server or other computer systems known to those skilled in the art. The computer or other system preferably includes the storage media described above, and a processor for accessing and manipulating the sequence data. Once the sequence data has been stored it may be manipulated and searched to locate those stored sequences which contain a desired nucleic acid sequence, those which encode a protein having a particular functional domain, or those that have a desired characteristic such as expression pattern, chromosomal location, etc. For example, the stored sequence information may be compared to other known sequences to identify homologies, motifs implicated in biological function, or structural motifs.

Programs which may be used to search or compare the stored nucleic acid or amino acid sequences include the MacPattern (EMBL), BLAST, and BLAST2 program series (NCBI), basic local alignment search tool programs for nucleotide (BLASTN) and peptide (BLASTX) comparisons (Altschul et al, J. Mol. Biol. 215: 403 (1990)) and FASTA (Pearson and Lipman, Proc. Natl. Acad. Sci. USA, 85: 2444 (1988), the disclosures of which are hereby incorporated by reference in their entireties). The BLAST programs then extend the alignments on the basis of defined match and mismatch criteria.

The genomic sequences of SEQ ID NOS:18-34, the cDNA sequences of SEQ ID NOS:35-51, or the polypeptide codes of SEQ ID NOS:52-68 may be stored and manipulated in a variety of data processor programs in a variety of formats. For example, the genomic sequences of SEQ ID NOS:18-34, the cDNA codes of SEQ ID NOS:35-51, or the polypeptide codes of SEQ ID NOS:52-68 may be stored as text in a word processing file, such as MICROSOFT WORD or WORDPERFECT or as an ASCII file in a variety of database programs familiar to those of skill in the art, such as DB2, SYBASE, or ORACLE. In addition, many computer programs and databases may be used as sequence comparers, identifiers, or sources of reference nucleotide or polypeptide sequences to be compared to the genomic sequences of SEQ ID NOS:18-34, the cDNA codes of SEQ ID NOS:35-51, or the polypeptide codes of SEQ ID NOS:52-68.

The following list is intended not to limit the invention but to provide guidance to programs and databases which are useful with the genomic sequences of SEQ ID NOS:18-34, the cDNA codes of SEQ ID NOS:35-51, or the polypeptide codes of SEQ ID NOS:52-68. The programs and databases which may be used include, but are not limited to: MACPATTERN (EMBL), DISCOVERY BASE (Molecular Applications Group), GENEMINE (Molecular Applications Group), LOOK (Molecular Applications Group), MACLOOK (Molecular Applications Group), BLAST and BLAST2 (NCBI), BLASTN and BLASTX (Altschul et al, J. Mol. Biol. 215: 403 (1990)), FASTA (Pearson and Lipman, Proc. Natl. Acad. Sci. USA, 85: 2444 (1988)), FASTDB (Brutlag et al. Comp. App. Biosci. 6:237-245, 1990), CATALYST (Molecular Simulations Inc.), CATALYST/SHAPE (Molecular Simulations Inc.), CERIUS².DBACCESS (Molecular Simulations Inc.), HYPOGEN (Molecular Simulations Inc.), INSIGHT II, (Molecular Simulations Inc.), DISCOVER (Molecular Simulations Inc.), CHARMm (Molecular Simulations Inc.), FELIX (Molecular Simulations Inc.), DELPHI, (Molecular Simulations Inc.), QUANTEMM, (Molecular Simulations Inc.), HOMOLOGY (Molecular Simulations Inc.), MODELER (Molecular Simulations Inc.), ISIS (Molecular Simulations Inc.), Quanta/Protein Design (Molecular Simulations Inc.), WEBLAB (Molecular Simulations Inc.), WEBLAB DIVERSITY EXPLORER (Molecular Simulations Inc.), GENE EXPLORER (Molecular Simulations Inc.), SEQFOLD (Molecular Simulations Inc.), and the EMBL/SWISSPROTEIN database.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

Phenotypic observations/measurements alone or together with nucleic acid sequence information may be entered into a computer database, so that the information is searchable based on mutant traits and/or nucleic acid sequence, and that the computer database may interface with a computer network (such as that disclosed in PCT/US01/13886 Supra). Numerous commercial databases are available that can provide the platform for practicing this aspect of the invention, e.g., FILEMAKER PRO and ORACLE databases.

A network may be used for allowing users to access, retrieve and view information in a relational database containing the database of plant records, in accordance with another aspect of the present invention. The Network includes a communication path through which a network server and a representative client are connected. For ease of illustration, only a representative client is shown; however, it will be apparent to those skilled in the art that many more clients can also be connected. The Network client uses the network to access the database of plant records and associated resources provided by the network server. The nature of the communication paths connecting the network client and the network server are not critical to the practice of the present invention. Such paths may be implemented as switched and/or non-switched paths using private and/or public facilities. Similarly, the topology of the network is not critical and may be implemented in a variety of ways including hierarchical and peer-to-peer networks. The network may be anyone of a number of conventional network systems, including a local area network (LAN) or a wide area network (WAN) using Ethernet or the like. The network includes functionality for packaging client calls in a standard format (e.g., URL) together with any parameter information into a format suitable for transmission across communication path for delivery to the server. The Network server may be a hypermedia server, perhaps operating in conformity with the Hypertext Transfer Protocol (HTTP). The server includes hardware and an operating system necessary for running software for (i) accessing records in a plant database in response to user requests, and (ii) presenting information to client computer. Such software may include, for example, a relational database management system that runs on the operating system. The server also typically includes a World Wide Web server and a World Wide Web application. The World Wide Web application includes executable code necessary for generation of database language statements (e.g., Standard Query Language (SQL) statements). The Application may also include a configuration file that contains pointers and addresses to the various software modules of the server, as well as to the database for servicing user requests. The Client computer includes hardware and appropriate software to connect to a network and run a standard Web browser which is used to access, view and interact with information provided by the server. For example, the client computer may be any conventional networked computer, such as a PC, a MACINTOSH, or a UNIX workstation running NETSCAPE NAVIGATOR or INTERNET EXPLORER.

Hardware found in a typical computer, which may be used to implement a network server and/or network client, is well known in the art. The Database is preferably arranged and configured to store the information contained on the plant records in relational format. Such a relational database supports a set of operations defined by relational algebra, and includes tables composed of rows and columns for the information. The database is relationally arranged so that a searched phenotypic trait can be associated with a plant having other phenotypic traits of interest or with a plant having a candidate gene sequence of interest, and so that a searched DNA sequence can be associated with a plant having phenotypic traits of interest.

Graphical User Interface (GUN)

Through the Web browser, a user is presented with a graphical user interface (GUI) which includes a plurality of screens (e.g., HTML pages) and a suite of functions for constructing and transmitting search requests, and selectively displaying data retrieved from the database. The functions are preferably in the form of standard GUI elements, such as buttons, pull down menus, scroll bars, text boxes, etc. displayed on the screens. The GUI includes a main menu page from which various lines of inquiry can be followed. From the main menu, a user is able to navigate to a screen that includes a database search engine function. Such a screen includes a text box that is capable of receiving a user-specified search request, such as a mutant trait or DNA sequence, for searching the database. The search request is transmitted to the server and converted by the Web application component of the server to an SQL query. That query is then used by the relational database management system component of the server to search and extract relevant data from the database and provide that data to the server in an appropriate format. The Server then generates a new HTML page displaying the retrieved information on the Web browser running on the client.

In one embodiment, the retrieved information is initially displayed as a hyper linked list individually identifying plant records retrieved from the database. The user then clicks on one of the hyperlink identifiers to display the information contained in a particular plant record in a new HTML page, which includes a plant image that is linked to the relevant data in the database. In one embodiment, such information includes plant identification number, an image or visual representation of the plant, a hyper linked list identifying additional phenotypic and/or genotypic information regarding the plant. For example, the list may have links to biochemical and biological mutant trait information associated with the plant. For at least some records, the list further includes a candidate gene sequence link (i.e., to a candidate gene whose expression has been modified). The GUI of the present invention is particularly advantageous in that it allows a user to easily associate a searched mutant trait with a plant having other mutant traits or with a plant having modified expression of a candidate gene sequence. It also allows a user to associate a searched DNA sequence with a plant having specific mutant traits.

In a preferred embodiment, the rice lines can be used as a marker for a particular chromosome. This can be useful to determine the chromosomal location of various genes of interest in lines of rice. For instance, by having multiple lines of rice, each line with an insertion on a separate, known, chromosome of the rice genome, one is able to determine the chromosomal location of the genes of novel phenotypes by observing how the phenotypes of those genes segregate with the known inserts in the rice lines. For example, if the phenotype segregates with the insertion at a frequency which is significantly higher than would be expected from random segregation, the gene responsible for the phenotype lies on the chromosome on which the insertion is located. The predicted chromosomal location of the genes of the invention, along with the protein encoded by the gene, are listed below in Table 1. The chromosomal locations were predicted by comparing the sequences of the present invention to a database of rice sequences whose chromosomal locations were known. In some cases, more than one chromosome contained a sequence with significant homology to the sequences of the present invention. The ability to identify the location of such genes of interest is of critical importance for plant genomes. This is due primarily to the fact that many plant genomes contain huge amounts of duplications within their genomes, making traditional sequencing methods dubious, and techniques such as shotgun sequencing subject to some suspicion. By being able to identify the chromosome upon which a gene is located, one is able to greatly reduce the number of possible false positives that may be responsible for the desired phenotype of a gene of interest.

TABLE 1

| Internal | | | |
|---|---|---|---|
| Code for | | | |
| Gene and | Putative Protein | Chromo- | Figure |
| Line | Encoded by Gene | some(s) | Number |
| 1b-115-22 | Germin-like protein | 1 | 3 |
| 1b-164-43 | alternative oxidase (AOX1a) | 4 | 4 |
| | protein | | |
| 1b-192-40 | XA21-like protein kinase gene | 2 | 5 |
| | | X | |
| 1b-207-27 | receptor-like protein kinase | 1 | 6 |
| 1b-138-07 | methylmalonate semi-aldehyde | 2 | 7 |
| | dehydrogenase (MMSDH1) | 4 | |
| 1d-059-12 | homolog of the RNA-binding | 4 | 8 |
| | protein LAH1 | | |
| 1c-087-40 | vacuolar ATP synthase subunit C | 4 | 9 |
| 1c-017-14 | cinnamic acid 4-hydroxylase | 4 | 10 |
| | | 2 | |
| 1c-038-56 | H-protein promoter binding | 10 | 11 |
| | factor-2a | | |
| 1c-041-47 | flap endonuclease (FEN-1) | 5 | 12 |
| 1c-064-20 | heat shock protein Hsp70 | 3 | 13 |
| 1c-109-35 | ammonium transporter | 3 | 14 |
| 1c-109-51 | ATP-dependent RNA helicase | 2 | 15 |
| 1c-056-07 | glucose-6-phosphate/phosphate | 4 | 16 |
| | transporter | | |
| 1c-100-32 | RNA methyltransferase | 4 | 17 |
| 1c-142-27 | actin depolymerizing factor 5 | 4 | 18 |
| | | 10 | |
| 1c-140-04 | beta-glucosidase | 9 | 19 |
| | | 2 | |

The sequencing of an organism's genome does not automatically inform one of where a particular active gene is located. One issue that arises is that while a particular gene may be found in multiple copies throughout an organism's genome, whether or not all of these genes function in producing a phenotype is a separate issue. One manner of determining functionality is by deleting or altering the gene of interest and determining whether or not there is a physiological change in the organism. However, this technique is fairly disruptive and may be lethal. Alternatively, by using the rice lines of a preferred embodiment of the current invention one is able to observe whether or not a particular gene, on a particular chromosome, is the one that is responsible for a given phenotype. In other words, in a preferred embodiment, the rice lines of the current invention can be used to determine if the phenotype of a gene of interest is the result of a gene on one chromosome versus a similar copy of the gene on another chromosome.

The rice lines of another preferred embodiment of the current invention can be used to monitor chromosome duplication in rice. Chromosome duplication is one of the methods by which rice increases its opportunities for genetic diversity. Similarly, polyploid plants, which may have many commercially favorable characteristics, involve chromosome duplication. As such, there is a need to be able to identify which chromosome or chromosomes have been duplicated. Since the rice lines of the preferred embodiment can have an unique insert, the lines provide a device that allows for the identification which chromosome has been duplicated, without worrying about the risk that the natural markers in the chromosome may have been previously duplicated across multiple chromosomes of the genome.

The rice lines of another preferred embodiment of the current invention can be used as a background control marker to ensure that rice is properly identified with its source. For instance, the inserts which are present in the rice lines of the preferred embodiment can be used to identify the source of the rice line, a feature that is useful for both scientific and commercial reasons. In one embodiment, genetic inserts can be used as a sort of molecular identifier, allowing people to police how their lines of rice are being used. Alternatively, a line of rice with an artificial insert presents a useful background for field experiments. For instance, if the experiment is carried out in one of the lines of the preferred embodiment, one can verify at the conclusion of the experiment that the final line of rice was derived from the initial line. This is useful, not only in situations where there is a great likelihood of contamination (such as outdoor work), but also in situations where one may wish to screen large numbers of potential candidates for resistance to certain factors or induce genetic changes through external stimuli. In all of these situations, it would be of great advantage to be able to verify, with relative certainty, that the final rice line is the same or was derived from the initial rice line.

As will be appreciated by one of skill in the art, there are many other uses for the rice lines of the current invention. Several examples are listed below.

Use of Rice Lines as a Chromosomal Marker

Each line of rice of the current invention allows for the localization of various genes of interest. The lines of rice of the current invention can be used to correlate a gene of interest onto a particular chromosome that is marked with the insertion of the current invention. By using the lines of rice of the current invention, one is able to easily identify which chromosome contains a novel gene of interest.

A rice line containing one of the inserts of the preferred embodiment is first developed as described above. Preferably one line is produced for each chromosome, although the number of lines needed may correspond with the complexity of the problem to be addressed, and in some situations a single line may be enough. In the simplest example, only a single insert is added to a single chromosome to create a single rice line. Mutations or other genetic modifications are then applied to the rice line by any number of techniques known in the field. Rice lines which display phenotypes that are interesting are then crossed with a wild-type line (not containing the same known insertion) to yield an F1 progeny line. One then tests the F1 progeny for both the desired phenotype and the known insert or “marker.”

Examination of the insert or marker can occur in many different ways. One possible manner is by the molecular detection of the sequence of interest on that particular chromosome, for instance by sequencing, PCR, complementary nucleic acid hybridization or antibodies. For example, to use PCR-based methods to detect or follow the known insert, synthetic PCR primer oligonucleotides are designed. The forward primer is designed from an endogenous portion of the rice gene that has the insert. The endogenous sequences of the rice genes having the insert are shown in SEQ ID NOS:18-34. The reverse primer is designed from a portion of the T-DNA insert sequence. The reverse primer may also be designed from a region spanning a segment of the T-DNA insert sequence and a segment of the rice gene having the insert. The nucleic acid sequences of these spanning regions can be found, for example, in SEQ ID NOS:1-17.

Alternatively, one may add markers to the insert of the current invention which allow for the visualization of a chemical product or byproduct of the insert. Such markers would include molecules that can be viewed directly (i.e. GFP) or molecules that are easy to detect through secondary chemical reactions (i.e. GUS) or the molecule's influence on the plant itself. If the gene of interest is located on the same chromosome as the insert of the preferred embodiment, then the frequency of the F 1 progeny which contains both the phenotype of interest and the insert of interest will be significantly higher than the frequency that would be expected if the phenotype and the insert segregated randomly. On the other hand, if the insert is not located on the same chromosome as the gene which produces the phenotype, there will be no correlation between the presence of the phenotype and the presence of the insert, and the frequency of the F1 progeny having both the insert and the phenotype will be that which would be expected from random segregation. As will be appreciated by one of skill in the art, the more crosses that are performed, the greater the certainty one will have regarding the location of a particular gene of interest. As such, additional generations, F2, F3 . . . and so on may be evaluated to enhance the certainty of the result.

One advantage this technique has over other possible techniques is that it allows one to locate a phenotypically relevant gene to a particular chromosome, whereas a simple sequence comparison may lead one to many sequences that are structurally similar, but not functionally relevant, either due to the location of the gene, point mutations in the genes, or differences in the noncoding sections of the genes. In addition, this technique may facilitate efforts to clone the gene associated with the phenotype by focusing the cloning effort on a library derived from the appropriate chromosome.

Chromosome Duplication

The rice lines of one embodiment may be used to track chromosome duplication in plants. One commercially profitable form of duplication may be the induction of a polyploid state. Polyploidization can be induced in many ways with many different stimulants. In one embodiment, a chemical such as Colchicine, or any antimitotic agent, can be used to induce a polyploid state in one of the rice lines of the preferred embodiment. Once a polyploid state has been induced, the insert or marker in the rice lines can be examined, by a variety of techniques, to verify that the chromosome has been duplicated or to determine which chromosome or chromosomes have been duplicated. As will be appreciated by one of skill in the art, this process can be used to monitor any chromosome or fragment of a chromosome duplication.

Marked Rice Lines

The insert of the rice lines of the preferred embodiment can be used as an internal control for plant experiments. The rice lines of the preferred embodiment contain an insert that is known and is unique relative to sequences in other rice genomes. This rice line is used as the background for all of the experiments for a particular project. At the end of the experiment, the presence of the marker is verified by any number of techniques, either directly through the sequence or structure of the insert, or indirectly through the influence of the insert. This allows one to confirm that the plants obtained at the end of the experiment were derived from the starting line.

Further, the rice genes found by the method described herein may be used to transform plants to have increased expression of the gene, decreased expression of the gene, or altered patterns of expression from that of the wild-type plant. Plants overexpressing, underexpressing, or having an altered expression pattern of the genes found in this invention may be of agronomic importance. For example, such plants may possess environmental stress protection, altered secondary pathways, increased nutritional quality of grain, increased harvesting characteristics, increased storage qualities of grain, increased desirable qualities of grain (shape, taste, cooking qualities, stickiness, etc), decreased use of agricultural pesticides or herbicides, increased efficiency of fertilizer application, increased yield, altered seed fill qualities (i.e., timing of onset, rate of seed fill, influence of environmental qualities such as nutrient availability, light, etc. on seed fill), and altered germination rates.

Organ Preferential Polynucleotides of the Invention

Embodiments of the present invention provide isolated or purified nucleic acid sequences of SEQ ID NOS:18-34 or SEQ ID NOS:35-51. Embodiments of the invention also provide any isolated polynucleotide sequence encoding a polypeptide having the amino acid sequence of SEQ ID NOS:52-68. The term “isolated” as used herein includes polynucleotides substantially free of other nucleic acids, proteins, lipids, carbohydrates or other materials with which it is naturally associated.

As used herein, the term “isolated” means that the nucleic acid sequence is adjacent to “backbone” nucleic acid to which it is not adjacent in its natural environment. Additionally, to be “enriched” the nucleic acid sequence will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched nucleic acid sequences represent 15% or more of the number of nucleic acid inserts in the population of recombinant backbone molecules. More preferably, the enriched nucleic acid sequences represent 50% or more of the number of nucleic acid inserts in the population of recombinant backbone molecules. In a highly preferred embodiment, the enriched nucleic acid sequence represent 90% or more of the number of nucleic acid inserts in the population of recombinant backbone molecules.

As used herein, the term “isolated” requires that the material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “purified” does not require absolute purity; rather, it is intended as a relative definition. Individual nucleic acid clones isolated from a library have been conventionally purified to electrophoretic homogeneity. The sequences obtained from these clones could not be obtained directly either from the library or from total genomic DNA. Purification of starting material or natural material to at least one order of magnitude, preferably two or three orders, and more preferably four or five orders of magnitude is expressly contemplated.

Polynucleotide sequences of the invention include DNA, cDNA and RNA sequences which encode SEQ ID NOS:52-68. It is understood that polynucleotides encoding all or varying portions of SEQ ID NOS:52-68 are included herein, as long as they encode a polypeptide with enzymatic or functional activity. Such polynucleotides include naturally occurring, synthetic, and intentionally manipulated polynucleotides as well as splice variants. For example, portions of the mRNA sequence may be altered due to alternate RNA splicing patterns or the use of alternate promoters for RNA transcription. As used herein, the terms “polynucleotides” and “nucleic acid sequences” refer to DNA, RNA and cDNA sequences.

Polynucleotides of the present invention include polynucleotides consisting essentially of SEQ ID NOS:18-34 or SEQ ID NOS:35-51. The term “consisting essentially of” requires that the protein encoded by the nucleic acid has the activity or function as set forth in Table 2.

Polynucleotides of the present invention include polynucleotides having alterations in the nucleic acid sequence of SEQ ID NOS:18-34 or SEQ ID NOS:35-51 where such polynucleotides are still able to encode a polypeptide having the general function of the native gene product. Alterations in the nucleic acids SEQ ID NOS:18-34 or SEQ ID NOS:35-51 within the scope of the present invention include, but are not limited to, intragenic mutations such as point mutations, nonsense (stop) mutations, antisense, splice site and frameshift mutations, as well as heterozygous or homozygous deletions. Such alterations may be detected by standard methods known to those of skill in the art including sequence analysis, Southern blot analysis, PCR based analyses (e.g., multiplex PCR, sequence tagged sites (STSs)) and in situ hybridization. Embodiments of the invention also include anti-sense polynucleotide sequences, where an antisense sequence may be complementary to the entire sequence, or any fragment thereof.

The polynucleotides described herein include sequences that are degenerate as a result of the genetic code. There are 20 natural amino acids, most of which are specified by more than one codon. Therefore, all degenerate nucleotide sequences are included in the invention as long as the polypeptide encoded by such nucleotide sequences retains enzymatic or functional activity. A “functional polynucleotide” denotes a polynucleotide which encodes a functional polypeptide as described herein. Embodiments of the invention include polynucleotides encoding a polypeptide having the biological activity of the polypeptides having the amino acid sequence of SEQ ID NOS:52-68 and having at least one epitope for an antibody immunoreactive with SEQ ID NOS:52-68.

In one embodiment, the polynucleotides encoding the polypeptides of the invention include the nucleotide sequences of SEQ ID NOS:18-34 or SEQ ID NOS:35-51 and nucleic acid sequences complementary thereto. A complementary sequence may include an antisense nucleotide. When the sequence is RNA, the deoxyribonucleotides A, G, C, and T of SEQ ID NOS:18-34 or SEQ ID NOS:35-51 are replaced by ribonucleotides A, G, C, and U, respectively. Embodiments of the invention include fragments or “probes” of the above-described nucleic acid sequences, wherein the fragments or probes are at least 10, 15, 20, 25, 30, 35, 40, 50, 75, 100, 150, 200, 300, 400, 500, 750, 1000, 1250, or 1500 bases in length, which is presumed to be sufficient to permit the probe to selectively hybridize to DNA encoding the proteins of the invention.

One embodiment of the present invention is homologous genomic nucleic acids. By “homologous genomic nucleic acid” is meant a nucleic acid homologous to a nucleic acid selected from the group consisting of SEQ ID NOS:18-34 or a portion thereof. In some embodiments, the homologous genomic nucleic acid may have at least 97%, at least 95%, at least 90%, at least 85%, at least 80%, or at least 70% nucleotide sequence identity to a nucleotide sequence selected from the group consisting of SEQ ID NOS:18-34 and fragments comprising at least 10, 15, 20, 25, 30, 35, 40, 50, 75, 100, 150, 200, 300, 400, 500, 750, 1000, 1250, or 1500 consecutive nucleotides thereof. In other embodiments the homologous genomic nucleic acids may have at least 97%, at least 95%, at least 90%, at least 85%, at least 80%, or at least 70% nucleotide sequence identity to a nucleotide sequence selected from the group consisting of the nucleotide sequences complementary to one of SEQ ID NOS:18-34 and fragments comprising at least 10, 15, 20, 25, 30, 35, 40, 50, 75, 100, 150, 200, 300, 400, 500, 750, 1000, 1250, or 1500 consecutive nucleotides thereof. Identity may be measured using BLASTN version 2.0 with the default parameters or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

The term “homologous genomic nucleic acid” also includes nucleic acids comprising nucleotide sequences which encode polypeptides having at least 99%, 95%, at least 90%, at least 85%, at least 80%, at least 70%, at least 60%, at least 50%, at least 40% or at least 25% amino acid identity or similarity to a polypeptide comprising the amino acid sequence of one of SEQ ID NOS:52-68 or fragments comprising at least 5, 10, 15, 20, 25, 30, 35, 40, 50, 75, 100, 200, 400, 600, 800, or 1000 consecutive amino acids thereof as determined using the FASTA version 3.0t78 algorithm with the default parameters. Alternatively, protein identity or similarity may be identified using BLASTP with the default parameters, BLASTX with the default parameters, TBLASTN with the default parameters, or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

The term “homologous genomic nucleic acid” also includes nucleic acids which hybridize under stringent conditions to a nucleic acid selected from the group consisting of the nucleotide sequences complementary to one of SEQ ID NOS:18-34 and coding nucleic acids comprising nucleotide sequences which hybridize under stringent conditions to a fragment comprising at least 10, 15, 20, 25, 30, 35, 40, 50, 75, 100, 150, 200, 300, 400, 500, 750, 1000, 1250, or 1500 consecutive nucleotides of the sequences complementary to one of SEQ ID NOS:18-34. As used herein, “stringent conditions” means hybridization to filter-bound nucleic acid in 6×SSC at about 45° C. followed by one or more washes in 0.1×SSC/0.2% SDS at about 68° C. Other exemplary stringent conditions may refer, e.g., to washing in 6×SSC/0.05% sodium pyrophosphate at 37° C., 48° C., 55° C., and 60° C. as appropriate for the particular probe being used.

The term “homologous genomic nucleic acid” also includes nucleic acids comprising nucleotide sequences which hybridize under moderate conditions to a nucleotide sequence selected from the group consisting of the sequences complementary to one of SEQ ID NOS:18-34 comprising nucleotide sequences which hybridize under moderate conditions to a fragment comprising at least 10, 15, 20, 25, 30, 35, 40, 50, 75, 100, 150, 200, 300, 400, 500, 750, 1000, 1250, or 1500 consecutive nucleotides of the sequences complementary to one of SEQ ID NOS:18-34. As used herein, “moderate conditions” means hybridization to filter-bound DNA in 6× sodium chloride/sodium citrate (SSC) at about 45° C. followed by one or more washes in 0.2×SSC/0.1% SDS at about 42-65° C.

The term “homologous genomic nucleic acids” also includes nucleic acids comprising nucleotide sequences which encode a gene product whose activity may be complemented by a nucleic acid comprising a nucleotide sequence selected from the group consisting of SEQ ID NOS:18-34. In some embodiments, the homologous genomic nucleic acids may encode a gene product whose activity is complemented by the gene product encoded by a nucleic acid comprising a nucleotide sequence selected from the group consisting of SEQ ID NOS:35-51.

Polynucleotide sequences of the invention may be obtained by several methods. For example, the polynucleotide can be isolated using hybridization or computer-based techniques which are well known in the art including, but not limited to: 1) hybridization of genomic or cDNA libraries with probes to detect homologous nucleotide sequences; 2) antibody screening of expression libraries to detect cloned DNA fragments encoding polypeptides with shared structural features; 3) polymerase chain reaction (PCR) on genomic DNA or cDNA using primers capable of annealing to the DNA sequence of interest; 4) computer searches of sequence databases for similar sequences; and 5) differential screening of a subtracted DNA library.

Embodiments of the present invention provide the complete cDNA sequences (SEQ ID NOS:35-51) encoding the proteins (SEQ ID NOS:52-68) of the invention. Also included in embodiments of the invention are nucleotide sequences that are greater than 70% homologous with the sequence of SEQ ID NOS:35-51, but still retain enzymatic activity or functional activity in plants. Other embodiments of the invention include nucleotide sequences that are greater than 75%, 80%, 85%, 90% or 95% homologous with the sequence of SEQ ID NOS:35-51, but still retain enzymatic activity or functional activity in plants.

Polynucleotides of the present invention include polynucleotides consisting essentially of SEQ ID NOS:35-51, wherein the term “consisting essentially of” requires that the protein encoded by the coding nucleic acid has the activity or function as set forth in Table 2.

The present invention includes homologous coding nucleic acid sequences, homologous coding nucleic acid sequences, and homologous polypeptide sequences. By “homologous coding nucleic acid” is meant a nucleic acid homologous to a nucleic acid having at least 97%, at least 95%, at least 90%, at least 85%, at least 80%, or at least 70% nucleotide sequence identity to a nucleotide sequence selected from the group consisting of SEQ ID NOS:35-51 and fragments comprising at least 10, 15, 20, 25, 30, 35, 40, 50, 75, 100, 150, 200, 300, 400, 500, 750, 1000, 1250, or 1500 consecutive nucleotides thereof. In other embodiments the homologous coding nucleic acids may have at least 97%, at least 95%, at least 90%, at least 85%, at least 80%, or at least 70% nucleotide sequence identity to a nucleotide sequence selected from the group consisting of the nucleotide sequences complementary to one of SEQ ID NOS:35-51 and fragments comprising at least 10, 15, 20, 25, 30, 35, 40, 50, 75, 100, 150, 200, 300, 400, 500, 750, 1000, 1250, or 1500 consecutive nucleotides thereof. Identity may be measured using BLASTN version 2.0 with the default parameters or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

The term “homologous coding nucleic acid” also includes nucleic acids comprising nucleotide sequences which encode polypeptides having at least 99%, 95%, at least 90%, at least 85%, at least 80%, at least 70%, at least 60%, at least 50%, at least 40% or at least 25% amino acid identity or similarity to a polypeptide comprising the amino acid sequence of one of SEQ ID NOS:52-68 or fragments comprising at least 5, 10, 15, 20, 25, 30, 35, 40, 50, 75, 100, 200, 400, 600, 800, or 1000 consecutive amino acids thereof as determined using the FASTA version 3.0t78 algorithm with the default parameters. Alternatively, protein identity or similarity may be identified using BLASTP with the default parameters, BLASTX with the default parameters, TBLASTN with the default parameters, or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

The term “homologous coding nucleic acid” also includes coding nucleic acids which hybridize under stringent conditions to a nucleic acid selected from the group consisting of the nucleotide sequences complementary to one of SEQ ID NOS:35-51 and coding nucleic acids comprising nucleotide sequences which hybridize under stringent conditions to a fragment comprising at least 10, 15, 20, 25, 30, 35, 40, 50, 75, 100, 150, 200, 300, 400, 500, 750, 1000, 1250, or 1500 consecutive nucleotides of the sequences complementary to one of SEQ ID NOS:35-51. As used herein, “stringent conditions” means hybridization to filter-bound nucleic acid in 6×SSC at about 45° C. followed by one or more washes in 0.1×SSC/0.2% SDS at about 68° C. Other exemplary stringent conditions may refer, e.g., to washing in 6×SSC/0.05% sodium pyrophosphate at 37° C., 48° C., 55° C., and 60° C. as appropriate for the particular probe being used.

The term “homologous coding nucleic acid” also includes coding nucleic acids comprising nucleotide sequences which hybridize under moderate conditions to a nucleotide sequence selected from the group consisting of the sequences complementary to one of SEQ ID NOS:35-51 comprising nucleotide sequences which hybridize under moderate conditions to a fragment comprising at least 10, 15, 20, 25, 30, 35, 40, 50, 75, 100, 150, 200, 300, 400, 500, 750, 1000, 1250, or 1500 consecutive nucleotides of the sequences complementary to one of SEQ ID NOS:35-51. As used herein, “moderate conditions” means hybridization to filter-bound DNA in 6× sodium chloride/sodium citrate (SSC) at about 45° C. followed by one or more washes in 0.2×SSC/0.1% SDS at about 42-65° C.

The term “homologous coding nucleic acids” also includes nucleic acids comprising nucleotide sequences which encode a gene product whose activity may be complemented by a gene encoding a polypeptide comprising an amino acid sequence selected from the group consisting of SEQ ID NOS:52-68. In some embodiments, the homologous coding nucleic acids may encode a gene product whose activity is complemented by the gene product encoded by a nucleic acid comprising a nucleotide sequence selected from the group consisting of SEQ ID NOS:35-51 and SEQ ID NOS:genomic SEQUENCES.

Hybridization Methods

The invention also includes polynucleotides, preferably DNA molecules, that hybridize under stringent or moderate conditions to one of the nucleic acids of SEQ ID NOS:18-34, SEQ ID NOS:35-51, fragment comprising at least 10, 15, 20, 25, 30, 35, 40, 50, 75, 100, 150, 200, 300, 400, 500, 750, 1000, 1250, or 1500 consecutive nucleotides thereof, or the complements of any of the preceding nucleic acids. The term “hybridization” refers to the process by which a nucleic acid strand joins with a complementary strand through base pairing. Hybridization reactions can be sensitive and selective so that a particular sequence of interest can be identified even in samples in which it is present at low concentrations. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

Screening procedures which rely on nucleic acid hybridization make it possible to isolate any gene sequence from any organism, provided the appropriate probe is available. Oligonucleotide probes corresponding to any part of a nucleotide sequence encoding a protein comprising an amino acid sequence selected from the group consisting of SEQ ID NOS:52-68 can be synthesized chemically. The DNA sequence encoding the protein can be deduced from the genetic code, and the degeneracy of the code may be taken into account when designing the probe. When the sequence is degenerate, it is possible to perform a mixed addition reaction, which includes a heterogeneous mixture of denatured double-stranded DNA. For screening procedures, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. By using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucl. Acid Res., 9:879, 1981), the disclosure of which is incorporated herein by reference in its entirety. Alternatively, a subtractive library, as illustrated herein is useful for elimination of non-specific cDNA clones.

Hybridization may be under stringent or moderate conditions as defined herein or under other conditions which permit specific hybridization. The nucleic acid molecules of the invention that hybridize to these DNA sequences include oligodeoxynucleotides (“oligos”) which hybridize to the target gene under highly stringent or stringent conditions. In general, for oligos between 14 and 70 nucleotides in length the melting temperature (Tm) is calculated using the formula:
Tm(° C.)=81.5+16.6(log[monovalent cations(molar)]+0.41(% G+C)−(500/N)
where N is the length of the probe. If the hybridization is carried out in a solution containing formamide, the melting temperature may be calculated using the equation:
Tm(° C.)=81.5+16.6(log[monovalent cations(molar)]+0.41(% G+C)−(0.61)(% formamide)−(500/N)
where N is the length of the probe. In general, hybridization is carried out at about 20-25 degrees below Tm (for DNA-DNA hybrids) or about 10-15 degrees below Tm (for RNA-DNA hybrids).

Other hybridization conditions are apparent to those of skill in the art (see, for example, Ausubel, F. M. et al., eds., 1989, Current Protocols in Molecular Biology, Vol. 1, Green Publishing Associates, Inc. and John Wiley & Sons, Inc., New York, at pp. 6.3.1-6.3.6 and 2.10.3, the disclosure of which is incorporated herein by reference in its entirety).

For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 n/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

“Selective hybridization” as used herein refers to hybridization under moderately stringent or highly stringent physiological conditions (See, for example, the techniques described in Maniatis et al., 1989, Molecular Cloning A Laboratory Manual, Cold Spring Harbor Laboratory, N.Y., incorporated herein by reference), which distinguishes related from unrelated nucleotide sequences. Among the standard procedures for isolating cDNA sequences of interest is the formation of plasmid- or phage-carrying cDNA libraries which are derived from reverse transcription of mRNA from donor cells that have a high level of genetic expression. When used in combination with polymerase chain reaction technology, even low-abundance expression products can be cloned. In those cases where significant portions of the amino acid sequence of the polypeptide are known, the production of labeled single or double-stranded DNA or RNA probe sequences duplicating a sequence putatively present in the target cDNA may be employed in hybridization procedures carried out on copies of the cDNA which have been denatured to give single-stranded molecules (Jay, et al., Nucl. Acid Res., 11:2325, 1983, the disclosure of which is incorporated herein by reference in its entirety).

Library Screening for Homologous Genes

Homologous genomic sequences or homologous coding sequences may be identified by screening genomic or cDNA libraries from organisms other than rice. Standard molecular biology techniques are used to generate genomic or cDNA libraries from various cells or microorganisms. In one aspect, the libraries are generated and bound to nitrocellulose paper. The identified exogenous nucleic acid sequences of the present invention can then be used as probes to screen the libraries for homologous sequences.

For example, the libraries may be screened to identify homologous coding nucleic acids or homologous genomic nucleic acids comprising nucleotide sequences which hybridize under stringent conditions to a nucleic acid selected from the group consisting of SEQ ID NOS:18-34 and SEQ ID NOS:35-51, nucleic acids comprising nucleotide sequences which hybridize under stringent conditions to a fragment comprising at least 10, 15, 20, 25, 30, 35, 40, 50, 75, 100, 150, 200, 300, 400, 500, 750, 1000, 1250, or 1500 consecutive nucleotides of one of SEQ ID NOS:18-34 and SEQ ID NOS:35-51, nucleic acids comprising nucleotide sequences which hybridize under stringent conditions to a nucleic acid complementary to one of SEQ ID NOS:18-34 and SEQ ID NOS:35-51, nucleic acids comprising nucleotide sequences which hybridize under stringent conditions to a fragment comprising at least 10, 15, 20, 25, 30, 35, 40, 50, 75, 100, 150, 200, 300, 400, 500, 750, 1000, 1250, or 1500 consecutive nucleotides of the sequence complementary to one of SEQ ID NOS:18-34 and SEQ ID NOS:35-51.

The libraries may also be screened to identify homologous nucleic coding nucleic acids or homologous genomic sequences comprising nucleotide sequences which hybridize under moderate conditions to a nucleic acid selected from the group consisting of SEQ ID NOS:18-34 and SEQ ID NOS:35-51; nucleic acids comprising nucleotide sequences which hybridize under moderate conditions to a fragment comprising at least 10, 15, 20, 25, 30, 35, 40, 50, 75, 100, 150, 200, 300, 400, 500, 750, 1000, 1250, or 1500 consecutive nucleotides of one of SEQ ID NOS:18-34 and SEQ ID NOS:35-51; nucleic acids comprising nucleotide sequences which hybridize under moderate conditions to a nucleic acid complementary to one of SEQ ID NOS:18-34 and SEQ ID NOS:35-51; or nucleic acids comprising nucleotide sequences which hybridize under moderate conditions to a fragment comprising at least 10, 15, 20, 25, 30, 35, 40, 50, 75, 100, 150, 200, 300, 400, 500, 750, 1000, 1250, or 1500 consecutive nucleotides of the sequence complementary to one of SEQ ID NOS:18-34 and SEQ ID NOS:35-51.

The preceding methods may be used to isolate homologous coding nucleic acids or homologous genomic nucleic acids comprising a nucleotide sequence with at least 97%, at least 95%, at least 90%, at least 85%, at least 80%, or at least 70% nucleotide sequence identity to a nucleotide sequence selected from the group consisting of one of the sequences of SEQ ID NOS:18-34; SEQ ID NOS:35-51, fragments comprising at least 10, 15, 20, 25, 30, 35, 40, 50, 75, 100, 150, 200, 300, 400, 500, 750, 1000, 1250, or 1500 consecutive nucleotides thereof, and the sequences complementary thereto.

Identity may be measured using BLASTN version 2.0 with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety). For example, the homologous polynucleotides may comprise a sequence which is a naturally occurring allelic variant of one of the sequences described herein. Such allelic variants may have a substitution, deletion or addition of one or more nucleotides when compared to the nucleic acids of SEQ ID NOS:18-34 or SEQ ID NOS:35-51 or the nucleotide sequences complementary thereto.

Additionally, the above procedures may be used to isolate homologous coding nucleic acids which encode polypeptides having at least 99%, 95%, at least 90%, at least 85%, at least 80%, at least 70%, at least 60%, at least 50%, at least 40% or at least 25% amino acid identity or similarity to a polypeptide comprising the sequence of one of SEQ ID NOS:52-68 or fragments comprising at least 5, 10, 15, 20, 25, 30, 35, 40, 50, 75, 100, 200, 400, 600, 800, or 1000 consecutive amino acids thereof as determined using the FASTA version 3.0t78 algorithm with the default parameters. Alternatively, protein identity or similarity may be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

Gene Expression Arrays and Microarrays

In another embodiment of the present invention, gene expression arrays and microarrays can be employed to evaluate the transcription levels or transcription patterns of the nucleic acids of SEQ ID NOS:18-34 and SEQ ID NOS:35-51. Gene expression arrays are high density arrays of DNA samples deposited at specific locations on a glass chip, nylon membrane, or the like. Such arrays can be used by researchers to quantify relative gene expression under different conditions or in different tissues or organs. Gene expression arrays are used by researchers to help identify optimal drug targets, profile new compounds, and determine disease pathways. An example of this technology is found in U.S. Pat. No. 5,807,522, the disclosure of which is incorporated herein by reference in its entirety.

It is possible to study the expression of many genes using a single array. For example, the arrays may consist of 12×24 cm nylon filters containing PCR products corresponding to ORFs or fragments of ORFs from many genes of interest, including the nucleic acids of SEQ ID NOS:18-34 and SEQ ID NOS:35-51. In an example of a typical array, 10 ngs of each PCR product are spotted every 1.5 mm on the filter. Single stranded labeled cDNAs are prepared for hybridization to the array (no second strand synthesis or amplification step is done) and placed in contact with the filter. Thus the labeled cDNAs are of “antisense” orientation. Quantitative analysis is done by phosphorimager.

Hybridization of cDNA made from a sample of total cell mRNA to such an array followed by detection of binding by one or more of various techniques known to those in the art results in a signal at each location on the array to which cDNA hybridized. The intensity of the hybridization signal obtained at each location in the array thus reflects the amount of mRNA for that specific gene that was present in the sample. Comparing the results obtained for mRNA isolated from plants grown under different conditions thus allows for a comparison of the relative amount of expression of each individual gene during growth under the different conditions. Likewise, comparing the results obtained for mRNA obtained from different tissues or organs allows a comparison of the expression levels in different organs or tissues. In cases where the source of nucleic acid deposited on the array and the source of the nucleic acid being hybridized to the array are from two different organisms, gene expression arrays can identify homologous nucleic acids in the two organisms.

The present invention also contemplates additional methods for screening other plant species for genes related to the rice genes described in the present invention. For example, a homologous nucleic acid from a rice gene of interest may be found in another plant species. Examples of monocotyledonous plants that may be screened for similar nucleic acid sequences include, but are not limited to, monocot species such as asparagus, field and sweet corn, barley, wheat, rice, sorghum, onion, bamboo, dates, pearl millet, rye and oats, sugar cane, pineapple, and banana. Examples of dicotyledonous plants that may be screened for similar nucleic acid sequences include, but are not limited to tomato, tobacco, cotton, rapeseed, grape, field beans, soybeans, oregano, basil, peppers, lettuce, peas, alfalfa, clover, cole crops or Brassica oleracea (e.g., cabbage, broccoli, cauliflower, brussel sprouts), radish, carrot, beet, eggplant, spinach, cucumber, squash, potato, melon, cantaloupe, sunflower and various ornamentals. Examples of tree crops which may be useful include, but are not limited to avocado, apple, citrus, plum, cherry, almond, peach, pear, papaya, and mango. Examples of woody species which may be useful include, but are not limited to poplar, pine, sequoia, cedar, and oak.

Antisense Nucleotides:

In some embodiments of the present invention, a cell may be transformed with a vector which facilitates the transcription of an antisense nucleic acid or a “homologous antisense nucleic acid” which reduces the expression level or activity of a desired polypeptide within the cell or within a plant generated from the cell. The term “homologous antisense nucleic acid” includes nucleic acids comprising a nucleotide sequence having at least 97%, at least 95%, at least 90%, at least 85%, at least 80%, or at least 70% nucleotide sequence identity to a nucleotide sequence which is complementary to a nucleotide sequence selected from the group consisting of one of the sequences of SEQ ID NOS:18-34 and SEQ ID NOS:35-51 and fragments comprising at least 10, 15, 20, 25, 30, 35, 40, 50, 75, 100, 150, 200, 300, 400, 500, 750, 1000, 1250, or 1500 consecutive nucleotides thereof. Nucleic acid identity may be determined as described above.

The term “homologous antisense nucleic acid” also includes antisense nucleic acids comprising nucleotide sequences which hybridize under stringent conditions to a nucleotide sequence complementary to one of SEQ ID NOS:18-34, SEQ ID NOS:35-51 and antisense nucleic acids comprising nucleotide sequences which hybridize under stringent conditions to a fragment comprising at least 10, 15, 20, 25, 30, 35, 40, 50, 75, 100, 150, 200, 300, 400, 500, 750, 1000, 1250, or 1500 consecutive nucleotides of the sequence complementary to one of SEQ ID NOS:18-34 and SEQ ID NOS:35-51.

The term “homologous antisense nucleic acid” also includes antisense nucleic acids comprising nucleotide sequences which hybridize under moderate conditions to a nucleotide sequence complementary to one of SEQ ID NOS:18-34; SEQ ID NOS:35-51; and antisense nucleic acids comprising nucleotide sequences which hybridize under moderate conditions to a fragment comprising at least 10, 15, 20, 25, 30, 35, 40, 50, 75, 100, 150, 200, 300, 400, 500, 750, 1000, 1250, or 1500 consecutive nucleotides of the sequence complementary to one of SEQ ID NOS:18-34 and SEQ ID NOS:35-51.

In some embodiments of the present invention, a cell may be transformed with a nucleic acid complementary to a nucleic acid which encodes a polypeptide comprising an amino acid sequence selected from the group consisting of SEQ ID NOS:52-68, a nucleic acid complementary to a nucleic acid which encodes at least 5, 10, 15, 20, 25, 30, 35, 40, 50, 75, 100, 200, 400, 600, 800, or 1000 consecutive amino acids of a polypeptide sequence selected from the group consisting of SEQ ID NOS:52-68, a nucleic acid complementary to a homologous coding nucleic acid, a nucleic acid complementary to at least 10, 15, 20, 25, 30, 35, 40, 50, 75, 100, 150, 200, 300, 400, 500, 750, 1000, 1250, or 1500 consecutive nucleotides of a homologous coding nucleic acid, a nucleic acid complementary to a nucleic acid which encodes a homologous polypeptide, or a nucleic acid complementary to a nucleic acid which encodes at least 5, 10, 15, 20, 25, 30, 35, 40, 50, 75, 100, 200, 400, 600, 800, or 1000 consecutive amino acids of a homologous polypeptide.

PCR

Embodiments of the invention may utilize techniques such as polymerase chain reaction. As used herein, the term “polymerase chain reaction” (“PCR”) refers to the method of K. B. Mullis U.S. Pat. Nos. 4,683,195 and 4,683,202, hereby incorporated by reference, which describe a method for increasing the concentration of a segment of a template sequence in a mixture of genomic DNA without cloning or purification. This process for amplifying the template sequence consists of introducing a large excess of two oligonucleotide primers to the DNA mixture containing the desired template sequence, followed by a precise sequence of thermal cycling in the presence of a DNA polymerase. The two primers are complementary to their respective strands of the double stranded template sequence. To effect amplification, the mixture is denatured and the primers then annealed to their complementary sequences within the template molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired template sequence. The length of the amplified segment of the desired template sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the template sequence become the predominant sequences (in terms of concentration) in the mixture, they are said to be “PCR amplified”.

PCR techniques make it possible to amplify a single copy of a specific template sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The exact lengths of the primers will depend on many factors, including temperature, source of primer and the use of the method.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

As used herein, the term “template,” refers to nucleic acid that is to acted upon, such as nucleic acid that is to be mixed with polymerase. In some cases “template” is sought to be sorted out from other nucleic acid sequences. “Substantially single-stranded template” is nucleic acid that is either completely single-stranded (having no double-stranded areas) or single-stranded except for a proportionately small area of double-stranded nucleic acid (such as the area defined by a hybridized primer or the area defined by intramolecular bonding). “Substantially double-stranded template” is nucleic acid that is either completely double-stranded (having no single-stranded region) or double-stranded except for a proportionately small area of single-stranded nucleic acid (such as the area defined at the ends of telomeric DNA).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acids. Amplification techniques have been designed primarily for this sorting out.

As used herein, the term “amplifiable nucleic acid” is used in reference to nucleic acids which may be amplified by any amplification method, including but not limited to PCR.

As used herein, the terms “PCR product”, “PCR fragment” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

Polypeptides

The present invention includes isolated or purified polypeptides comprising the amino acid sequences of SEQ ID NOS:52-68 or fragments comprising at least 5, 10, 15, 20, 25, 30, 35, 40, 50, 75, 100, 200, 300, 400, 500, 750, 1000, 1250, or 1500 consecutive amino acids thereof. The present invention also includes amino acid sequences substantially the same as sequences set forth in SEQ ID NOS:52-68. The term “substantially the same” refers to amino acid sequences that retain the protein activity as described in Table 2 herein. The term “protein activity” as described herein is defined as having a similar general function as that of the native protein or its homologs. Examples of protein activity may include but are not limited to enzymatic activity, DNA binding activity, RNA binding activity, protein binding activity, activity in biochemical pathways, activity in signalling pathways, activity in subcellular transport mechanisms, and activity in cellular scaffolding mechanisms. Polypeptides of the invention include conservative variations of the polypeptide sequence that produce sequences that are substantially the same as the sequence set forth in SEQ ID NOS:52-68. The term “conservative variation” as used herein denotes the replacement of an amino acid by another biologically similar residue. Examples of conservative variations include the substitution of one hydrophobic residue such as isoleucine, valine, leucine or methionine for another, or the substitution of one polar residue for another, such as the substitution of arginine for lysine, glutamic acid for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

TABLE 2

| | SEQ ID NO | | SEQ ID NO | |
|---|---|---|---|---|
| | of genomic | | of complete | |
| Internal | DNA with | SEQ ID NO | open reading | SEQ ID NO |
| Code for | portion | of genomic | frame | of poly- |
| Gene and | of T-DNA | DNA without | (coding | peptide |
| Line | insert | insert | sequence) | sequence |
| 1b-115-22 | 1 | 18 | 35 | 52 |
| 1b-164-43 | 2 | 19 | 36 | 53 |
| 1b-192-40 | 3 | 20 | 37 | 54 |
| 1b-207-27 | 4 | 21 | 38 | 55 |
| 1b-138-07 | 5 | 22 | 39 | 56 |
| 1d-059-12 | 6 | 23 | 40 | 57 |
| 1c-087-40 | 7 | 24 | 41 | 58 |
| 1c-017-14 | 8 | 25 | 42 | 59 |
| 1c-038-56 | 9 | 26 | 43 | 60 |
| 1c-041-47 | 10 | 27 | 44 | 61 |
| 1c-064-20 | 11 | 28 | 45 | 62 |
| 1c-109-35 | 12 | 29 | 46 | 63 |
| 1c-109-51 | 13 | 30 | 47 | 64 |
| 1c-056-07 | 14 | 31 | 48 | 65 |
| 1c-100-32 | 15 | 32 | 49 | 66 |
| 1c-142-27 | 16 | 33 | 50 | 67 |
| 1c-140-04 | 17 | 34 | 51 | 68 |

The term “substantially pure” as used herein refers to a polypeptide which is substantially free of other proteins, lipids, carbohydrates or other materials with which it is naturally associated. Thus, the term “substantially pure” does not encompass a polypeptide which is present on an electrophoretic separation medium along with a significant amount of other proteins. One skilled in the art can purify the polypeptide using standard techniques for protein purification. The purity of the polypeptide can also be determined by amino-terminal amino acid sequence analysis.

Polypeptides of the present invention include polypeptides consisting essentially of SEQ ID NOS:52-68, wherein the term “consisting essentially of” requires that the protein formed by the amino acid sequence has the activity or function as set forth in Table 2.

Embodiments of the present invention also include polypeptides that are homologous to SEQ ID NOS:52-68. The term “homologous polypeptide” includes polypeptides having at least 99%, 95%, at least 90%, at least 85%, at least 80%, at least 70%, at least 60%, at least 50%, at least 40% or at least 25% amino acid identity or similarity to a polypeptide comprising one of the amino acid sequences of SEQ ID NOS:52-68. NOS: or by a homologous antisense nucleic acid, or polypeptides having at least 85%, at least 80%, at least 70%, at least 60%, at least 50%, at least 40% or at least 25% amino acid identity or similarity to a polypeptide to a fragment comprising at least 5, 10, 15, 20, 25, 30, 35, 40, 50, 75, 100, 200, 400, 600, 800, or 1000 consecutive amino acids of a polypeptide comprising one of the amino acid sequences of SEQ ID NOS:52-68. Identity or similarity may be determined using the FASTA version 3.0t78 algorithm with the default parameters. Alternatively, protein identity or similarity may be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

Embodiments of the invention also include functional polypeptides, and functional fragments thereof. As used herein, the term “functional polypeptide” refers to a polypeptide which possesses biological function or activity which is identified through a defined functional assay and which is associated with a particular biologic, morphologic, or phenotypic alteration in the cell. The term “functional fragments of a polypeptide”, refers to all fragments of the polypeptide that retain activity including, but not limited to, the functions listed in Table 2. Biologically functional fragments, for example, can vary in size from a polypeptide fragment as small as an epitope capable of binding an antibody molecule to a large polypeptide capable of participating in the characteristic induction or programming of phenotypic changes within a cell.

The activity of the polypeptide, as well as its role in biosynthetic or biological pathways can be utilized in bioassays to identify biologically active fragments, mutants, and variants of the polypeptide and related polypeptides. Assays can be performed to detect the enzymatic activity of the polypeptide.

Minor modifications of the primary amino acid sequence may result in proteins which have substantially equivalent activity to the polypeptide described herein in SEQ ID NOS:52-68. Such modifications may be deliberate, as for example by site-directed mutagenesis, or may be spontaneous. Modified polypeptides produced by these modifications having biological activity as listed in Table 2 are included herein. Further, deletion of one or more amino acids can also result in a modification of the structure of the resultant molecule without significantly altering its activity. This can lead to the development of a smaller active molecule which could have broader utility.

Polypeptides of the invention can be analyzed by standard methods of analysis including, but not limited to, immunoprecipitation, SDS-PAGE, immunoblotting, and chromatography. In addition, the in vitro synthesized (IVS) protein assay as described in the present examples can be used to analyze the protein product.

Another aspect of the invention includes polypeptides or fragments thereof having at least about 70%, at least about 80%, at least about 85%, at least about 90%, at least about 95%, or more than about 95% homology to one of the polypeptides of SEQ ID NOS:52-68, and sequences substantially identical thereto, or a fragment comprising at least 5, 10, 15, 20, 25, 30, 35, 40, 50, 75, 100, 150, 200, 300, 400, 500, 600 or more consecutive amino acids thereof. Homology may be determined using any of the methods described herein which align the polypeptides or fragments being compared and determines the extent of amino acid identity or similarity between them. It will be appreciated that amino acid “homology” includes conservative amino acid substitutions such as those described above.

The polypeptides or fragments having homology to one of the polypeptides of SEQ ID NOS:52-68, and sequences substantially identical thereto, or a fragment comprising at least about 5, 10, 15, 20, 25, 30, 35, 40, 50, 75, 100, 150, 200, 300, 400, 500, 600 or more consecutive amino acids thereof may be obtained by isolating the nucleic acids encoding them using the techniques described herein.

Alternatively, the homologous polypeptides or fragments may be obtained through biochemical enrichment or purification procedures. The sequence of potentially homologous polypeptides or fragments may be determined by proteolytic digestion, gel electrophoresis and/or microsequencing. The sequence of the prospective homologous polypeptide or fragment can be compared to one of the polypeptides of SEQ ID NOS:52-68, and sequences substantially identical thereto, or a fragment comprising at least about 5, 10, 15, 20, 25, 30, 35, 40, 50, 75, 100, 150, 200, 300, 400, 500, 600 or more consecutive amino acids thereof using any of the programs described above.

Homologous amino acid or nucleotide sequences of the present invention preferably comprise enough of the amino acid sequence of a polypeptide or the nucleotide sequence of a gene to afford identification of that polypeptide or gene, either by manual evaluation of the sequence by one skilled in the art, or by computer-automated sequence comparison and identification using algorithms such as BLAST (Basic Local Alignment Search Tool) (for a review see Altschul, et al., Meth Enzymol. 266:460, 1996; and Altschul, et al., Nature Genet. 6:119, 1994, the disclosures of which are hereby incorporated by reference in their entireties). BLAST is the heuristic search algorithm employed by the programs blastp, blastn, blastx, tblastn, and tblastx using the statistical methods of Karlin and Altschul (available at www.ncbi.nih.gov/BLAST) Altschul, et al., J. Mol. Biol. 215:403, 1990). The BLAST programs may be tailored for sequence similarity searching, for example to identify homologues to a query sequence. The BLAST pages offer several different databases for searching. Some of these databases, such as ecoli, dbEST and month, are subsets of the NCBI (National Center for Biotechnology Information) databases, while others, such as SwissProt, PDB and Kabat are compiled from outside sources. Protein BLAST allows one to input protein sequences and compare these against other protein sequences.

The five BLAST programs available at Internet website:www.ncbi.nlm.nih.gov perform the following tasks:

blastp—compares an amino acid query sequence against a protein sequence database.

blastn—compares a nucleotide query sequence against a nucleotide sequence database.

blastx—compares the six-frame conceptual translation products of a nucleotide query sequence (both strands) against a protein sequence database.

tblastn—compares a protein query sequence against a nucleotide sequence database dynamically translated in all six reading frames (both strands).

tblastx—compares the six-frame translations of a nucleotide query sequence against the six-frame translations of a nucleotide sequence database.

Other computer program methods to determine identity and similarity between the two sequences include but are not limited to the GCG program package (Devereux, et al., Nucl. Acids Res. 12:387, 1984, the disclosure of which is hereby incorporated by reference in its entirety) and FASTA (Atschul, et al., J. Molec. Biol. 215:403, 1990, the disclosure of which is hereby incorporated by reference in its entirety). By “percentage identity” is meant % of identical amino acids between the two compared proteins. By “% similarity” is meant the percentage of similar amino acids between the two compared proteins.

Antibodies

The invention also provides antibodies immunoreactive with any polypeptide, or antigenic fragments thereof. In some embodiments, the antibody may consist essentially of polyclonal antibodies, pooled monoclonal antibodies with different epitopic specificities, as well as distinct monoclonal antibody preparations is provided. Monoclonal antibodies are made from antigen containing fragments of the protein by methods well known to those skilled in the art (Kohler, et al., Nature, 256:495, 1975, the disclosure of which is hereby incorporated by reference in its entirety).

The term “antibody” as used in this invention includes intact molecules as well as fragments thereof, such as Fab, F(ab′)2, and Fv capable of binding to an epitopic determinant present in polypeptide. Such antibody fragments retain some ability to selectively bind with its antigen or receptor.

Methods of making these fragments are known in the art. (See for example, Harlow and Lane, Antibodies: A Laboratory Manual, Cold Spring Harbor Laboratory, New York (1988), incorporated herein by reference).

As used in this invention, the term “epitope” refers to an antigenic determinant on an antigen to which the paratope of an antibody binds. Epitopic determinants often consist of chemically active surface groupings of molecules such as amino acids or sugar side chains and usually have specific three dimensional structural characteristics, as well as specific charge characteristics.

Antibodies which bind to the polypeptide of the invention can be prepared using an intact polypeptide or fragments containing small peptides of interest as the immunizing antigen. For example, it may be desirable to produce antibodies that specifically bind to the N- or C-terminal domains of the polypeptide. The polypeptide or peptide used to immunize an animal may be derived from translated cDNA or may be chemically synthesized, and may further be conjugated to a carrier protein, if desired. Commonly used carriers which are chemically coupled to an immunizing peptide include keyhole limpet hemocyanin (KLH), thyroglobulin, bovine serum albumin (BSA), and tetanus toxoid.

Polyclonal or monoclonal antibodies can be further purified, for example, by binding to and eluting from a matrix to which the polypeptide or a peptide to which the antibodies were raised is bound. Those of skill in the art are familiar with various techniques common in the immunology arts for purification and/or concentration of polyclonal antibodies, as well as monoclonal antibodies (See for example, Coligan, et al., Unit 9, Current Protocols in Immunology, Wiley Interscience, 1994, incorporated by reference).

It is also possible to use the anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

A cDNA expression library such as lambda gt11, can be screened indirectly for polypeptides using antibodies specific for epitopes of polypeptides of the invention. Such antibodies may be polyclonally or monoclonally derived, and may be used to detect expression product indicative of the presence of cDNA sequences of the invention.

Screening for Molecules that Interact or Bind with Genes or Polypeptides of the Invention

Other embodiments of the present invention provide methods of screening or identifying proteins, small molecules or other compounds which are capable of inducing or inhibiting the expression of the genes and proteins. The assays may be performed in vitro using transformed or non-transformed cells, immortalized cell lines, or in vivo using transformed plant models enabled herein. In particular, the assays may detect the presence of increased or decreased expression of genes or proteins on the basis of increased or decreased mRNA expression, increased or decreased levels of protein products, or increased or decreased levels of expression of a marker gene (e.g., beta-galactosidase, green fluorescent protein, alkaline phosphatase or luciferase) operably joined to a 5′ regulatory region in a recombinant construct. Cells known to express a particular polypeptide, or transformed to express a particular polypeptide, are incubated and one or more test compounds are added to the medium. After allowing a sufficient period of time, e.g., anywhere from 0-72 hours or longer, for the compound to induce or inhibit the expression of the gene, any change in levels of expression from an established baseline may be detected using any of the techniques described above.

Additional embodiments of the present invention provide methods for identifying proteins and other compounds which bind to, or otherwise directly interact with, the sequences of the invention. The proteins and compounds will include endogenous cellular components which interact with the sequences of the invention in vivo and which, therefore, provide new targets for agricultural products, as well as recombinant, synthetic and otherwise exogenous compounds which may have binding capacity and, therefore, may be candidates for plant growth modulators. Thus, in one series of embodiments, high throughput screen (HTS) protein or DNA chips, cell lysates or tissue homogenates may be screened for proteins or other compounds which bind to one of the normal or mutant genes. Alternatively, any of a variety of exogenous compounds, both naturally occurring and/or synthetic (e.g., libraries of small molecules or peptides), may be screened for capacity to bind to the sequences of the invention.

In various embodiments, an assay is conducted to detect binding of a polypeptide selected from the group consisting of SEQ ID NOS:52-68 to another moiety. The polypeptide in these assays may be any polypeptide comprising or derived from a normal or mutant protein, including functional domains or antigenic determinants. Binding may be detected by non-specific measures (e.g., transcription modulation, altered chromatin structure, peptide production or changes in the expression of other downstream genes which can be monitored by differential display, 2D gel electrophoresis, differential hybridization, or SAGE methods) or by direct measures such as immunoprecipitation, the Biomolecular Interaction Assay (BIAcore) or alteration of protein gel electrophoresis. The preferred methods involve variations on the following techniques: (1) direct extraction by affinity chromatography; (2) co-isolation of the polypeptide components and bound proteins or other compounds by immunoprecipitation; (3) BIAcore analysis; and (4) yeast two-hybrid systems.

Additional embodiments of the present invention provide methods of identifying proteins, small molecules and other compounds capable of modulating the activity of normal or mutant polypeptide.

Additional embodiments of the present invention provide methods of identifying compounds on the basis of their ability to affect the expression of the gene sequences of the invention, the activity of the polypeptides of the invention, the activity of other genes regulated by polypeptides of the invention, the activity of proteins that interact with normal or mutant proteins, the intracellular localization of the polypeptides of the invention, changes in transcriptional activity, the presence or levels of the polypeptides, or other biochemical, histological, or physiological markers which distinguish cells bearing normal and modulated activity in plants and in animals. Methods of identifying compounds with activity toward the gene or the protein may be practiced using normal cells or plants, the transformed cells and plant models of the present invention, or cells obtained from subjects bearing normal or mutant genes.

In accordance with another aspect of the invention, the proteins of the invention can be used as starting points for rational chemical design to provide ligands or other types of small chemical molecules. Alternatively, small molecules or other compounds identified by the above-described screening assays may serve as “lead compounds” in design of modulators of biological pathways in plants.

Expression Vectors and their Use for Gene Expression and Protein Production

The sequences of the present invention can be expressed in vitro by transfer of the gene sequences into a suitable host cell. “Host cells” are cells in which a vector containing a coding region can be propagated and its DNA expressed. The term also includes any progeny or graft material, for example, of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

The polynucleotide sequences according to the present invention may be inserted into a recombinant expression vector. The terms “recombinant expression vector” or “expression vector” refer to a plasmid, virus or other vehicle known in the art that has been manipulated by insertion or incorporation of the genetic sequence. Such expression vectors contain a promoter sequence which facilitates the efficient transcription of the inserted sequence. The expression vector typically contains an origin of replication, a promoter, and one or more genes that allow phenotypic selection of the transformed cells.

Methods well known to those skilled in the art can be used to construct expression vectors containing the coding sequence and appropriate transcriptional/translational control signals. These methods include in vitro recombinant DNA techniques, synthetic techniques, and in vivo recombination/genetic techniques.

A variety of host-expression vector systems may be utilized to express the coding sequence in numerous types of organisms. These include, but are not limited to, microorganisms such as bacteria transformed with recombinant bacteriophage DNA, plasmid DNA or cosmid DNA expression vectors containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence; insect cell systems infected with recombinant virus expression vectors (e.g., baculovirus) containing the coding sequence; or animal cell systems infected with recombinant virus expression vectors (e.g., retroviruses, adenovirus, vaccinia virus) containing the coding sequence, or transformed animal cell systems engineered for stable expression.

Any of a number of suitable transcription and translation elements, including constitutive and inducible promoters, transcription enhancer elements, and/or transcription terminators, may be used in the expression vector (see e.g., Bitter, et al., Methods in Enzymology 153:516, 1987, the disclosure of which is incorporated herein by reference in its entirety). The choice of these elements will vary depending on the host/vector system utilized. The particular promoter selected should be capable of causing sufficient expression to result in the production of an effective amount of the gene product. The promoters used in the vector constructs of the present invention may be modified, if desired, to affect their control characteristics.

For example, when cloning in bacterial systems, inducible promoters such as pL of bacteriophage λ, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Suitable promoters for use in plant host cells include, for example, CaMV 35S promoters, the Agrobacterium-derived promoters nopaline synthase (NOS) and octopine synthase (OCS), the rice a tubulin OsTubA1 promoter, heat shock promoters such as soybean hsp17.5-E or hsp17.3-B, inducible or tissue-specific promoters, as well as the native promoter of the gene of interest. Promoters produced by recombinant DNA or synthetic techniques may also be used to provide for transcription of the inserted coding sequence.

Following expression of the protein encoded by the identified exogenous nucleic acid according to the methods described above, the protein may be purified and may method described above may be used, for example, for structural characterization studies, protein-protein interaction studies, protein-nucleic acid interaction studies, and the like. Isolation and purification of recombinantly expressed polypeptide, or fragments thereof, may be carried out by conventional means including preparative chromatography and immunological separations involving monoclonal or polyclonal antibodies. Examples of suitable methods are described below.

Protein purification techniques are well known in the art. Proteins encoded and expressed from identified exogenous nucleic acids can be partially purified using precipitation techniques, such as precipitation with polyethylene glycol. Alternatively, epitope tagging of the protein can be used to allow simple one step purification of the protein. In addition, chromatographic methods such as ion-exchange chromatography, gel filtration, use of hydroxyapatite columns, immobilized reactive dyes, chromatofocusing, and use of high-performance liquid chromatography, may also be used to purify the protein. Electrophoretic methods such as one-dimensional gel electrophoresis, high-resolution two-dimensional polyacrylamide electrophoresis, isoelectric focusing, and others are contemplated as purification methods. Also, affinity chromatographic methods, comprising antibody columns, ligand presenting columns and other affinity chromatographic matrices are contemplated as purification methods in the present invention.

The purified proteins produced from the gene encoding a polypeptide comprising one of SEQ ID NOS:52-68, and sequences substantially identical thereto, or a fragment comprising at least 5, 10, 15, 20, 25, 30, 35, 40, 50, 75, 100, 150, 200, 300, 400, 500, 600 or more consecutive amino acids thereof can be used in a variety of protocols to generate useful reagents. In one embodiment of the present invention, antibodies are generated against the proteins expressed from the vectors. Both monoclonal and polyclonal antibodies can be generated against the expressed proteins. Methods for generating monoclonal and polyclonal antibodies are well known in the art. Also, antibody fragment preparations prepared from the produced antibodies discussed above are contemplated.

Another application for the purified proteins of the present invention is to screen small molecule libraries for candidate compounds active against the various target proteins of the present invention. Advances in the field of combinatorial chemistry provide methods, well known in the art, to produce large numbers of candidate compounds that can have a binding, or otherwise inhibitory effect on a target protein. Accordingly, the screening of small molecule libraries for compounds with binding affinity or inhibitory activity for a target protein produced from an identified gene is contemplated by the present invention.

Vectors for Genetic Modification of Plants with Genes of the Invention

Vector(s) employed in the present invention for transformation of a plant cell include a nucleic acid sequence encoding or a sequence which reduces the activity or level of a protein comprising one of the amino acid sequences of SEQ ID NOS:52-68. For example, the activity or level of a protein comprising one of the amino acid sequences of SEQ ID NOS:52-68 may be an antisense nucleic acid as described above, a homologous antisense nucleic acid as described above, a ribozyme (Welch et al., (1998) Curr Opin. Biotechnol. 9:486-496; Samarsky, et al., (2000), Curr. Issues Mol. Biol. 2:87-93); or a double stranded RNA (Sharp, (1999), Genes Dev. 13:139-141, the disclosures of which are hereby incorporated by reference in their entireties), operably associated with a promoter. To commence a transformation process in accordance with the present invention, it is first necessary to construct a suitable vector and properly introduce it into the plant cell. Details of the construction of vectors utilized herein are known to those skilled in the art of plant genetic engineering.

Genetically modified plants of the present invention are produced by contacting a plant cell with a vector including at least one nucleic acid sequence encoding one of the amino acid sequences of SEQ ID NOS:52-68. To be effective once introduced into plant cells, the nucleic acid sequence must be operably associated with a promoter which is effective in the plant cells to cause transcription of the gene. Additionally, a polyadenylation sequence or transcription control sequence recognized in plant cells may be employed. It is preferred that the vector harboring the nucleic acid sequence to be inserted also contain one or more selectable marker genes so that the transformed cells can be selected from non-transformed cells in culture, as described herein.

One of skill in the art will be able to select an appropriate vector as needed for introducing the desired nucleic acid sequence in a relatively intact state. Thus, any vector which will produce a plant carrying the introduced DNA sequence should be sufficient. Even use of a naked piece of DNA would be expected to confer the properties of this invention, though at low efficiency. The selection of the vector, or whether to use a vector, is typically guided by the method of transformation selected.

Vectors for gene expression in plants may contain any of a number of promoters that are functional in plants. Many types of plant-derived promoters as well promoters derived from other sources that are functional in plants are now known. Some types of plant-derived promoters may be constantly active. Others may be active only in certain circumstances or cell types. Examples of this later group include tissue-specific, developmentally specific, stress-specific, or environmentally specific promoters. Additionally, developmental, tissue-specific, and environmentally inducible promoters may be combined at the upstream regulatory region of a gene sequence to carefully regulate the spatial and temporal production of polypeptide in order to produce novel, desirable plant phenotypes.

The term “operably associated” refers to functional linkage between a promoter sequence and the nucleic acid sequence regulated by the promoter. The operably linked promoter controls the expression of the nucleic acid sequence.

The expression of proteins comprising one of the amino acid sequences of SEQ ID NOS:52-68 may be driven by a number of promoters. The endogenous, or native promoter of a structural gene of interest may be utilized for transcriptional regulation of the gene, or the promoter may be a foreign regulatory sequence. For plant expression vectors, suitable viral promoters include the 35S RNA and 19S RNA promoters of CaMV (Brisson, et al., 1984, Nature, 310:511, 1984; Odell, et al., Nature, 313:810, 1985); the full-length transcript promoter from Figwort Mosaic Virus (FMV) (Gowda, et al., J. Cell Biochem., 13D: 301, 1989) and the coat protein promoter to TMV (Takamatsu, et al., EMBO J. 6:307, 1987), the disclosures of which are incorporated herein by reference in their entireties. Alternatively, plant promoters such as the light-inducible promoter from the small subunit of ribulose bis-phosphate carboxylase (ssRUBISCO) (Coruzzi, et al., EMBO J., 3:1671, 1984; Broglie, et al., Science, 224:838, 1984); mannopine synthase promoter (Velten, et al., EMBO J., 3:2723, 1984) nopaline synthase (NOS) and octopine synthase (QCS) promoters (carried on tumor-inducing plasmids of Agrobacterium tumefaciens) or heat shock promoters, e.g., soybean hsp17.5-E or hsp17.3-B (Gurley, et al., Mol. Cell. Biol., 6:559, 1986; Severin, et al., Plant Mol. Biol., 15:827, 1990), the disclosures of which are incorporated herein by reference in their entireties, may be used.

Promoters useful in the invention include both natural constitutive and inducible promoters as well as engineered promoters. The CaMV promoters are examples of constitutive promoters. To be most useful, an inducible promoter should 1) provide low expression in the absence of the inducer; 2) provide high expression in the presence of the inducer; 3) use an induction scheme that does not interfere with the normal physiology of the plant; and 4) have no effect on the expression of other genes. Examples of inducible promoters useful in plants include those induced by chemical means, such as the yeast metallothionein promoter which is activated by copper ions (Mett, et al., Proc. Natl. Acad. Sci., U.S.A., 90:4567, 1993), the disclosure of which is incorporated herein by reference in its entirety; In2-1 and In2-2 regulator sequences which are activated by substituted benzenesulfonamides, e.g., herbicide safeners (Hershey, et al., Plant Mol. Biol., 17:679, 1991), the disclosure of which is incorporated herein by reference in its entirety; and the GRE regulatory sequences which are induced by glucocorticoids (Schena, et al., Proc. Natl. Acad. Sci., U.S.A., 88:10421, 1991), the disclosure of which is incorporated herein by reference in its entirety. Other promoters, both constitutive and inducible will be known to those of skill in the art.

The particular promoter selected should be capable of causing sufficient expression to result in the production of an effective amount of protein or a sufficient amount of a transcript which reduces the activity or level of a protein comprising an amino acid sequence of SEQ ID NOS:52-68. The promoters used in the vector constructs of the present invention may be modified, if desired, to affect their control characteristics.

Tissue specific promoters may also be utilized in the present invention. An example of a tissue specific promoter is the promoter active in shoot meristems (Atanassova, et al., Plant J., 2:291, 1992), the disclosure of which is incorporated herein by reference in its entirety. Other tissue specific promoters useful in transgenic plants, including the cdc2a promoter and cyc07 promoter, will be known to those of skill in the art. (See for example, Ito, et al., Plant Mol. Biol., 24:863, 1994; Martinez, et al., Proc. Natl. Acad. Sci. USA, 89:7360, 1992; Medford, et al., Plant Cell, 3:359, 1991; Terada, et al., Plant Journal., 3:241, 1993; Wissenbach, et al., Plant Journal, 4:411, 1993), the disclosures of which are incorporated herein by reference in their entireties.

Many types of inducible promoters are known, including those that are induced by environmental conditions such as drought, cold, salt stress, heat, or nutrient stress. Promoters which are induced by exogenous applications of a compound may also be operably linked to the gene. Other modifications could be made to comply with specific environmental or developmental needs of the crop to be modified. Any of these may be linked to the nucleic acid of interest to create plants with desired expression characteristics in the transformed plant.

The nucleic acid of interest may also be operably linked to both tissue-specific and environmentally inducible promoters to produce crops with agricultural characteristics that are regulated by environmental conditions. For example, the nucleic acid of interest could be linked to both cold-specific promoters and seed specific promoters. Alternatively, the nucleic acid of interest could be linked to root-specific promoters and drought-specific promoters such that, upon water stress, growth is focused toward more root growth to increase water uptake. This may result in increased survival under poor environmental conditions.

The upstream regions that control transcription of the nucleic acid of interest gene may contain more than one promoter, and may additionally contain one or more enhancer elements. Such regions may be present, for example, in activation-tagging vectors (Weigel, et al., Plant Physiol. 122:1003, 2000), the disclosure of which is incorporated herein by reference in its entirety, which contain multimerized transcriptional enhancers from the cauliflower mosaic virus (CaMV) 35S gene. In this method, the activation tagging sequence serves to upregulate endogenous genes that are downstream of the insertion site.

Optionally, a selectable marker may be associated with the nucleic acid sequence to be inserted. As used herein, the term “marker” refers to a gene encoding a trait or a phenotype which permits the selection of, or the screening for, a plant or plant cell containing the marker. The marker gene may be an antibiotic resistance gene whereby the appropriate antibiotic can be used to select for transformed cells from among cells that are not transformed. Examples of suitable selectable markers include adenosine deaminase, dihydrofolate reductase, hygromycin-B-phospho-transferase, thymidine kinase, xanthine-guanine phospho-ribosyltransferase and amino-glycoside 3′-O-phospho-transferase II (kanamycin, neomycin and G418 resistance). Other suitable markers will be known to those of skill in the art.

As can be seen from the above discussion, there are many options for the components of the vector suitable for gene transfer to plants. The vector to be used for plant transformation may comprise additional sequences as desired for the particular application. One of skill in the art will be able to design a suitable vector strategy to deliver the gene of interest to the plant. Once the desired vector containing the gene of interest is prepared, the construct can be introduced to plant cells by a variety of methods, including but not limited to those described below.

Plant Transformation with Genes of the Invention

The term “genetic modification” as used herein refers to the introduction of one or more heterologous nucleic acid sequences, e.g., a protein-encoding sequence or a sequence which reduces the activity or level of a protein comprising one of the amino acid sequences of SEQ ID NOS:52-68, into one or more plant cells which can then be used to generate whole, sexually competent, viable plants. The term “genetically modified” as used herein refers to a plant which has been generated through the aforementioned process. Genetically modified plants of the invention are capable of self-pollinating or cross-pollinating with other plants of the same species so that the foreign gene, carried in the germ line, can be inserted into or bred into agriculturally useful plant varieties. The term “plant cell” as used herein refers to protoplasts, gamete-producing cells, and cells which regenerate into whole plants. Accordingly, a seed comprising multiple plant cells capable of regenerating into a whole plant, is included in the definition of “plant cell”.

As used herein, the term “plant” refers to either a whole plant, a plant part, a plant cell, or a group of plant cells, such as plant tissue, for example. Plantlets are also included within the meaning of “plant”. Plants included in the invention are any plants amenable to transformation techniques, including angiosperms, gymnosperms, monocotyledons and dicotyledons.

Examples of monocotyledonous plants include, but are not limited to, asparagus, field and sweet corn, barley, wheat, rice, sorghum, onion, bamboo, dates, pearl millet, rye and oats, sugar cane, pineapple, and banana. Examples of dicotyledonous plants include, but are not limited to Arabidopsis, tomato, tobacco, cotton, rapeseed, grape, field beans, soybeans, oregano, basil, peppers, lettuce, peas, alfalfa, clover, cole crops or Brassica oleracea (e.g., cabbage, broccoli, cauliflower, brussel sprouts), radish, carrot, beet, eggplant, spinach, cucumber, squash, potato, melon, cantaloupe, sunflower and various ornamentals. Examples of tree crops which may be useful include, but are not limited to avocado, apple, citrus, plum, cherry, almond, peach, pear, papaya, and mango. Examples of woody species which may be useful include, but are not limited to poplar, pine, sequoia, cedar, and oak.

The term “heterologous nucleic acid sequence” as used herein refers to a nucleic acid foreign to the recipient plant host or, native to the host if the native nucleic acid is substantially modified from its original form. For example, the term includes a nucleic acid originating in the host species, where such sequence is operably linked to a promoter that differs from the natural or wild-type promoter. In the broad method of the invention, at least one nucleic acid sequence encoding a polypeptide of the invention is operably linked with a promoter. It may be desirable to introduce more than one copy of the polynucleotide into a plant for enhanced gene expression. For example, multiple copies of the gene would have the effect of increasing gene expression and/or production of the encoded polypeptides in the plant.

It may also be desirable to decrease levels of gene expression in the plant. Any method to downregulate gene expression may be used, but typical examples include antisense technology, cosuppression, RNA inhibition (RNAi), and ribozyme inhibition. In the antisense method, for example, antisense molecules are introduced into cells that contain a certain gene, for example, and may function by decreasing the amount of polypeptide production in a cell, or may function by a different mechanism. Antisense polynucleotides useful for the present invention are complementary to specific regions of a corresponding target mRNA. An antisense polynucleotide can be introduced to a cell by introducing an expressible construct containing a nucleic acid segment that codes for the polynucleotide. Antisense polynucleotides in context of the present invention may include short sequences of nucleic acid known as oligonucleotides, usually 10-50 bases in length, as well as longer sequences of nucleic acid that may exceed the length of the gene sequence itself.

The nucleic acid sequences utilized in the present invention can be introduced into plant cells using Ti plasmids of Agrobacterium tumefaciens, root-inducing (Ri) plasmids, and plant virus vectors. (For reviews of such techniques see, for example, Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9, and Horsch, et al., Science, 227:1229, 1985, all incorporated herein by reference). In addition to plant transformation vectors derived from the Ti or root-inducing (Ri) plasmids of Agrobacterium, alternative methods may involve, for example, the use of liposomes, electroporation, chemicals that increase free DNA uptake, transformation using viruses or pollen and the use of microprojection.

Transformation of plants in accordance with the invention may be carried out in essentially any of the various ways known to those skilled in the art of plant molecular biology. (See, for example, Methods of Enzymology, Vol. 153, 1987, Wu and Grossman, eds., Academic Press, incorporated herein by reference). As used herein, the term “transformation” means alteration of the genotype of a host plant by the introduction of the nucleic acid sequence.

For example, a nucleic acid sequence can be introduced into a plant cell utilizing Agrobacterium tumefaciens containing the Ti plasmid, as mentioned briefly above. In using an A. tumefaciens culture as a transformation vehicle, it is most advantageous to use a non-oncogenic strain of Agrobacterium as the vector carrier so that normal non-oncogenic differentiation of the transformed tissues is possible. It is also preferred that the Agrobacterium harbor a binary Ti plasmid system. Such a binary system comprises 1) a first Ti plasmid having a virulence region essential for the introduction of transfer DNA (T-DNA) into plants, and 2) a chimeric plasmid. The latter contains at least one border region of the T-DNA region of a wild-type Ti plasmid flanking the nucleic acid to be transferred. Binary Ti plasmid systems have been shown effective to transform plant cells (De Framond, Biotechnology, 1: 262, 1983; Hoekema, et al., Nature, 303:179, 1983), the disclosures of which are incorporated herein by reference in their entireties.

Methods involving the use of Agrobacterium in transformation according to the present invention include, but are not limited to: 1) co-cultivation of Agrobacterium with cultured isolated protoplasts; 2) transformation of plant cells or tissues with Agrobacterium; or 3) transformation of seeds, apices or meristems with Agrobacterium.

In addition, gene transfer can be accomplished by in planta transformation by Agrobacterium, as described by Bechtold, et al., (C. R. Acad. Sci. Paris, 316:1194, 1993), the disclosure of which is incorporated herein by reference in its entirety, and exemplified in the Examples herein. This approach is based on the vacuum infiltration of a suspension of Agrobacterium cells.

The preferred method of introducing nucleic acid into plant cells is to infect such plant cells, an explant, a meristem or a seed, with transformed Agrobacterium tumefaciens as described above. Under appropriate conditions known in the art, the transformed plant cells are grown to form shoots, roots, and develop further into plants.

Alternatively, nucleic acid sequences according to the present invention can be introduced into a plant cell using mechanical or chemical means. For example, the nucleic acid can be mechanically transferred into the plant cell by microinjection using a micropipette. Alternatively, the nucleic acid may be transferred into the plant cell by using polyethylene glycol which forms a precipitation complex with genetic material that is taken up by the cell.

Nucleic acid sequences can also be introduced into plant cells by electroporation (Fromm, et al., Proc. Natl. Acad. Sci., U.S.A., 82:5824, 1985, which is incorporated herein by reference). In this technique, plant protoplasts are electroporated in the presence of vectors or nucleic acids containing the relevant nucleic acid sequences. Electrical impulses of high field strength reversibly permeabilize membranes allowing the introduction of nucleic acids. Electroporated plant protoplasts reform the cell wall, divide and form a plant callus. Selection of the transformed plant cells with the transformed gene can be accomplished using phenotypic markers as described herein.

Another method for introducing nucleic acid into a plant cell is by means of high velocity ballistic penetration by small particles with the nucleic acid to be introduced contained either within the matrix of such particles, or on the surface thereof (Klein, et al., Nature 327:70, 1987, the disclosure of which is hereby incorporated by reference in its entirety). Bombardment transformation methods are also described in Sanford, et al. (Techniques 3:3, 1991) and Klein, et al. (Bio/Techniques 10:286, 1992), the disclosures of which are incorporated herein by reference in their entireties. Although typically, only a single introduction of a new nucleic acid sequence is required, this method particularly provides for multiple introductions.

Cauliflower mosaic virus (CaMV) may also be used as a vector for introducing nucleic acid into plant cells (U.S. Pat. No. 4,407,956), which is incorporated herein by reference in its entirety. CaMV viral DNA genome is inserted into a parent bacterial plasmid creating a recombinant DNA molecule which can be propagated in bacteria. After cloning, the recombinant plasmid again may be cloned and further modified by introduction of the desired nucleic acid sequence. The modified viral portion of the recombinant plasmid is then excised from the parent bacterial plasmid, and used to inoculate the plant cells or plants.

As used herein, the term “contacting” refers to any means of introducing nucleic acid into the plant cell, including chemical and physical means as described above. Preferably, contacting refers to introducing the nucleic acid or vector into plant cells (including an explant, a meristem or a seed), via Agrobacterium tumefaciens transformed with the nucleic acid as described above.

Plant Regeneration

Normally, a plant cell is regenerated to obtain a whole plant from the transformation process. The immediate product of the transformation is referred to as a “transgenote”. The term “growing” or “regeneration” as used herein means growing a whole plant from a plant cell, a group of plant cells, a plant part (including seeds), or a plant piece (e.g., from a protoplast, callus, or tissue part).

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxins and cytokinins. It is sometimes advantageous to add glutamic acid and proline to the medium, especially for plant species such as corn and alfalfa. Efficient regeneration will depend on the medium, on the genotype, and on the history of the culture. If these variables are controlled, regeneration is reproducible.

Regeneration also occurs from plant callus, explants, organs or parts. Transformation can be performed in the context of organ or plant part regeneration (see Methods in Enzymology, Vol. 118, and Klee, et al., Annu. Rev. Plant Physiol., 38:467, 1987), the disclosure of which is incorporated herein by reference in its entirety. Utilizing the leaf disk-transformation-regeneration method of Horsch, et al. (Science, 227:1229, 1985), the disclosure of which is incorporated herein by reference in its entirety, disks are cultured on selective media, followed by shoot formation in about 2-4 weeks. Shoots that develop are excised from calli and transplanted to appropriate root-inducing selective medium. Rooted plantlets are transplanted to soil as soon as possible after roots appear. The plantlets can be repotted as required, until reaching maturity.

In vegetatively propagated crops, the mature transgenic plants are propagated by utilizing cuttings or tissue culture techniques to produce multiple identical plants. Selection of desirable transgenic plants is made and new varieties are obtained and propagated vegetatively for commercial use.

In seed propagated crops, the mature transgenic plants can be self crossed to produce a homozygous inbred plant. The resulting inbred plant produces seed containing the newly introduced foreign gene(s). These seeds can be grown to produce plants that would produce the selected phenotype.

Parts obtained from one or more regenerated plants, such as flowers, seeds, leaves, branches, roots, fruit, and the like are included in the invention, provided that these parts comprise cells that have been transformed as described. Progeny and variants, and mutants of the regenerated plants are also included within the scope of the invention, provided that these parts comprise the introduced nucleic acid sequences. The invention includes plants produced by the method of the invention, as well as plant tissue and seeds.

In yet another embodiment, the invention provides a method for producing a genetically modified plant cell such that a plant regenerated from said cell exhibits a modified phenotype as compared with a wild-type plant. The method includes contacting the plant cell with a nucleic acid sequence to obtain a transformed plant cell; growing the transformed plant cell under conditions suitable for regeneration, and obtaining a plant having the modified phenotype. Progeny may be derived by asexual propagation, apomictic reproduction, or sexual reproduction of the regenerated plant containing the nucleic acid. Conditions such as environmental and promoter-inducing conditions vary from species to species, and optional conditions can be determined by one of ordinary skill in the art.

In another aspect of the invention, it is envisioned that increased expression of genes of the present invention in a plant cell or in a plant, increases resistance of that cell/plant to plant pests or plant pathogens. In addition, increased expression of genes of the invention may also act as a herbicide safener by increasing the plant's resistance to pesticides. By the term “safener” is meant a gene that responds to specific chemicals (such as a pesticide) by activating natural plant pathways.

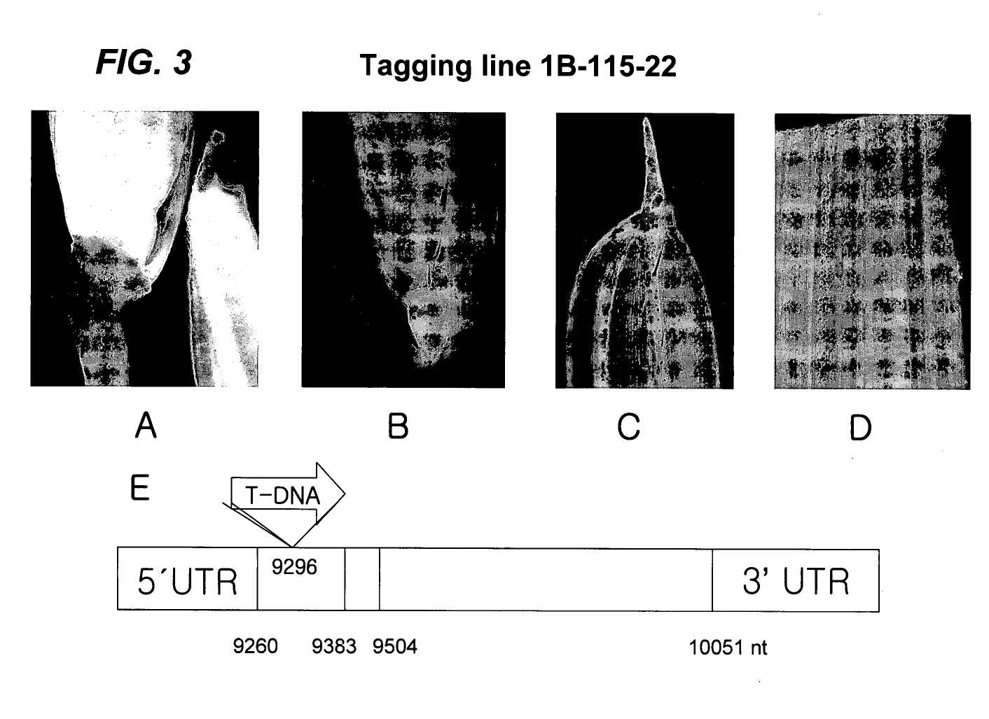

FIGS. 3 through 19 show 17 specific examples of GUS tagged genes that are preferentially expressed. The locations of the T-DNA inserts, as well as images detailing the GUS-positive expression, are shown. The paragraphs below list several genes and their encoded polypeptides that were found using the method of the invention.

Description of the Genes and Polypetides of the Invention, their Gus-Tagged Expression Characteristics in Rice, and Potential Agronomic Uses of These Genes

1b-115-22: (SEQ ID NO:18 comprises the genomic sequence, SEQ ID NO:1 comprises a portion of the genomic sequence with a portion of the insert sequence, SEQ ID NO:35 comprises the coding sequence, SEQ ID NO:52 comprises the amino acid sequence.) This gene encodes a protein with homology to Germins. Germins are a family of homopentameric cereal glycoproteins expressed during germination which may play a role in altering the properties of cell walls during germinative growth. Accordingly, in some embodiments, the gene may be used to alter glycoprotein levels or increase resistance to fungal pathogens in rice grains. A diagram showing the insertion site of the T-DNA/GUS insert and an image showing the expression characteristics of the tagged gene are shown in FIG. 3.

Some germins have been shown to have oxalate oxidase activity (Lane et al, 1993, J. Biol. Chem. 268: 12239-12242), the disclosure of which is incorporated herein by reference in its entirety. The oxalate oxidase activity generates H₂O₂ from the oxidative breakdown of oxalate to H₂O₂ and CO₂. Germins have been found to accumulate during embryogenesis, germination, salt stress, pathogen elicitation, or heavy metal stress.

The generation of H₂O₂ by germins is thought to play a role in plant defense responses against pathogens (for a review, see Patnaik and Khurana, 2001, Indian Jour. Exp. Biol. 39:191-200), the disclosure of which is incorporated herein by reference in its entirety. Crop plants transformed with a gene encoding a germin having oxalate oxidase activity were found to have an increased resistance to fungal pathogens that utilize oxalic acid as a toxin (Thompson et al, 1995, Euphytica, 85, 169-172), the disclosure of which is incorporated herein by reference in its entirety. Other findings suggest germins are involved in the response of plants to both biotic and abiotic stress (Woo et al, 2000, Nature Structural Biology, 7: 1036-1040), the disclosure of which is incorporated herein by reference in its entirety.

The protein encoded by the gene found in the present invention has “germin-like” amino acid sequence, and thus may have properties similar to germins. The GUS localization studies of the invention showed that gene encoding the rice germin-like protein is expressed in several types of trichomes (such as, for example, in leaves, pedicel, rachila, palea, and lemma) indicate further that this particular protein may have an important role in protecting rice plants from environmental incursions. Therefore, rice or other plants overexpressing the gene of the invention may have increased levels of resistance to several plant stresses.

In one embodiment of the invention, it may be useful to genetically engineer plants to have high levels of expression of this gene, either on a constitutive or inducible basis. Plants that always have high levels of the protein in their trichomes may be more resistant to pathogen attack. Alternatively, in some situations it may be useful to create plants that have high levels of expression of the gene, but only upon pathogen attack.

One suggested role for germin-like oxalate oxidases is that they are involved in cell death mechanisms (Lane, 2000, Biochem Jour. 349:309-321), the disclosure of which is incorporated herein by reference in its entirety. Thus, genetic modification of the expression of this protein in plants could alter cell death mechanisms. Overexpression of the gene, for example, linked to pathogen specific inducible promoters could yield plants that have organs or regions that are programmed to die upon infection with problematic pathogens, perhaps inhibiting the movement of infective agents further throughout the plant.

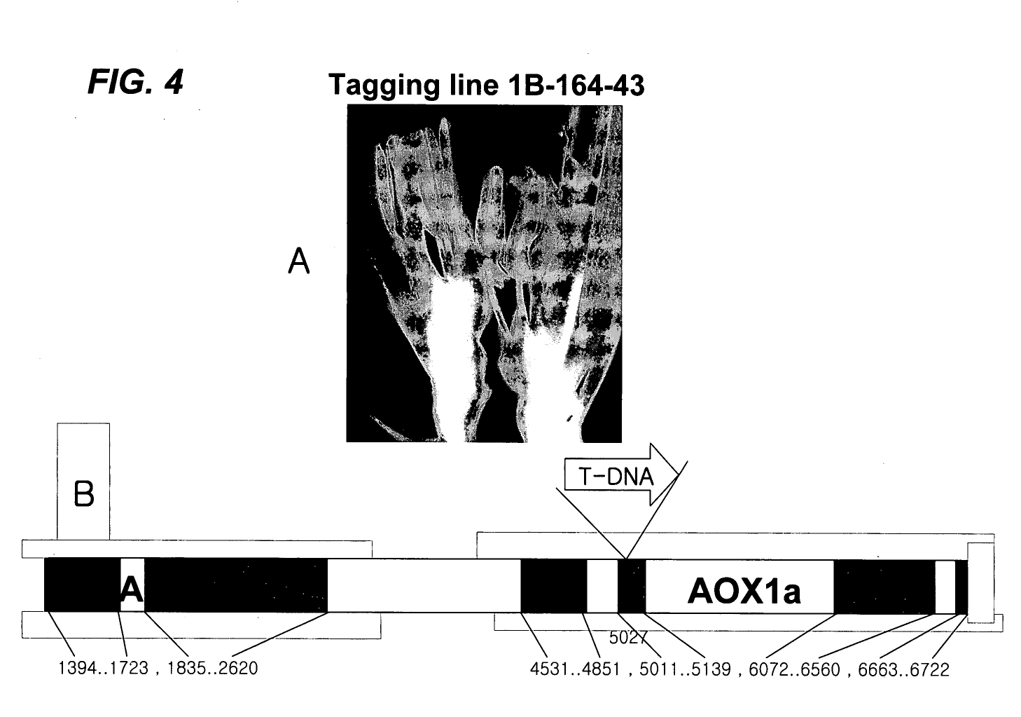

1b-164-43: (SEQ ID NO:19 comprises the genomic sequence, SEQ ID NO:2 comprises a portion of the genomic sequence with a portion of the insert sequence, SEQ ID NO:36 comprises the coding sequence, SEQ ID NO:53 comprises the amino acid sequence.) This gene encodes a protein having homology to alternative oxidase (AOX1a) proteins and can, in some embodiments, confer an increased protective effect on developing pollen grains. A diagram showing the insertion site of the T-DNA/GUS insert and an image showing the expression characteristics of the tagged gene are shown in FIG. 4. The GUS-positive expression of this gene was found in the anther.

Alternative oxidase is used as a second terminal oxidase in the mitochondria, where it diverts electrons from the standard electron transfer chain. The electrons are transferred directly from reduced ubiquinol to oxygen, forming water. The free energy that is released during electron movement through the AOX pathway is lost as heat. Thus, this pathway may be thought as a heat producing mechanism. Interestingly, expression of rice alternative oxidase transcripts is increased in response to low temperature.

The use of alternative oxidase pathways rather than the standard electron transport chain may be beneficial in decreasing the production of active oxygen intermediates (for a review, see Seidow and Day, (2000), in Biochemistry and Molecular Biology of Plants, American Society of Plant Physiologists, B. Buchanan, Ed., pp 696-706), the disclosure of which is incorporated herein by reference in its entirety. Thus, in certain physiological states, or under certain environmental conditions, the AOX pathway may be preferred to the standard pathway. Genetic engineering to alter the expression levels or the inducible characteristics of this gene may be agronomically useful. For example, increasing the expression of the gene during anther development may have an increased protective effect on developing pollen grains. In another example, the gene could be altered such that is produced in other plant tissues in addition to the anther. The possibility that AOX acts as a heat producing mechanism may be useful, for example, to protect developing pollen grains from low temperature damage by slightly increasing the temperature of the tissue. Through genetic manipulation, it is possible that the AOX gene expression could be increased during cold stress in developing anther tissue. This may act to increase the temperature of the anther tissue under cold stress, perhaps protecting the developing pollen grains from damage related to cold temperatures.

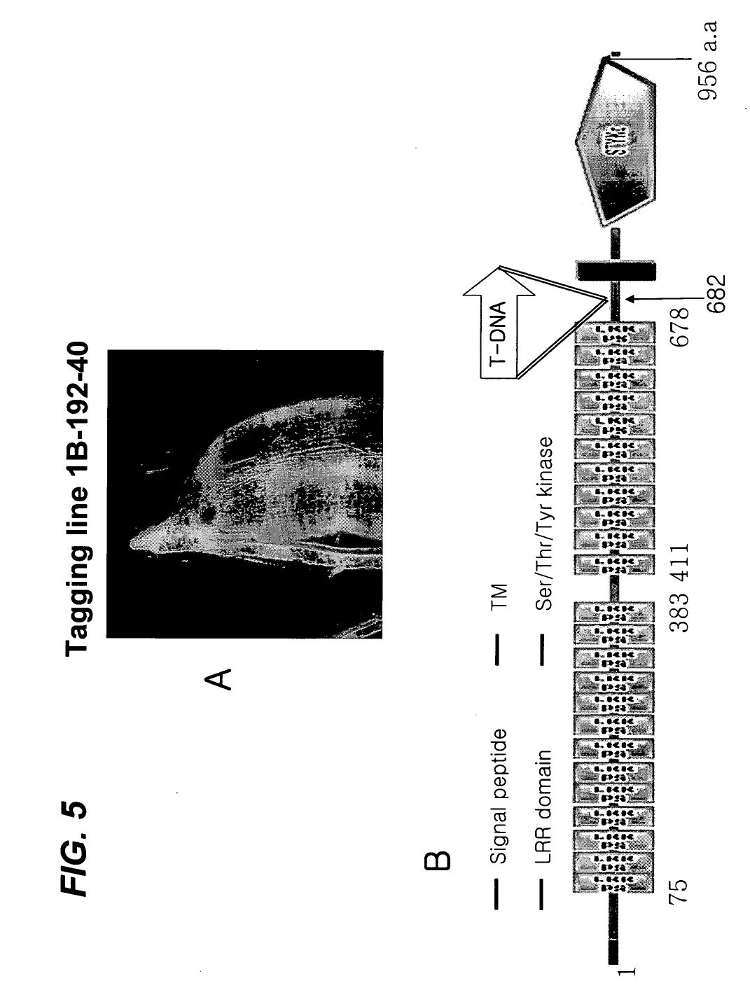

1b-192-40: (SEQ ID NO:20 comprises the genomic sequence, SEQ ID NO:3 comprises a portion of the genomic sequence with a portion of the insert sequence, SEQ ID NO:37 comprises the coding sequence, SEQ ID NO:54 comprises the amino acid sequence.) This gene encodes an XA21-like protein kinase gene and can, in some embodiments, be used to increase disease resistance in rice plants. The similar Xa21 protein is thought to be involved in disease resistance mechanisms, since similar proteins have been found to be involved in pathogen defense processes. A diagram showing the insertion site of the T-DNA/GUS insert and an image showing the expression characteristics of the tagged gene are shown in FIG. 5.

Gene for gene resistance to pathogens is conferred by a group of genes termed resistance genes or “R” genes, some of which encode kinases. The rice bacterial blight disease resistance gene, Xa21, encodes a kinase involved in resistance to bacterial blight (Liu, et al., JBC Papers in press, pub date: Apr. 1, 2002, as Manuscript # M110999200), the disclosure of which is incorporated herein by reference in its entirety. The protein encoded by the gene contains a leucine-rich repeat region as well as a kinase domain. The kinase domain of Xa21 has been found to autophosphorylate multiple serine and threonine residues.

The protein encoded by the gene found in the present invention has homology to Xa21, and therefore may confer similar resistance to bacterial pathogens. Thus, it may be useful to overexpress the protein in rice plants to be grown in areas where bacterial blight may be especially problematic. It may be useful to engineer the gene so that it is induced in response to pathogen attack, or so that it is expressed under conditions that are often present prior to pathogen attack (such as temperature changes, or nutrient stress, for example).

Finally, the gene found in the present invention is localized to the palea and lemma of the developing rice flower. If, indeed, the protein is involved in pathogen resistance, it may be desirable to tailor the expression of this transgene so that it is expressed at high levels preferentially in tissues that are most likely to be infected with the pathogen, while not being expressed in tissues that are not likely to become infected.

1b-207-27: (SEQ ID NO:21 comprises the genomic sequence, SEQ ID NO:4 comprises a portion of the genomic sequence with a portion of the insert sequence, SEQ ID NO:38 comprises the coding sequence, SEQ ID NO:55 comprises the amino acid sequence.) This gene encodes a protein with homology to receptor-like protein kinases and can, in some embodiments, be used to alter rice grain development. The gene is expressed in the ovary and lodicule. The kinase domain is at the c-terminal half of the protein.

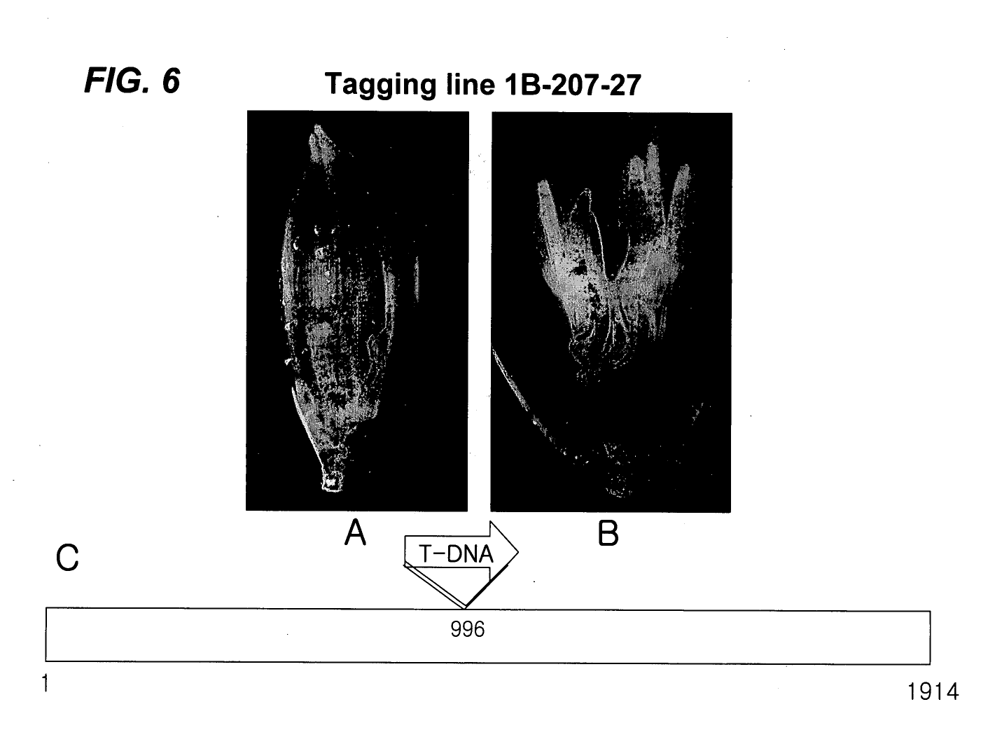

A diagram showing the insertion site of the T-DNA/GUS insert and an image showing the expression characteristics of the tagged gene are shown in FIG. 6. The gene was expressed in the palea/lemma region of the flower, as well as in the ovary and lodicule. This protein may function as a receptor of various environmental and developmental stimuli. Because the kinase is present in the ovary, its genetic modification may alter signalling mechanisms affecting such events as fruit development or seed development, resulting in plants with altered phenotypes.

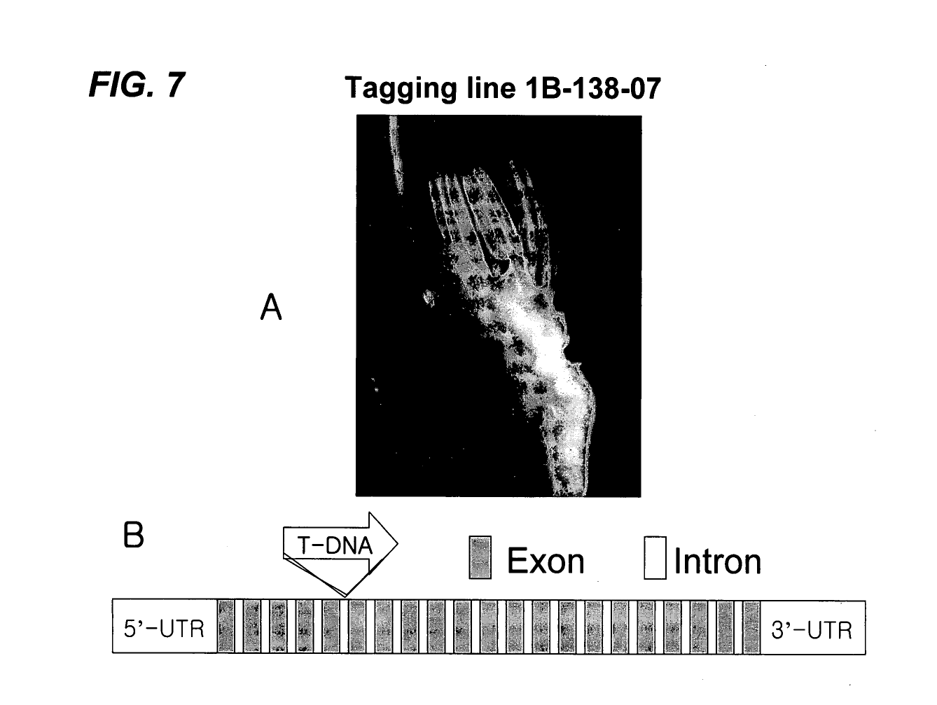

1b-138-07: (SEQ ID NO:22 comprises the genomic sequence, SEQ ID NO:5 comprises a portion of the genomic sequence with a portion of the insert sequence, SEQ ID NO:39 comprises the coding sequence, SEQ ID NO:56 comprises the amino acid sequence.) This gene encodes methylmalonate semi-aldehyde dehydrogenase (MMSDH1) which may be involved in amino acid degradation pathways, and can in some embodiments, be used to confer protection from cold stress, or to aid in nutrient partitioning (such as, for example, during grain fill). The method of the invention localized the expression of this gene to leaves, anther, and rachilla of rice. A diagram showing the insertion site of the T-DNA/GUS insert and an image showing the expression characteristics of the tagged gene are shown in FIG. 7.

Methylmalonate-semialdehyde dehydrogenases belong to a broad class of oxidoreductases. Methylmalonate-semialdehyde dehydrogenases act on either aldehyde or oxo groups of donor molecules. The acceptor molecule is NAD+ or NADP+. MMSDH is thought to function in the catalysis of the irreversible oxidative decarboxylation of malonate and methylmalonate semialdehydes to acetyl- and propionyl-CoA, respectively. MMSDH is the only aldehyde dehydrogenase known to require CoA. This group of enzymes [EC: 1.2.1.27] is thought to be important for metabolic processes, such as carbohydrate metabolism; inositol metabolism, and propanoate metabolism. More specifically, the enzyme is considered to be involved in the degradation of the amino acid valine (part of the valine, leucine, and isoleucine degradation pathway).

The enzyme is induced by cold stress in wheat, suggesting that it may be involved in protection from cold stress. Because the enzyme is thought to be involved in the degradation of certain amino acids such as valine, genetic modification of the levels of this enzyme would alter amino acid compositions or metabolic pathways. Because the protein is induced upon cold stress, it may be part of plant stress protective pathways. Overexpression of the gene may result in plants that better suited to survival under low temperature conditions.

Further, the involvment of MMSDH in amino acid degradation pathways, combined with the above-mentioned cold induction findings, indicated that it may be involved in nutrient partitioning during the senescence process. If so, rice plants could be modified to produce increased levels of this protein in tissues that will senesce upon cold or drought stress, thus more efficiently recycling nitrogen and other important molecules to the parts of the plant that will remain alive, such as the seed.

1d-059-12: (SEQ ID NO:23 comprises the genomic sequence, SEQ ID NO:6 comprises a portion of the genomic sequence with a portion of the insert sequence, SEQ ID NO:40 comprises the coding sequence, SEQ ID NO:57 comprises the amino acid sequence.) This gene encodes a protein that has homology to the RNA-binding protein LAH1 (for La protein homolog 1) (also termed LHP1, YLA1). The gene can, in some embodiments, be used to alter cellular processes leading to protein production.

In eukaryotes, the La protein binds to the 3′ end of many types of RNA transcripts. In yeast, the La protein LHP1 participates in the processing of tRNA to maturity (Yoo and Wolin, 1997, Cell 89:393-402), the disclosure of which is incorporated herein by reference in its entirety. LAH1 binds to the 3′ end of nascent RNA polymerase III transcripts, protecting the transcripts from degradation (Xue et al., 2000, EMBO J., 19:1650-1660), the disclosure of which is incorporated herein by reference in its entirety. The yeast La protein is thought to act as a molecular chaperone for nascent RNA polymerase III transcripts (Pannone, et al., 1998, EMBO J., 17:7442-7453), the disclosure of which is incorporated herein by reference in its entirety. The yeast La protein has also been found to be involved in snRNP assembly, perhaps by assisting with RNA folding, RNA stabilization, and RNA interactions with other proteins (Xue, 2000, supra).

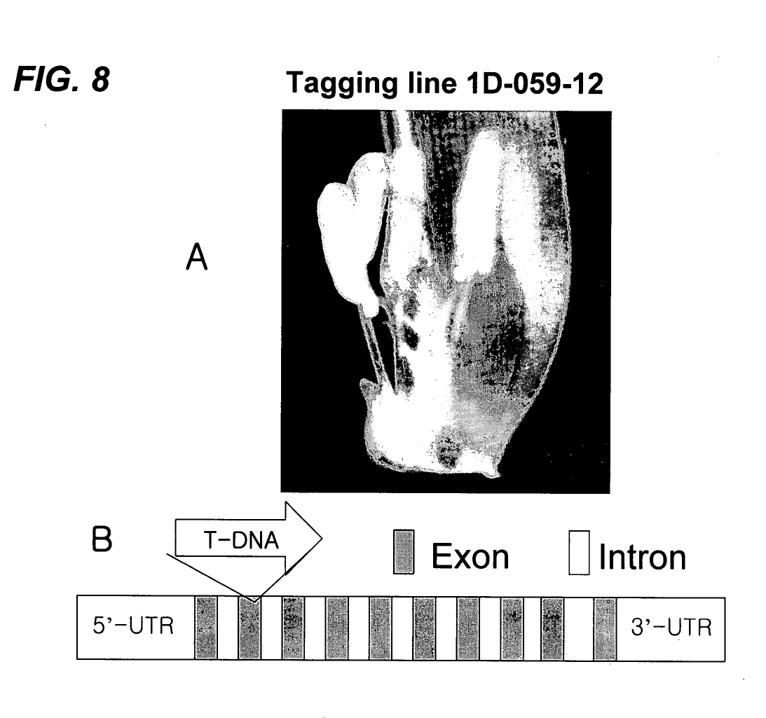

A diagram showing the insertion site of the T-DNA/GUS insert and an image showing the expression characteristics of the tagged gene are shown in FIG. 8. The GUS-tagged expression of this gene was found to be present in the ovary. Therefore, the endogenous encoded protein may be involved in the development of the ovary. With this in mind, it may be of agronomic utility to increase or otherwise alter the ovary-specific expression of this gene to create plants with altered characteristics, such as desirable grain characteristics.

The identification of a gene encoding a similar protein in rice may be of agronomic utility. For example, plants with higher levels of this protein may have RNA transcripts with increased stability. It may also be possible to transform plants with a gene that encodes an altered protein such that it functions to maintain RNA stability at altered temperatures, or under other stress conditions.

Alternatively, it may be useful to genetically modify plants so that the LAH1 protein production is decreased, either temporally, developmentally, or constitutively. Plants with a decrease in LAH1 protein levels would be expected to have altered transcription characteristics, which would likely result in altered growth characteristics and altered morphologies. For example, an antisense construct of the LAH1 gene of the invention could be transformed to a plant to result in a plant with modified transcriptional processes.

1c-087-40: (SEQ ID NO:24 comprises the genomic sequence, SEQ ID NO:7 comprises a portion of the genomic sequence with a portion of the insert sequence, SEQ ID NO:41 comprises the coding sequence, SEQ ID NO:58 comprises the amino acid sequence.) This gene encodes a protein that has homology to vacuolar ATP synthase subunit C (also known as V-type ATPase subunit C), and can, in some embodiments, be used to alter growth and development of rice plants.

Vacuolar ATPases are located at the vacuolar membrane, and pump protons into the vacuole using energy derived from ATP hydrolysis. The vacuole pH is thus kept low (typically between pH 3.0 and pH 5.0). Most vacuolar proteins work optimally at this lower pH. The vacuolar ATPase complex is somewhat similar to other membrane ATPases, having an integral membrane region (F₀), and a cytoplasmic region (F₁). The “C” subunit of this complex is an integral membrane polypeptide. Several “C” subunits form a multimer integral membrane protein complex.

The DET3 gene encodes a similar protein present in Arabidopsis. In Arabidopsis, the protein has been found to play a role in both cell expansion and in meristematic growth. det3 mutants were found to have a light-grown phenotype even when grown in the dark, cell elongation defects, and is somewhat insensitive to brassinosteriods (Schumacher et al., 1999, Genes Dev. 13:3259-3270), the disclosure of which is incorporated herein by reference in its entirety. Therefore, the gene plays a role in plant growth and development.

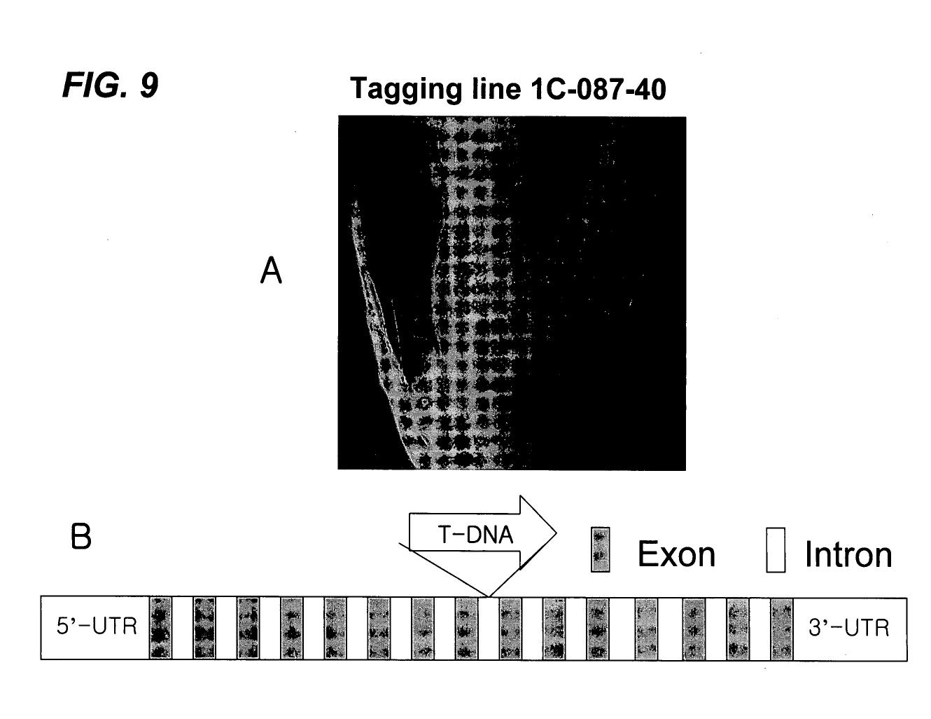

A diagram showing the insertion site of the T-DNA/GUS insert and an image showing the expression characteristics of the tagged gene are shown in FIG. 9. The GUS-tagged expression of the gene of the present invention was found to be present in the ovary. This suggests that the gene of the present invention may be involved in the development of the ovary, rather than being involved in plant growth as a whole. With this in mind, it may be of agronomic utility to increase or otherwise alter the ovary-specific expression of this gene. For example, since the gene has been found to be involved in development and cell elongation (see Schumacher, 1999, supra), and further since the gene in the present invention appears to be expressed in an ovary-specific manner, it may be possible to create rice plants with altered grain size, shape, or processing characteristics by altering expression of this gene. For example, since the ovary matures into outer brown layer of the rice grain (commonly termed “bran”), ovary-tissue specific overexpression of this gene may be performed to perhaps create larger or faster growing grains, or grains with altered bran characteristics.

Because this gene is expressed in ovary tissue, the disruption of the ovary-specific expression of the gene could perhaps result in plants deficient in ovary maturation processes. This may be beneficial, for example, for certain crops wherein a fruit or seed is not desirable (e.g. plants that tend to bolt prematurely, such as basil), and delay of its formation would be valued. Ovary-specific disruption of the gene could be achieved by linking the 5′ promoter of the identified gene to the antisense sequence of the gene, followed by plant transformation.

In rice, antisense-based disruption of the gene expression in the ovary might result in a less prominent ovary wall. When the grain matures, the ovary wall becomes the “bran” of the rice grain (outer brown layer). During processing from brown rice to white rice, this bran is often discarded from the white portion of the grain. Thus, it may be of agronomic usefulness to create rice grains with less prominent ovary wall/bran tissue by downregulating ovary-specific expression of this gene.

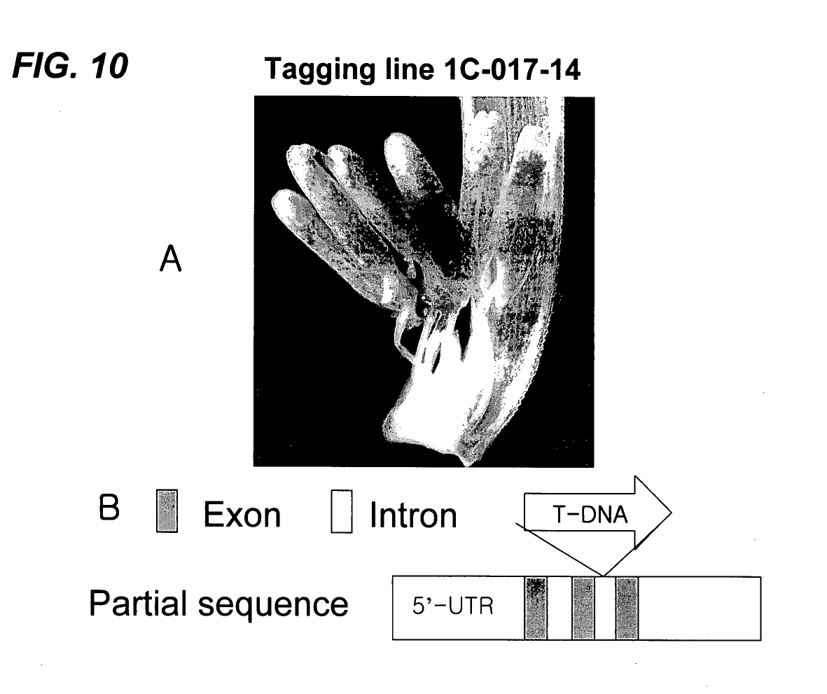

1c-017-14: (SEQ ID NO:25 comprises the genomic sequence, SEQ ID NO:8 comprises a portion of the genomic sequence with a portion of the insert sequence, SEQ ID NO:42 comprises the coding sequence, SEQ ID NO:59 comprises the amino acid sequence.) This gene encodes a protein with homology to cinnamic acid 4-hydroxylase which may play an essential role in the regulation of the phenylpropanoid pathway. A diagram showing the insertion site of the T-DNA/GUS insert and an image showing the expression characteristics of the tagged gene are shown in FIG. 10. The gene can, in some embodiments, be used to engineer plants with increased resistance to pathogen attack.

In the early steps of the phenylpropanoid pathway, phenylalanine is converted to cinnamic acid by PAL (phenylalanine ammonia lyase). Subsequently, the enzyme cinnamic acid 4-hydroxylase adds a hydroxyl group to cinnamic acid to create p-coumaric acid. Subsequent steps and branches lead to several important groups of phenolic compounds in plants. Because the cinnamic acid 4-hydroxylase enzyme is an early member of the general phenylpropanoid pathway, it plays an essential role in many types of plant processes. For example, the phenylpropanoid pathway is responsible for such diverse plant functions as lignin synthesis, flower pigments, signalling molecules, and a large spectrum of compounds involved in plant defense against pathogens and UV light.

The cinnamic acid 4-hydroxylase gene of the invention was localized to pollen. Because it is a precursor to many different compounds, it may have several roles in the pollen grain. In fact, any of the above mentioned functions may be important for pollen development and viability. Of particular importance may be the role of phenylpropanoid pathway products in the in protection of the pollen grain from UV light damage. Further, the enzyme may be involved in the formation of the outer pollen wall components.

Of particular agronomic usefulness may be the increased expression of this gene, coupled to its own promoter, so that it has increased expression levels in developing pollen. Such increased levels of the enzyme could result in increased UV protection, or in increased strength of the pollen wall (this may result in the increased viability of the pollen grain, especially under suboptimal environmental conditions).

Downregulating the expression of this gene in pollen may be useful in some situations. For example, one may wish to produce transgenic rice plants with pollen that, though initially viable, degrades more readily than wild-type pollen when exposed to UV light In this way, it would be more difficult for transgenic pollen to remain viable enough to pollinate other, non-transgenic crops that may be some distance from the transgenic crops. The pollen would presumably remain viable for nearby pollination, but would be less likely to survive extended time in the sunlight or environmental extremes. This type of system would provide an additional safety guard for use in combination with other transgenic plant safety systems.

It would also be possible to link the gene of the invention to another tissue-specific promoter other than the endogenous pollen-specific promoter. For example, linking the gene to either a palea/lemma specific promoter, or an ovary-specific promoter, or a seed-specific promoter of a rice plant could produce rice grains that are more resistant to disease, damage, or other unfavorable conditions.

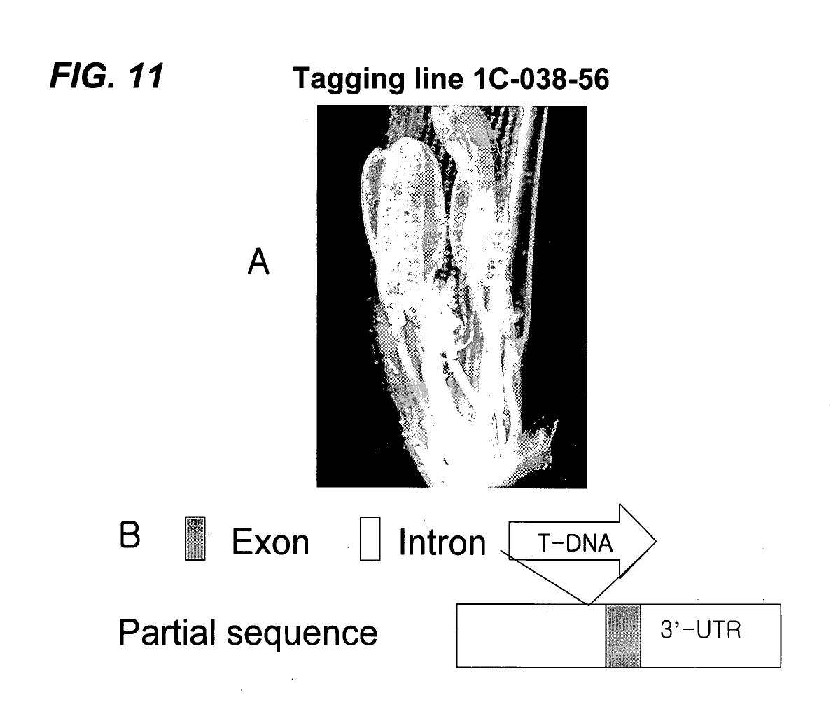

1c-038-56: (SEQ ID NO:26 comprises the genomic sequence, SEQ ID NO:9 comprises a portion of the genomic sequence with a portion of the insert sequence, SEQ ID NO:43 comprises the coding sequence, SEQ ID NO:60 comprises the amino acid sequence.) This gene encodes a protein with homology to H-protein promoter binding factor-2a. A diagram showing the insertion site of the T-DNA/GUS insert and an image showing the expression characteristics of the tagged gene are shown in FIG. 11. GUS-tagged expression of this gene was found to be located in pollen tissue. The protein has a 79% identity to gi/15451553, which is an H-protein promoter binding factor-2a that is involved in transcription, affecting the photorespiration of mitochondria. Therefore, genetic modification of this gene in rice may alter transcriptional activities. Because the gene is expressed preferentially in pollen tissue, it may be possible, for example, to alter pollen characteristics, such as germination rates, pollen development, or pollen tube growth.

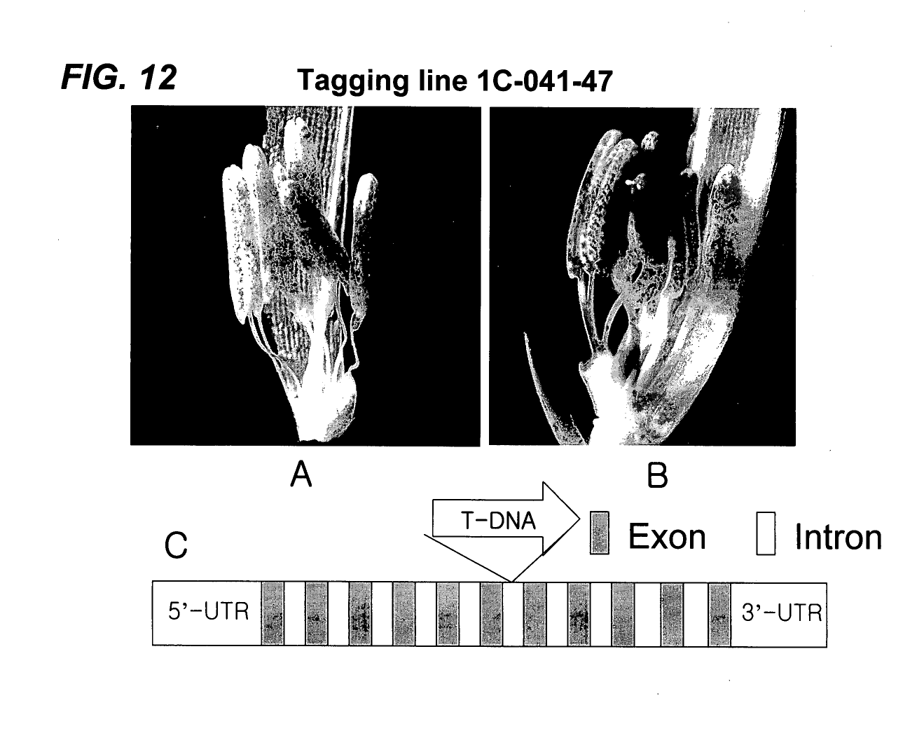

1c-041-47: (SEQ ID NO:27 comprises the genomic sequence, SEQ ID NO:10 comprises a portion of the genomic sequence with a portion of the insert sequence, SEQ ID NO:44 comprises the coding sequence, SEQ ID NO:61 comprises the amino acid sequence.) This gene encodes a protein with homology to flap endonuclease (FEN-1), and can, in some embodiments, be used to increase plant resistance to environmental mutagens. A diagram showing the insertion site of the T-DNA/GUS insert and an image showing the expression characteristics of the tagged gene are shown in FIG. 12.

FEN-1 is a key enzyme in both DNA replication and in DNA repair processes. FEN-1 plays a role in removal of the 5′ ends of Okazaki fragments of the lagging strand during DNA replication processes (see Lewin, (2000), Genes VII, Oxford University Press, Inc., New York, p. 393), the disclosure of which is incorporated herein by reference in its entirety, and also removes 5′ overhanging flaps during DNA repair. It is thought that FEN-1 acts as an endonuclease during DNA repair, but as an exonuclease during DNA replication. FEN-1 has been proposed to act in concert with other proteins, such as DNA polymerase 6, proliferating cell nuclear antigen (PCNA), and replication protein A (RP-A) in the processing of Okazaki fragments during DNA replication processes in mammals (Maga, et al., 2001, Proc. Natl. Acad. Sci. 98: 14298-14303), the disclosure of which is incorporated herein by reference in its entirety. The FEN-1 protein localizes in the nucleus during the S-phase of DNA synthesis and also in response to DNA damage (Qiu, et al., 2001, J. Biol. Chem. 276: 4901-4908), the disclosure of which is incorporated herein by reference in its entirety.

Yeast cells having a loss of FEN-1 function exhibited increased sensitivity to chemical or other mutagens, thus increasing the mutation rate. Further, because FEN-1 is essential for DNA replication, complete loss of function mutations are unlikely to be viable in mammalian cells (see Qiu, et al., 2002, Jour. Biol. Chem. (published May 1, 2002, as Manuscript # M111941200), the disclosure of which is incorporated herein by reference in its entirety.

The method of the present invention localized the FEN-1 gene expression to pollen. Several possible functions in this plant cell type can be envisioned. For example, since pollen grains are exposed to UV light, a potential for DNA damage exists. The FEN-1 might be present in pollen to protect the pollen grain from any DNA damage that may occur due to excess exposure to the environment before pollination can occur. Another possibility is that the FEN-1 is present in the pollen to assist in DNA replication processes. Either way, overexpression of the FEN-1 gene, linked to its own pollen specific promoter, could result in pollen that is more viable or less likely to have DNA damage after being subjected to excess environmental conditions such as UV light.

Alternatively, it may be useful to downregulate the expression of this gene in pollen. For example, in some cases it may be useful to have plants that mutagenize more readily to commonly used chemical mutagens such as ethane methylsulphonate (EMS). Mutagenesis methods are used in plant research to determine the function of genes and to find new and useful phenotypes. Therefore, it may be especially useful to have a line of plants that mutates more readily in order to generate higher numbers of mutant plants for screening purposes.

Further, it may be useful to link the gene of the invention with a constitutive promoter so that plants transformed with the construct will have an increased overall DNA repair system and thus an overall protection from UV damage to cellular DNA. This may be important, for example, for crops that are especially sensitive to spontaneous mutations or UV light.

1c-064-20: (SEQ ID NO:28 comprises the genomic sequence, SEQ ID NO:11 comprises a portion of the genomic sequence with a portion of the insert sequence, SEQ ID NO:45 comprises the coding sequence, SEQ ID NO:62 comprises the amino acid sequence.) This gene encodes a protein with homology to heat shock protein Hsp70, and can, in some embodiments, be used to engineer plants with increased protection from heat stress or other stresses. Hsp70 proteins act as molecular chaperones to allow newly synthesized polypeptide chains to fold in the proper orientation by stabilizing the nascent chains to protect them from aggregation while they are elongating on the ribosome (for a review, see Hartl and Hayer-Hartl, 2002, Science 295:1852-1858), the disclosure of which is incorporated herein by reference in its entirety. Hsp70 proteins act on nascent polypeptide chains in an ATP-dependent manner by cycling through the steps of polypeptide binding and polypeptide release. The release from the polypeptide allows it to fold in a native state. Hsp70 may also be involved in the transfer of proteins to chaperonin complexes for further processing. Further, DnaK, the bacterial homolog of Hsp70, has been shown to have peptide bond isomerase activity (Schiene-Fischer et al., Nat. Struct. Biol., 2002, published online May 20, 2002), the disclosure of which is incorporated herein by reference in its entirety. If eukaryotic Hsp70 proteins are also found to possess this property, the protein may have an even more important and complex role in protein processing. Thus, Hsp70 proteins are important for the proper folding and function of simple, singular polypeptide units, as well as intricate, multimeric protein complexes, because the proper folding of each component of a protein complex may be necessary for proper function of the complex as a whole.

Hsp70 proteins may also be involved in general protection for the nuclear machinery during embryogenesis in plants (Testillano, et al., 2000, J. Struct. Biol., 129:223-232), the disclosure of which is incorporated herein by reference in its entirety. Other types of Hsp70 proteins have been found to be involved in protein import into mitochondria and plastids (Rial, et al, 2000, Eur. J. Biochem. 267:6239-6248; Zhang and Glaser, 2002, Trends Plant Sci. 7:14-21), the disclosures of which are incorporated herein by reference in their entireties.

Heat shock proteins are often upregulated by heat shock or other stresses in many types of organisms. Measurement of HSP accumulation may be used to determine the level of stress an organism has previously been exposed to (see U.S. Pat. No. 5,232,833 to Sanders, which is hereby incorporated by reference in its entirety). Thus, one utility of the gene of the present invention is its use as a probe to determine stress levels in rice pollen or other plant tissues.

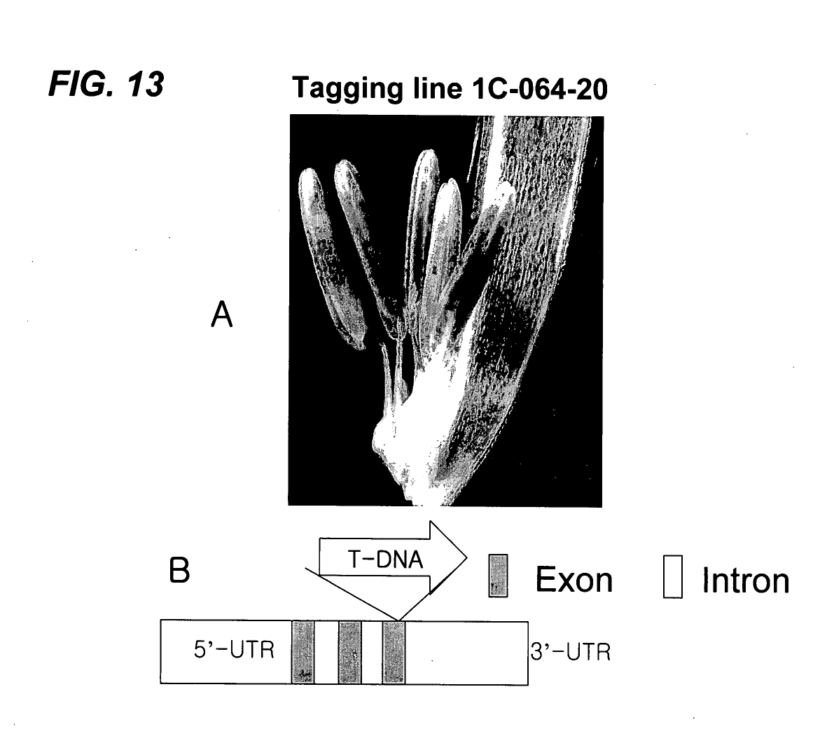

A diagram showing the insertion site of the T-DNA/GUS insert and an image showing the expression characteristics of the tagged gene are shown in FIG. 13. The GUS-tagging method of the invention localized expression of the gene to pollen. The occurrence of Hsp70 in pollen may be related to protection of the nascent protein during synthesis on the ribosome. If so, transgenic plants having increased levels of Hsp70 may have increased protection from heat stress or other stresses.

It may be useful to transform plants with the Hsp70 of the invention, coupled to its own promoter, to enhance stress protection and/or protection of the nascent proteins during synthesis in the pollen grain. The pollen-specific Hsp70 may play a role in protecting from aggregation of cellular proteins during the water loss period as the pollen grain matures. Therefore, perhaps overexpressing this gene in a pollen specific manner may increase the length of time that the pollen grain can maintain viability. In other embodiments, it may be useful to transform plants with the Hsp70 gene of the invention coupled to a constitutive promoter, so that expression of the gene will be at high levels prior to a stress event. This would provide the plant with immediate protection from stress-related cellular damage. For an example of the use of heat shock proteins derived from stress-resistant blue-green algae to offer increased stress protection when transformed to plants, see Japanese patent application JP2001078603A2, the disclosure of which is incorporated herein by reference in its entirety.

In some situations, it may be desirable to transform plants to decrease or alter the Hsp70 activity. For example, engineering plants with an antisense construct of the Hsp70 gene of the invention, coupled with its own pollen-specific promoter, would result in plants that have a deficiency in Hsp70 expression in the pollen only. Such plants may have reduced viability. Growers of transgenic crops may wish to produce these plants so that transgenes of interest will not be spread to nearby crops or related weeds.

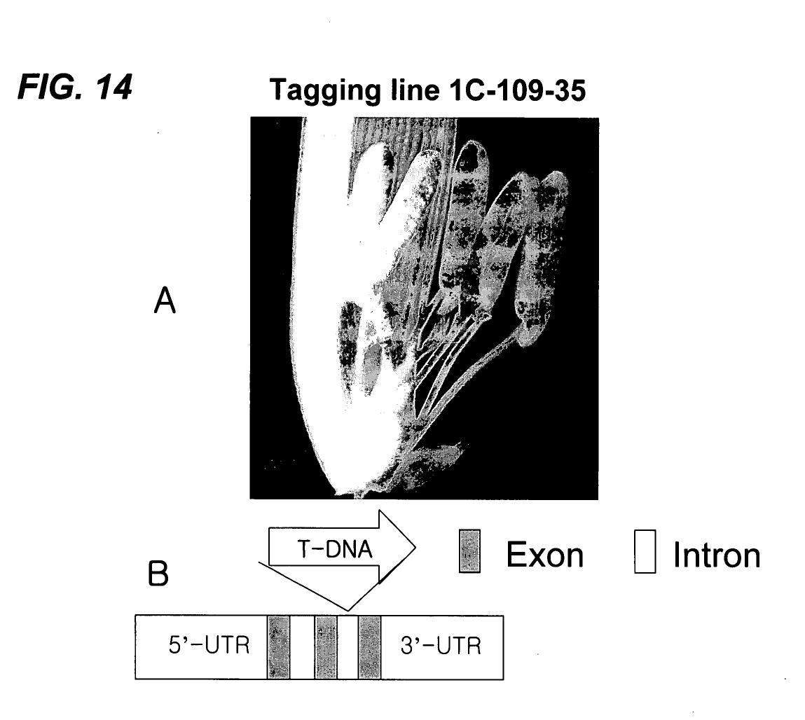

1c-109-35: (SEQ ID NO:29 comprises the genomic sequence, SEQ ID NO:12 comprises a portion of the genomic sequence with a portion of the insert sequence, SEQ ID NO:46 comprises the coding sequence, SEQ ID NO:63 comprises the amino acid sequence.) This gene encodes a protein with homology to ammonium transporters, and was found to be expressed in pollen tissue of rice. A diagram showing the insertion site of the T-DNA/GUS insert and an image showing the expression characteristics of the tagged gene are shown in FIG. 14. The gene can, in some embodiments, be used to increase ammonium uptake during pollen germination.

Nitrogen is an essential nutrient for plant growth, being a component of all amino acids and many other plant molecules. Though this is an essential nutrient, it may not always be available in the soil. Plants have developed mechanisms to respond to the presence or absence of nitrogen levels in the soil by upregulating or downregulating nitrogen transporters, as well as enzymes involved in nitrogen assimilation. Uptake of NO₃ and NH4⁺ are regulated by membrane transport mechanisms (von Wiren et al, 1997, Plant Soil 196: 191-199), the disclosure of which is incorporated herein by reference in its entirety. When nitrate is present in the soil, nitrogen assimilation enzymes such as nitrate reductase and nitrite reductase (and many others) are upregulated. However, when ammonium is present, membrane proteins capable of transporting ammonium across cellular membranes may be upregulated. The Arabidopsis ammonium transporter AMT1;1 has been found to be induced by nitrogen starvation (Gazzarrini et al., 1999, Plant Cell 11:937-947), the disclosure of which is incorporated herein by reference in its entirety. Conversely, microarray analysis has shown that the AMT1;1 gene is strongly downregulated by high nitrogen concentrations (Wang et al., 2000, Plant Cell 12:1491-1509), the disclosure of which is incorporated herein by reference in its entirety.

Ammonium transporters are preferentially expressed in root hairs (Lauter, et al., 1996, Proc. Natl. Acad. Sci., USA 93:8139-8144), the disclosure of which is incorporated herein by reference in its entirety. In Arabidopsis, several ammonium transporters have been found, each responding to different nitrogen conditions. AtAMT1;2 mRNA expression was found to be constitutive, while AtAMT1;1 mRNA levels were induced by nitrogen starvation., and a further ammonium transporter, AtAMT1;3 was postulated to be a link between nitrogen assimilation and carbon availability (Gazzarrini et al., 1999, supra). Another Arabidopsis ammonium transporter, AtAMT2, was found to be more highly expressed in shoots than in roots (Sohlenkamp, et al., 2000, FEBS Lett. 467:273-278), the disclosure of which is incorporated herein by reference in its entirety.

The finding that the gene of the present invention is expressed preferentially in pollen tissue indicates that it has a different function than the uptake of nitrogen from the soil. The transporter may function to take up nitrogen from the surrounding stigma and style tissue of the target ovary. Alternatively, the ammonium transporter may allow import of nitrogen from the surrounding anther tissue as the pollen grains are developing. Alterations in expression of this gene in pollen could be of agronomic utility. For example, a pollen-specific knock-out of the ammonium transporter gene expression could be accomplished by plant transformation with an antisense construct of the ammonium transporter gene linked to its own pollen-specific promoter. This may be useful, for example, in creating male-sterile plants that may be valuable for outdoor transgenic crop safety.

In contrast, increasing the pollen-specific expression of this gene may be useful, for example, to increase nitrogen uptake (and thus growth and development) during either pollen grain development or during pollen germination. Pollen tube growth has been shown to increase when polyamines such as spermine are added to germinating pollen tubes (Cetin et al., 2000, Can. Jour. Plant Sci., 80:241-245), the disclosure of which is incorporated herein by reference in its entirety. Accordingly, it may be possible to increase pollen growth rates (and thus fertilization rates) by increasing nitrogen transporters such as the ammonium transporter of the pollen tube by transforming plants with the ammonium transporter gene linked to its own promoter and to upstream enhancer sequences.

1c-109-51: (SEQ ID NO:30 comprises the genomic sequence, SEQ ID NO:13 comprises a portion of the genomic sequence with a portion of the insert sequence, SEQ ID NO:47 comprises the coding sequence, SEQ ID NO:64 comprises the amino acid sequence.) This gene encodes a protein with homology to ATP-dependent RNA helicases, and can, in some embodiments, be used to alter the efficiency of pollen development.

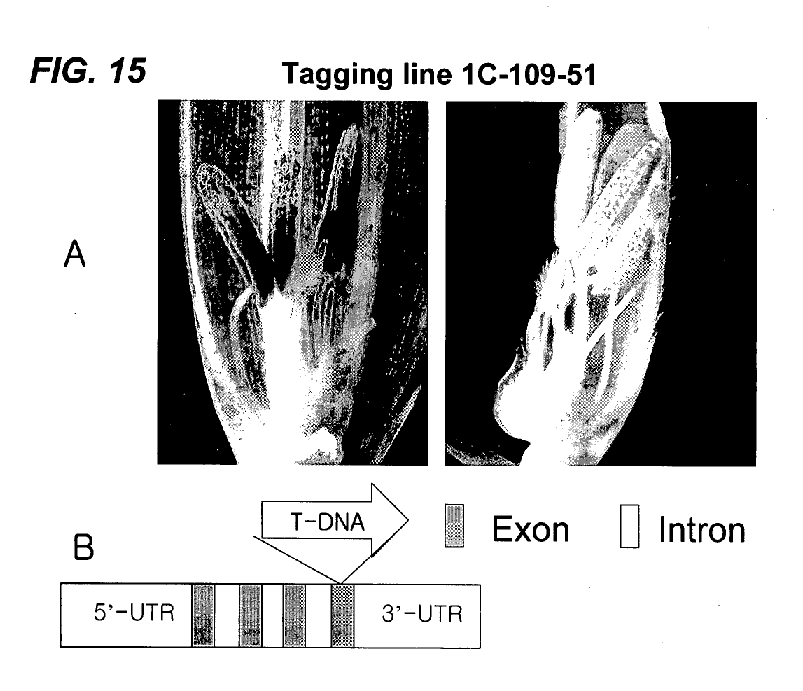

A diagram showing the insertion site of the T-DNA/GUS insert and an image showing the expression characteristics of the tagged gene are shown in FIG. 15. The gene is expressed in rice pollen. Members of the RNA-dependent helicase group of proteins are involved in aspects of the initiation of translation in eukaryotes. These enzymes have been found to unwind the double-stranded RNA structure that is present at the 5′ end of mRNA to allow for binding of the ribosomal subunits and subsequent translation of the mRNA. Other RNA-dependent helicases have been found to be involved in RNA metabolism, pre-mRNA splicing, ribosomal biogenesis, and transport between the cytoplasm and the nucleus.

RNA-dependent helicases are important for cellular developmental processes. The pollen-specific expression of this gene indicates that the RNA helicase may play an essential role in maturation and viability of pollen. Therefore, of possible agronomic utility is the pollen-specific knockout of this gene in the pollen grain, accomplished by linking the antisense construct of the gene to its pollen-specific promoter, then expressing it in a plant to create plants that cannot reproduce sexually. The pollen of such plants would be likely to be nonviable or have a reduced viability. As mentioned above, this may be useful when it is not desirable to spread transgenes to nearby crops or related weedy species.

Alternatively, it may be useful to alter expression of the gene in plants so that pollen-specific expression of the gene is increased as compared to wild-type plants. This may increase viability of the pollen grain, or even shorten the time required for pollen development. The pollen-specific regulatory region of the gene could be linked to other regulatory regions (such as hormone responsive promoters, or environmental stress-specific promoters) to further modify expression.

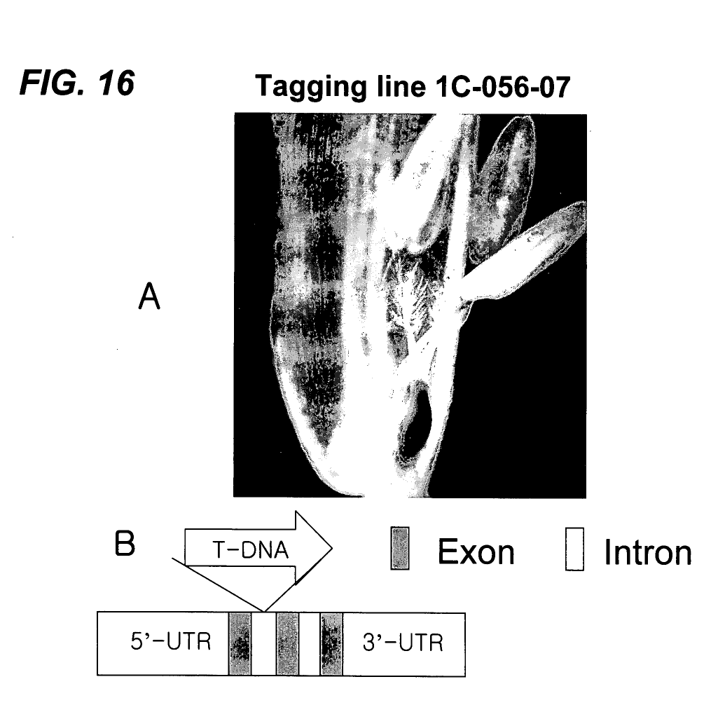

1C-056-07: (SEQ ID NO:31 comprises the genomic sequence, SEQ ID NO:14 comprises a portion of the genomic sequence with a portion of the insert sequence, SEQ ID NO:48 comprises the coding sequence, SEQ ID NO:65 comprises the amino acid sequence) This gene encodes a protein with homology to glucose-6-phosphate/phosphate transporters, involved in carbohydrate metabolism. The gene can, in some embodiments, be used to increase grain yields. A diagram showing the insertion site of the T-DNA/GUS insert and an image showing the expression characteristics of the tagged gene are shown in FIG. 16. The gene is expressed in leaf, filament, and ovary tissue.

Glucose-6-phosphate is an important player in carbohydrate metabolism. Certain plastids, such as amyloplasts and leucoplasts transport glucose-6-phosphate across the plastid double membrane system using a membrane-localized glucose-6-phosphate/phosphate transporter system. This inner membrane localized transporter protein allows movement of glucose-6-phosphate in one direction as Pi is transported in the opposite direction (see Dennis and Blakeley, p 632, in Biochemistry and Molecular Biology of Plants, 2000, supra). The transporter is especially important in developing seeds and starch-storing organs.

Genetic modification to increase the ovary-specific expression of this gene may result in increased transfer of glucose-6-phosphate to the rice grain. This may result in higher yields of grain or faster seed fill. It may be possible to alter expression of the gene so that ovary-specific expression of the gene is upregulated upon a specific environmental stress, such as a water deficit, or cold temperature. This may allow plants to turn on seed fill mechanisms in response to changing environmental conditions. For example, plants that are not cold tolerant may die upon colder weather at the beginning of the winter season. It may be possible to modify those plants so that upon the first onset of cold weather events, the plants can switch quickly from a vegetative growth stage to a seed-fill stage, translocating metabolites from the vegetative part of the plant to the seed by increasing expression of genes encoding glucose-6-phosphate translocators. This could speed the seed-fill time and perhaps increase the grain yield, especially in cold sensitive varieties.

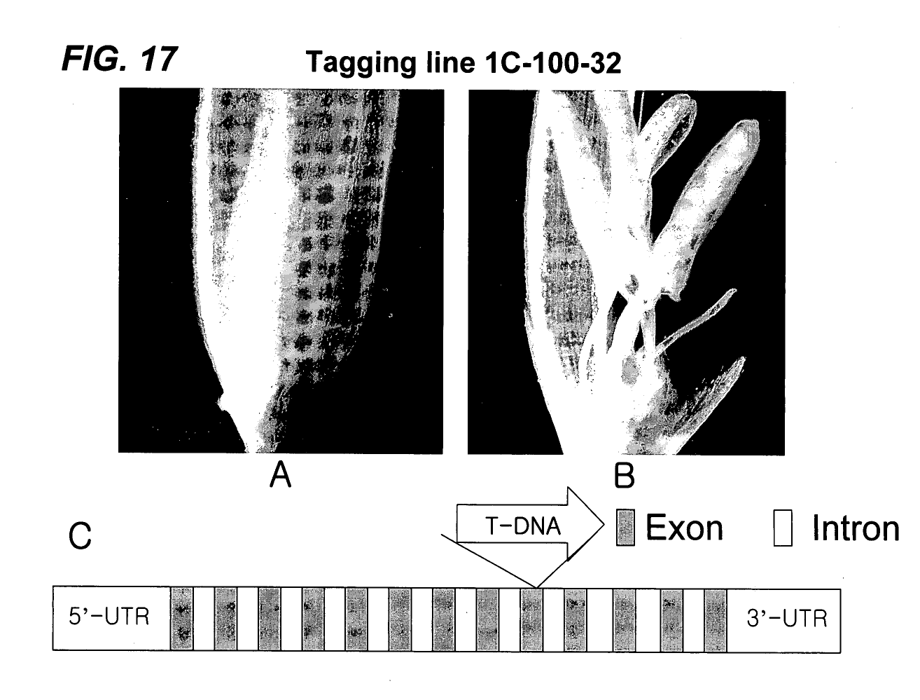

1c-100-32: (SEQ ID NO:32 comprises the genomic sequence, SEQ ID NO:15 comprises a portion of the genomic sequence with a portion of the insert sequence, SEQ ID NO:49 comprises the coding sequence, SEQ ID NO:66 comprises the amino acid sequence.) This gene is expressed preferentially in ovary tissue and pollen grains. The protein may function in aminophosphonate metabolism. A diagram showing the insertion site of the T-DNA/GUS insert and an image showing the expression characteristics of the tagged gene are shown in FIG. 17.

This gene encodes a protein with homology to RNA methyltransferases, and can, in some embodiments, be used to alter protein production leading to grain development. RNA methyltransferases transfer a methyl group to specific ribonucleic acids. The function of the methylation of RNA is not yet known, but it may affect rRNA stability or alter the protein translation process. Nop2p, a yeast nucleolar protein which acts as an RNA methyltransferase, has been found to function in rRNA processing and in the biogenesis of the 60S ribosomal subunit in addition to its RNA methyltransferase activity (Hong, et al., 2001, Nuc. Acids Res., 29:2927-2937), the disclosure of which is incorporated herein by reference in its entirety. Methylation of the RNA occurs at the 2′-O-hydroxyl position of the ribose sugars, and is part of the processing step of the 27S pre-rRNA to 5.8S and 25S rRNAs, which will then become a part of the 60S subunit. Temperature sensitive mutations in the yeast nop2 alleles were found to be defective in synthesis of the 25S rRNA and in the assembly of the 60S subunit (Hong et al., supra). Other RNA methyl transferases include the yeast Trm1p and the E. coli. Fmu. These two proteins methylate specific cytosines.

The E. coli FtsJ/RrmJ heat shock protein has been shown to be a 23S Ribosomal RNA methyltransferase. The protein acts on either pre-ribosomal ribonucleoprotein particles or in the 50S bacterial ribosomal subunit.

The RNA methyltransferase found in the present invention may also be involved in rRNA processing and in ribosomal assembly. If so, then it may be possible to genetically modify plants to increase ribosomal synthesis rates in the ovary tissue by increasing the expression of this gene coupled to its own promoter, or an ovary-specific promoter. Increasing ribosomal assembly may result in an increased rate of protein synthesis. One benefit of increasing the rate of protein synthesis in the ovary tissue of rice may be, for example, an increased grain size or yield, or increased level of proteins in the grain or its surrounding tissues.

Alternatively, it may be useful to inhibit protein synthesis in certain organs. For example, in some crop species where the crop value is obtained only from the vegetative tissues rather than the reproductive tissues, the development of the ovary and pollen could be reduced or stopped altogether by transforming the plant with an antisense construct of the RNA methyltransferase of the invention, coupled to its own ovary and pollen tissue-specific promoter. The plant would grow normally in a vegetative state, but would fail to form reproductive tissues. This may be especially useful to prevent “bolting” of certain plants such as spinach, lettuce, and herbs such as sage, basil, and thyme.

Methylation of rRNAs has been found to alter the susceptibility of ribosomes to antibiotics that target them (Cundiffe, 1990, in The Ribosome: structure, Function, and Evolution (Hill et al., eds; Am Soc. Microbiol; Washington, D.C.) 182, pp 479-490), the disclosure of which is incorporated herein by reference in its entirety. Therefore, plants with altered RNA methyltransferase expression may have altered resistance to antibiotics. This could be useful to prepare transformed plants that are more resistant to a given selectable marker, and thus can be selected more readily from the pool of potentially transformed plants.

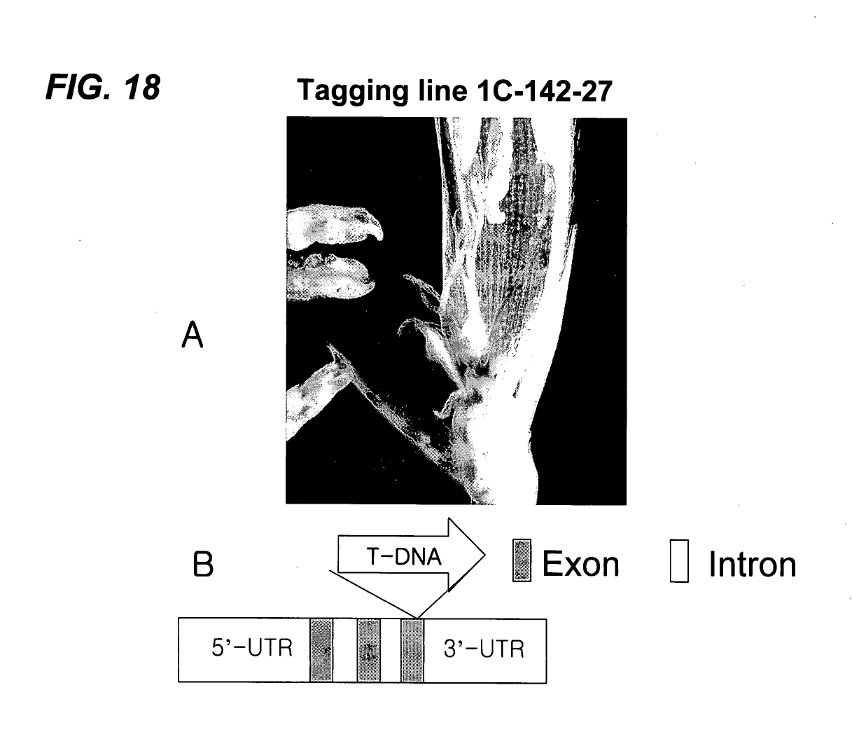

1c-142-27: (SEQ ID NO:33 comprises the genomic sequence, SEQ ID NO:16 comprises a portion of the genomic sequence with a portion of the insert sequence, SEQ ID NO:50 comprises the coding sequence, SEQ ID NO:67 comprises the amino acid sequence.) This gene encodes a protein with homology to actin depolymerizing factor 5, and can, in some embodiments, be used to alter grain size. The protein is thought to be essential for rapid F-actin turnover, stabilizing a pre-existing F-actin angular conformation. A diagram showing the insertion site of the T-DNA/GUS insert and an image showing the expression characteristics of the tagged gene are shown in FIG. 18. The gene is expressed in the pollen and ovary tissue of rice.

One of the main cytoskeleton components is the actin filament. Actin filaments are long units of polymerized actin monomers. The filaments are polar, having a slow-growing minus end and a fast growing plus end. The cell typically contains both free actin and polymerized actin filaments. The free actin units are either ADP or ATP bound. Once the free actin units are bound to ATP, they are able to polymerize to the plus end of actin filaments, with the concomitant hydrolysis of ATP to ADP.

Actin is often associated with various types of actin binding or actin cross-linking proteins. One type of protein associated with actin is the actin depolymerizing factor, (ADF), which is involved in dissassembling the actin filament. The ADF protein depolymerizes F-actin by inducing a large tilt in the angle of the actin subunits, severing the filaments and binding to the actin monomers (Galkin et al., 2001, Jour. Cell Biol., 153:75-86), the disclosure of which is incorporated herein by reference in its entirety.

In plants, the actin cytoskeleton is thought to play a key role in cell division, cell elongation, pollen tube germination, root hair growth, trichome growth, and in stomatal guard cell action. During pollen development in maize, the organization of the actin network changes as the developed pollen grain germinates and forms a pollen tube. The actin network forms a fibrillar network around the pollen aperture upon germination, and is present in the pollen tube to direct vesicle traffic to the tip of the pollen tube. Actin depolymerizing proteins are able to bind to filamentous actin (F-actin) or G-actin, to depolymerize the actin filaments so that they can be distributed where necessary. For example, the maize ZmADF3 redistributes to the growing tip of elongating root hairs (Jiang, et al., 1997, Plant Jour., 12:1035-1043), the disclosure of which is incorporated herein by reference in its entirety. ADF has also been found to associate with depolymerized actin in dormant pollen grains, presumably as a storage form of actin that is utilized upon germination (Smertenko, et al., 2001, Plant Jour., 25:203-212), the disclosure of which is incorporated herein by reference in its entirety.

In Arabidopsis, constitutive overexpression of an ADF-encoding gene resulted in reduced cell and organ growth and caused irregular cellular morphogenesis. In contrast, antisense expression of the gene resulted in increased cell expansion, increased organ growth, and delayed flowering (Dong et al., 2001, Plant Cell, 13:1333-1346), the disclosure of which is incorporated herein by reference in its entirety.

The genetic modification of ADF genes in plants could create plants with many types of useful morphological alterations, such as altered flowering, altered growth rates, altered germination, and altered organ growth. Since the downregulation of an ADF in maize resulted in increased organ growth (as noted above), it may be possible to increase rice grain size by downregulating the expression of the ADF gene during ovary development. This could be accomplished by linking an antisense construct of the gene to an ovary-specific promoter. This may result in an increase in the growth of the ovary. This may result in an increase in rice grain size or even in an increase in overall crop yield of rice grain.

Alternatively, pollen-specific alterations in expression of the ADF gene could alter pollen grain dormancy and pollen grain germination characteristics. Further, since the ADF protein has been implicated in cell expansion, it may be possible to alter growth of specific plant organs by up or downregulating the gene in specific organs.

1C-140-04: (SEQ ID NO:34 comprises the genomic sequence, SEQ ID NO:17 comprises a portion of the genomic sequence with a portion of the insert sequence, SEQ ID NO:51 comprises the coding sequence, SEQ ID NO:68 comprises the amino acid sequence.) This gene encodes a beta-glucosidase gene, and can, in some embodiments, be used to increase resistance to insect attack. Beta glucosidases are a group of glycoside hydrolases which are involved in a variety of cellular processes including defense responses, cell wall biology, and the activation of conjugated hormones.

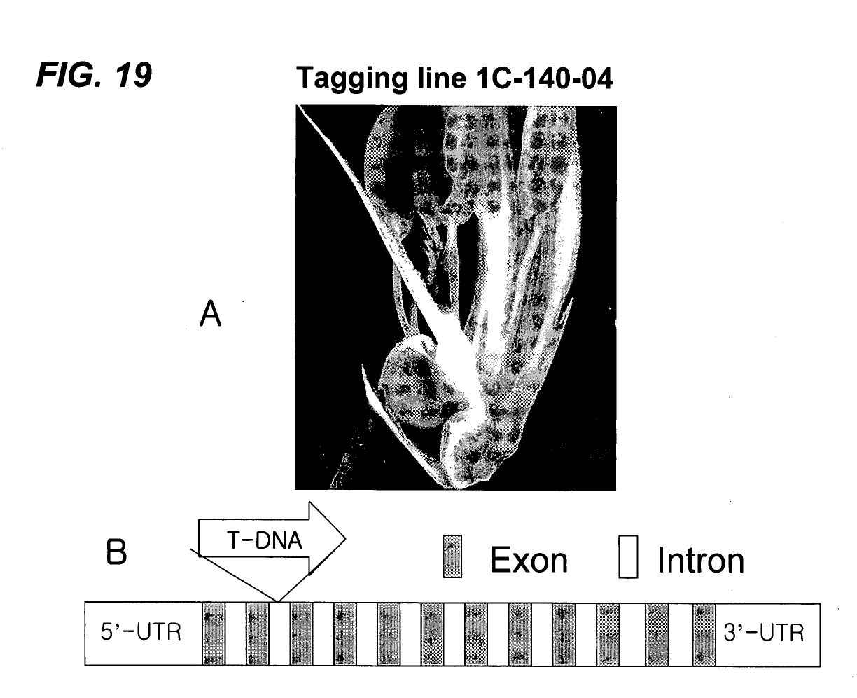

The gene is expressed in the ovary, stigma, and style, and additionally in the anther and lodicule. A diagram showing the insertion site of the T-DNA/GUS insert and an image showing the expression characteristics of the tagged gene are shown in FIG. 19.

In plants, one function of the beta-glucosidase enzyme is in the activation of storage forms of hormones or other signaling molecules. Plant hormones such as abscisic acid (ABA) and salicylic acid (SA) may be stored or transported throughout the plant as inactive conjugates, to be activated by enzymes such as glucosidases. Barley has been found to have extracellular beta-glucosidase activity that is able to hydrolyze the hormone ABA from its transport form as a glucose conjugate (Dietz, et al., 2000, Jour. Exp. Bot., 51:937-944), the disclosure of which is incorporated herein by reference in its entirety.

Beta-glucosidases accumulate in response to insect attack, and play a role in plant defense responses. Plants store various toxic chemicals for protection from insect or other predators. These chemicals are typically stored as conjugates in separate vesicles from the glucosidases. Upon pest damage leading to cell breakage, the stored conjugates can come into contact with the glucosidases, and the toxin is released to kill the predator. Example toxins include thiocyanates, nitrites, alkaloids, saponins, benzaldehydes, and cyanide. Accordingly, it may be useful to transform plants to upregulate the expression of this gene in rice to increase the insect resistance ability of rice. This expression could either be tissue-specific (such as in the reproductive organs, to protect developing rice grains) or wound-inducible expression, based on the chosen promoter.

The beta-glucosidase of the invention could be useful as an additive for food processing purposes. The gene could be overexpressed in a plant or plant tissue, harvested, isolated, and added to specific food manufacturing processes as a natural, plant-derived enzyme (rather than bacterial or fungal derived enzymes that may be in current use). Alternatively, the gene of the invention could be transformed to bacteria or yeast to be expressed and isolated from these cultures.

Beta-glucosidase is able to hydrolyze glucose-conjugated hormones to their free, active form. It may be possible to overexpress the beta-glucosidase gene of the invention in the same tissues by transforming plants with the gene of the invention coupled to its own promoter, with additional enhancer sequences upstream of the native promoter sequence. This may produce altered phenotypes such as reproductive tissue that is more sensitive to an ABA-conjugate signal arriving from the phloem. Ovary tissue that more readily responds to a drought-induced ABA signal in this manner may be able to switch to a seed fill/seed maturity phase faster than wild type plants. Further, expressing the gene linked to an ABA-inducible promoter may result in plants that are more ABA responsive throughout the plant.

Plant-associated fungi have also been found to have beta-glucosidase activity, presumably to attack the plant cell wall in order to obtain nutrients from the plant. With this in mind, it may be useful to produce rice plants with tissue-specific, cytoplasmic or cell wall-localized antisense expression of the beta-glucosidase gene in tissues that may be especially susceptible to fungal attack. The antisense gene may be able to interact with the fungal-derived sense transcript, inhibiting the production of the fungal glucosidase.

The above disclosure generally describes the present invention. A more complete understanding can be obtained by reference to the following specific examples which are provided herein for purposes of illustration only and are not intended to limit the scope of the invention.

### EXAMPLE 1

Construction of Vectors for Rice Insertional Mutagenesis

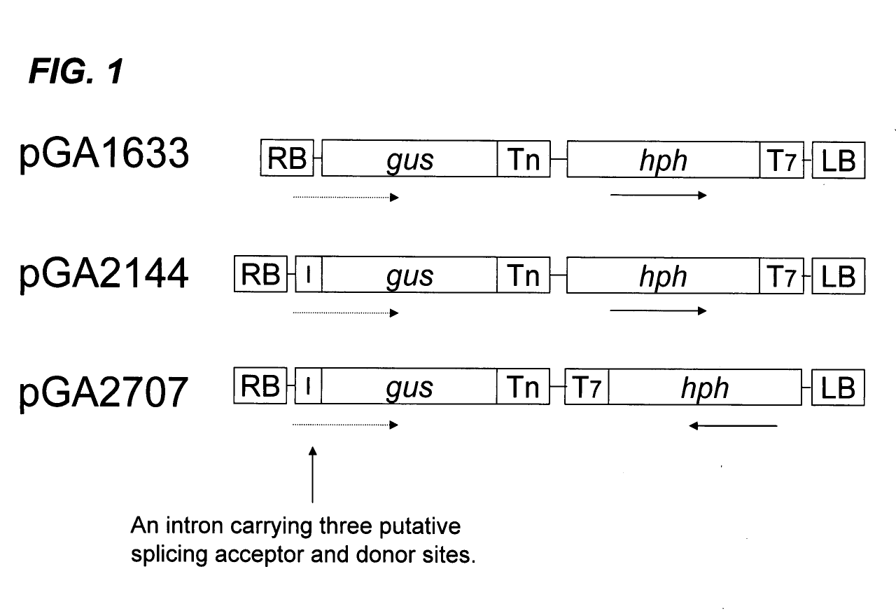

Three binary vectors, pGA1633, pGA2144, and pGA2707, were constructed for T-DNA insertional mutagenesis of rice (FIG. 1). The first plasmid, pGA1633, contains the promoterless GUS gene immediately next to the right border and the cauliflower mosaic virus (CaMV) 35S promoter-hygromycin phosphotransferase (hph) chimeric gene as a selectable marker. The pGA1633 vector was constructed by insertion of the GUS gene derived from pB101.1 into the BamHI site of pGA1605 (Lee et al., 1999, the disclosure of which is incorporated herein by reference in its entirety), which contains multi-cloning sites, BamHI, HindIII, XbaI, SacI, HpaI, Asp718 and ClaI, and 35S-hph. There is no translation initiation or stop codon between the right border of the T-DNA and BamHI site in pGA1633.

The second plasmid, pGA2144, was constructed to increase the gene trap efficiency. In this plasmid, an intron carrying three putative splicing donors and acceptors (the modified intron3 of OsTubA1, accession number AF182523) was inserted at the 5′ end of the GUS gene. Additionally, the selectable marker gene hph was modified by replacement of its operably linked CaMV 35S promoter with the strong promoter from the rice α-tubulin gene (OsTubA1), along with its first intron (as described above).

The OsTubA1 intron 3 was used as a template. The PCR primers used were 5′GGGTCGACGAGG-TACAAGGTACAAGGTACAGACTTGTATCCTT3′ (SEQ ID NO:70) and 5′-CGGGTACCACCTGCATATAACCTGCATATAACCTGCACATTA-GCAATAAA3′ (SEQ ID NO:71). The underlined sequences correspond to SalI and Asp718 sites. The primers were designed according to the splicing donor and acceptor sites of Sundaresan et al., 1995, Genes Dev. 9: 1797-1810, the disclosure of which is incorporated herein by reference in its entirety. The amplified fragment was digested with SalI and Asp718, and then cloned between XhoI and Asp718 in front of GUS in pGA1942, which contains multi-cloning sites (SacI, XhoI, Asp718 and ClaI) and 0.5 kb of OsTubA1 promoter-OsTubA1 intron 1-hph. The resulting plasmid was named pGA2020. Finally, the GA2144 plasmid was constructed from pGA2020 by replacing the 0.5 kb OsTubA1 promoter fragment with the 1.0 kb OsTubA1 promoter.

In the third plasmid, pGA2707, the hph gene and its promoter has been inserted in the reverse direction from the GUS gene. A modified OsTubA1 intron 2 was inserted in front of the GUS gene. Modification was achieved by PCR using the OsTubA1 gene as a template and primers carrying three putative splicing donor sequence or acceptor sequence. The PCR primers were 5′-GGATCCGAGGTACCAGGTACCAGGTG-AGTTCCATTCTTAC-3′ (SEQ ID NO:72) and 5′-CCCGGGACCTGCATA-TAACCTGCATATAACCTGTAAAGATTTAGCAC-3′ (SEQ ID NO:73). The underlined sequences correspond to BamHI and SmaI sites. The amplified fragment was cloned between BamHI and SmaI in front of the GUS gene. The resulting plasmid was named pGA2665. The terminator of the chimaeric hph gene was the OsTubA1 terminator. The terminator of OsTubA1 was amplified by PCR using the primers, 5′-GAAGATCTAGAGGAGTCGTCGTCGTCT-3′ (SEQ ID NO:74) and 5′-CCATCGATAGGCTAGTCATGGTGA-3′ (SEQ ID NO:75). The underlined sequences indicate BglII and ClaI site, respectively. The PCR product was cloned between ClaI and BglII of pGA2665, resulting in construction of the plasmid pGA2667. pGA2675 was made by killing the EcoRI site of pGA2667. The about 3 kb between SphI and BglII of pGA2675 was cloned between SphI and BglII of a binary vector, pGA2670 including multiple-cloning sites (BglII, EcoRI, XbaI, HindIII, ScaI, MluI, and XhoI). The resulting plasmid was named pGA2682. About 1 kb BamHI fragment carrying the hph gene cut out from pGA883 and cloned in the BglII site of the pGA2682. The resulting plasmid was named pGA2686. Finally, the pGA2707 plasmid was constructed from pGA2686 by the replacing the 5′ region (0.3 kb) of the hph gene by EcoRI digestion with the 2.6 kb fragment of pGA2144, which contains the 5′ region of the hph gene-OsTubA1 intron 1-OsTubA1 promoter. The T-DNA portion of the pGA2707 plasmid is shown in SEQ ID NO:69.

### EXAMPLE 2

Production of T-DNA-Tagged Transgenic Rice Plants

Rice transformation was performed by Agrobacterium-mediated co-cultivation methods as previously described (Jeon et al., 1999; Lee et al., 1999, the disclosures of which are incorporated herein by reference in their entireties). Scutellum-derived embryonic calli were co-cultivated with Agrobacterium tumefaciens LBA4404 carrying the binary tagging vector. Approximately 20-40% of the co-cultivated calli produced hygromycin-resistant cells. The frequency of plant regeneration from the calli ranged from 50-85%. Agrobacterium-mediated rice transformation procedures have been developed using the system based on the super-virulent strain and super-binary vectors carrying the virulence region of pTiBo542 (reviewed in Hiei et al., 1997, the disclosure of which is incorporated herein by reference in its entirety). The results showed that the transformation efficiency of this system was as high as the super-binary vector system, indicating that the Agrobacterium strain LBA4404 and a common binary vector can be used for efficient transformation of rice. With this system, 1590 transgenic plants transformed with pGA1633 and 20500 transgenic plants transformed with pGA2144 have been produced. These include the lines described in Table 2.

### EXAMPLE 3

Selection and Testing of Progeny for Hygromycin Resistance

The transgenic rice plants were selected for the presence of the selectable marker gene by regeneration on medium containing hygromycin B at a concentration of 40 mg per liter. The regenerated plants were grown in a greenhouse of typically 30° C. during the day and 20° C. at night. The light/dark cycle in the greenhouse was 14/10 h.

The progeny of the transformants were tested for hygromycin resistance using a higher concentration of hygromycin than the amount used for selection. Sterilized seeds were sown on a 70 mg per liter hygromycin B-containing MS medium and cultured under continuous illumination. Hygromycin resistance was scored 14 days after germination.

### EXAMPLE 4

Morphological Evaluation and Data Collection

Morphology assessments are made at several stages of plant development. T1 plants are observed at 4-5 weeks (vegetative stage), 6-7 weeks (flowering), and 8-9 weeks (fruiting). T2 pools of plants are observed weekly, with observations recorded after about week 4.

Observations are recorded using automated data collection means, e.g., a “Palm Pilot” which has a bar code scanner. Exemplary information for entry into a Palm Pilot includes plant flat (identified by a bar code and which contains 8 pools), pool information, date of planting for the flat; seed collection date, source and storage location of the seed (identified by plant ID/bar code) and when applicable, tissue collection date, type (either leaf or whole plant) and storage location.

Data synchronization may be accomplished by connecting a Palm Pilot to a computer using, e.g., the HotSync application on the Palm Pilot to download data into the computer. Photographs are taken using a digital camera (e.g., a, Kodak DC 260 or 265 digital camera) to document images of all plants according to their pool location within a designated flat at 4-5 weeks after germination and to download images into the computer database, as well as to capture images of plants with an mutant trait at any stage.

Bulk seed is collected from mature plants by rubbing mature siliques with fingers to release seed, using a sieve to remove chaff and pouring clean seed through a funnel into storage tubes to which are added desiccant, e.g., drierite chips.

In general, observations, measurements and the associated dates, tissue collections dates, seed collection dates, etc. are recorded and input into the database, such that individual plants may be identified and correlated with the various information that has been entered.

### EXAMPLE 5

Quantitation of Fertility of Primary Transformants

The seed fertility of the primary transgenic plants varied significantly, ranging from complete sterility to full fertility. Of the 22090 primary transgenic plants, 1338 lines (84%) of pGA1633 and 17020 lines (83%) of pGA2144 produced fertile seeds. Seventeen percent of the population was sterile, and 13% generated fewer than 10 seeds. About half of the population produced more than 100 seeds and 8% generated 50-100 seeds. The remaining plants generated 10-50 seeds. The pGA1633 lines were amplified, and the majority of the transgenic plants became fully fertile in the next generation. However, approximately one half of the transgenic plants, which showed partial sterility (fewer than 50 seeds) at the primary generation remained partially sterile, suggesting that the low fertility was due to genetic alteration by either T-DNA or other mutations. The pGA2144 lines are being amplified to produce enough seeds to be utilized for further studies.

### EXAMPLE 6

Morphology Screen and Propagation of Rice Plants with Mutant Traits

In an exemplary application of the method, T1 seeds are planted in flats, the flats put in cold storage for three or four days and are then placed in a greenhouse or growth room for germination and growth. The resulting T1 plants are observed at regular intervals, e.g., weekly, with observations made in notebooks or recorded using a Palm Pilot, and images recorded such that observations and/or measurements are recorded in a database. A percentage of the “interesting” T1 lines showing morphological mutant traits are selected based upon observations made of the T1 plants. In the case that an interesting T1 plant is sterile, tissue is collected for DNA extraction and gene isolation. Otherwise, T2 seed is produced from the interesting line. T2 seed collected from TI plants can be grown to produce T2 plants for observation, analysis and T3 seed production. T3 seed may then be used to produce T3 plants to confirm the mutant trait. DNA can then be extracted for use in gene isolation. It is also possible, after observing a mutant trait; to re-plant T2 seed from the collection for the production of T2 plants. The T2 plants can be used either as a source of tissue for DNA extraction and subsequent gene isolation or to make FI hybrid seed when crossed with wild type plants. Crosses are carried out by taking 4 or 5 flowers from each of the selected individual plants, using T2 pollen as the male parent and wild type flowers as the female parent. The resulting F1 seed from each cross is pooled, planted and may be subjected to selection. Segregation is recorded and phenotype observed. F1 hybrid seed can then be used to produce F2 seed from which segregating F2 populations can be grown segregation recorded and phenotype observed. These populations can also serve as a source of plant tissue for extraction of DNA and subsequent gene isolation activities.

### EXAMPLE 7

Screening of Transformed Rice Lines for Fungal, Bacterial, Viral and Insect Resistance

An exemplary screen for bacterial resistance is carried out by growing healthy plants from T2 seed and wild type untransformed control seed. Plants that have not been transformed can serve as susceptible control plants for the bacterial screen. The seedlings are grown to a given stage in development, whereupon one flat of wild type rice seedlings is sprayed with inoculum (positive control), and the other with Mock inoculum (negative control).

In general, bacterial inoculum are prepared from −80° C. stocks of bacterial isolates stored in 50% glycerol, using virulent and avirulent strains of the particular pathogen. Glycerol stocks are removed from the −80° C. freezer, streaked onto selective media plates with rifampicin (100 mg/L) using a sterile inoculation loop, then incubated for 3 days at 28° C. These starter cultures are used to inoculate larger liquid cultures for use in inoculating plants. The OD600 nm of 1 mL of each overnight culture is measured, with cultures that reach OD 0.5-0.8 units (mid-log phase actively growing culture) used for scale-up of inoculum. Once scaled-up, inocula are diluted as appropriate to obtain 108 bacterial colony forming units (cfu) per 1 ml.

Mock inoculations (negative controls) are carried out by drenching the plant leaf surface of each plant to be tested. Bacterial inoculations and incubation are carried out by drenching the plant leaf surface with a given inoculum diluted as set forth above.

In general, plants are scored for bacterial disease resistance at 24 hours post-inoculation, by evaluation of bacterial disease symptoms. There is a “phenotypic window” separating a resistance and a susceptible interaction. The goal of the resistance screen is to identify those individuals that display a resistance phenotype (relatively soon after infection) as opposed to a diseased (susceptible) phenotype which occurs later in the disease cycle. It will be understood that the ability to distinguish between these phenotypes is different for each pathogen/plant combination being tested.

Typically, the interaction between a plant pathogenic bacteria and the resistant plant occurs relatively quickly (16-28 hrs post-inoculation, “hpi”). This is why it is critical to evaluate the plant relatively soon after inoculation (24 hours). Leaves on the resistant plant display what is known as a hypersensitive response (“HR”). At 24 hpi a small lesion forms on the inoculated leaf surface formed by collapse of the cells immediately surrounding the bacterial entry site. The resistant (or incompatible) condition is maintained throughout the subsequent 7 day evaluation period. The HR is tightly limited to the necrotic lesion which completely dries out and has sharp border between the green healthy tissue and the necrotic lesion. There is no chlorosis beyond the margin of the necrotic lesion. The resistant (incompatible) and the susceptible (compatible) interaction phenotypes differ in two respects: (1) timing of appearance of symptoms and (2) the type of symptoms displayed. Typically, the resistant plants display a restricted necrosis (HR) surrounding the inoculation point at 24 hpi, while no symptoms are visible in the susceptible plants at this time. The compatible interaction (susceptible) phenotype begins to appear at around 72 hpi. It is characterized by water-soaked chlorotic margins surrounding a dry necrotic tissue. Over the course of the 7 day evaluation period, these lesions continue to enlarge at the chlorotic margins and become necrotic in the middle.

The transformed rice lines and wild type rice lines are observed in a growth room at 24 hours post-inoculation and plants visually identified that display a hypersensitive response, with the HR symptoms comparable to the symptoms displayed on the avirulent bacteria-inoculated wild type plants. Susceptible plants do not show any symptoms at this time. Observations are recorded using a Palm Pilot hand held scanner.

Resistant plants are flagged and putative resistant plants monitored during the course of the evaluation period to verify that the HR condition is maintained.

The observation steps are repeated at approximately 48 and 72 hrs post-inoculation, with observations performed in the growth room where the plants are being maintained. Flags are removed from flats if disease symptoms appear in a previously flagged T2 plant. The wild type plants that have been inoculated with a virulent pathogen (positive controls) are used as a visual reference standard for identifying disease symptoms.

At 72 hrs (3 days) post-inoculation, all flats are moved to a greenhouse to continue incubating the inoculated plants. T2 lines which were earlier identified as putative resistant lines are observed further and if the HR condition is maintained over the entire 7 day course of evaluation (i.e. the resistance phenotype (dry tightly limited necrotic lesions) is still displayed at 7 days post-inoculation), the T2 line is scored as resistant. Again observations are recorded using a Palm Pilot hand held scanner and the individuals from a T2 line scored as resistant photographed using a Kodak DC265 camera. In addition, tissue is harvested from putative disease resistant plants which are grown in the greenhouse under long day conditions to promote flowering of the plants with seed collected as further described above. Plants that pass this initial resistance test are re-screened using a disease resistance confirmatory test, are further analyzed by gene isolation and identification and are crossed to wild type plants for subsequent rescreen of F2 plants.

It will be appreciated that the details of a given bacterial screen may vary dependent upon the bacteria/plant combination being tested and this example serves as a general description of such a bacterial screen. Additional examples of such a bacterial screen are generally known in the art.

### EXAMPLE 8

Screening of Transformed Rice Lines for Environmental Stress Resistance

Rice lines of the invention may be analyzed for desirable characteristics using directed screens. In this example, directed screens are described that are performed in order to identify genes involved in resistance to stress.

A T2 screen for drought resistance is performed. Seeds of either the transformed rice lines and control plants are planted following any suitable method. Watering, and applications of fertilizer, etc. are carefully recorded and indicate where the treatment of one pot, line, or flat might differ from the rest. Temperature, light, and humidity are also recorded in a Palm Pilot. The plants are cared for as evenly as possible across flats and experiments. At a given time after germination, watering ceases (half of the wild type controls receive normal watering). Plants are evaluated for interesting morphologies at the time watering is stopped. After several days, or when the “no water” wild type plants are noticeably wilted, lines are evaluated for drought tolerance, and tolerant lines are marked. One leaf from each plant in marked lines is collected, and leaves from each line are pooled in 2 ml cryo-vials, which are labeled and placed in −80° C. freezer. Leaves from each plant in marked lines is then collected, and leaves from each line are pooled in 50 ml falcon tubes, which are barcode labeled. These pooled leaves (“samples”) are weighed on an analytical balance; for each line, the line ID and this “fresh weight” (FW) are recorded in the Palm Pilot. Samples are replaced in 50 ml tubes, 25 ml DI water is added to each tube, and the tubes are placed at 5° C. After 18-24 hours, tubes are removed from the cold. Each leaf is carefully removed from the water and gently blotted to dry its surface. Samples are weighed, and weights are recorded as “turgid weight” (TW). Samples are placed into aluminum weighing dishes and put into a 70-80° C. incubator. After 7 days, samples are re-weighed, and weights are recorded as “dry weight” (DW). The relative water content (RWC) is calculated using the formula: RWC=(FW−DW)/(TW−DW)×100.

Plants are recovered from drought conditions. After 3-5 days, recovery is evaluated. This is determined by presence of new growth, recovery of leaf color in older leaves, and may utilize RWC or other analyses. Lines showing no variation from wild type, in either general morphology or drought tolerance/recovery, will not be followed, and will be discarded after this analysis.

Following recovery, interesting lines are marked for seed collection and re-screening. Seeds from marked lines are collected either individually or as a T3 seed pool. In general, for lines showing interesting phenotypes, tissue is harvested and seed collected from individuals or pooled siblings in a line. Where T3 seed is not available, T2 seed is recovered. Seed from each line of interest is planted alongside wild type seed. The drought resistance screen is repeated as described above for re-screening.

### EXAMPLE 9

Germination Assay to Screen for Altered Levels of Salt Tolerance in Transformed Rice Lines

A salt tolerance screen is performed to identify and isolate gene(s) that confer salt (NaCl) tolerance. A primary screen is conducted with T1 plants, using a germination assay. T1 seed is planted in a suitable media supplemented with a suitable amount of NaCl. For negative and positive controls, wild type seed is planted either with or without, respectively, the supplemental NaCl. The seeds are allowed to germinate under typical rice growing conditions. It is expected that a range of phenotypes, of varying intensities, will be observed in the germination assay. Salt tolerant germination is classified in five stages: 1) imbibition, emergence of radicle; 2) expansion and greening of cotyledons; 3) elongation of the hypocotyl; 4) elongation of the root and formation of root hairs; 5) development of true leaves. A high stringency screen requires seedlings to progress through all five stages. In the event that such mutants are not observed, low stringency criteria are then used. For a low stringency screen, not all of the criteria will need to be met, and any putative positives (i.e., salt resistant plants) are examined in a secondary screen. Salt tolerance is scored, as is the segregation ratio of tolerance.

### EXAMPLE 10

DNA Gel-Blot Analysis

Genomic DNA was isolated from mature leaves at the heading stage as described previously (Dellaporta et al., 1983), the disclosure of which is incorporated herein by reference in its entirety. Genomic DNA (5 μg) was digested with EcoRI, separated on a 0.7% agarose gel, blotted onto a nylon membrane, and hybridized with a 32P-labeled probe. The GUS probe was prepared from the 1.8 kb BamHI-EcoRI fragment and the hph probe was from the 0.7 kb EcoRI fragment. All blot analysis procedures were carried out as described previously (Kang et al., 1998), the disclosure of which is incorporated herein by reference in its entirety.

### EXAMPLE 11

Molecular Characterization of T-DNA Integration Pattern in Transgenic Rice Plants

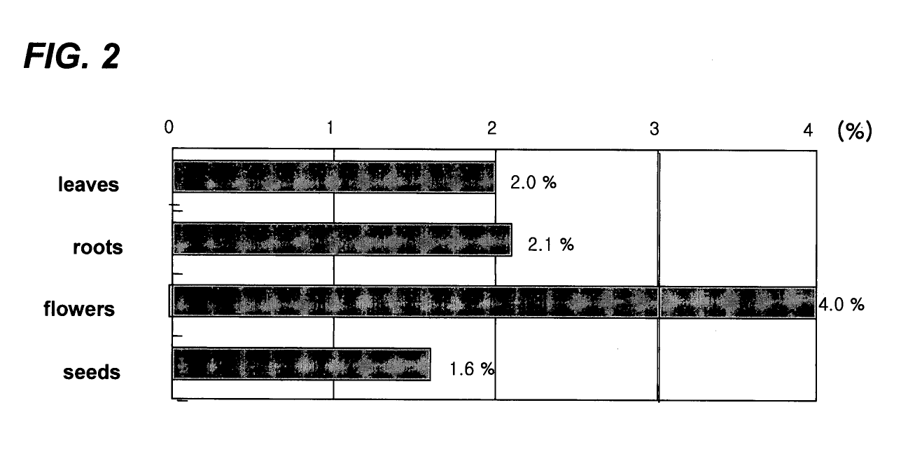

The number of integrated T-DNA in each plant was estimated from randomly selected primary transformants (FIG. 2). Table 2 is a summary of the genomic DNA gel-blot analysis using the GUS or hph coding region as a probe. Among the 34 transgenic lines examined, 11 lines carried a single copy of the GUS gene and 13 carried a single copy of the hph gene. The remaining lines carried two or more copies of GUS or hph. This result indicates that approximately 35% of the transgenic lines carry a single T-DNA insert. In several lines, the numbers of GUS and hph genes were different from each other, probably due to T-DNA re-arrangement during the transformation process (Ohba et al., 1995; see below), the disclosure of which is incorporated herein by reference in its entirety.

The number of T-DNA insertion loci was analyzed by scoring hygromycin-resistant progeny (T2) of the primary transgenic plants (T1). Twenty-four of 34 lines appeared to carry T-DNA at one locus, while the remaining 10 lines contained unlinked T-DNA insertion (Table 2). This indicates that transgenic plants contain an average of 1.4 loci of T-DNA inserts. These data are quite similar to the results observed in Arabidopsis indicating that T-DNA tagged plants contain an average of 1.4 inserts (Feldmann, 1991), the disclosure of which is incorporated herein by reference in its entirety. The number of insertion loci that was estimated by hygromycin resistance was smaller than the number of T-DNA copies evaluated by the DNA gel-blot analysis (Table 2). This result was probably due to tandem integration of two or more T-DNA copies into a single chromosome as observed previously in dicot plants (Krizkova and Hrouda, 1998), the disclosure of which is incorporated herein by reference in its entirety. A PCR approach was undertaken to investigate T-DNA arrangement of the lines that carry multiple T-DNAs at a single chromosome. The result showed that T-DNA copies were arranged in direct or inverted repeats. Sequence analysis of the regions between the T-DNA borders from six lines that carry multiple T-DNA copies at a single locus was carried out. The results revealed that two lines did not contain any DNA sequences between the T-DNAs. The remaining four lines carried 6-488 bp of filler DNA. Interestingly, the 488 bp of the longest filler DNA in the 81558 line was found be a portion of the GUS gene. A DNA gel-blot analysis confirmed that the B1558 line had one more copy of GUS than hph (Table 2). Such a partial T-DNA was previously reported from dicots, such as tobacco (Krizkova and Hrouda, 1998), the disclosure of which is incorporated herein by reference in its entirety. It may be explained by the suggestion that the formation of repeated T-DNA copies might result from co-integration of several inter-mediates into one target site.

It has previously been reported that a majority of the T-DNA insertions occur within the right border at a specific locus (reviewed in Tinland, 1996), the disclosure of which is incorporated herein by reference in its entirety. To examine whether the same was true for our tagging lines, the junction regions between rice genomic DNA and the T-DNA right border were sequenced (FIG. 1c). The sequencing results revealed that the boundaries in most of the rice lines did not correspond to the T-DNA nicking position found in Arabidopsis and tobacco transgenic plants. In dicot species, most T-DNAs were nicked after the first or second base of the right border. In our tagging lines, five were similar to those of Arabidopsis and tobacco. However, the most frequent junction point (11 out of 32 lines) was after the third base of the right border. In seven lines, the junction was at the boundary between T-DNA and the right border. The remaining nine lines showed deletion of 1-12 bases of T-DNA. It was previously reported that two of three right boundaries in transgenic rice plants and four of ten in transgenic maize plants carried three bases originated from the right border (Hiei et al., 1994; Ishida et al., 1996, the disclosures of which are incorporated herein by reference in their entireties).

### EXAMPLE 12

Histochemical GUS Staining Method and Microscopy

Histochemical GUS staining was performed according to Dai et at. (1996), the disclosure of which is incorporated herein by reference in its entirety, except for addition of 20% methanol to the staining solution. After staining, tissues were fixed in a solution containing 50% ethanol, 5% acetic acid and 3.7% formaldehyde, and embedded in a Paraplast (Sigma). The samples were sectioned to 10 μm thickness and observed under a microscope using dark-field illumination.

### EXAMPLE 13

Evaluation of Organ Preferential Gus Gene Expression in Transgenic Rice Plants

To evaluate the efficiency of the gene trap system, the GUS expression pattern was examined from various organs of primary transgenic plants transformed with pGA2144. GUS activities in the leaves and roots were analyzed in 5353 lines, mature flowers in 7026 lines, and developing seeds in 1948 lines. The results revealed that the efficiency of GUS staining was 2.0% ( 106/5353) for leaves, 2.1% ( 113/5353) for roots, 1.9% ( 133/7026) for flowers, and 1.6% ( 31/1948) for immature seeds (Table 2). Among the 106 GUS-positive lines in leaves, 15 (14.2%) were leaf-specific. Likewise, 25 (22.1%) lines were root-specific among the 113 GUS-positive lines in roots. Data was also obtained indicating that the efficiency of GUS expression in pGA1633 lines was 1.1% ( 8/750) for leaves and 0.9% ( 7/750) for roots. These values are lower than that of pGA2144, indicating that the modified OsTubA1 intron increased GUS tagging efficiency.

The staining patterns of the 106 lines that showed GUS activity in leaves were observed in detail (FIGS. 3-19). The vein-preferential GUS staining pattern was the most frequently observed (43.4%), and 14 (13.2%) lines were stained preferentially in mesophyll cells between veins. In most samples, GUS staining was observed strongly in boundary regions exposed by cutting. It is likely that a high concentration of cellulose, lignin, silica cells, and wax in rice leaves could have obstructed penetration of the GUS substrates. A majority of the lines showed GUS staining in the area of cell differentiation, and more than half of the lines exhibited GUS activity in the area of cell elongation or cell division. The GUS staining patterns in transgenic flowers was also characterized. Among the 133 lines that showed GUS activity in flowers, 50 (37.6%) displayed intense GUS staining primarily in the palea and lemma. One line exhibited GUS activity only in glumes, eight lines showed GUS activity only in lodicules, and four lines only in a carpel. Of the 11 lines exhibiting stamen-specific GUS activity, seven showed pollen-specific GUS staining. The developing seeds were also subjected to GUS staining 5-10 days after flowering. A large portion of these lines showed a tissue-preferential expression pattern. For example, line G930726 exhibited an aleurone layer-preferential GUS staining pattern, indicating that the trapped gene might be involved in formation of the aleurone layer or in a specific function in the tissue.

### EXAMPLE 14

Isolation of the Sequence Flanking T-DNA and the Junction Sequence Between Two Integrated T-DNAs

To identify the endogenous gene containing the T-DNA/GUS insertion, a PCR-based method was used. The sequence flanking T-DNA was isolated by thermal asymmetric interlaced PCR as previously described (Liu and Whittier, 1995, the disclosure of which is incorporated herein by reference in its entirety). The specific primer for the first cycle was 5′GCCGTAATGAGTGACCGCATCG3′ (Gus1) (SEQ ID NO:76); the second was 5′ATCTGCATCGGCGAACTGATCG3′ (Gus2) (SEQ ID NO:77); and the third was 5′CACGGGTTGGGGTTTCTACAGG3′ (Gus3) (SEQ ID NO:78). The junction between two integrated T-DNAs was amplified by PCR using primers Gus3 and 5′GCTTGGACTATAATACCTGAC3′ (T7) (SEQ ID NO:79). PCR products were sequenced using the BigDye Terminator Cycle Sequencing Ready Reaction Kit (PE Applied Biosystems, Foster City, Calif., USA).

### EXAMPLE 15

Inverse PCR to Determine the Upstream Regulatory Sequences of the Genes of the Invention

The genes of the invention are expressed in an organ-specific manner. Accordingly, the identification of the 5′ upstream regulatory sequences that control expression of these genes could be useful for genetically modifying plants to obtain altered phenotypes. For example, isolated 5′ regulatory regions could confer the same organ-specific expression to heterologous genes when operably linked to these heterologous genes and transformed into plants. To determine the 5′ regulatory sequence of the gene, the technique of inverse polymerase chain reaction, described briefly below, can be utilized.

The technique of inverse polymerase chain reaction can be used to extend the known nucleic acid sequence identified as described herein. The inverse PCR reaction is described generally by Ochman et al., in Ch. 10 of PCR Technology: Principles and Applications for DNA Amplification, (Henry A. Erlich, Ed.) W.H. Freeman and Co. (1992), the disclosure of which is incorporated herein by reference in its entirety. Traditional PCR requires two primers that are used to prime the synthesis of complementary strands of DNA. In inverse PCR, only a core sequence need be known.

To practice this technique, rice genome DNA is isolated, digested with PstI so as to create fragments of nucleic acid that contain T-DNA as well as unknown sequences that flank T-DNA. These fragments are then self-ligated to create a circularized molecule that becomes the template for the PCR reaction. A PCR primer corresponding to the gene of interest, directed towards the 5′ regulatory region, is designed based on the downstream known sequence. Another primer, upstream of the unknown sequence and typically based on a sequence present in the flanking plasmid DNA, is prepared. The primers direct nucleic acid synthesis away from the known sequence and toward the unknown sequence contained within the circularized template. After the PCR reaction is complete, the resulting PCR products can be sequenced so as to extend the sequence of the identified gene past the core sequence of the identified exogenous nucleic acid sequence identified. In this manner, the full sequence of each novel gene can be identified. Additionally, the sequences of adjacent coding and noncoding regions can be identified. Promoters can be identified using databases or promoter reporter vectors as described below.

5′ regulatory region primer:

| | | |
|---|---|---|
| 1^{st} | 5′-TTGGGGTTTCTACAGGACGTAAC-3′ | (SEQ ID NO: 80) |
| | (23mer) | |
| 2^{nd} | 5′-CAAGTTAGTCATGTAATTAGCCAC-3′ | (SEQ ID NO: 81) |
| | (24mer) | |

Another primer:

| | | |
|---|---|---|
| 1^{st} | 5′-CCATGTAGTGTATTGACCGATTC-3′ | (SEQ ID NO: 82) |
| | (23mer) | |
| 2^{nd} | 5′-TCGTCTGGCTAAGATCGGCCGCA-3′ | (SEQ ID NO: 83) |
| | (23mer) | |

Additionally, other PCR-based methods may be used to determine nucleic acid sequences flanking the genes of the invention. The following exemplary procedure describes a general method of determining upstream sequences from genomic DNA. Sequences derived from sequencing of the tagged genes of the invention may be used to isolate the promoters of the corresponding genes using chromosome walking techniques. In one chromosome walking technique, which utilizes the GenomeWalker™ kit available from Clontech, five complete genomic DNA samples are each digested with a different restriction enzyme which has a 6 base recognition site and leaves a blunt end. Following digestion, oligonucleotide adapters are ligated to each end of the resulting genomic DNA fragments.

For each of the five genomic DNA libraries, a first PCR reaction is performed according to the manufacturer's instructions (which are incorporated herein by reference) using an outer adaptor primer provided in the kit and an outer gene specific primer. The gene specific primer should be selected to be specific for gene of interest and should have a melting temperature, length, and location which is consistent with its use in PCR reactions. Each first PCR reaction contains 5 ng of genomic DNA, 5 μl of 10× Tth reaction buffer, 0.2 mM of each dNTP, 0.2 μM each of outer adaptor primer and outer gene specific primer, 1.1 mM of Mg(OAc)₂, and 1 μl of the Tth polymerase 50× mix in a total volume of 50 μl. The reaction cycle for the first PCR reaction is as follows: 1 min @ 94° C./2 sec @ 94° C., 3 min @ 72° C. (7 cycles)/2 sec @ 94° C., 3 min @ 67° C. (32 cycles)/5 min @ 67° C.

The product of the first PCR reaction is diluted and used as a template for a second PCR reaction according to the manufacturer's instructions using a pair of nested primers which are located internally on the amplicon resulting from the first PCR reaction. For example, 5 μl of the reaction product of the first PCR reaction mixture may be diluted 180 times. Reactions are made in a 50 μl volume having a composition identical to that of the first PCR reaction except the nested primers are used. The first nested primer is specific for the adaptor, and is provided with the GenomeWalker™ kit. The second nested primer is specific for the particular gene for which the promoter is to be cloned and should have a melting temperature, length, and location in the gene sequence which is consistent with its use in PCR reactions. The reaction parameters of the second PCR reaction are as follows: 1 min @ 94° C./2 sec @ 94° C., 3 min @ 72° C. (6 cycles)/2 sec @ 94° C., 3 min @ 67° C. (25 cycles)/5 min @ 67° C.

The product of the second PCR reaction is purified, cloned, and sequenced using standard techniques. Alternatively, two or more rice genomic DNA libraries can be constructed by using two or more restriction enzymes. The digested genomic DNA is cloned into vectors which can be converted into single stranded, circular, or linear DNA. A biotinylated oligonucleotide comprising at least 15 nucleotides from the gene sequence is hybridized to the single stranded DNA. Hybrids between the biotinylated oligonucleotide and the single stranded DNA containing gene sequence are isolated. Thereafter, the single stranded DNA containing the gene sequence is released from the beads and converted into double stranded DNA using a primer specific for the gene sequence or a primer corresponding to a sequence included in the cloning vector. The resulting double stranded DNA is transformed into bacteria. DNAs containing the gene sequence are identified by colony PCR or colony hybridization.

Once the upstream genomic sequences have been cloned and sequenced as described above, prospective promoters and transcription start sites within the upstream sequences may be identified by comparing the sequences upstream of the gene sequence with databases containing known transcription start sites, transcription factor binding sites, or promoter sequences. In addition, promoters in the upstream sequences may be identified using promoter reporter vectors as described in Example 18, below.

### EXAMPLE 16

Examination of Regulatory Regions in Cloned Upstream Sequences

Once the 5′ regulatory sequences of the genes of the invention are identified, they can be isolated and ligated to the 5′ region of GUS or other reporter genes to confirm the tissue-specific expression characteristics, dissect promoter regulatory regions, and determine the boundary of the regulatory region. The genomic sequences upstream of the genes of the invention are cloned into a suitable promoter reporter vector, such as the pSEAP-Basic, pSEAP-Enhancer, pβgal-Basic, pβgal-Enhancer, or pEGFP-1 Promoter Reporter vectors available from Clontech. Briefly, each of these promoter reporter vectors include multiple cloning sites positioned upstream of a reporter gene encoding a readily assayable protein such as secreted alkaline phosphatase, β galactosidase, or green fluorescent protein. The upstream sequence of the gene of the invention or fragments thereof is inserted into the cloning sites upstream of the reporter gene in both orientations and transformed to a plant cell. Whole plants are regenerated from the transformants so that organ-specific expression can be examined. The level of reporter protein is assayed and compared to the level obtained from a vector which lacks an insert in the cloning site. The presence of an elevated expression level in the vector containing the insert with respect to the control vector indicates the presence of a promoter in the insert. If necessary, the upstream sequences can be cloned into vectors which contain an enhancer for augmenting transcription levels from weak promoter sequences. Appropriate host cells for the promoter reporter vectors may be chosen based on the results of the above described determination of expression patterns of the gene of the invention. A significant level of expression above that observed with the vector lacking an insert indicates that a promoter sequence is present in the inserted upstream sequence.

Promoter sequences within the upstream genomic DNA may be further defined by constructing nested deletions in the upstream DNA using conventional techniques such as Exonuclease III digestion. The resulting deletion fragments can be inserted into the promoter reporter vector to determine whether the deletion has reduced or obliterated promoter activity. In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on the organ-preferential transcription levels may be determined by inserting the mutations into the cloning sites in the promoter reporter vectors.

Following the identification of promoter sequences using the procedures of Examples proteins which interact with the promoter may be identified as described in Example 17 below.

### EXAMPLE 17

Identification of Proteins which Interact with Promoter Sequences, Upstream Regulatory Sequences, or mRNA

Sequences within the promoter region which are likely to bind transcription factors may be identified by homology to known transcription factor binding sites or through conventional mutagenesis or deletion analyses of reporter plasmids containing the promoter sequence. For example, deletions may be made in a reporter plasmid containing the promoter sequence of interest operably linked to an assayable reporter gene. The reporter plasmids carrying various deletions within the promoter region are transfected into an appropriate host cell and the effects of the deletions on expression levels is assessed. Transcription factor binding sites within the regions in which deletions reduce expression levels may be further localized using site directed mutagenesis, linker scanning analysis, or other techniques familiar to those skilled in the art. Nucleic acids encoding proteins which interact with sequences in the promoter may be identified using one-hybrid systems such as those described in the manual accompanying the Matchmaker One-Hybrid System kit available from Clontech (Catalog No. K1603-1), the disclosure of which is incorporated herein by reference. Briefly, the Matchmaker One-hybrid system is used as follows. The target sequence for which it is desired to identify binding proteins is cloned upstream of a selectable reporter gene and integrated into the yeast genome. Preferably, multiple copies of the target sequences are inserted into the reporter plasmid in tandem.

A library comprised of fusions between cDNAs to be evaluated for the ability to bind to the promoter and the activation domain of a yeast transcription factor, such as GAL4, is transformed into the yeast strain containing the integrated reporter sequence. The yeast are plated on selective media to select cells expressing the selectable marker linked to the promoter sequence. The colonies which grow on the selective media contain genes encoding proteins which bind the target sequence. The inserts in the genes encoding the fusion proteins are further characterized by sequencing. In addition, the inserts may be inserted into expression vectors or in vitro transcription vectors. Binding of the polypeptides encoded by the inserts to the promoter DNA may be confirmed by techniques familiar to those skilled in the art, such as gel shift analysis or DNAse protection analysis.

### EXAMPLE 18

Plants Transformed with Chimeric Genes Having Organ-Preferential Promoters of the Invention Operably Linked to Heterologous Gene Coding Sequences

The promoters and other regulatory sequences located upstream of the genes of the invention may be used to design expression vectors capable of directing the expression of any inserted gene in a desired organ-preferential, temporal, developmental, or quantitative manner. A promoter capable of directing the desired organ-preferential, temporal, developmental, and quantitative patterns may be selected using the results of an expression analysis study. For example, if a promoter which confers a high level of expression in pollen is desired, the promoter sequence upstream of a gene of the invention that is expressed at high levels in pollen may be used in the expression vector.

Any gene of interest may be inserted downstream of the above-described organ-preferential promoter. Preferably, the desired promoter is placed near multiple restriction sites to facilitate the cloning of the desired insert downstream of the promoter, such that the promoter is able to drive expression of the inserted gene. The vectors may also include a polyA signal downstream of the multiple restriction sites for directing the polyadenylation of mRNA transcribed from the gene inserted into the expression vector. The vector is transformed to plant cells, and plants are regenerated. Organ-preferential expression of the gene of interest is determined, and altered phenotypes, such as increased or decreased organ size, altered viability, changes in disease resistance, and changes in stress responses can then be determined.

### EXAMPLE 19

Expression and Subsequent Purification of the Rice Proteins of the Invention

The following is provided as an exemplary method to express the rice proteins of the invention. The rice proteins of the invention can be produced by overexpression in rice or other plants. The transgenic plants having the gene of interest may be grown on a small scale (e.g., a laboratory or greenhouse), or on a larger scale, such as a large scale crop system. In this situation it may be helpful to target the protein expression to an easily isolatable tissue, such as, for example, the rice grain. The proteins can be expressed in other plant species, or in plant cell cultures. The protein may then be isolated and purified from the plant tissue.

Alternatively, the rice proteins may also be expressed in other organisms such as, for example, bacterial, yeast, insect, mammalian systems, or other systems known in the art. In some embodiments, the proteins encoded by the identified nucleotide sequences described above (including one of the polypeptides of SEQ ID NOS:52-68 encoded by one of the genes of SEQ ID NOS:18-34 or one of the genes of SEQ ID NOS:35-51) may be full length, or may be disrupted. The nucleic acids of SEQ ID NOS:35-51 encoded by the nucleic acids of SEQ ID NOS:18-34 are expressed using any suitable expression system. First, the initiation and termination codons for the gene are identified. If desired, methods for improving translation or expression of the protein are well known in the art. For example, if the nucleic acid encoding the polypeptide to be expressed lacks a methionine codon to serve as the initiation site, a strong Shine-Delgamo sequence, or a stop codon, these nucleotide sequences can be added. Similarly, if the identified nucleic acid lacks a transcription termination signal, this nucleotide sequence can be added to the construct by, for example, splicing out such a sequence from an appropriate donor sequence. In addition, the coding sequence may be operably linked to a strong constitutive promoter or an inducible promoter if desired. The identified nucleic acid or portion thereof encoding the polypeptide to be expressed is obtained by, for example, PCR from the bacterial expression vector or the rice genome using oligonucleotide primers complementary to the identified nucleic acid or portion thereof and containing restriction endonuclease sequences appropriate for inserting the coding sequences into the vector such that the coding sequences can be expressed from the vector's promoter. Alternatively, other conventional cloning techniques may be used to place the coding sequence under the control of the promoter. In some embodiments, a termination signal may be located downstream of the coding sequence such that transcription of the coding sequence ends at an appropriate position.

Several expression vector systems for protein expression in E. coli are well known and available to those knowledgeable in the art. The coding sequence may be inserted into any of these vectors and placed under the control of the promoter. The expression vector may then be transformed into DH5α or some other E. coli strain suitable for the over expression of proteins. The expressed protein can be modified to include a protein tag that allows for differential cellular targeting, such as to the periplasmic space of Gram negative or Gram positive expression hosts or to the exterior of the cell (i.e., into the culture medium). In some embodiments, the osmotic shock cell lysis method described in Chapter 16 of Current Protocols in Molecular Biology, Vol. 2, (Ausubel, et al., Eds.) John Wiley & Sons, Inc. (1997) may be used to liberate the polypeptide from the cell. In still another embodiment, such a protein tag could also facilitate purification of the protein from either fractionated cells or from the culture medium by affinity chromatography. Each of these procedures can be used to express a rice protein of the invention.

The expressed rice proteins are then purified using conventional techniques such as ammonium sulfate precipitation, standard chromatography, immunoprecipitation, immunochromatography, size exclusion chromatography, ion exchange chromatography, and HPLC. Alternatively, the polypeptide may be secreted from the host cell in a sufficiently enriched or pure state in the supernatant or growth media of the host cell to permit it to be used for its intended purpose without further enrichment. The purity of the protein product obtained can be assessed using techniques such as SDS PAGE, which is a protein resolving technique well known to those skilled in the art. Coomassie, silver staining or staining with an antibody are typical methods used to visualize the protein of interest.

The protein encoded by the identified nucleic acid of interest or portion thereof can be purified using standard immunochromatography techniques. In such procedures, a solution containing the secreted protein, such as the culture medium or a cell extract, is applied to a column having antibodies against the secreted protein attached to the chromatography matrix. The secreted protein is allowed to bind the immunochromatography column. Thereafter, the column is washed to remove non-specifically bound proteins. The specifically-bound secreted protein is then released from the column and recovered using standard techniques. These procedures are well known in the art.

In an alternative protein purification scheme, the identified nucleic acid of interest or portion thereof can be incorporated into expression vectors designed for use in purification schemes employing chimeric polypeptides. In such strategies the coding sequence of the identified nucleic acid of interest or portion thereof is inserted in-frame with the gene encoding the other half of the chimera. The other half of the chimera can be maltose binding protein (MBP) or a nickel binding polypeptide encoding sequence. A chromatography matrix having maltose or nickel attached thereto is then used to purify the chimeric protein. Protease cleavage sites can be engineered between the MBP gene or the nickel binding polypeptide and the identified expected gene of interest, or portion thereof. Thus, the two polypeptides of the chimera can be separated from one another by protease digestion. One useful expression vector for generating maltose binding protein fusion proteins is pMAL (New England Biolabs), which encodes the malE gene. In the pMal protein fusion system, the cloned gene is inserted into a pMal vector downstream from the malE gene. This results in the expression of an MBP-fusion protein. The fusion protein is purified by affinity chromatography. These techniques as described are well known to those skilled in the art of molecular biology.

### EXAMPLE 20

Production of an Antibody to an Isolated Protein

Antibodies capable of specifically recognizing the protein of interest can be generated using synthetic peptides using methods well known in the art. See, Antibodies: A Laboratory Manual, (Harlow and Lane, Eds.) Cold Spring Harbor Laboratory (1988). For example, 15-mer peptides having an amino acid sequence encoded by the appropriate identified gene sequence of interest or portion thereof can be chemically synthesized. The synthetic peptides are injected into mice to generate antibodies to the polypeptide encoded by the identified nucleic acid sequence of interest or portion thereof. Alternatively, samples of the protein expressed from the expression vectors discussed above can be purified and subjected to amino acid sequencing analysis to confirm the identity of the recombinantly expressed protein and subsequently used to raise antibodies. Substantially pure protein or polypeptide (including one of the polypeptides of SEQ ID NOS:52-68) is isolated from the transformed cells as described in Example 19. The concentration of protein in the final preparation is adjusted, for example, by concentration on a 10,000 molecular weight cut off AMICON filter device (Millipore, Bedford, Mass.), to the level of a few micrograms/ml. Monoclonal or polyclonal antibody to the protein can then be prepared following the methods described below.

Monoclonal antibody to epitopes of any of the polypeptides of the invention can be prepared from murine hybridomas according to the classical method of Kohler, G. and Milstein, C., Nature 256:495 (1975), the disclosure of which is hereby incorporated by reference in its entirety, or any of the well-known derivative methods thereof. Briefly, a mouse is repetitively inoculated with a few micrograms of the selected protein or peptides derived therefrom over a period of a few weeks. The mouse is then sacrificed, and the antibody-producing cells of the spleen isolated. The spleen cells are fused by means of polyethylene glycol with mouse myeloma cells, and the excess unfused cells are destroyed by growth of the system on selective medium comprising aminopterin (HAT medium). The successfully-fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as described by Engvall, E., “Enzyme immunoassay ELISA and EMIT,” Meth. Enzymol. 70:419 (1980), the disclosure of which is hereby incorporated by reference in its entirety, and derivative methods thereof. Selected positive clones can be expanded and their monoclonal antibody product harvested for use. Detailed procedures for monoclonal antibody production are described in Davis, L. et al. Basic Methods in Molecular Biology Elsevier, New York. Section 21-2; the disclosure of which is hereby incorporated by reference in its entirety.

Polyclonal antiserum containing antibodies to heterogeneous epitopes of a single protein or a peptide can be prepared by immunizing suitable animals with the expressed protein or peptides derived therefrom described above, which can be unmodified or modified to enhance immunogenicity. Effective polyclonal antibody production is affected by many factors related both to the antigen and the host species. For example, small molecules tend to be less immunogenic than larger molecules and can require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J. Clin. Endocrinol. Metab. 33:988-991 (1971), the disclosure of which is hereby incorporated by reference in its entirety.

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973), the disclosure of which is hereby incorporated by reference in its entirety. Plateau concentration of antibody is usually in the range of 0.1 to 0.2 mg/ml of serum (about 12 μM). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980), the disclosure of which is hereby incorporated by reference in its entirety. Antibody preparations prepared according to either protocol are useful in quantitative immunoassays which determine concentrations of antigen-bearing substances in biological samples; they are also used semi-quantitatively or qualitatively to identify the presence of antigen in a biological sample.

### EXAMPLE 21

Creation of Modified Plants Using cDNA

A cDNA clone containing a section of the coding region is subcloned into a plant transformation vector. The plant transformation vector contains a cauliflower mosaic virus 35S promoter and a polyadenylation site to allow for expression of the gene in plants (Schardl, C. L. et al., 1987, “Design and construction of a versatile system for the expression of foreign genes in plants,” Gene, 61: 1-11, the disclosure of which is incorporated herein by reference in its entirety). The plasmid is then introduced into Agrobacterium tumefaciens cells by electroporation, and the bacterial transformants are selected using a kanamycin selection marker. Agrobacterium cells carrying the complementary DNA are then used to infect plants by the leaf disk transformation method of Horsch et al. (Science, 1985, 227:1229, the disclosure of which is hereby incorporated by reference in its entirety). The same plasmid, lacking the DNA, is used as a control. Disks are cultured on media containing kanamycin, followed by shoot formation. The shoots are then transplanted to root-inducing selective medium. Rooted plantlets are transplanted to soil. In order to study the effect of the complementary DNA clone on the phenotype of the transformed plants, transformed plantlets are examined for the presence of possible modified phenotypes. Plants of interest are further selected and grown to maturity. Plants having an altered phenotype are thus obtained.

### EXAMPLE 22

Overexpression of a Gene of Interest in Plants

A plasmid is constructed to place the gene coding sequences downstream of the CaMV 35S promoter, to result in high level expression of the inserted gene when transformed into rice plants. The resulting plasmid is transformed into Agrobacterium tumefaciens as described in Example 21. Agrobacterium cells carrying the coding sequence of the gene of interest are used to transform rice plants by the leaf disk method as described above. The same plasmid, lacking the coding sequence of the gene of interest, is used as a control.

The transformed rice plantlets are tested for a altered phenotypes, and plants with the desired phenotypes are selected for further study. Rice plant lines are obtained that display an altered phenotype as compared to control rice plants. Alternatively, any plant may be transformed with the rice genes of the invention to produce increased levels of the encoded protein and to create altered phenotypes.

### EXAMPLE 23

Creation of a Plant Lacking Expression of a Gene of Interest Using Cosuppression

A truncated DNA fragment corresponding to a section of the coding region of a gene of interest is subcloned into a plant transformation vector. The plant transformation vector contains the cauliflower mosaic virus 35S promoter and a polyadenylation site to allow for expression of the gene in plants as described in Example 8. The plasmid is introduced into Agrobacterium tumefaciens cells by electroporation as described above, and the bacterial transformants are selected using a selection marker such as kanamycin. Agrobacterium cells carrying the desired fragment are used to infect rice plants using the leaf disk method as described above. The same plasmid, lacking the desired DNA, is used as a control. The transformed plantlets are examined for the presence of the desired phenotype.

### EXAMPLE 24

Creation of a Rice Plant Lacking Expression of a Gene of Interest Using Antisense Technology

The cDNA or a portion thereof of the gene of interest is subcloned into a plant transformation vector, oriented in the reverse orientation. The plant transformation vector contains the cauliflower mosaic virus 35S promoter and a polyadenylation site. The vector additionally contains a selectable marker gene such as NPTII, which confers resistance to kanamycin. The prepared plant transformation vector is introduced into Agrobacterium tumefaciens. Scutellum-derived rice embryonic calli are co-cultivated with Agrobacterium tumefaciens harboring the plant transformation vector. Plantlets are regenerated from the embryonic calli, and positive transformants are selected using kanamycin treatment. The kanamycin-resistant plants are examined for the presence of the desired phenotype.

### EXAMPLE 25

Screening Transformed Plants for the Presence of an Altered Phenotype

Plants transformed with the genes of the invention are grown to maturity in a greenhouse environment. Nontransformed plants, as well as plants transformed with the plant transformation vector without the gene of interest, are used as controls. Specific phenotyes relating to plant size can be visually observed and measurements can be taken using a ruler. Alternatively, whole plants or plant organs can be harvested, weighed to determine the fresh weight, then dried and weighed again to determine the dry weight. Further, protein analysis to determine changes in protein quality or protein levels can be performed, as well as analytical measurements to determine any differences in the accumulation of secondary products in the transformed plants. To test for responses to certain stresses, the plants are treated with the specific stress for a given time, then morphological characterstics, plant size, fresh weight, etc. are measured as described above.

What is claimed is:

1. An isolated or purified nucleic acid comprising a nucleotide sequence having at least 70% homology as measured by BLASTN version 2.0 set at the default parameters with a nucleotide sequence selected from the group consisting of SEQ ID NOS:18-34 and the nucleotide sequences complementary to SEQ ID NOS:18-34 or fragments comprising at least 10, 15, 20, 25, 30, 35, 40, 50, 75, 100, 150, 200, 300, 400, 500, 750, 1000, 1250, or 1500 consecutive nucleotides thereof.

2. The nucleic acid according to claim 1, wherein the nucleic acid has at least 80% homology.

3. The nucleic acid according to claim 1, wherein the nucleic acid has at least 85% homology.

4. The nucleic acid according to claim 1, wherein the nucleic acid has at least 90% homology.

5. The nucleic acid according to claim 1, wherein the nucleic acid has at least 95% homology.

6. The nucleic acid according to claim 1, wherein the nucleic acid has at least 97% homology.

7. The nucleic acid according to claim 1, wherein the nucleic acid has 100% homology.

8. An isolated or purified nucleic acid comprising a nucleotide sequence having at least 70% homology as measured by BLASTN version 2.0 set at the default parameters with a nucleotide sequence selected from the group consisting of SEQ ID NOS:35-51 and the nucleotide sequences complementary to SEQ ID NOS:35-51, or fragments comprising at least 10, 15, 20, 25, 30, 35, 40, 50, 75, 100, 150, 200, 300, 400, 500, 750, 1000, 1250, or 1500 consecutive nucleotides thereof.

9. The nucleic acid according to claim 8, wherein the nucleic acid has at least 80% homology.

10. The nucleic acid according to claim 8, wherein the nucleic acid has at least 85% homology.

11. The nucleic acid according to claim 8, wherein the nucleic acid has at least 90% homology.

12. The nucleic acid according to claim 8, wherein the nucleic acid has at least 95% homology.

13. The nucleic acid according to claim 8, wherein the nucleic acid has at least 97% homology.

14. The nucleic acid according to claim 8, wherein the nucleic acid has 100% homology.

15. An isolated or purified nucleic acid encoding a polypeptide having at least 25% amino acid identity as measured by BLASTP, BLASTX, or TBLASTN with default parameters with an amino acid sequence selected from the group consisting of SEQ ID NOS:52-68, or fragments comprising at least 5, 10, 15, 20, 25, 30, 35, 40, 50, 75, 100, 200, 400, 600, 800, or 1000 consecutive amino acids thereof.

16. The nucleic acid according to claim 15, wherein the polypeptide has at least 40% homology.

17. The nucleic acid according to claim 15, wherein the polypeptide has at least 50% homology.

18. The nucleic acid according to claim 15, wherein the polypeptide has at least 60% homology.

19. The nucleic acid according to claim 15, wherein the polypeptide has at least 70% homology.

20. The nucleic acid according to claim 15, wherein the polypeptide has at least 80% homology.

21. The nucleic acid according to claim 15, wherein the polypeptide has at least 85% homology.

22. The nucleic acid according to claim 15, wherein the polypeptide has at least 90% homology.

23. The nucleic acid according to claim 15, wherein the polypeptide has at least 95% homology.

24. The nucleic acid according to claim 15, wherein the polypeptide has at least 99% homology.

25. The nucleic acid according to claim 15, wherein the polypeptide has 100% homology.

26. An isolated or purified polypeptide having at least 25% amino acid identity as measured by BLASTP, BLASTX, or TBLASTN set at default parameters with an amino acid sequence selected from the group consisting of SEQ ID NOS:52-68, or fragments comprising at least 5, 10, 15, 20, 25, 30, 35, 40, 50, 75, 100, 200, 400, 600, 800, or 1000 consecutive amino acids thereof.

27. The polypeptide according to claim 26, wherein the polypeptide has at least 40% homology.

28. The polypeptide according to claim 26, wherein the polypeptide has at least 50% homology.

29. The polypeptide according to claim 26, wherein the polypeptide has at least 60% homology.

30. The polypeptide according to claim 26, wherein the polypeptide has at least 70% homology.

31. The polypeptide according to claim 26, wherein the polypeptide has at least 80% homology.

32. The polypeptide according to claim 26, wherein the polypeptide has at least 90% homology.

33. The polypeptide according to claim 26, wherein the polypeptide has at least 95% homology.

34. The polypeptide according to claim 26, wherein the polypeptide has at least 99% homology.

35. The polypeptide according to claim 26, wherein the polypeptide has 100% homology.

36. A recombinant nucleic acid comprising a nucleotide sequence encoding a polypeptide comprising an amino acid sequence selected from the group consisting of SEQ ID NOS:52-68 operably linked to a promoter.

37. A genetically modified rice plant wherein a gene selected from the group consisting of germin-like protein, alternative oxidase (AOX1a) protein, XA21-like protein kinase gene, receptor-like protein kinase, methylmalonate semi-aldehyde dehydrogenase (MMSDH1), homolog of the RNA-binding protein LAH1, vacuolar ATP synthase subunit C, cinnamic acid 4-hydroxylase, H-protein promoter binding factor-2a, flap endonuclease (FEN-1), heat shock protein Hsp70, ammonium transporter, ATP-dependent RNA helicase, glucose-6-phosphate/phosphate transporter, RNA methyltransferase, actin depolymerizing factor 5, and beta-glucosidase has been disrupted.

38. A genetically modified rice plant wherein a gene comprising a nucleotide sequence selected from the group consisting of SEQ ID NOS:18-34 has been disrupted.

39. A genetically modified rice plant wherein a gene encoding a polypeptide comprising an amino acid sequence selected from the group consisting of SEQ ID NOS:52-68: has been disrupted.

40. A genetically modified rice plant selected from the group consisting of 1b-115-22, 1b-164-43, 1b-192-40, 1b-207-27, 1b-138-07, 1d-059-12, 1c-087-40, 1c-017-14, 1c-038-56, 1c-041-47, 1c-064-20, 1c-109-35, 1c-109-51, 1c-056-07, 1c-100-32, 1c-142-27, and 1c-140-04.

41. A genetically modified rice plant which overexpresses or underexpresses a polypeptide comprising an amino acid sequence selected from the group consisting of SEQ ID NOS:52-68.

42. A method of screening a rice plant for a desirable characteristic comprising:
a) obtaining a rice plant wherein a gene selected from the group consisting of SEQ ID NOS:18-34 has been disrupted; and
b) exposing said rice plant to conditions which permit plants possessing said desirable characteristic to be identified.

43. The method of claim 42, wherein said desirable characteristic to be identified is selected from the group consisting of: altered photosynthetic capacity, altered response to biotic stress, allelopathy, altered response to abiotic stress, altered morphology, altered grain yield, altered nutritional content of grain, altered growth rates, altered secondary product pathways, altered pesticide resistance, altered grain characteristics such as grain shape or taste, cooking quality, altered harvesting qualities, altered optimal growth temperatures, altered resistance to herbicides, altered flowering time, altered seed fill characteristics, altered hormone biosynthetic/degradation pathways, or altered responses to hormones.

44. A method of producing a genetically modified plant having an altered phenotype as compared to a wild-type plant, comprising:
a) contacting a plant cell with a nucleic acid sequence which increases or decreases the expression or activity of a protein comprising an amino acid sequence selected from the group consisting of SEQ ID NOS:52-68 relative to a wild type plant to obtain a transformed plant cell;
b) producing a plant from said transformed plant cell; and
c) selecting a plant expressing said protein.

45. The method of claim 44, wherein the contacting is by physical means.

46. The method of claim 44, wherein the contacting is by chemical means.

47. The method of claim 44, wherein the plant cell is selected from the group consisting of protoplasts, gamete producing cells, and cells which regenerate into whole plants.

48. The method of claim 44, wherein the nucleic acid sequence is operably linked to a promoter selected from the group consisting of a constitutive promoter, a tissue specific promoter, an organ specific promoter, a developmentally specific promoter, and an inducible promoter.

49. The method of claim 44, wherein the promoter is selected from the group consisting of an endogenous promoter and a heterologous promoter.

50. The genetically modified plant of claim 44, wherein the amino acid comprises at least 90% amino acid identity as measured by BLASTP, BLASTX, or TBLASTN with default parameters to a polypeptide comprising an amino acid sequence selected from the group consisting of SEQ ID NOS:52-68.

51. The genetically modified plant of claim 44, wherein the amino acid comprises at least 95% amino acid identity as measured by BLASTP, BLASTX, or TBLASTN with default parameters to a polypeptide comprising an amino acid sequence selected from the group consisting of SEQ ID NOS:52-68.

52. The genetically modified plant of claim 44, wherein said nucleic acid sequence encoding the protein comprises a nucleotide sequence selected from the group consisting of SEQ ID NOS:18-34 and SEQ ID NOS:35-51.

53. The genetically modified plant of claim 44, wherein the plant is a dicotyledonous plant.

54. The genetically modified plant of claim 44, wherein the plant is a monocotyledonous plant.

55. A genetically modified seed, into which a nucleic acid sequence encoding a polypeptide having at least 80% amino acid identity as measured by BLASTP, BLASTX, or TBLASTN set at default parameters to an amino acid sequence selected from the group consisting of SEQ ID NOS:52-68 has been introduced.

56. The genetically modified seed of claim 55, wherein the nucleic acid encodes a polypeptide having at least 85% amino acid identity as measured by BLASTP, BLASTX, or TBLASTN set at default parameters to an amino acid amino acid sequence selected from the group consisting of SEQ ID NOS:52-68.

57. The genetically modified seed of claim 55, wherein the nucleic acid encodes a polypeptide having at least 90% amino acid identity as measured by BLASTP, BLASTX, or TBLASTN set at default parameters to an amino acid amino acid sequence selected from the group consisting of SEQ ID NOS:52-68.

58. The genetically modified seed of claim 55, wherein the nucleic acid encodes a polypeptide having at least 95% amino acid identity as measured by BLASTP, BLASTX, or TBLASTN set at default parameters to an amino acid amino acid sequence selected from the group consisting of SEQ ID NOS:52-68.

59. An antibody which binds to an isolated polypeptide comprising an amino acid sequence selected from the group consisting of of SEQ ID NOS:52-68 or fragments thereof.

60. A method of expressing a gene in a desired tissue or organ of a rice plant comprising:
a) obtaining the promoter which directs the transcription of a sequence selected from the group consisting of SEQ ID NOS:18-34;
b) operably linking said promoter to said gene; and
c) introducing said promoter operably linked to said gene into a rice plant.

61. A computer readable medium comprising a nucleotide sequence comprising a nucleotide sequence selected from the group consisting of SEQ ID NOS:18-34, the nucleotide sequences complementary to SEQ ID NOS:18-34, or fragments comprising at least 10, 15, 20, 25, 30, 35, 40, 50, 75, 100, 150, 200, 300, 400, 500, 750, 1000, 1250, or 1500 consecutive nucleotides thereof stored thereon.

62. A computer readable medium of claim 61, wherein the computer readable medium further comprises data indicating the tissue or organ in which the nucleic acid sequences are transcribed.

63. A computer readable medium comprising a nucleotide sequence comprising a nucleotide sequence selected from the group consisting of SEQ ID NOS:35-51, the nucleotide sequences complementary to SEQ ID NOS:35-51, or fragments comprising at least 10, 15, 20, 25, 30, 35, 40, 50, 75, 100, 150, 200, 300, 400, 500, 750, 1000, 1250, or 1500 consecutive nucleotides thereof stored thereon.

64. A computer readable medium of claim 63, wherein the computer readable medium further comprises data indicating the tissue or organ in which mRNA having the coding sequence is expressed.

65. A computer readable medium comprising an amino acid sequence comprising an amino acid sequence selected from the group consisting of SEQ ID NOS:52-68, the nucleotide sequences complementary to SEQ ID NOS:52-68, or fragments comprising at least 5, 10, 15, 20, 25, 30, 35, 40, 50, 75, 100, 200, 400, 600, 800, or 1000 consecutive amino acids thereof stored thereon.

66. A computer readable medium of claim 65, wherein the computer readable medium further comprises data indicating the tissue or organ in which the amino acid sequence is present.
